SEQUENCE LISTING (33 sequences)

SEQ ID NO: 1 - DNA, 23 bp, Artificial
  Oligonucleotide primer
tctccacagcttcctaaccaaga

SEQ ID NO: 2 - DNA, 20 bp, Artificial
  Oligonucleotide primer
ctggacccactcctcactgg

SEQ ID NO: 3 - DNA, 24 bp, Artificial
  Oligonucleotide primer
gatgctattcttggatatcctgag

SEQ ID NO: 4 - DNA, 20 bp, Artificial
  Oligonucleotide primer
gtgcagagaggacctggttg

SEQ ID NO: 5 - DNA, 23 bp, Artificial
  Oligonucleotide primer
cctagattctcatacctggagac

SEQ ID NO: 6 - DNA, 20 bp, Artificial
  Oligonucleotide primer
aatcatgcaggtctccactg

SEQ ID NO: 7 - DNA, 20 bp, Artificial
  Oligonucleotide primer
atgcaggtctccactgctgc

SEQ ID NO: 8 - DNA, 21 bp, Artificial
  Oligonucleotide primer
tcaggcactcygctcyaggtc

SEQ ID NO: 9 - DNA, 22 bp, Artificial
  Oligonucleotide primer
ctgcccttgcygtcctcctctg

SEQ ID NO: 10 - DNA, 20 bp, Artificial
  Oligonucleotide primer
aggtcrctgacrtatttctg

SEQ ID NO: 11 - DNA, 21 bp, Artificial
  Nucleic acid probe sequence
aggccggcaggtctgtgctga

SEQ ID NO: 12 - DNA, 21 bp, Artificial
  Oligonucleotide probe
gagccaaggtcacggaagccc

SEQ ID NO: 13 - DNA, 18 bp, Artificial
  Oligonucleotide probe
cctgggtcctagaatcac

SEQ ID NO: 14 - DNA, 58 bp, Artificial
  Oligonucleotide probe
gtgggatgagcagagaacaaaaacaaaataatccagtgagaaaagcccgtaaataaag

SEQ ID NO: 15 - DNA, 23 bp, Artificial
  Oligonucleotide probe
ctattaacatactcgtgaaccac

SEQ ID NO: 16 - DNA, 18 bp, Artificial
  Oligoncleotide probe
gttggtttaagttggctt

SEQ ID NO: 17 - DNA, 54 bp, Artificial
  Oligonucleotide probe
tagaatttctaatataaaattctattaacatactcgtgaaccacaaacggtcta

SEQ ID NO: 18 - DNA, 24 bp, Artificial
  Oligocnucleotide probe
caaaaagaaggtcttcattacacc

SEQ ID NO: 19 - DNA, 24 bp, Artificial
  Oligonucleotide probe
agtgttcgggtgtctataaaggac

SEQ ID NO: 20 - DNA, 29 bp, Artificial
  Oligonucleotide probe
gcggccgcttatgcacagggtggaacaag

SEQ ID NO: 21 - DNA, 24 bp, Artificial
  Oligonucleotide probe
tctagaccacttgagtccgtgtca

SEQ ID NO: 22 - DNA, 58 bp, Artificial; identical to SEQ ID NO: 14
  Olignucleotide probe

SEQ ID NO: 23 - DNA, 23 bp, Artificial
  Olignucleotide probe
gataattgtatgagcacttggtg

SEQ ID NO: 24 - DNA, 48 bp, Artificial
  Olignucleotide probe
cagagaacaaaaacaaaataatccagtgagaaaagcccgtaaataaag

SEQ ID NO: 25 - DNA, 23 bp, Artificial
  Olignucleotide probe
gataattgtatgagcacttggtg

SEQ ID NO: 26 - DNA, 30 bp, Artificial
  Olignucleotide probe
ttgccttcttagagatcacaagccaaagct

SEQ ID NO: 27 - DNA, 49 bp, Artificial
  Olignucleotide probe
cccacacagatgctcaccacccaatattattgttctctgtaaacggaga

SEQ ID NO: 28 - DNA, 23 bp, Artificial
  Olignucleotide probe
ggttaatgtgaagtccaggatcc

SEQ ID NO: 29 - DNA, 44 bp, Artificial
  Olignucleotide probe
cattaagtgtattgaaggcgaaaagaatcagagaacagttgatc

SEQ ID NO: 30 - DNA, 54 bp, Artificial
  Oligonucleotide probe
aacagttcttctttttaagttgagcttaaaataagctgagaatagatctggttt

SEQ ID NO: 31 - DNA, 23 bp, Artificial
  Olignucleotide probe
ggttaatgtgaagtccaggatcc

SEQ ID NO: 32 - DNA, 31 bp, Artificial
  Olignucleotide probe
gatgggaaacctgtttagctcacccgtgagc

SEQ ID NO: 33 - DNA, 25 bp, Artificial
  Olignucleotide probe
catcccactacacagaatctgttag

(19) United States
(12) Patent Application Publication
(10) Pub. No.: US 2008/0311572 A1
(43) Pub. Date: Dec. 18, 2008

(54) Methods and Compositions For Correlating Ccl3l1/Ccr5 Genotypes With Disorders

(76) Inventors: Sunil K. Ahuja, San Antonio, TX (US); Matthew Dolan, San Antonio, TX (US); Hemant Kulkarni, San Antonio, TX (US); Enrique Gonzalez, San Antonio, TX (US)

Correspondence Address: MYERS BIGEL SIBLEY & SAJOVEC, PO BOX 37428, RALEIGH, NC, 27627, US

(21) Appl. No.: 11/791,403

(22) PCT Filed: Jul. 6, 2005
(86) PCT No.: PCT/US2005/023830

§ 371 Date: Nov. 12, 2007

Related U.S. Application Data
(60) Provisional application No. 60/631,292, filed on Nov. 26, 2004.
(60) Provisional application No. 60/681,131, filed on May 16, 2005.

(51) Int. Cl. C12Q 1/68

(52) U.S. Cl. 435/6

(57) ABSTRACT

The present invention provides compositions and methods for identifying persons at an increased risk of infection by, transmission of, or accelerated progression of a disease caused by an HIV-1 virus. Diagnostic and therapeutic kits are also provided.

### STATEMENT OF PRIORITY

This application claims the benefit, under 35 U.S.C. § 119(e), of U.S. Provisional Application Ser. No. 60/631,292, filed Nov. 26, 2004 and U.S. Provisional Application Ser. No. 60/680,131, filed May 12, 2005, the entire contents of each of which are incorporated herein by reference.

### STATEMENT OF GOVERNMENT SUPPORT

The U.S. government owns rights in the present invention pursuant to grant number AI046326 from the National Institutes of Health.

### BACKGROUND OF THE INVENTION

1. Field of the Invention

The present invention relates generally to the fields of molecular biology and genetics. More particularly, it provides compositions and methods for identifying persons at an increased risk of infection by, transmission of, or accelerated progression of a disorder associated with a detrimental CCL3L1/CCR5 genotype, such as infection with human immunodeficiency virus (HIV).

2. Background Art

Novel ways to identify individuals with enhanced susceptibility to HIV infection or development of acquired immune deficiency syndrome (AIDS) is of high public health significance and is great importance for the clinical care of infected patients. In the clinical setting of HIV infection, both the steady-state viral load (VL), known as the viral set point, and CD4⁺ T cell counts, are widely regarded as the strongest predictors of disease progression. These laboratory markers are also the focus of current guidelines for deciding when Highly Active Anti-Retroviral Therapy (HAART) should be initiated, and HAART is usually recommended when CD4⁺ cell counts are less than 200 or 350 cells/μl and VLs are greater than 55,000 copies/ml.

Both the viral set point and rate of CD4⁺ T cell decline display variations of several orders of magnitude between patients. Despite intensive research, the host and viral factors that are responsible for the observed variation remain poorly understood. Additionally, although they are important clinical tools, these laboratory markers have four significant limitations in the risk-assessment of infected patients.

First, not all persons at high risk of an accelerated disease course are identified by these laboratory markers. For example, in analyses of 1,132 HIV-infected subjects followed prospectively at the Wilford Hall Medical Center (WHMC), although baseline CD4+ T cell counts or viral loads (viral set point) had prognostic value in predicting risk of rapid disease progression, infected individuals having similar levels of these two laboratory markers displayed highly variable rates of disease progression. Exemplifying this variability, ˜44% of subjects with baseline CD4⁺ counts above 700 cells/μl developed AIDS at the same rate as did individuals with baseline counts lower than 200 cells/μl. Similarly, 40% of individuals with low viral set points (<20,000 copies/ml) progressed to AIDS in ˜5 years. These findings indicate that although a low baseline CD4⁺ count or a high viral set point heavily favors the possibility of an increased risk of progressing rapidly to AIDS, the converse is not true, i.e., a high baseline CD4⁺ count or low viral set point does not exclude the possibility of an accelerated disease course.

Second, there is a strong correlation between baseline CD4⁺ T cell counts and viral set point (Spearman rho=−0.2439, P<0.0001), baseline CD4⁺ T cell counts and rate of CD4⁺ T cell decline (rho=−0.1763, P<0.0001), and viral set point and rate of CD4⁺ T cell decline (rho=−0.1904, P=0.0006) in this cohort of infected adults. These findings indicate that the laboratory markers of prognostication capture overlapping components of AIDS risk.

Third, by computing the log likelihood from the Cox proportional hazards models to estimate the amount of variation (R_{M}²) in the rate of progression to AIDS that is explained by baseline CD4⁺ T cell counts or viral set points, the R_{M}² values were found to be comparably low (˜5%) for each laboratory marker. These findings indicate that despite being statistically significant, and sometimes impressive relative hazards for the association between different baseline CD4+ T cell counts and VL strata; these markers of disease progression explain only a small fraction of the overall variation in clinical course of an HIV+ individual. This emphasizes the need to identify additional independent markers of disease progression.

Fourth, clinical decision-making in HIV medicine oftentimes hinges on the serial determinations of the laboratory markers to provide meaningful prognostication. Thus, single time-point estimates of these two laboratory markers may provide a static snapshot of the disease process, but may not correlate fully with the future trajectory of the clinical course of patients.

Collectively, these findings support the urgent need for population-based data to identify host-centric risk factors that can i) predict the future risk of AIDS independent of baseline CD4+ T cell counts and VLs; and/or ii) provide clues into the immune correlates of the observed inter-individual variation in T cell loss and the viral set point. Knowledge of such host-centric vulnerability factors will not only aid in the global risk assessment and clinical management of infected patients, but will also assist in rational vaccine design. The present invention overcomes previous shortcomings in the art by providing compositions and methods for identifying subjects having an increased susceptibility to certain disorders that can be correlated with the presence of a particular genotype of the dual genetic marker, CCL3L1/CCR5.

### BRIEF DESCRIPTION OF THE DRAWINGS

FIGS. 1A-F. Influence of variations in CCL3L1 or CCR5 on HIV-1 disease course, viral set point, and rate of CD4+ T cell loss prior to accounting for their combined effects. The findings are from the combined analyses of the EA− and AA components of the HIV+ Wilford Hall Medical Center (WHMC) cohort of infected adults. (A-C) Kaplan-Meier (KM) curves of the development of AIDS for individuals possessing CCL3L1^{high} versus CCL3L1^{low} before (A), or after stratifying for the baseline VL (setpoint) (B) and the rate of CD4+ T cell loss (C). (D-F) KM curves of the development of AIDS for individuals possessing CCR5^{non-det} versus CCR5^{det} before (D) or after stratifying for the baseline VL (E) and the rate of CD4+ T cell decline (F). P and RH (95% confidence interval) indicate the significance value by log-rank test and the relative hazard.

FIGS. 2A-I. Influence of variations in CCL3L1 or CCR5 on rate of CD4+ T cell decline, HIV-1 disease course, and risk of acquiring HIV-1 infection after accounting for their combined effects. (A) Genetic stratification system for analyses of the individual or combined effects of variation in CCL3L1 and CCR5: (i) CCL3L1^{low}CCR5^{non-det}, reflecting the independent (detrimental) effects of population-specific low CCL3L1 dose; (ii) CCL3L1^{high} CCR5^{det}, reflecting the independent (detrimental) effects of population-specific CCR5 genotypes; (iii) CCL3L1^{low}CCR5^{det}, reflecting the combined (detrimental) effects of CCL3L1 and CCR5; and (iv) CCL3L1^{high}CCR5^{non-det}, which is the reference group. The population-specific CCL3L1 dose that corresponds to CCL3L1^{low} or CCL3L1^{high} in the EA and AA population (pop1ⁿ) is indicated; det, detrimental; non-det, non-detrimental. (B) Rate of CD4+ T cell decline in the four CCL3L1/CCR5 genotypic groups. The plots represent median (±1.7 SD of median) values. Note, relative to a low negative number (e.g., −0.30), a higher negative number (e.g., −0.10), reflects a slower rate of decline in CD4+ T cells. CCL3L1^{low}CCR5^{det} was associated with the fastest rate of CD4+ T cell decline. P indicates the results of the overall Kruskal-Wallis test. (C-D) KM curves of the development of AIDS in EAs and AAs from the entire (C) or seroconverting portion (D) of the EA and AA infected adult cohort after stratifying for the different CCL3L1/CCR5 genotypic groups shown in panel A. Inset, frequency distribution of genotypic groups. P and RH (95% confidence interval) indicate the significance value by Cox regression and the relative hazard, respectively. (E-G) Changes in the frequency distribution of CCL3L1/CCR5 genotypic groups in the HIV+ WHMC cohort for individuals with varying AIDS-free survival times (E-F), and differences in the distribution between HIV+ and HIV− adults (G). (H-I) Varying risk (OR, odds ratio) of acquiring HIV-1 in the setting of horizontal (H) and vertical (I) transmission in individuals possessing the indicated CCL3L1/CCR5 genotypic groups. In panels E-H to ensure appropriate ethnic-matching for the comparisons of the frequency distributions between the HIV+ and HIV− individuals, these analyses are for the EA, AA and Hispanic American portions of the infected cohort. In children exposed perinatally to HIV-1, possession of less than 2 CCL3L1 gene copies and the CCR5-HHE haplotype (11) are associated with an increased risk of acquiring HIV-1, and thus, correspond to CCL3L1^{low} and CCR5^{det} in panel I.

FIGS. 3A-M. CCL3L1^{low}CCR5^{det}: prognostic genetic marker of acdelerated disease progression and rapid decline in CD4+ and CD8+ T cells independent of baseline CD4+ T cell counts and VLs (setpoint). (A-H) KM plots of the development of AIDS prior to (indicated as overall in panels A and E) and after accounting for different baseline CD4+ T cell counts (B-D) or viral setpoints (F-H). P and RH (95% confidence interval) indicate the significance value by log-rank test and the relative hazard with respect to the reference group, respectively. (I) Relative hazards (95% CI) of the four multivariate Cox proportional hazards models for time to AIDS for the CCL3L1/CCR5 genetic risk groups: (i) before adjustment for the rate of change in CD4+ T cell counts in the entire cohort (model #1); (ii) after adjustment for rate of CD4+ T cell decline in the entire cohort (model #2) or the seroconverting portion of the cohort (model #3); and (iii) after adjustment for the rate of CD4+ T cell decline and baseline VLs in the seroconverting portion of the cohort (model #4). The rate of CD4+ T cell decline was adjusted by including it as a covariate and the initial VL was adjusted by including it as a time-varying covariate. (J-M) Regression lines showing the monthly rate of change in CD4+ (J-K) and CD8+ T cell (L-M) counts in the three genetic risk groups before stratifying for baseline CD4+ T cell counts and VLs.

FIGS. 4A-F. Comparison of the prognostic value of baseline CD4+ T cell count and VLs as well as genetic risk groups in predicting AIDS at the level of a prospectively followed cohort. The prognostic value was determined by calculating the likelihood ratios (LR) and 95% confidence intervals (CI) for development of AIDS in HIV-infected subjects followed prospectively with different laboratory or genetic markers. (A-C) LRs for different strata of baseline CD4+ counts and VLs, before (A) and after (B-C) accounting for the genetic risk groups. (D-E) Relationship between duration of follow-up (abscissa) and LRs of the risk of developing AIDS for the indicated categories of baseline CD4+ T cell counts and VLs as well as genetic risk groups.

FIGS. 5A-C. Comparison of the prognostic value of baseline CD4+ T cell count and VLs as well as genetic risk groups in predicting AIDS at the level of a nested case-control study. (A-C) LRs for different strata of baseline CD4+ counts and VLs, before (A) and after (B-C) accounting for the genetic risk groups.

FIGS. 6A-C. Distribution of the mean CCL3L1 gene-containing segmental duplications in human populations and CCL3L ortholog(s) in chimpanzees. (A) The human populations are labeled below the figure and their geographic affiliations are shown above it. CCL3L1 copy number was determined by real-time Taqman PCR assays. (B) The frequency, mean, variance, standard deviation (SD), median, and interquartile range (IQR) of the CCL3L1 copy numbers in the indicated populations are shown. (C) The order of the abbreviations. (geographic regions shown in panel A and chimpanzee (CH)) matches the order of the cumulative frequency curves from left to right.

FIGS. 7A-H. CCL3L1 gene dose and risk of acquiring HIV-1. (A to D) Histograms and the cubic—spline smoothed frequency curves (insets) show that the distribution of the CCL3L1 gene copy numbers (x axis) in HIV⁺ versus HIV⁻ (open bars in inset) individuals is markedly different (X² and P values above insets; n=number of individuals in each group). Vertical arrow (copy number at which the HIV⁺/HIV⁻ ratio switched from >1 to ≦1) indicates the switch point. The cohort of Argentinean children is comprised of children exposed perinatally to HIV (4). The HIV⁺ adults from the indicated ethnic groups (noted on the right) are from the Wilford Hall Medical Center (WHMC) cohort (14) and are compared to an ethnically matched control group from the general population (4). (E to H) Risk of acquiring HIV relative to the population-specific median (horizontal arrow; odds ratio (OR)=1) was determined by multivariate logistic regression analyses. *, Jewell correction (4); #, CCL3L1 gene copy number; CI, confidence interval; P, significance value.

FIGS. 8A-Q. Disease-influencing and functional phenotypic effects associated with CCL3L1 gene copy numbers. (A and B) Kaplan-Meier (KM) survival curves of the development of AIDS in AAs (A) and EAs (B) from the adult WHMC HIV⁺ cohort who possess CCL3L1 gene copies equal to, or lower than the population-specific median (copy numbers noted adjacent to KM curves). Note, as the population-specific median number of CCL3L1 copies was 3 and 4 in HIV⁺ and HIVAAs, respectively, these two gene copy numbers were used as the reference genetic strata in A; the reference group in EAs is 2 copies. P and relative hazard (RH) below the KM curves were determined by Cox proportional hazard models. (C) Relationship between CCL3L1 copy numbers and percentage of CD4⁺/CCR5⁺ cells in unstimulated (open bars) or anti-CD3/CD28-stimulated peripheral blood mononuclear cells (black bars). Numbers inside the bars denote the number of individual blood samples studied with the indicated copy numbers. K-W P, overall Kruskal-Wallis test P value. Vertically oriented numbers indicate P values by the Mann-Whitney test for comparison of possession of 0-2 versus 3-4 or 5-7 CCL3L1 gene copies within each experimental condition. (D to F) Second-order polynomial regression curves show (D) CCL3/CCL3L1 concentrations in supernatants of freshly isolated peripheral blood mononuclear cells (n=no. of individuals), (E) baseline log viral RNA (viral set point), and (F) monthly CD4⁺ T cell loss have a threshold-type association with CCL3L1 gene copies (4). D and E depict medians (±1.7 standard deviation of medians), and F depicts 95% CI around the point estimates of the regression coefficients obtained by the General Estimating Equations (GEE) method (4). P linear and quadratic (quad) indicate significance values for the linear and quadratic terms in the polynomial regression equation, respectively (4). (G to L) KM curves of the development of AIDS in HIV⁺ AAs and EAs with different CCL3L1 gene copy numbers. The disease-influencing effects associated with possession of (G and H) median, (I) half-median, and (J) low/null CCL3L1 gene doses were similar in EAs and AAs. However, the disease-influencing effects of possession of (K) two copies in AAs (half-median dose in HIV⁻ AAs) and EAs (median dose) or (L) three copies in AAs (median in HIV⁺ AAs) and one copy in EAs (half-median in EAs) were not equivalent. Numbers adjacent to the population designators AA and EA indicate the number of gene copies (e.g., AA4 implies four copies in AAs). P values indicate significance value by log-rank test. =, >, or < signifies the direction of the associated effects. (M and N) Direction and magnitude of the changes in CD3+, CD4+ and CD8+ T cell counts are similar in HIV⁺ EAs and AAs who possess CCL3L1 gene copies equal to, or lower than the population-specific median (error bars indicate 95% CI). (O and P) Results of discrete-time Markov modeling of the evolution of changes in the frequency distribution of CCL3L1 gene copy numbers in infinite-sized AA and EA cohorts over 15 years. Numbers adjacent to the curves indicate CCL3L1 gene copy numbers. (Q) Schema of phenotypic equivalency of transmission- and disease-influencing effects of population-specific CCL3L1 gene doses in EAs and AAs.

FIGS. 9A-L. Disease- and transmission-influencing effects associated with variations in CCL3L1 and/or CCR5. (A) Genetic stratification system. In each population (poplⁿ), CCL3 μl dose and CCR5 genotypes were dichotomized based on whether they were associated with an accelerated disease course. CCL3L1^{low} and CCL3L1^{high} denote copy numbers < or ≧ population-specific median, respectively. CCR5^{det} and CCR5^{non-det} denote population-specific, disease accelerating, i.e., detrimental (det, indicates detrimental), or non-detrimental CCR5 genotypes, respectively. Compared with possession of CCL3L1^{high} or CCR5^{non-det}, CCL3L1^{low} or CCR5^{det} was associated with an accelerated disease course. These dichotomized compound genotypes were used to stratify the cohort further into four mutually exclusive GRGs, which reflected (i) the independent disease-accelerating effects associated with population-specific low CCL3L1 gene doses (CCL3L1^{low}CCR5^{non-det}) or detrimental CCR5 genotypes (CCL3L1^{high}CCR5^{de}); or (ii) their combined effects (CCL3L1^{low}CCR5^{det}), all relative to CCL3L1^{high}CCR5^{non-det}. (B) CD4⁺ and (C) CD8⁺ T cell changes associated with the GRGs are depicted as 95% CI around the point estimates of the regression coefficients obtained by the GEE method (4). (D) Baseline log viral RNA (viral set point; median (±1.7 standard deviation of the median)) associated with the GRGs. P values reflect significance values for differences between CCL3L1^{high}CCR5^{non-det} and CCL3L1^{low}CCR5^{det} by Student's t-test in B and C and the Mann-Whitney test in D. (E and F) KM curves of the development of AIDS in EAs and AAs from the entire (E) or seroconverting portion (F) of the HIV⁺ adult cohort after stratifying for the GRGs. Inset, frequency distribution of the GRGs. (G) Proportions of individuals within each GRG that developed AIDS. (H and I) Risk of horizontal (H) and vertical (I) transmission associated with the indicated GRGs. (J and K) Changes in the frequency distributions of the GRGs and test of linear trend for individuals with varying follow-up times. (L) Differences in the frequency distribution of GRGs between HIV⁺ and HIV adults. In H and J, to ensure appropriate matching of ethnicity for the comparisons of the frequency distributions between HIV⁺ and HIV individuals, these analyses are for the EA, AA and HA portions of the infected adult cohort (4).

FIG. 10. Attributable fractions (AFs) of CCL3L1/CCR5 GRGs for risk of acquiring HIV and rate of disease progression relative to CCL3L1^{high}CCR5^{non-det} in the indicated clinical settings. Vertical bars represent the point estimate, whereas error bars represent the 95% CI around the point estimate of the AF.

FIGS. 11A-F. Conceptual models by which CCL3L1/CCR5 GRGs can influence HIV disease as well as vaccine-related endpoints (A and B), and the influence of the GRGs on CMI in HIV⁺ adults (C to F) in the WHMC cohort. The GRGs were categorized into low, moderate and high risk groups and their genetic composition is shown at the bottom of F. (A) Means by which GRGs might affect proximal and distal events in HIV infection. X, denotes an undefined pathway(s) by which the GRGs might mediate their independent effects. (B) Influence of GRGs on vaccine-related epidemiological endpoints. The endpoint Pc is composed of different components, and those that are influenced by GRGs are shown in green letters, i.e., Ro, e and f. The conceptual model assumes a vaccine that requires the induction of CMI. (C) Best DTH responses in subjects who possess the low, moderate and high risk GRGs. A four antigen panel was used in the DTH skin tests. Best DTH indicates the maximal number of positive skin tests of the four tested at any given time during the disease course of a HIV⁺ subject. P values indicate the significance values of unpaired Student's t test for the difference between the means. (D) Risk of developing anergy in subjects with the different GRGs determined by logistic regression analyses. In C and D, error bars indicate 95% confidence intervals (CI) around the point estimates. (E) Kaplan-Meier (KM) curves for time to progression to anergy/hypoergy. Anergy/hypoergy denotes subjects with zero or one positive skin test of the four tested. This analysis was restricted to subjects who presented with two or more positive skin tests. P and relative hazard (RH) were determined by Cox proportional hazards models. C to E represents analyses for the entire cohort (F) Multivariate Cox proportional hazards model containing the explanatory variables best DTH responses (DTH), bCD4 and VL-sp. The multivariate model with these variables was analyzed in all seroconverters (overall) and in serocoverters with the indicated GRGs.

FIGS. 12A-I. Relationship between GRGs and CD4⁺ T cell depletion in HIV⁺ adults. (A) The cumulative CD4⁺ T cell count (cCD4, 100,000 cell-days/μl) of an individual subject was defined as the area under the curve of CD4⁺ T cell counts versus time and its relationship to bCD4 and VL-sp was determined by Spearman's correlation coefficients. Numbers adjacent to the arrows indicate p values with P values in parenthesis. (B) KM curves for rates of progression to AIDS for upper, middle and lower tertiles of cCD4. (C) cCD4 values in subjects with the indicated GRGs are depicted as 95% CI around the point estimates of mean cCD4. (D) Mean cCD4 values in subjects with the indicated GRGs and the three VL-sp strata (copies/ml; k, ×10³) (E) The relationship between cCD4 and VL-sp in subjects with the indicated GRGs was analyzed using a nonlinear exponential decay model. P values show that the decay in cCD4 as a function of VL-sp is statistically significant only in subjects with the moderate and high risk GRGs. (F to H) Time-trends of the CD4⁺ T cell counts (cells/μl×10²) estimated using the generalized estimating equations (GEE) method in subjects with the indicated GRGs and VL-sp strata. Monthly rates of change (standard error) in CD4⁺ T cells are indicated as color-coded numbers; a negative number, indicates a progressive decrease in cell counts. P values indicate differences between the rates of change in CD4⁺ cells in the moderate and high risk GRGs relative to the low risk GRG as estimated by the Student t test. Rates of change in CD4⁺ T cell counts in subjects as a function of the GRGs but prior to stratification for VL-sp is shown in FIG. S5. (I) A two-way analysis of variance (ANOVA) was used to examine differences in cCD4 as a function of both VL-sp and GRGs with and without an interaction term.

FIGS. 13A-G. Effects of GRGs on VL trajectories in HIV⁺ adults. (A) Loess curves of VL trends in HIV⁺ adults with or without AIDS (upper) and in the indicated GRGs (lower). (B) Mean nadirs of the VL (nVL) in subjects with the indicated GRGs and VL-sp strata. (C) Results of a two-way analysis of variance (ANOVA) used to examine differences in VL nadirs as a function of both VL-sp and GRGs with and without an interaction term. (D) Proportion of HIV⁺ subjects with different GRGs who had at least one VL that was lower than the VL cut-off values indicated to the right of the colored curves. Student's t test with one-tailed P values was used to determine the difference between the proportions, and the alpha value is 0.00625 after Bonferroni correction. AUC, indicates area under the normalized curves. (E) Time trends for the CD4⁺ T cell counts (cells/month) in subjects with the indicated GRGs who had attained VL suppression and did not progress to AIDS. (F and G) Time trends for CD4⁺ T cell counts (cells/month) in subjects with the indicated GRGs during the therapy era and during the HAART era. The values to the right of the regression plots represent the monthly rates of change in CD4⁺ T cells (standard error) before and after adjustment for different covariates. The covariates in F were VL-sp, bCD4, nCD4. Because of the smaller sample size, the same covariates were used in G except the VL-sp. The reference group for the comparisons of the rates of change in CD4⁺ cells is the low risk GRG.

FIGS. 14A-I. Independent effects of GRGs on AIDS prognostication in the seroconverting component of the WHMC cohort. (A) Relative hazards (95% CI) from the ten multivariate Cox proportional hazards models for time to AIDS for the CCL3L1/CCR5 GRGs before and after adjustment for the following covariates: C, bCD4; V, VL-sp; T, therapy era; D, DTH response; P, baseline % CD4⁺; N, nCD4. VL-sp and nCD4 were included as time-varying covariates. The last column is the analysis for these two GRGs combined into one group. The reference group for all the comparisons is the low risk GRG. (B) KM curves of the development of AIDS in subjects with the different GRGs with baseline CD4⁺ cells greater than 700 (upper), or VL-sp of less than 55,000 copies (lower). The values below the KM plots denote the RH, 95% CI and P with respect to the reference group (RH=1). (C to E) GRGs provide additive prognostic information in HIV⁺ adults. (C) Prognostic risk groups (Gp) generated by the classification and regression tree (CART) approach are designated as A to E. The best-fitting nodal points for the bCD4 (C), VL-sp (V) and GRGs (G) for these groups are shown. (D) KM curves for rates of progression to AIDS and (E) pre- and post-test probability of development of AIDS in subjects with the indicated CART-generated risk groups. The RH, 95% CI and P for rate of progression to AIDS for the CART-generated risk groups A to E is shown to the right of the KM curves, and the reference group for this analyses (RH=1) is those with CD4⁺ cells greater than 453 cells/μl and a VL cut-off of less than 17,500 copies/ml (Group B in panel C). In panel E, “overall” refers to the pre-test probability of developing AIDS in the seroconverting adults, and ΔP is the change from this pre-test probability. (F to I) Prognostic performance of GRGs in a risk scoring system that uses cut-offs for CD4⁺ and VLs that are frequently employed to make decisions of when to initiate therapy. (F) The risk scoring system. (G) The prognostic performance of the different predictors was examined by nested Cox proportional hazards models alone (C, V or G) or in combination (C+V or C+V+G). (H and I) KM curves for rate of progression to AIDS for different risk score groups after (H) and before (I) inclusion of the GRGs in the risk scoring system.

FIGS. 15A-F. Modeling the effects of the GRGs on epidemiological endpoints of a therapeutic vaccine that relies on elicitation of effective CMI responses. (A) Ro for the nine population groups (#) stratified based on the GRGs of the infected-uninfected partners. Prop., estimated proportion of the population groups. (B) Simulated epidemic growth in the population groups shown in A. Inset, epidemic trajectory of the overall population unstratified for the population groups. (C) Attributable fractions (AF), critical response time (CRT) and Pc for the population groups. For computing these Pc estimates, the vaccine efficacy was fixed at 0.5. (D) Pc in the population groups at different vaccine efficacies. Vertical dashed line corresponds to the Pc values shown in C. Pc values greater than unity (gray horizontal line) indicates the point at which repeated mass vaccination might be necessary. (E-F) Vaccine efficacy estimates if misallocation of subjects based on their GRGs occurs in randomized controlled trial arms. E, depicts the estimates of vaccine efficacy (as a fraction) for varying degrees (%) of misallocation (m) and true vaccine efficacy. F shows the difference (relative error) between the true and estimated vaccine efficacy as a percentage of the true vaccine efficacy for varying values of m. For example, in a trial in 500 subjects with a vaccine whose true efficacy is 50%, misallocation of only 5% will lead to a vaccine efficacy estimate of 44% (95% CI: 41%-45%), which is a relative error of ˜12%.

FIGS. 16A-G. Replication of the disease-influencing effects associated with CCL3L1 copy number and CCR5 genotype. (A) CCL3L1 and CCR5 genotype and VL trajectories in perinatally-infected Argentinean children. Loess curves of VL trends and rates of change in VLs after 10 years of age derived from the GEE estimates (SE) in HIV⁺ children with the indicated genotypes. To limit the number of VL trajectories depicted on a single graph, CCR5 genotypes are depicted as +/+ or Δ32/+ and reflect subjects that lack or are heterozygous for this mutation. CCL3L1^{low} or CCL3L1^{high} indicates those who possess less than (low), or two or more copies (9). Differences in VL trajectories became evident only after a few years of life and notably, in HIV⁺ children who were null for CCL3L1 (CCL3L1⁰) their inability to suppress virus began nearly 5 years earlier, and the VLs were higher than in children with the other genotypes shown. Note, left and right Y-axes denote log VL and proportion of HIV-infected children receiving ART and HAART, respectively. (B to H) CCL3L1 and CCR5 genotype and changes in CD4⁺ T cell counts in adult EAs from the San Diego component of the AIEDRP cohort. (B and C) Loess curves for time trends in CD4⁺ T cells in subjects who did or did not receive HAART stratified by possession of one, two or greater than two CCL3L1 copy numbers (indicated by superscripts). (D and E) Loess curves for time trends in CD4⁺ T cells in subjects who received HAART and possessed or lacked the (D) CCR5 HHC haplotype, or (E) HHE/HHG*2 or non-HHE/HHG*2 genotype. The black-colored curve in D indicates the change in CD4⁺ T cells in all HAART recipients prior to stratification by any genotype. (F and G) Percentage change from baseline CD4⁺ cell counts at 6, 12, and 24 months post-HAART in subjects that have the same CCR5 genotypes as those shown in D (HHC vs. non-HHC) and E (HHE/HHG*2 vs non-HHE/HHG*2). P values are estimated using the Mann-Whitney test. (H) Risk of failure to respond to HAART (SOM section 6.11) in subjects with the indicated CCR5 genotypes. The results are from multivariate logistic regression.

FIGS. 17A-C. CCR5 gene map.

FIG. 18. Unique CCR5 haplotypes.

FIG. 19A-D. CCR5 haplotype pairs and haplotype pairs that influence HIV transmission and progression.

FIG. 20. Human CCR5 nucleic acid sequence. CCR5 numbering is based on GenBank Accession numbers AF031236 and AF031237 (See also CCR5 genomic DNA clone-GenBank Accession number AF009962)

### SUMMARY OF THE INVENTION

As noted above, the present invention overcomes previous shortcomings in the art by providing improved methods for identifying individuals and populations that are at an increased risk of infection by HIV, an increased risk of transmission of HIV, and/or an increased risk of accelerated HIV disease progression.

HIV entry requires a cell receptor called CC chemokine receptor 5 (CCR5). This receptor interacts with chemokines such as CCL3L1, which has potent HIV activity. The present inventors have found that the gene dose of CCL3L1 varies significantly in different populations of humans, and that this variation is associated with variable susceptibility to HIV/AIDS. The inventors have identified variations in the dual genetic marker, CCL3L1/CCR5, that allows for prediction of the development of AIDS independent of CD4⁺ cell count and viral load, which are the conventional laboratory markers used for risk assessment and clinical care of patients with HIV infection. The predictive capacity of this dual marker CCL3L1/CCR5 genotype is evident at all stages of the disease, and can also allow for prediction of poor CD4⁺ cell responses in individuals who are receiving potent anti-retroviral therapies.

Thus, the present invention provides a dual-component genetic marker, designated CCL3L1^{low}CCR5^{det} that reflects the combined adverse effects of detrimental genotypes of CCR5, the major coreceptor for HIV, and low gene dose of CCL3L1, the most potent CCR5 agonist and anti-HIV chemokine. CCL3L1^{low}CCR5^{det} predicts a significantly higher risk of acquiring HIV, as well as accelerated disease progression and development of AIDS. Notably, the prognostic power of CCL3L1^{low}CCR5^{det} is equivalent to, but independent of, baseline CD4⁺ T-cell counts and viral loads, which are the current standard-of-care laboratory markers used to assess HIV disease vulnerability and guide clinical care. Thus, host genetic prognostication in HIV infection is feasible, and CCL3L1^{low}CCR5^{det} is the first genetic marker with sufficient predictive power to guide contemporary HIV clinical management, as well as aid in the identification and exploitation of novel mechanisms underlying the pathogenesis of HIV clinical phenotypes independent of CD4⁺ T-cell loss and viral set point. The present invention also allows for the identification of immunological correlates of an effective vaccine and identification of individuals who will fail vaccines.

The advances provided by the present invention include genetic markers for the prediction of increased susceptibility to HIV/AIDS, genetic markers for identifying responsiveness to therapy and vaccines, and genetic markers for identifying correlates of an effective vaccine. The present invention also provides for the development of a CCL3L1 based therapy for HIV/AIDS and the invention has further applications for other immunological or different diseases in which CCL3L1 and CCR5 play a role.

In certain embodiments, the present invention provides PCR-based assays to quantify CCL3L1 gene copies in humans and kits comprising reagents for carrying out these assays. These assays have very low inter- and intra-assay variability, and are thus very robust for high throughput applications.

The present invention also provides a method to stratify CCL3L1/CCR5 genotypes for purposes of risk stratification into those with low, moderate and high HIV/AIDS risk groups.

The present invention also provides methods wherein the CCL3L1/CCR5 genotype can be used to identify individuals at high risk of HIV/AIDS when the conventional markers used for clinical care, i.e., CD4⁺ cell count and viral load, would have predicted a low risk. The present invention therefore has use in predicting which HIV-infected individuals will develop AIDS and their responses to therapy, as well as in predicting which individuals will respond to particular vaccine compositions.

Thus, in some embodiments, the present invention provides a method of identifying a subject at increased risk of developing a disorder associated with a detrimental CCL3L1/CCR5 genotype (e.g., HIV infection, AIDS, Kawasaki disease), comprising detecting in a subject the presence of a CCL3L1/CCR5 genotype associated with increased risk of developing a particular disorder associated with a detrimental CCL3L1CCR5 genotype.

### DETAILED DESCRIPTION OF THE INVENTION

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The present invention is based on the unexpected discovery of a correlation between the presence in a subject of a CCL3L1/CCR5 genotype and increased susceptibility to certain disorders associated with a detrimental CCL3L1/CCR5 genotype. As used herein, a “detrimental CCL3L1/CCR5 genotype” is any CCL3L1/CCR5 genotype that is or has been identified to be correlated with certain disorders in a manner that allows for the identification of subjects and/or entire populations of subjects having an increased risk of developing the associated disorder and/or an increased susceptibility to the associated disorder (e.g., more rapid progression to advanced stages of the disorder, shorter life span, poor prognosis, etc.) by detecting the presence of this genotype in the nucleic acid of the subject and/or of the population of subjects.

Thus, in one embodiment of this invention, a method is provided of identifying a subject at increased risk of developing a disorder associated with a detrimental CCL3L1/CCR5 genotype, comprising detecting in a subject the presence of a CCL3L1/CCR5 genotype associated with increased risk of developing a disorder associated with a detrimental CCL3L1/CCR5 genotype. The disorder of this invention can be, but is not limited to, human immunodeficiency virus (HIV) infection, acquired immune deficiency syndrome (AIDS), autoimmune diseases such as systemic lupus erythematosis (SLE), rheumatoid arthritis and Kawasaki disease (KD), infectious disorders such as tuberculosis and cardiovascular disorders such as atherosclerosis and coronary artery disease, as well as any other disorder now known or later identified to be associated with a detrimental CCL3L1/CCR5 genotype.

In one particular embodiment, the present invention provides a method of identifying a subject at increased risk of infection with HIV, comprising: detecting in the subject the presence of a CCL3L1/CCR5 genotype correlated with increased risk of infection with HIV.

Also provided herein is a method of identifying an HIV-infected subject at increased risk of developing acquired immune deficiency syndrome (AIDS), comprising detecting in the subject the presence of a CCL3L1/CCR5 genotype correlated with increased risk of developing AIDS.

In other embodiments, the present invention provides a method of identifying an HIV-infected subject at increased risk of developing a disorder associated with AIDS, comprising detecting in the subject the presence of a CCL3L1/CCR5 genotype correlated with increased risk of developing a disorder associated with AIDS, which can be but is not limited to a disorder such as Pneumocystis carinii pneumonia, Mycobacterium infection, cytomegalovirus infection, etc.

Further provided herein is a method of identifying an HIV-infected subject having an increased likelihood of a poor prognosis and/or reduced life expectancy, comprising detecting in the subject the presence of a CCL3L1/CCR5 genotype correlated with increased likelihood of a poor prognosis and/or reduced life expectancy.

In other embodiments, the present invention provides a method of identifying an HIV-infected subject having an increased likelihood of effectively responding to anti-retroviral therapy (such as highly active anti-retroviral therapy or HAART, as known in the art); comprising detecting in the subject a CCL3L1/CCR5 genotype correlated with an increased likelihood of effectively responding to anti-retroviral therapy.

In addition, the present invention provides a method of identifying an HIV-infected subject having a decreased likelihood of effectively responding to anti-retroviral therapy (e.g., HAART), comprising detecting in the subject a CCL3L1/CCR5 genotype correlated with a decreased likelihood of effectively responding to an anti-retroviral therapy.

In some embodiments, the present invention provides a method of identifying a subject having an increased likelihood of responding effectively to a vaccine against HIV, comprising detecting in the subject a CCL3L1/CCR5 genotype correlated with an increased likelihood of responding effectively to a vaccine against HIV.

Also provided herein is a method of identifying a subject having a decreased likelihood of responding effectively to a vaccine against HIV, comprising detecting in the subject a CCL3L1/CCR5 genotype correlated with a decreased likelihood of responding effectively to a vaccine against HIV.

In yet other embodiments, the present invention provides a method of identifying a subject having a low, medium or high risk of HIV infection and/or more rapid progression of HIV-associated disease (e.g., AIDS), comprising detecting in the subject a CCL3L1/CCR5 genotype correlated with a low, medium or high risk of HIV infection and/or more rapid progression of HIV-associated disease. For example, a subject is identified as having a low risk of HIV infection if the subject has a CCL3L1^{high}CCR5^{non-det} genotype. A subject is identified as having a moderate risk of HIV infection and/or more rapid progression of HIV-associated disease if the subject has either a CCL3L1^{high}CCR5^{det} or a CCL3L1^{low}CCR5^{non-det} genotype and a subject is identified as having a high risk of HIV infection and/or more rapid progression of HIV-associated disease if the subject has a CCL3L^{low}CCR5^{def} genotype.

The CCL3L1^{high}CCR5^{non-det} CCL3L1^{low}CCR5^{non-det} and CCL3L1^{low}CCR5^{det} genotypes are defined herein relative to the population of subjects analyzed. Studies to identify these genotypes in different populations are described in the Examples section provided herein. As one example, according to the classification system described in Figure S16, the following definitions were used to combine the CCR5 genotypes: CCR5^{non-det} was defined in European American (EA) subjects as possession of HHC-containing haplotypes and/or HHG*2-containing genotypes that lack HHE. All the remaining CCR5 genotypes were combined into the group designated as CCR^{det}. Thus, the HHE/HHG*2 subjects were not included in the CCR5^{non-det} group. Then, based on the possession of the varying copies of the CCL3L1 gene risk scoring system was designed as follows (Fig. S16A):

1) Low risk: CCL3L1^{high}CCR5^{non-det} contains HHC-containing genotypes and HHG*2-containing genotypes that lack HHG*2/HHE AND two or more copies of CCL3L1.
2) Moderate risk: CCL3L1^{high}CCR5^{det} or CCL3L1^{low}CCR5^{non-det} groups are those that possess either less than two copies of CCL3L1 OR non-HHC/non-HHC and non-HHG*2/HHG*2 genotypes.
3) High risk: CCL3L1^{low}CCR5^{det} are those that possess less than two copies of CCL3L1 AND non-HHC/non-HHC and non-HHG*2/HHG*2 genotypes.

In further embodiments of the present invention, a method of identifying a subject at increased risk of developing a disorder associated with a detrimental CCL3L1/CCR5 genotype is provided, comprising: a) correlating the presence of a CCL3L1/CCR5 genotype in a test subject with a disorder; and b) detecting the CCL3L1/CCR5 genotype of step (a) in the subject.

In particular, a method is provided herein of identifying a subject at increased risk of infection with HIV, comprising: a) correlating the presence of a CCL3L1/CCR5 genotype in a test subject infected with HIV; and b) detecting the CCL3L1/CCR5 genotype of step (a) in the subject.

Additionally provided is a method of identifying an HIV-infected subject at increased risk of developing acquired immune deficiency syndrome (AIDS), comprising: a) correlating the presence of a CCL3L1/CCR5 genotype in a test subject with AIDS; and b) detecting the CCL3L1/CCR5 genotype of step (a) in the subject.

Also provided herein is a method of identifying an HIV-infected subject at increased risk of developing a disorder associated with AIDS, comprising: a) correlating the presence of a CCL3L1/CCR5 genotype in a test subject with a disorder associated with AIDS; and b) detecting the CCL3L1/CCR5 genotype of step (a) in the subject.

Furthermore, the present invention provides a method of identifying an HIV-infected subject having an increased likelihood of a poor prognosis and/or reduced life expectancy, comprising: a) correlating the presence of a CCL3L1/CCR5 genotype in a test subject infected with HIV and having a poor prognosis and/or reduced life expectancy; and b) detecting the CCL3L1/CCR5 genotype of step (a) in the subject.

In yet further embodiments, the present invention provides a method of identifying an HIV-infected subject having an increased likelihood of effectively responding to anti-retroviral therapy; comprising: a) correlating the presence of a CCL3L1/CCR5 genotype in a test subject infected with HIV and effectively responding to anti-retroviral therapy; and b) detecting the CCL3L1/CCR5 genotype of step (a) in the subject.

Additionally provided herein is a method of identifying an HIV-infected subject having a decreased likelihood of effectively responding to anti-retroviral therapy, comprising: a) correlating the presence of a CCL3L1/CCR5 genotype in a test subject infected with HIV and not responding effectively to anti-retroviral therapy; and b) detecting the CCL3L1/CCR5 genotype of step (a) in the subject.

The present invention further provides a method of identifying a subject having an increased likelihood of responding effectively to a vaccine against HIV, comprising: a) correlating the presence of a CCL3L1/CCR5 genotype in a test subject effectively responding to a vaccine against HIV; and b) detecting the CCL3L1/CCR5 genotype of step (a) in the subject.

Also provided is a method of identifying a subject having a decreased likelihood of responding effectively to a vaccine against HIV, comprising: a) correlating the presence of a CCL3L1/CCR5 genotype in a test subject not responding effectively to a vaccine against HIV; and b) detecting the CCL3L1/CCR5 genotype of step (a) in the subject.

In other embodiments, the present invention is directed to a method of identifying a population having an increased risk of HIV infection, comprising identifying in the population a CCL3L1/CCR5 genotype correlated with an increased risk of HIV infection.

Also provided is a method of identifying a population having an increased risk of HIV infection, comprising: a) correlating the presence of a CCL3L1/CCR5 genotype in HIV-infected members of the population; and b) detecting the CCL3L1/CCR5 genotype of step (a) in the population.

In addition, the present invention provides a method of identifying a population having an increased likelihood of responding effectively to a vaccine against a disorder associated with a CCL3L1/CCR5 genotype, comprising identifying in the population a CCL3L1/CCR5 genotype correlated with an effective response to the vaccine against the disorder associated with a CCL3L1/CCR5 genotype.

Further provided is a method of identifying a population having an increased likelihood of responding effectively to a vaccine against a disorder associated with a CCL3L1/CCR5 genotype, comprising: a) correlating the presence of a CCL3L1/CCR5 genotype in members of the population who respond effectively to the vaccine; and b) detecting the CCL3L1/CCR5 genotype of step (a) in the population.

In further embodiments, the present invention provides a method of identifying a population having an increased likelihood of responding effectively to a vaccine against HIV infection, comprising identifying in the population a CCL3L1/CCR5 genotype correlated with an effective response to the vaccine against HIV infection.

Also provided herein is a method of identifying a population having an increased likelihood of responding effectively to a vaccine against HIV infection, comprising: a) correlating the presence of a CCL3L1/CCR5 genotype in members of the population who respond effectively to the vaccine against HIV infection; and b) detecting the CCL3L1/CCR5 genotype of step (a) in the population.

Further provided is a method of identifying a CCL3L1/CCR5 genotype correlated with increased risk of developing a disorder associated with a CCL3L1/CCR5 genotype, comprising: a) identifying a subject having the disorder; b) detecting in the subject the presence of a CCL3L1/CCR5 genotype; and c) correlating the presence of the CCL3L1/CCR5 genotype of step (b) with the presence of the disorder associated with a CCL3L1/CCR5 genotype, thereby identifying a CCL3L1/CCR5 genotype correlated with increased risk of developing a disorder associated with a CCL3L1/CCR5 genotype.

The present invention also provides a method of identifying a CCL3L1/CCR5 genotype correlated with increased risk of HIV infection, comprising: a) identifying a subject infected with HIV; b) detecting in the subject the presence of a CCL3L1/CCR5 genotype; and c) correlating the presence of the CCL3L1/CCR5 genotype of step (b) with the presence of HIV infection, thereby identifying a CCL3L1/CCR5 genotype correlated with increased risk of HIV infection.

The present invention further provides a method of identifying a CCL3L1/CCR5 genotype correlated with increased risk of developing acquired immune deficiency syndrome (AIDS), comprising: a) identifying a subject having AIDS; b) detecting in the subject the presence of a CCL3L1/CCR5 genotype; and c) correlating the presence of the CCL3L1/CCR5 genotype of step (b) with the presence of AIDS, thereby identifying a CCL3L1/CCR5 genotype correlated with increased risk of developing AIDS.

In addition, the present invention provides a method of identifying a CCL3L1/CCR5 genotype correlated with increased risk of developing a disorder associated with AIDS, comprising: a) identifying a subject having a disorder associated with AIDS; b) detecting in the subject the presence of a CCL3L1/CCR5 genotype; and c) correlating the presence of the CCL3L1/CCR5 genotype of step (b) with the presence of the disorder associated with AIDS, thereby identifying a CCL3L1/CCR5 genotype correlated with increased risk of developing a disorder associated with AIDS.

Furthermore, the present invention provides a method of identifying a CCL3L1/CCR5 genotype correlated with increased likelihood of a poor prognosis and/or reduced life expectancy due to a disorder associated with a CCL3L1/CCR5 genotype, comprising: a) identifying a subject having a disorder associated with a CCL3L1/CCR5 genotype; b) detecting in the subject the presence of a CCL3L1/CCR5 genotype; and c) correlating the presence of the CCL3L1/CCR5 genotype of step (b) with the presence of the disorder associated with a CCL3L1/CCR5 genotype, thereby identifying a CCL3L1/CCR5 genotype correlated with increased likelihood of a poor prognosis and/or reduced life expectancy due to a disorder associated with a CCL3L1/CCR5 genotype.

A method is also provided herein of identifying a CCL3L1/CCR5 genotype correlated with increased likelihood of effectively responding to anti-retroviral therapy, comprising: a) identifying a subject infected with HIV who is effectively responding to anti-retroviral therapy; b) detecting in the subject the presence of a CCL3L1/CCR5 genotype; and c) correlating the presence of the CCL3L1/CCR5 genotype of step (b) with the effective response to anti-retroviral therapy, thereby identifying a CCL3L1/CCR5 genotype correlated with increased likelihood of an effective response to anti-retroviral therapy.

Furthermore, a method is provided herein of identifying a CCL3L1/CCR5 genotype correlated with a decreased likelihood of effectively responding to anti-retroviral therapy, comprising: a) identifying a subject infected with HIV who is not effectively responding to anti-retroviral therapy; b) detecting in the subject the presence of a CCL3L1/CCR5 genotype; and c) correlating the presence of the CCL3L1/CCR5 genotype of step (b) with the lack of effective response to anti-retroviral therapy, thereby identifying a CCL3L1/CCR5 genotype correlated with a decreased likelihood of an effective response to anti-retroviral therapy.

Also provided herein is a method of identifying a CCL3L1/CCR5 genotype correlated with an increased likelihood of effectively responding to a vaccine against a disorder associated with a CCL3L1/CCR5 genotype, comprising: a) identifying a subject who is effectively responding to a vaccine against a disorder associated with a CCL3L1/CCR5 genotype; b) detecting in the subject the presence of a CCL3L1/CCR5 genotype; and c) correlating the presence of the CCL3L1/CCR5 genotype of step (b) with the effective response to the vaccine, thereby identifying a CCL3L1/CCR5 genotype correlated with an increased likelihood of effectively responding to a vaccine against a disorder associated with a CCL3L1/CCR5 genotype.

Additionally provided is a method of identifying a CCL3L1/CCR5 genotype correlated with a decreased likelihood of effectively responding to a vaccine against a disorder associated with a CCL3L1/CCR5 genotype, comprising: a) identifying a subject who is not effectively responding to a vaccine against a disorder associated with a CCL3L1/CCR5 genotype; b) detecting in the subject the presence of a CCL3L1/CCR5 genotype; and c) correlating the presence of the CCL3L1/CCR5 genotype of step (b) with the lack of effective response to the vaccine, thereby identifying a CCL3L1/CCR5 genotype correlated with a decreased likelihood of effectively responding to a vaccine against a disorder associated with a CCL3L1/CCR5 genotype.

Further provided herein is a method of identifying a CCL3L1/CCR5 genotype correlated with an increased likelihood of effectively responding to a vaccine against HIV infection, comprising: a) identifying a subject who is effectively responding to a vaccine against HIV infection; b) detecting in the subject the presence of a CCL3L1/CCR5 genotype; and c) correlating the presence of the CCL3L1/CCR5 genotype of step (b) with the effective response to the vaccine, thereby identifying a CCL3L1/CCR5 genotype correlated with an increased likelihood of effectively responding to a vaccine against HIV infection.

In addition, the present invention provides a method of identifying a CCL3L1/CCR5 genotype correlated with a decreased likelihood of effectively responding to a vaccine against HIV infection, comprising: a) identifying a subject who is not effectively responding to a vaccine against HIV infection; b) detecting in the subject the presence of a CCL3L1/CCR5 genotype; and c) correlating the presence of the CCL3L1/CCR5 genotype of step (b) with the lack of effective response to the vaccine, thereby identifying a CCL3L1/CCR5 genotype correlated with a decreased likelihood of effectively responding to a vaccine against HIV infection.

In additional embodiments, the present invention provides a method of identifying a subject at increased risk of having Kawasaki disease, comprising detecting in the subject a CCL3L1/CCR5 genotype correlated with increased risk of having Kawasaki disease, as well as a method of identifying a subject at increased risk of having Kawasaki disease, comprising: a) correlating the presence of a CCL3L1/CCR5 genotype in a test subject with Kawasaki disease; and b) detecting the CCL3L1/CCR5 genotype of step (a) in the subject.

Further provided herein is a method of identifying a CCL3L1/CCR5 genotype correlated with increased risk of having Kawasaki disease, comprising: a) identifying a subject with Kawasaki disease; b) detecting in the subject the presence of a CCL3L1/CCR5 genotype; and c) correlating the presence of the CCL3L1/CCR5 genotype of step (b) with the presence of Kawasaki disease, thereby identifying a CCL3L1/CCR5 genotype correlated with increased risk of having Kawasaki disease.

In addition, the present invention provides a method of identifying a CCL3L1/CCR5 genotype correlated with a reduced risk of having Kawasaki disease, comprising: a) identifying a subject without Kawasaki disease; b) detecting in the subject the presence of a CCL3L1/CCR5 genotype; and c) correlating the presence of the CCL3L1/CCR5 genotype of step (b) with the absence of Kawasaki disease, thereby identifying a CCL3L1/CCR5 genotype correlated with decreased risk of having Kawasaki disease.

It is further contemplated that the methods of this invention can be carried out to identify CCL3L1/CCR5 genotypes correlated with AIDS prognostication indicators, such as cell-mediated immunity, CD4⁺ cell depletion and therapy-induced changes in viral loads and CD4⁺ cell counts. These genotypes can be detected in HIV-infected subjects according to the methods described herein for use in developing and guiding HIV clinical care, vaccine trials and prevention programs, as described herein.

Thus, the present invention further provides a method of identifying a subject having a beneficial (e.g., protective) or a detrimental response (e.g., an immune response, a pharmacological response, etc.) to an agent that treats and/or prevents a disorder associated with a detrimental CCL3L1/CCR5 genotype, comprising detecting in a subject the presence of a CCL3L1/CCR5 genotype associated with a beneficial (e.g., protective) or detrimental response (e.g., an immune response, a pharmacological response, etc.) to an agent that treats and/or prevents a disorder associated with a detrimental CCL3L1/CCR5 genotype

Additionally provided herein is a method of identifying a subject having a beneficial (e.g., protective) or a detrimental response (e.g., an immune response, a pharmacological response, etc.) to an agent that treats and/or prevents a disorder associated with a detrimental CCL3L1/CCR5 genotype, comprising: a) correlating the presence of a CCL3L1/CCR5 genotype in a test subject with a beneficial or a detrimental response to an agent that treats and/or prevents a disorder associated with a detrimental CCL3L1/CCR5 genotype; and b) detecting the CCL3L1/CCR5 genotype of step (a) in the subject.

In further embodiments, the present invention provides a method of identifying a CCL3L1/CCR5 genotype correlated with a beneficial (e.g., protective) or a detrimental response (e.g., an immune response, a pharmacological response, etc.) to an agent that treats and/or prevents a disorder associated with a CCL3L1/CCR5 genotype, comprising: a) identifying in a subject a beneficial or a detrimental response to an agent that treats and/or prevents a disorder associated with a CCL3L1/CCR5 genotype; b) detecting in the subject the presence of a CCL3L1/CCR5 genotype; and c) correlating the presence of the CCL3L1/CCR5 genotype of step (b) with the beneficial or detrimental response to an agent that treats and/or prevents a disorder associated with a CCL3L1/CCR5 genotype, thereby identifying a CCL3L1/CCR5 genotype correlated with a beneficial or detrimental response to an agent that treats and/or prevents a disorder associated with a CCL3L1/CCR5 genotype

A beneficial or detrimental response to an agent of this invention (e.g., a vaccine, anti-retroviral drug, etc.) is detected by evaluation, according to known protocols, of various immune functions (e.g., cell-mediated immunity, humoral immune response, CD4⁺ cell depletion, etc.) and pharmacological and biological functions (e.g., change in viral load, change in symptoms or other clinical parameters of the disorder, etc.

By applying the methods of this invention, it is possible to design vaccine and clinical trials, as well as prevention programs on the basis of the genotype information of the subjects of the trial and/or program. For example, by employing the methods of this invention, subjects can be identified who may not need to be vaccinated against and/or treated for a particular disorder. Alternatively, subjects can be identified who will need more than one vaccination or treatment against a particular disorder. The use of the methods of this invention for the design of such studies and programs is well within the scope of this invention.

The methods of this invention, wherein a CCL3L1/CCR5 genotype is identified in a subject and in a population and a correlation is identified between various genotypes with susceptibility to specific disorders are exemplified in the Examples section set forth herein, as well as in PCT Publication No. WO 01/27330 and U.S. Provisional application Ser. No. 60/631,292, the entire contents of each of which are incorporated by reference herein. Data from studies conducted to demonstrate the methods of this invention are provided herein and specific studies carried out as described herein are within the scope of the embodiments of this invention.

Thus, the present invention also provides a method of determining the number of CCL3L1 gene copies in a subject, comprising: a) contacting nucleic acid from the subject with an oligonucleotide primer pair, wherein a sense primer comprises at least 10 contiguous nucleotides of a first nucleotide sequence, and an antisense primer comprises at least 10 contiguous nucleotides of a second nucleotide sequence, wherein the sense and antisense nucleotide sequences comprise a pair of sequences selected from the group consisting of:

1) (sense)TCTCCACAGCTTCCTAACCAAGA (SEQ ID NO. ______)
(antisense) CTGGACCCACTCCTCACTGG (SEQ ID NO: ______)
2) (sense) GATGCTATTCTTGGATATCCTGAG (SEQ ID NO: ______)
(antisense) GTGCAGAGAGGACCTGGTTG (SEQ ID NO: ______)
3) (sense) CCTAGATTCTCATACCTGGAGAC (SEQ ID NO: ______)
(antisense) AATCATGCAGGTCTCCACTG (SEQ ID NO: ______)
4) (sense) ATG CAG GTC TCC ACT GCT GC (SEQ ID NO: ______)
(antisense) TCA GGC ACT CYG CTC YAG GTC (SEQ ID NO: ______);
5) (sense) CTG CCC TTG CYG TCC TCC TCT G (SEQ ID NO: ______)
(antisense) AGG TCR CTG ACR TAT TTC TG (SEQ ID NO: ______, singly and/or any combination and any ratio of any combination thereof, and wherein the sense primer is from 10 to 50 nucleotides in length and the antisense primer is from 10 to 50 nucleotides in length, under conditions whereby an amplification product is produced; b) detecting the amplification product of step (a); and c) quantifying the amount of amplification product detected in step (b), thereby determining the number of CCL3L1 gene copies in the subject.

In certain embodiments, the amplification product of the method of this invention can be detected by hybridization with a nucleic acid probe comprising at least 10 contiguous nucleotides of the nucleotide sequence AGGCCGGCAGGTCTGTGCTGA (SEQ ID NO: ______) and wherein the nucleic acid probe is from 10 to 200 nucleotides in length. In some embodiments, the nucleic acid can be detectably labeled with any known detectable label, several of which are well known and available in the art.

In further embodiments, the present invention provides a method of determining the CCR5 genotype of the subject either alone or in combination with a method of determining the number of CCL3L1 gene copies of the subject.

For example, in some embodiments of this invention, either in conjunction with a method of determining the number of CCL3L1 gene copies of the subject, or independently, the CCR5 genotype of the subject can be determined by contacting nucleic acid from the subject with a set of nucleic acid segments, in any combination and/or any ratio of a combination thereof, wherein the set of nucleic acid segments comprises at least one nucleic acid segment capable of detecting each of the following haplotype groups, each CCR5 haplotype group (haplogroup) being defined in terms of the nucleotides at positions 29, 208, 303, 627, 630, 676 and 927 of the human CCR5 sequence provided herein, with definition of the amino acid at position 64 and the presence or absence of the Δ32 deletion of the human CCR5 sequence, as follows:

Nucleotide position in
CCR5 sequence

| | | | | | | | |
|---|---|---|---|---|---|---|---|
| Haplogroup | 29 | 208 | 303 | 627 | 630 | 676 | 927 |

| | | | | | | | | |
|---|---|---|---|---|---|---|---|---|
| HHA: | A | G | G | T | C | A | C | |
| HHB: | A | T | G | T | C | A | C | |
| HHC: | A | T | G | T | C | G | C | |
| HHD: | A | T | G | T | T | A | C | |
| HHE: | A | G | A | C | C | A | C | |
| HHF*1: | A | G | A | C | C | A | T | |
| HHF*2: | A | G | A | C | C | A | T | isoleucine at amino acid 64 |
| HHG*1: | G | G | A | C | C | A | C | |
| HHG*2: | G | G | A | C | C | A | C | has Δ32, 32 base pair deletion |

In particular embodiments, the CCR5 haplotype is detected by contacting the nucleic acid from the subject with a pair of oligonucleotides selected from the group consisting of:

1)
5′-GAGCCAAGGTCACGGAAGCCC-3′
3′-CCTGGGTCCTAGAATCAC-5′ (CCR5-A29G)
2)
5′GTGGGATGAGCAGAGAACAAAAACAAAATAATCCAGTGAGAAAAGCCC
GTAAATAAAG-3′
3′-CTATTAACATACTCGTGAACCAC-5′ (CCR5-627T)
3)
5′-GTTGGTTTAAGTTGGCTT-3′
3′TAGAATTTCTAATATAAAATTCTATTAACATACTCGTGAACCACAAAC
GGTCTA-5′ (CCR5-C927T)
4)
5′-CAAAAAGAAGGTCTTCATTACACC-3′
3′-AGTGTTCGGGTGTCTATAAAGGAC-5′ (CCR5-Δ32)
5)
5′-GCGGCCGCTTATGCACAGGGTGGAACAAG-3′
5′-TCTAGACCACTTGAGTCCGTGTCA-3′ (Human CCR5 ORF)
6)
5′GTGGGATGAGCAGAGAACAAAAACAAAATAATCCAGTGAGAAAAGCCC
GTAAATAAAG-3′
5′-GATAATTGTATGAGCACTTGGTG-3′ (CCR5 T627C)
7)
5′CAGAGAACAAAAACAAAATAATCCAGTGAGAAAAGCCCGTAAATAAA
G-3′
5′-GATAATTGTATGAGCACTTGGTG-3′ (CCR5 T627C)
8)
5′-TTGCCTTCTTAGAGATCACAAGCCAAAGCT3′
5′-CCCACACAGATGCTCACCACCCAATATTATTGTTCTCTGTAAACGGA
GA-3′ (CCR5 G208T)
9)
5′-GGTTAATGTGAAGTCCAGGATCC-3′
5′-CATTAAGTGTATTGAAGGCGAAAAGAATCAGAGAACAGTTGATC-3′
(CCR5A676G)
10)
5′AACAGTTCTTCTTTTTAAGTTGAGCTTAAAATAAGCTGAGAATAGATC
TGGTTT
5′-GGTTAATGTGAAGTCCAGGATCC3′ (CCR5 C639T)
11)
5′-GATGGGAAACCTGTTTAGCTCACCCGTGAGC3′
5′-CATCCCACTACACAGAATCTGTTAG-3′ (CCR5 G303A)

Thus, it is further contemplated that the compositions of this invention can be provided in a kit format, said kit comprising an oligonucleotide primer pair, wherein a sense primer comprises at least 10 contiguous nucleotides of a first nucleotide sequence and an antisense primer comprises at least 10 contiguous nucleotides of a second nucleotide sequence, wherein the sense and antisense nucleotide sequence comprise a pair of sequences selected from the group consisting of:

1) (sense)TCTCCACAGCTTCCTAACCAAGA (SEQ ID NO. ______)
(antisense) CTGGACCCACTCCTCACTGG (SEQ ID NO: ______)
2) (sense) GATGCTATTCTTGGATATCCTGAG (SEQ ID NO: ______)
(antisense) GTGCAGAGAGGACCTGGTTG (SEQ ID NO: ______)
3) (sense) CCTAGATTCTCATACCTGGAGAC (SEQ ID NO: ______)
(antisense) AATCATGCAGGTCTCCACTG (SEQ ID NO: ______)
4) (sense) ATG CAG GTC TCC ACT GCT GC (SEQ ID NO: ______)
(antisense) TCA GGC ACT CYG CTC YAG GTC (SEQ ID NO: ______);
5) (sense) CTG CCC TTG CYG TCC TCC TCT G (SEQ ID NO: ______)
(antisense) AGG TCR CTG ACR TAT TTC TG (SEQ ID NO: ______); and any combination in any ratio of combinations, and wherein the sense primer is from 10 to 50 nucleotides in length and the antisense primer is from 10 to 50 nucleotides in length.

The kit of this invention can further comprise a nucleic acid probe (e.g., a detectably labeled probe) comprising at least 10 contiguous nucleotides of the nucleotide sequence AGGCCGGCAGGTCTGTGCTGA (SEQ ID NO: ______) and wherein the nucleic acid probe is from 10 to 200 nucleotides in length.

A kit of this invention can also comprise, either separately or in combination with the kit described above, a pair of oligonucleotides selected from the group consisting of:

1)
5′-GAGCCAAGGTCACGGAAGCCC-3′
3′-CCTGGGTCCTAGAATCAC-5′ (CCR5-A29G)
2)
5′-GTGGGATGAGCAGAGAACAAAAACAAAATAATCCAGTGAGAAAAGCC
CGTAAATAAAG-3′
3′-CTATTAACATACTCGTGAACCAC-5′ (CCR5-627T)
3)
5′-GTTGGTTTAAGTTGGCTT-3′
3′TAGAATTTCTAATATAAAATTCTATTAACATACTCGTGAACCACAAAC
GGTCTA-5′′ (CCR5-C927T)
4)
5′-CAAAAAGAAGGTCTTCATTACACC-3′
3′-AGTGTTCGGGTGTCTATAAAGGAC-5′ (CCR5-Δ32)
5)
5′-GCGGCCGCTTATGCACAGGGTGGAACAAG-3′
5′-TCTAGACCACTTGAGTCCGTGTCA-3′ (Human CCR5 ORF)
6)
5′GTGGGATGAGCAGAGAACAAAAACAAAATAATCCAGTGAGAAAAGCCC
GTAAATAAAG-3′
5′-GATAATTGTATGAGCACTTGGTG-3′ (CCR5 T627C)
7)
5′CAGAGAACAAAAACAAAATAATCCAGTGAGAAAAGCCCGTAAATAAA
G-3′
5′-GATAATTGTATGAGCACTTGGTG-3′ (CCR5 T627C)
8)
5′-TTGCCTTCTTAGAGATCACAAGCCAAAGCT3′
5′-CCCACACAGATGCTCACCACCCAATATTATTGTTCTCTGTAAACGGA
GA-3′ (CCR5 G208T)
9)
5′-GGTTAATGTGAAGTCCAGGATCC-3′
5′-CATTAAGTGTATTGAAGGCGAAAAGAATCAGAGAACAGTTGATC-3′
(CCR5A676G)
10)
5′AACAGTTCTTCTTTTTAAGTTGAGCTTAAAATAAGCTGAGAATAGATC
TGGTTT
5′-GGTTAATGTGAAGTCCAGGATCC3′ (CCR5 C639T)
11)
5′-GATGGGAAACCTGTTTAGCTCACCCGTGAGC3′
5′-CATCCCACTACACAGAATCTGTTAG-3′ (CCR5 G303A),
singly, and/or in any combination or any ratio of
any combination.

Some aspects of the present invention are directed to isolated DNA segments that hybridize to one or more coding or non-coding regions of the human CCR5 and/or CCL3L1 gene(s). As used herein, the term “DNA segment” refers to a DNA molecule that has been isolated free of total genomic DNA of a particular species. Therefore, for example, a DNA segment that hybridizes to one or more coding or non-coding regions of the human CCR5 and/or CCL3L1 gene(s) refers to a DNA segment that is isolated away from, or purified free from, total genomic DNA. Included within the term “DNA segment” are DNA segments and smaller fragments of such segments, such as probes and primers, and the like, that are chemically synthesized.

Excepting flanking regions, and allowing for the degeneracy of the genetic code, sequences that have between about 70% and about 79%; or between about 80% and about 89%; or, between about 90% and about 99%; of nucleotides that are identical to the nucleotides of the disclosed nucleic acid sequences will be sequences that are “essentially as set forth in” these sequences.

Sequences that are essentially the same as those set forth in the disclosed nucleic acid sequences may also be functionally defined as sequences that are capable of hybridizing to a nucleic acid segment containing the complement of the disclosed nucleic acid sequences under relatively stringent conditions. Suitable relatively stringent hybridization conditions will be well known to those of skill in the art, as disclosed herein.

For applications requiring high selectivity, one will typically desire to employ relatively stringent conditions to form the hybrids, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and would be particularly suitable for isolating specific genes or detecting specific mRNA transcripts. It is generally appreciated that conditions can be rendered more stringent by the addition of increasing amounts of formamide.

For certain applications, for example, substitution of nucleotides by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Another exemplary, but not limiting, standard hybridization is incubated at 42° C. in 50% formamide solution containing dextran sulfate for 48 hours and subjected to a final wash in 0.5×SSC, 0.1% SDS at 65° C.

The present invention also encompasses DNA segments that are complementary, or essentially complementary, to the sequence set forth in the disclosed nucleic acid sequences. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the disclosed nucleic acid sequences under relatively stringent conditions such as those described herein.

The nucleic acid segments of the present invention, regardless of the length of the “hybridizing” or “complementary” sequence itself, may be combined with other DNA sequences, such as additional restriction enzyme sites, and the like, such that their overall length may vary somewhat.

For example, nucleic acid fragments may be prepared that include a short contiguous stretch identical to or complementary to the disclosed nucleic acid sequences, such as about 8, about 10 to about 14, or about 15 to about 20 nucleotides, and that are up to about 30, or about 50, or about 100 nucleotides in length, with segments of about 25 nucleotides being used in certain cases. DNA segments with total lengths of about 75, about 60, about 45, about 40 and about 35 nucleotides in length (including all intermediate lengths) are also contemplated to be useful.

It will be readily understood that “intermediate lengths,” in these contexts, means any length between the quoted ranges, such as 9, 10, 11, 12, 13, 16, 17, 18, 19, 21, 22, 23, 24, 26, 27, 28, 29, 31, 32, 33, 34, 36, 37, 38, 39, 41, 42, 43, 44, 46, 47, 48, 49, 51, 52, 53, etc.; 100, 101, 102, 103, etc. and the like.

The various primers designed around the disclosed nucleotide sequences of the present invention may be of any length. By assigning numeric values to a sequence, for example, the first residue is 1, the second residue is 2, etc., an algorithm defining all primers can be proposed: n to n+y, where n is an integer from 1 to the last number of the sequence and y is the length of the primer minus one, where n+y does not exceed the last number of the sequence. Thus, for a10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

Various protocols can be employed in the methods of this invention to amplify nucleic acid. As used herein, the term “oligonucleotide-directed amplification procedure” refers to template-dependent processes that result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term “template dependent process” refers to nucleic acid synthesis of an RNA or a DNA molecule wherein the sequence of the newly synthesized strand of nucleic acid is dictated by the well-known rules of complementary base pairing. Typically, vector mediated methodologies involve the introduction of the nucleic acid fragment into a DNA or RNA vector, the clonal amplification of the vector, and the recovery of the amplified nucleic acid fragment. Examples of such methodologies are provided in U.S. Pat. No. 4,237,224, specifically incorporated herein by reference in its entirety. Nucleic acids, used as a template for amplification methods, can be isolated from cells according to standard methodologies (Sambrook et al., 1989). The nucleic acid can be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to the CCR5 and/or CCL3L1 genes are contacted with the isolated nucleic acid under conditions that permit selective hybridization. The term “primer,” as defined herein, is meant to encompass any nucleic acid that is capable of priming the synthesis of a nascent nucleic acid in a template dependent process. Typically, primers are oligonucleotides from ten to twenty base pairs in length, but longer sequences can be employed. Primers may be provided in double-stranded or single-stranded form, although the single-stranded form is commonly used.

Once hybridized, the nucleic acid: primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (e.g., Affymax technology).

A number of template dependent processes are available to amplify the sequences present in a given template sample. One of the best-known amplification methods is the polymerase chain reaction (referred to as PCR), which is described in detail in U.S. Pat. Nos. 4,683,195, 4,683,202 and 4,800,159, each incorporated herein by reference in its entirety.

Briefly, in PCR, two primer sequences are prepared that are complementary to regions on opposite complementary strands of the target sequence. An excess of deoxynucleoside triphosphates is added to a reaction mixture along with a DNA polymerase, e.g., a Taq polymerase. If the particular target sequence is present in a sample, the primers will bind to the target sequence and the polymerase will cause the primers to be extended along the sequence by adding on nucleotides. By raising and lowering the temperature of the reaction mixture, the extended primers will dissociate from the target sequence to form reaction products, excess primers will bind to the target sequence and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermo stable, RNA-dependent DNA polymerases. These methods are described, for example, in WO 90/07641, filed Dec. 21, 1990, incorporated herein by reference in its entirety. Polymerase chain reaction methodologies are well known in the art.

Another method for amplification is the ligase chain reaction (“LCR”), disclosed in Eur. Pat. Appl. No. 320308, incorporated herein by reference in its entirety. In LCR, two complementary probe pairs are prepared and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Q beta Replicase (Q R), described in Intl. Pat. Appl. Publ. No. PCT/US87/00880, incorporated herein by reference, can also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]triphosphates in one strand of a restriction site may also be useful in the amplification of nucleic acids in the present invention.

Strand Displacement Amplification (SDA), described in U.S. Pat. Nos. 5,455,166, 5,648,211, 5,712,124 and 5,744,311, each incorporated herein by reference, is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present.

The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification method, as described in Great Britain Patent 2202328, and in Intl. Pat. Appl. Publ. No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact, available to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Gingeras et al., PCT Application WO 88/10315, incorporated herein by reference). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7, T3 or SP6. In an isothermal cyclic reaction, the RNAs are reverse transcribed into single stranded DNA, which is then converted to double-stranded DNA, and then transcribed once again with an RNA polymerase such as T7, T3 or SP6. The resulting products, whether truncated or complete, indicate target specific sequences.

Davey et al., Eur. Pat. Appl. No. 329822 (incorporated herein by reference in its entirety) discloses a nucleic acid amplification process involving cyclically synthesizing single stranded RNA (ssRNA), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA).

The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (dsDNA) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) discloses a nucleic acid sequence amplification scheme based on the hybridization of a promoter/primer sequence to a target single-stranded DNA (ssDNA) followed by transcription of many RNA copies of the sequence. This scheme is not cyclic, i.e., new templates are not produced from the resultant RNA transcripts. Other amplification methods include “RACE” and “one-sided PCR” (Frohman, 1990, incorporated by reference).

Methods based on ligation of two (or more) oligonucleotides in the presence of nucleic acid having the sequence of the resulting “di-oligonucleotide,” thereby amplifying the dioligonucleotide, may also be used in the amplification step of the present invention.

Following any amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods (Sambrook et al., 1989).

Alternatively, chromatographic techniques may be employed to effect separation. There are many kinds of chromatography that can be used in the present invention: such as, for example, adsorption, partition, ion exchange and molecular sieve, as well as many specialized techniques for using them including column, paper, thin-layer and gas chromatography.

Amplification products must be visualized in order to confirm amplification of the target sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified target sequence. The probe preferably is conjugated to a chromophore but may be radiolabeled. In another embodiment, the probe is conjugated to a binding partner, such as an antibody or biotin, and the other member of the binding pair carries a detectable moiety.

In other embodiments, detection is by Southern blotting and hybridization with a labeled probe. The techniques involved in Southern blotting are well known to those of skill in the art and can be found in many standard books on molecular protocols (Sambrook et al., 1989). Briefly, amplification products are separated by gel electrophoresis. The gel is then contacted with a membrane, such as nitrocellulose, permitting transfer of the nucleic acid and noncovalent binding. Subsequently, the membrane is incubated with a chromophore-conjugated probe that is capable of hybridizing with a target amplification product. Detection is by exposure of the membrane to x-ray film or ion-emitting detection devices. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Diagnostic and therapeutic kits comprising, in at least a first suitable container, one or more nucleic acid segment (s) or primer (s) specific for one or more human CCR5 and/or CCL3L1 genotypes, as defined herein, along with instructions that correlate the identified human CCR5 and/or CCL3L1 genotype with the risk of HIV-1 infection, transmission and/or disease progression, represent another aspect of the invention. Such nucleic acid primers can be DNA or RNA, and can be either native, recombinant, or mutagenized nucleic acid segments.

The kits can comprise a single container that contains a solution of the CCR5 and/or CCL3L1 nucleic acid segment or primer. The single container may contain a dry, or lyophilized, CCR5 and/or CCRL3L1 nucleic acid segment or primer, which may require pre-wetting before use.

Alternatively, the kits of the invention can comprise a distinct container for each component. In such cases, separate or distinct containers would contain the CCR5 and/or CCRL3L1 nucleic acid segments or primers, either as a sterile solution or in a lyophilized form. The kits may also comprise a third container for containing an acceptable buffer, diluent or solvent.

Such a solution may be required to formulate the CCR5 and/or CCL3L1 nucleic acid segment or nucleic acid primer compositions into a more suitable form for amplifying particular CCR5 and/or CCL3L1 genotype DNA segments. It should be noted, however, that all components of a kit could be supplied in a dry form (lyophilized). Thus, the presence of any type of buffer or solvent is not a requirement for the kits of the invention.

As the CCR5 and/or CCL3L1 nucleic acid segments or primers, along with the information correlating the completely identified CCR5 and/or CCL3L1 genotype with the risk of HIV-1 infection, transmission or disease progression, identify subjects that are at an increased risk of HIV-1 infection, transmission or disease progression and thus candidates for anti-retroviral therapy, in certain aspects of the present invention, the kits can further comprise one or more anti-retroviral therapeutic agents, including, but not limited to, reverse transcriptase inhibitors as described in detail herein.

The container (s) will generally be a container such as a vial, test tube, flask, bottle, syringe or other container, into which the components of the kit may be placed. The CCR5 and/or CCL3L1 nucleic acid segment (s) or primer (s) may also be aliquoted into smaller containers, should this be desired. The kits of the present invention may also include material for containing the individual containers in close confinement for commercial sale, such as, e.g., injection or blow-molded plastic containers into which the desired vials or syringes are retained.

It is further contemplated that the methods of this invention can be employed to identify a subject having increased risk of susceptibility to a disorder associated with a detrimental CCL3L1/CCR5 genotype for the purpose of providing preventative and/or therapeutic treatment to the subject. For example, in the case of HIV infection, subjects identified according to the methods of this invention as having an increased risk of HIV infection and/or an increased risk of more rapid progression to an HIV-associated disorder such as AIDS can be treated to prevent HIV infection and/or to prevent or slow the progression to an HIV-associated disorder.

As used herein, an “effective amount” refers to an amount of a compound or composition that is sufficient to produce a desired effect, which can be a therapeutic or beneficial effect. The effective amount will vary with the age, general condition of the subject, the severity of the condition being treated, the particular biologically active agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

Also as used herein, the terms “treat,” “treating” and “treatment” include any type of mechanism, action or activity that results in a change in the medical status of a subject, including an improvement in the condition of the subject (e.g., change or improvement in one or more symptoms and/or clinical parameters), delay in the progression of the condition, prevention or delay of the onset of a disease or illness, etc.

In some embodiments, such treatment can employ gene therapy protocols, as are well known in the art to treatment and/or prevention of genetically related disorders. For example a subject identified as having an increased risk of HIV infection and/or an increased likelihood of more rapidly progressing to AIDS can be administered a CCL3L1 gene to increase the number of CCL3L1 gene copies in the subject, thereby reducing the subject's susceptibility to HIV infection or more rapid progression to AIDS. Other disorders associated with a detrimental CCL3L1/CCR5 genotype can be treated therapeutically and/or prophylactically in the same way, e.g., by increasing the number of CCL3L1 gene copies and/or by increasing the expression of a non-detrimental CCR5 haplotype.

Embodiments are also included in the present invention wherein a subject identified by the methods of this invention to be in need of such treatment can be administered agents that provide the function of CCL3L1 (e.g., inhibition of CCR5), including, but not limited to, agents such as PSC-RANTES, an amino-terminus-modified analog of the chemokine, RANTES (Lederman et al. “Prevention of vaginal SHIV transmission in rhesus macaques through inhibition of CCR5” Science 306:485-487 (2004). Other agents that inhibit CCR5 activity are known in the art.

In other embodiments, subjects at increased risk of developing a disorder associated with a detrimental CCL3L1/CCR5 genotype can be treated therapeutically and/or prophylactically with drugs and immunological agents that target the disorder. For example, a population identified according to the methods of this invention as being at increased risk of HIV infection can be vaccinated against HIV to prevent infection and/or to diminish the likelihood of rapid progression to AIDS. As another example, an HIV-infected subject identified by the methods of this invention to be more likely to rapidly develop AIDS can be treated with anti-retroviral therapy to prevent or slow down the progression to AIDS.

As noted above, methods of this invention allow for the identification of subjects and populations as increased risk of HIV infection, transmission and/or disease progression, and who are therefore candidates for vaccines and/or treatment with one or more of the well-known anti-retroviral therapies, including reverse transcriptase inhibitors. Two pharmacological classes of inhibitor molecules, nucleoside and non-nucleoside, have been found to be effective in halting the enzymatic function of the reverse transcriptase (Larder, 1993). Nucleoside inhibitors such as AZT (zidovudine, azidothymidine; Boucher et al., 1993; Fischl et al., 1987, 1990; Lambert et al., 1990; Meng et al., 1990; Skowron et al., 1993; Furman et al., 1988; Yarchoan et al., 1986), ddC (Zalcitabine, 2′,3′-dideoxycytidine, Hivid), ddI (didanosine, 2′,3′-dideoxyinosine, Videx), and d4T (Stavudine, 2′,3′-didehydro-2′,3′-dideoxythymine) are chemically similar to the normal nucleosides and therefore can be converted to their triphosphate form and then used in the synthesis of DNA during reverse transcription. However, elongation of the DNA chain is blocked since these compounds lack a 3′-OH group that is essential for incorporation of additional nucleotides.

A number of pharmacologically active non-nucleoside inhibitors (NNI) have also been identified. Many of these inhibitors appear highly potent, relatively nontoxic, and specifically inhibit HIV reverse transcriptase. Examples of such compounds include, but are not limited to, nevirapine (BI-RG-587,11-cyclopropyl-5,11-dihydro-4-methyl-6H-dipyrido[3,2-b: 2′,3′] e(1, 4) diazepin-6-one), TIBO (Tetrahydroimidazo[4,5,1 jk][1,4] benzodiazepin-2(1H)-one), HEPT(1-[(2-hydroxyethoxymethyl)]-6-(phenylthio) thymine), BHAP (bis(heteroaryl) piperazine), and alpha-APA (alpha-anilinophenylacetamide).

Therapeutic compounds and reverse transcriptase inhibitors and metabolites thereof useful in any of the methods of the invention also include, but are not limited to dideoxynucleotide triphosphate analogs, including 2′,3′-dideoxynucleoside 5′-triphosphates (Izuta et al., 1991); including, for example, dideoxyinosine and dideoxycytidine (Shirasaka et al., 1990); anti-reverse transcriptase antibodies and sFvs; Carbovir (carbocyclic analog of 2′,3′-didehydro-2′,3′-dideoxyguanosine; White et al., 1990); 3′-azido-3′-deoxythymidine triphosphate, (Furman et al., 1986); 3′-azido-3′-deoxythymidine (Mitsuya et al., 1985; Tavares et al., 1987), thymidine 5′-[a, p-imido]-triphosphate, 3′-azido-3′-deoxythymidine5′-[a, ss-imido]-triphosphate, dideoxythymidine5′-[a, ss-imido]-triphosphate, 3′-azidothymidine5′-[ss,-imido]-triphosphate, thymidine5′-[a, (3:ss,-diimido]-triphosphate (Ma et al., 1992); R82913((+)-S-4,5,6,7-tetrahydro-9-chloro-5-methyl-6-(3-methyl-2-butenyl)-imidazo[4,5,1jk][1,4]-benzodiazepin-2(1H)-thione (a TIBO derivative); (White et al., 1991); 3′-deoxy-2′,3′-dideoxyribose moiety, nucleosides comprising a 2′,3′-didehydro-2′,3′-deoxyribose moiety, 2′,3′ dideoxythymidinene (ddE Thd) (Masood et al., 1989); galolyl derivatives of quinic acid, particularly 3′, 4′,5-tri-O-galoylquinic acid (Tri GQA), and 3,4-di-O-galloyl-5-digalloylquinic acid, Tetra GQA plus 3′-azido-3-deoxy thymidine triphosphate or phosphonoformic acid (Parker et al., 1989); Merck compound L-697,661 (Olsen et al., 1992); 3′-azido-2′, 3′dideoxyadenosine AZA (Shirasaka et al., 1990); 3′-azido-2′-3′-dideoxyguanosine (AZG), carbovir monophosphate; (-Et, -nPr, -nPre, -iPre, -Ce) 5′-triphosphates of 5′-substituted 2′deoxy-uridine; phosphonoacidic acid and phosphonoformic acid (Pei-Zhen, 1989); 3-aminothymidine 5′-triphosphate (Lacey et al., 1992); zidovudine monophosphate and diphosphate; 2′,3′-dideoxynucleosides; R 12913; Ribavirin poly (A)poly (U), (Hovanessian et al., 1991); AZT plus interferon; anhydro-AZT; phosphoformate (“Foscarnet”); deoxy-thiacytidine (Wainberg et al., 1990); anhydro-N3,-UdR and the nonnucleoside inhibitors shown in U.S. Pat. No. 5,917,033 (incorporated herein in its entirety by reference). Any combination of the above reverse transcriptase inhibitors can be used in the treatment methods disclosed herein.

The present invention contemplates the use of pharmaceutical compositions that comprise anti-retroviral agents, such as the reverse transcriptase inhibitors detailed above and/or immunological agents that provide a beneficial prophylactic and/or therapeutic effect.

In such compositions, the active agents can be dissolved or dispersed in a pharmaceutically acceptable carrier or aqueous medium. The terms “pharmaceutically acceptable” or “pharmacologically acceptable” refer to molecular entities and compositions that do not produce an adverse, allergic or other untoward reaction when administered to a subject. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional medium or agent is incompatible with the active ingredient, its use in the compositions of this invention is contemplated. Supplementary active ingredients can also be incorporated into the compositions, as are well known in the art.

Routes of administration of the compositions of this invention include intravenous and subcutaneous injection. Thus, the compositions can be administered “parenterally.” Parenteral administration also includes intramuscular or even intraperitoneal routes. The preparation of an aqueous composition that contains an anti-retroviral agent as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Anti-retroviral agents can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents such as, for example, sugars or sodium chloride. Prolonged absorption of the injectable compositions can be brought about by the use in the compositions of agents delaying absorption such as, for example, aluminum monostearate and gelatin.

Sterile injectable Solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for intramuscular injection is also contemplated. This is envisioned to have particular utility in e.g., facilitating the treatment of needle stick injuries of health care workers. In this regard, the use of DMSO as a solvent is possible as this will result in extremely rapid penetration, delivering high concentrations of the active agents to a small area.

Upon formulation, solutions will be administered in a manner compatible with the dosage formulation and in such an amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580, incorporated by reference herein in its entirety). Some variation in dosage will necessarily occur depending on the various parameters, such as the age, gender, race, size and overall condition of the subject being treated, as well as on the particular agent being administered and the condition to be treated. The person responsible for administration will, in any event, determine the appropriate dose for the individual subject, according to protocols well known in the art.

In addition to the compounds formulated for parenteral administration, such as intravenous or intramuscular injection, other pharmaceutically acceptable forms include, e.g., tablets or other solids for oral administration; time release capsules; and any other form currently used, including creams, lotions, mouthwashes, inhalants and the like. Upon formulation of any suitable pharmaceutical, administration of therapeutically effective amounts compatible with the dosage formulation will be known to those of ordinary skill in the art in light of the present disclosure.

In certain embodiments, active compounds can be administered orally. This is contemplated for agents that are generally resistant, or have been rendered resistant, to proteolysis by digestive enzymes. For oral administration, the active compounds may be administered, for example, with an inert diluent or with an assimilable edible carrier, or they may be enclosed in hard or soft shell gelatin capsule, or compressed into tablets, or incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Further exemplary suitable treatment methods include the use of nasal solutions or sprays, aerosols or inhalants. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines.

Inhalations and inhalants are pharmaceutical preparations designed for delivering a drug or compound into the respiratory tract of a patient. A vapor or mist is administered to deliver agents into the systemic circulation. Inhalations may be administered by the nasal or oral respiratory routes. Another group of products, also known as inhalations, and sometimes called insufflations, consists of finely powdered or liquid drugs that are carried into the respiratory passages by the use of special delivery systems, such as pharmaceutical aerosols, that hold a solution or suspension of the drug in a liquefied gas propellant. When released through a suitable valve and oral adapter, a metered dose of the inhalation is propelled into the respiratory tract of the patient.

The following examples are included to demonstrate various embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### EXAMPLES

### Example 1

### Prognostic Value of CCL3L1 and CCR5 Genotypes in HIV-1/AIDS

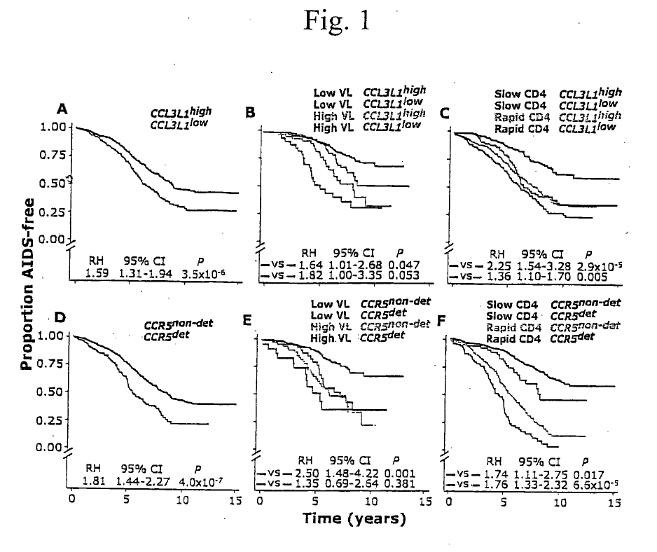

In the combined analyses of the European (EA)- and African (AA)-American subjects of the HIV-1-infected cohort, relative to possession of CCL3L1 gene copy numbers that are equal to or are greater than the population-specific median (designated here as CCL3L1^{high}), those with CCL3L1 gene copies lower than the population-specific median (CCL3L1^{low}) had a rapid rate of disease progression (FIG. 1A, and Table 1). Although CCL3L1^{low} was associated with a higher viral set point and rapid rate of CD4+ T cell decline, this genotype stratified individuals with high or low viral setpoints (FIG. 1B), as well as rapid or slow rates of CD4+ T cell declines (FIG. 1C) into distinct subsets. These findings indicated that CCL3L1^{low} may offer independent prognostic value that complements that conveyed by the well-established predictive laboratory markers of disease progression.

The CCR5 haplogroup pairs (genotypes) that influence rate of disease progression have been shown to be population-specific (10). These population-specific, disease-accelerating CCR5 haplogroup pairs were combined into a single category representing the “detrimental” CCR5 genotypes, and designated as CCR5^{det}; the remaining CCR5 genotypes were classified as CCR5^{det} (Table 2). Compared to possession of CCR5^{non-det}, CCR5^{det} was associated with a higher baseline CD4+ T cell count (P=4×10⁻⁵), a steeper rate of T cell decline (P=0.0321), higher viral set point (P=0.015), and rapid rate of disease progression (FIG. 1D). CCR5^{det} stratified individuals with low viral setpoints (FIG. 1E), as well as rapid or slow rates of CD4+ T cell declines (FIG. 1F) into distinct groups, suggesting that akin to CCL3L1^{low}, CCR5^{det} may also have independent prognostic value in HIV-1+ individuals.

To assess whether the strategy of dichotomizing the CCR5 genotypes and CCL3L1 gene copies was robust to sampling variations, bootstrap samples from the entire WHMC cohort were used, and a determination was made regarding whether the disease-influencing effects observed with the CCR5 and CCL3L1 risk groups in the entire cohort versus 1,000 bootstrap samples derived from 70% of the entire cohort (n=792) were similar. The 95% confidence intervals for the relative hazards for the risk of developing AIDS for the entire cohort and those for the bias-corrected estimates from the bootstrap samples were similar, suggesting that this approach of dichotomizing CCR5 genotypes and CCL3L1 gene copy numbers was both valid and robust (Table 3).

### CCL3L1^{low}CCR5^{det}: a Genetic Marker of Enhanced HIV-1 Susceptibility

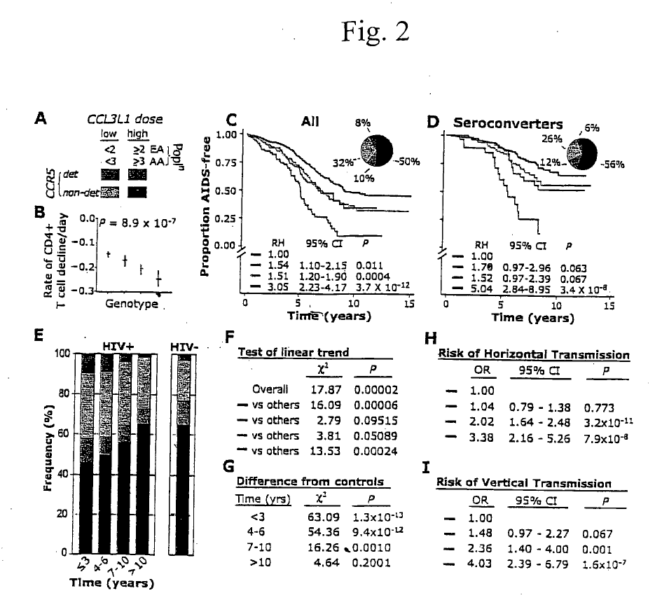

To determine the individual and combined disease- and transmission-influencing effects of variation in CCL3L1 and CCR5, we stratified the cohort into four mutually exclusive genotypic groups on the basis of possession of CCL3L1^{high} or CCL3L1^{low}, and CCR5^{non-det} or CCR5^{det} (FIG. 2A). CCL3L1^{low}CCR5^{det} reflects the combined adverse effects of a population-specific low CCL3L1 dose and detrimental CCR5 genotype, whereas CCL3L^{high}CCR5^{non-det} reflects the converse; CCL3L1^{low}CCR5^{non-det} and CCL3L1^{high}CCR5^{det} reflect the individual effects of CCL3L1 and CCR5, respectively. Of the four CCR5/CCL3L1 genotypic groups, CCL3L1^{high}CCR5^{non-det} and CCL3L1^{low}CCR5^{det} were at the two extremes of disease susceptibility as they were associated with the lowest and highest point estimates for baseline VLs, respectively, and slowest and fastest rates of CD4+ T cell declines (P=8.2×10⁻⁵; FIG. 2B), and rates of disease progression (FIG. 2, C-D). Strikingly, compared to CCL3L1^{low}CCR5^{non-det} or CCL3L1^{high}CCR5^{det}, possession of CCL3L1^{low}CCR5^{det} was associated with a nearly 2- to 3-times higher risk of progressing rapidly to AIDS (FIG. 2, C-D). These findings highlighted the additive detrimental effects of these two loci, demonstrating that in HIV-1 infection, the combined relationship of the receptor and ligand is more powerful than either genetic variable examined in isolation.

Within a prospective, well-characterized HIV-1+ cohort, slow and rapid progression to AIDS is characterized by distinct CCL3L1/CCR5-based distribution profiles (FIG. 2E). A genomic signature of rapid disease progression is over- and under-representation of CCL3L1^{low}CCR5^{det} and CCL3L1^{high}CCR5^{non-det} respectively (FIG. 2E). With increasing AIDS-free survival, maximal changes occur in the frequency distribution of these two genotypic groups (χ² of 13.53 and 16.09; FIG. 2F), and this is reflected in the progressive reduction in the frequency of CCL3L1^{low}CCR5^{det} and increase in the frequency of CCL3L1^{high}CCR5^{non-det} in persons living without AIDS for increasing lengths of time (FIG. 2E). Additionally, there is a step-wise decline in the chi-square and significance values for the comparisons of the frequency distributions of the CCL3L1/CCR5 genotypic groups in HIV-1+ individuals with increasing AIDS-free survival times and an ethnically-matched cohort of HIV-1-negative individuals (n=1,031; FIG. 2G). Thus, the distribution of CCL3L1/CCR5 genotypic groups in persons living without AIDS for increasing lengths of time will progressively change, and begin to resemble gradually that of HIV-1 negative individuals.

With increasing AIDS-free survival there was also a progressive reduction in the prevalence of CCL3L1^{low}CCR5^{non-det}, however, there were only modest, but non-significant changes in the individuals who also possessed low CCL3L1 gene copies. distribution of CCL3L1^{high}CCR5^{det} (FIGS. 2, E and F). Collectively, these findings indicate that within a representative HIV-1+ cohort, there is a hierarchy in the rate at which individuals progress to AIDS. This is dependent, in part, on their CCL3L1/CCR5 genetic make-up: CCL3L1^{low}CCR5^{det}>CCL3L1^{low}CCR5^{non-det}≧CCL3L1^{high}CCR5^{det}>CCL3L1^{high}CCR5^{non-det}. These findings also indicate that the effects of CCR5^{det} are evident mainly in the context of individuals who also possessed low CCL3L1 gene copies.

The hierarchy of the CCL3L1/CCR5 genotypic groups that influenced the risk of acquiring HIV-1 and rate of disease progression in adults was similar, and notably, only genotypes that contained CCL3L1^{low} were associated with a significantly higher risk of adult-to-adult (horizontal) transmission (FIG. 2H). Extending and confirming these findings, only CCL3L1^{low}-containing CCL3L1/CCR5 genotypic groups were associated with a significantly higher risk of acquiring HIV-1 than those containing only CCR5^{det} in a cohort of children exposed perinatally to HIV-1 (FIG. 2I). Thus, relative to genotypes that only contain CCR5^{det}, those containing CCL3L1^{low} with or without CCR5^{det} may play a more dominant role in modulating HIV-1 infection in populations from which these cohorts were derived.

A determination was made regarding whether it was optimal to use four versus three genetic risk groups for purposes of prognostication of the risk of rapid disease progression. This analysis indicated that a genetic risk group stratification system that has three CCL3L1/CCR5-based genotypic groups and in which CCL3L1^{high}CCR5^{det} and CCL3L1^{low}CCR5^{non-det} are placed into a single category, was optimal for determining the AIDS prognostic value of variations in CCL3L1 and CCR5.

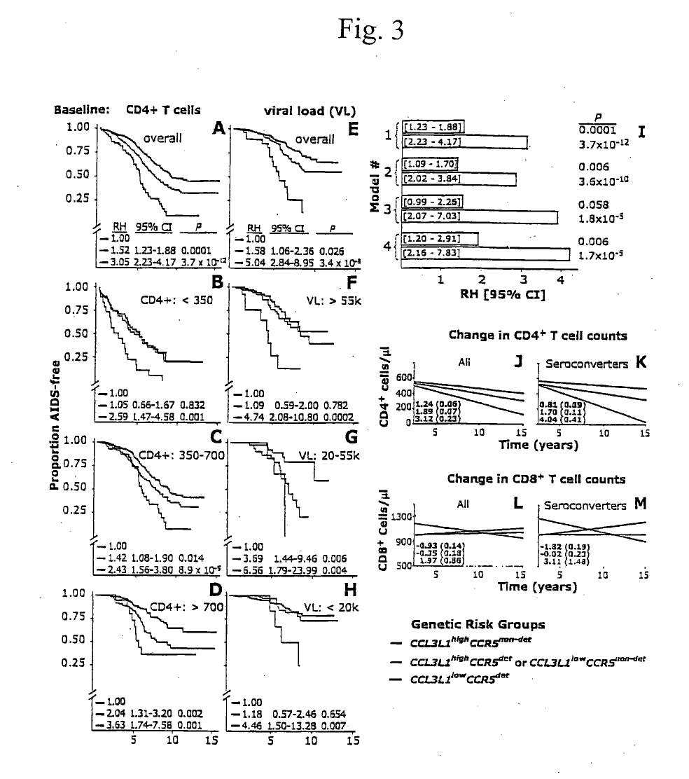

Possession of CCL3L1^{low}CCR5^{det} provided robust discrimination of both time to AIDS and death, with a nearly 2.43- and 6.56-fold greater risk of progression to AIDS before or after stratification for a range of baseline CD4+ T cell counts or VLs (FIG. 3, A-H). The disease-accelerating effects of CCL3L1^{low}CCR5^{det} were also independent of the rate of CD4+ T cell decline and the baseline VLs when it was included in the model as a time-varying covariate (FIG. 31). Notably, the disease-accelerating effects of CCL3L1^{low}CCR5^{det} were evident at CD4+ T cell counts (<350 or >700 cells/μl) or VLs (>55,000 or <20,000 copies/ml) that begin considerations for initiation of antiviral therapy or suggest a low risk for development of AIDS (FIG. 3, A-H). In some instances, the genetic risk group that reflects the individual effects of CCL3L1 or CCR5 (KM plots in FIG. 3, A-H) was also associated with disease-influencing effects that were independent of baseline CD4+ counts and VLs.

Although HIV-1-induced immune activation involves both CD4+ and CD8+ T cells, only peripheral blood total CD4+ T cell numbers decline gradually; total CD8+ T cell numbers typically remain elevated until late into HIV-1 infection (15). Higher baseline CD8+ T cells (16), and a subsequent rapid fall of circulating CD8+ T cells is a strong predictor of developing AIDS in longitudinal cohort studies (16, 17). In this context, it was notable that possession of CCL3L1^{low}CCR5^{det} was associated with a similar cellular profile: compared to the other genotypic groups, CCL3L1^{low}CCR5^{det} was associated with higher starting CD8+ T cell counts, and, in most instances, this was followed by a rapid decline in not only CD4+, but also CD8+ T cells before and after stratification of baseline CD4+ T cell counts and VLs (FIG. 3, J-M).

Likelihood ratios (LRs) are a widely used index for evaluating the prognostic performance of a diagnostic test at the level of the individual patient (18). To account, in part, for lead-time and length bias, the LRs of baseline CD4+ T cell counts, initial VLs, and the genetic risk groups were directly compared in two settings: first, in a prospective HIV+ cohort, and second, after matching AIDS cases with those who did not develop AIDS, with the expectation that the LRs estimated from prospectively derived data would be lower than those obtained from a nested case-control study.

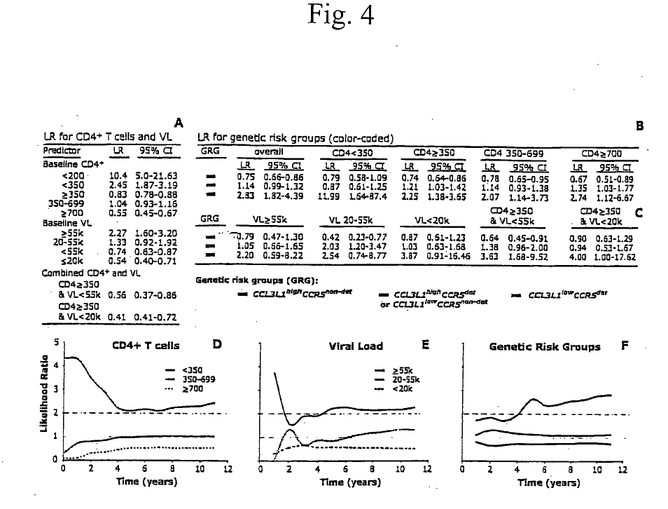

FIG. 4A shows the LRs for different strata of baseline CD4+ counts and/or VLs prior to consideration of the genetic background of the HIV+ individuals. Baseline CD4+ counts of more than 350 cells/μl or initial VLs of less than 55,000 copies/ml discriminated between those who did not develop AIDS versus those who did. However, as would be expected, the discriminatory power improved further at baseline CD4+ T cell counts higher than 700 cells/μl or VLs of less than 20,000 copies/ml.

To determine if the likelihood of developing AIDS in individuals with different genetic risk groups is independent of the laboratory markers, LRs were computed before and after stratifying for varying baseline CD4+ T cell counts and viral set points (FIG. 4, B-C). In both the unstratified (overall) and stratified analyses, possession of CCL3L1^{high}CCR5^{non-det} was associated with a lower likelihood of developing AIDS. Notably, the LR of this genetic risk group was similar to that for baseline CD4+ counts of more than 350 cells/μl or VLs lower than 55,000 copies/ml (FIG. 4, A-C). Additionally, the LR for baseline VLs between 20-55,000 copies/ml was not associated with meaningful prognostic information (1.33; 95% CI=0.92-1.92), however, individuals with these VLs that possessed CCL3L1^{high}CCR5^{non-det} had a significantly lower LR (0.42; 95% CI=0.23-0.77), indicating that this genetic group discriminates for a lower likelihood of developing AIDS when laboratory markers such as the baseline VL do not.

Possession of CCL3L1^{low}CCR5^{det} was associated with an increased likelihood of developing AIDS, before or after accounting for different baseline CD4+ counts or VLs (FIG. 4, B-C). Notably, CCL3L1^{low}CCR5^{det} identified individuals with a higher likelihood of developing AIDS when laboratory markers predicted a decreased likelihood of developing AIDS (e.g., CD4+ count ≧700 cells/μl) as well as when these laboratory markers failed to provide robust prognostic information (e.g., 350-699 CD4+ cells/μl or 20-55,000 HIV-1 copies/ml).

Among the different strata of laboratory markers examined (FIG. 4A), the least likelihood of developing AIDS was for individuals with both a low baseline VL (≦20,000 copies/ml) and a high baseline CD4+ count (≦350 cells/μl; FIG. 4A). However, these individuals can be genetically risk-stratified. Possession of CCL3L1^{low}CCR5^{det} swung the pendulum of susceptibility from a reduced to an enhanced risk of AIDS as it was associated with a nearly 10-times higher likelihood of developing AIDS (LR=4.0) relative to the lower likelihood predicted by a high CD4+/low VL setting (LR=0.41). At the other extreme of the susceptibility range, although CD4+ T cell counts of less than 350 cells/μl were associated with a nearly 2.45-times higher likelihood of developing AIDS, in this group CCL3L1^{low}CCR5^{det} identified a subset of individuals with a nearly 12 (LR=11.99) times higher likelihood of developing AIDS.

The genetic marker [CCL3L1^{high}CCR5^{det} or CCL3L1^{low}CCR5^{non-det}] was associated with only a modest increased likelihood of developing AIDS across most CD4+ T cell strata, and the highest LR (2.03) was in individuals with baseline VLs of 20,000-50,000 copies/ml (FIG. 4, B-C). Thus, the genetic markers CCL3L1^{high}CCR5^{non-det} and CCL3L1^{low}CCR5^{det} provided predictive information across the spectrum of AIDS vulnerability by consistently tracking individuals with reduced or increased likelihoods of developing AIDS, respectively, and also in instances when either the laboratory markers predict a contrary likelihood of developing of AIDS or are non-informative.

In the context of a prospective cohort, the time-sensitivity of the LRs for the laboratory and genetic markers were determined by computing their LRs at the end of each year of follow-up in the cohort (FIG. 4, D-F). The prognostic values of CD4+ T cell counts as well as viral setpoints were higher early after infection and then decreased (FIG. 4, D-E). In contrast, the LRs for the three genetic risk groups were stable over time, demonstrating the time-insensitivity of their prognostic value (FIG. 4F). Notably, the LRs for genetic risk groups associated with a high, intermediate or low likelihood of developing AIDS were comparable to the LRs for the three different CD4+ T cell and VL strata that are typically used as cut-offs for risk assessment of HIV+ patients (2, 3), indicating that the laboratory and genetic markers are providing quantitatively similar predictive value with respect to the likelihood of developing AIDS (FIG. 4, D-F).

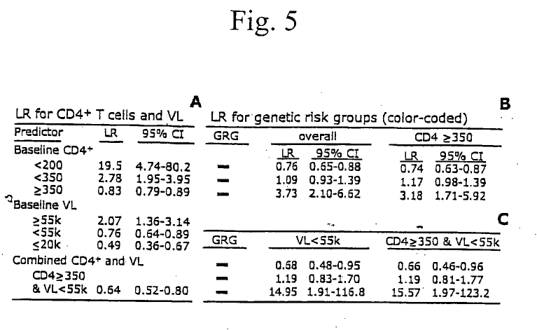

The LRs for the laboratory and genetic markers in the nested case-control study supported strongly the aforementioned in that CCL3L1/CCR5 genetic risk groups provided prognostic power independent of baseline CD4+ counts or VLs (FIG. 5). In particular, the possession of CCL3L1^{low}CCR5^{det} was associated with a nearly 24 times increased a posteriori likelihood (LR=15.57; FIG. 5C) of developing AIDS compared with an a priori LR of 0.64 that is based on a CD4+ count of greater than 350 cells/μl and a VL of less than 55,000 copies/ml (FIG. 5A). It is striking that the LR for developing AIDS in individuals with CCL3L1^{low}CCR5^{det} in this setting of a high CD4+/low VL (FIG. 5C) is nearly comparable to the LR of developing AIDS in persons with a CD4+ count of less than 200 cells/μl (FIG. 5A), again emphasizing the capacity of this genotype to change the compass of vulnerability from reduced to significantly enhanced AIDS risk. Conversely, CCL3L1^{high}CCR5^{non-det} was associated with a lower likelihood of developing AIDS at different baseline CD4+ counts and VLs (FIG. 5, B-C).

Three additional analyses (Tables 4, 5 and 6), each computing different aspects of risk stratification also highlighted the independent predictive value of the genetic and laboratory markers. First, the R_{M}² values were comparable for the genetic and laboratory markers (˜5 percent), indicating that they each accounted for a similar proportion of the variability in disease progression (Table 4). Additive effects on the R_{M}² were observed when the genetic risk groups were added to the model that included baseline CD4+ T cells and/or VLs, demonstrating that these three variables are tracking different components of HIV-1 disease pathogenesis (Table 4). Second, by estimating the Gaussian error term (σ; (19)) the predictive prognostic accuracy of the laboratory markers and genetic risk groups was found to be comparable before or after accounting for different baseline CD4+ T cell or VL cut-offs (Tables 4, 5 and 6).

Third, as a measure of clinical utility, the Hartz's overlap index (20) was also determined, which is derived from estimates of the area under receiver operating characteristic curve (21). The highest prognostic information content for predicting AIDS at 3 years by a single prognostic marker was by baseline CD4+ T cell counts (46.5%; Table 4). However, the hierarchy of the information content provided by these markers for their ability to predict risk of developing AIDS for the entire study period or at 7 years was viral setpoint>genetic risk>baseline CD4+ T cell count (Table 4).

Similar analyses were conducted after stratifying the study population based on their baseline CD4+ T cell counts or viral set points (Tables 5 and 6). In individuals with CD4+ T cell counts lower than 200 cells/μl, the baseline VLs and the genetic markers each accounted for 17.5% and 15.4%, respectively of the variability in rate of disease progression, whereas the contribution of the low CD4+ T cells to this variability was minimal (Tables 5 and 6). Remarkably, at this CD4+ T cell stratum, the information content of the laboratory and genetic markers together was 100%. At the other extreme, in individuals with baseline CD4+ T cell counts ≧700 cells/μl, along with CD4+ T cells and VL, the genetic markers served as an independent prognostic determinant with comparable contributions to the variability in an individual's disease progression, similar or higher precision in their predictive power, and importantly providing substantial prognostic information content (Table 5). Analogous findings were obtained when the study population was stratified according to baseline VLs (Table 6). This was especially striking in individuals whose baseline VLs were between 20,000-55,000 copies/ml (Table 6). In this group, genetic markers accounted for nearly 13% of the variability in an individual's disease course, whereas baseline VLs and CD4+ T cell counts accounted for only 4% and 7.5%, respectively (Table 6).

Collectively, these findings establish the cardinal importance of the CCL3L1-CCR5 double-punch in HIV-1 pathogenesis. Using several different approaches, in this prospective study of HIV-1 infected individuals, it was found that a compound genetic marker—CCL3L1^{low}CCR5^{det}—that reflects the joint effects of variation in CCR5 and CCL3L1 is a strong predictor of the risk of developing AIDS, rapid disease progression, and accelerated loss of CD4+ and CD8+ T cells. The predictive capacity of CCL3L1 and CCR5-based genetic risk stratification is not only independent of, but more importantly, comparable to the prognostic information provided by currently used predictors of AIDS risk, implying that genetic markers such as CCL3L1^{low}CCR5^{det} are associated with enhanced risk over and above that reflected by these measured laboratory traits.

These findings indicate that CCL3L1^{low}CCR5^{det} is linked to essential, but differing elements of disease pathology that mediate variable rates of T cell loss and/or the viral setpoint. These results have significant public health implications both in terms of the prediction of the risk of AIDS and in terms of the clinical management of HIV+ patients. Because these data indicate that CCL3L1/CCR5 genotypes may capture different aspects than the traditional components of AIDS risk reflected in the laboratory markers used to assess disease status, they support the hypothesis that genetic risk stratification of infected patients may have an important role in the global risk prediction of HIV-1 disease. Additionally, genetic risk stratification may help navigate better this dilemma in the clinical management of HIV-1 infection, especially since the prevalence of the genetic factors identified in HIV+ individuals is large enough (˜8%; insets in FIG. 2, C-D) to be of clinical relevance in this disease. The prognostic value of the CCL3L1/CCR5-based genetic markers may be especially helpful in individuals with baseline CD4+ T cell counts (350-700 CD4+ cells/μl) or VLs (20−55×10³ copies/ml) that can pose therapeutic dilemmas.

An additional advantage of genetic risk-stratification is the time-insensitive nature of the genetic markers, implying that a single measurement can provide lifelong prognostic information during any phase of the disease, including in patients who present early during their disease course with high baseline CD4+ T cells or low VLs.

The term “HIV-1/AIDS vulnerable individual” is proposed here to define subjects whose genetic makeup enhances their risk at time of virus exposure, i.e., for acquiring HIV-1, and this genetic-based risk persists post-infection. This concept of the “HIV-1 vulnerable individual” is highly analogous in perspective to the “cardiovascular vulnerable patient” who has a high likelihood of developing cardiac events, prompting the recent call to employ comprehensive risk-stratification tools to identify such patients (28, 29). By analogy, these findings suggest that post-infection, in addition to the traditional markers of vulnerability (baseline CD4+ T cell count/VLs and rate of CD4+ T cell loss), the inclusion of a measure of genetic risk might offer an adjunctive, and complementary risk-stratification tool capable of an improved method for identifying persons at high risk for future AIDS related events. These HIV-1 vulnerable individuals are ideal candidates for preventive HIV-1 care that may involve a closer follow-up of these individuals, and potentially, earlier initiation of HAART.

### REFERENCES FOR EXAMPLE 1

2. P. G. Yeni et al., Jama 288, 222 (2002).
3. M. Dybul, A. S. Fauci, J. G. Bartlett, J. E. Kaplan, A. K. Pau, MMWR Recomm Rep 51, 1 (2002).
10. E. Gonzalez et al., Proc Natl Acad Sci USA 96, 12004 (1999).
15. A. J. McMichael, S. L. Rowland-Jones, Nature 410, 980 (2001).
16. D. Kvale, P. Aukrust, K. Osnes, F. Muller, S. S. Froland, Aids 13, 195 (1999).
17. J. V. Giorgi, R. Detels, Clin Immunol Immunopathol 52, 10 (1989).
18. E. J. Gallagher, Ann Emerg Med 31, 391 (1998).
28. M. Naghavi et al., Circulation 108, 1772 (2003).
29. M. Naghavi et al., Circulation 108, 1664 (2003).

### Example II

### Prognostic Value of CCL3L1 and CCR5 Genotypes in HIV-1/AIDS

The contribution of variations in CCL3L1 and CCR5 in the variable risk of acquiring HIV-1 infection or rate of disease progression to AIDS or death was determined in the HIV-infected adult subjects from Wilford Hall Medical Center (WHMC; n=1,132) (1-4). This cohort of infected adults is a single-site cohort composed of individuals with equal access to health care and minimal loss to follow-up, minimizing these confounding factors (1).

To determine the influence of the variations of these two loci in the risk of acquiring HIV-1 in adults, the distribution of the different CCL3L1 and CCR5 genotypic groups in HIV+ EAs (n=621), AAs (n=410) and HAs (N=69) and an ethnically-matched cohort of HIV-1-negative individuals (n=1,031) was compared. The latter HIV-1-negative cohort was also derived from WHMC. The influence of CCL3L1/CCR5 genotypic groups in the risk vertical transmission in a cohort of 558 children exposed perinatally to HIV-1 infection (HIV+n=324; HIV−n=234) was also examined.

Detailed descriptions of the WHMC HIV+ and HIV− cohorts as well the cohort of children exposed perinatally to HIV-1 are as described previously (2, 5). Additionally, the nomenclature for CCR5 haplotypes (6), and their associated phenotypic effects in infected adults (2) have been described and are briefly reviewed below to provide the appropriate context for the approach used in this study. The voluntary, fully informed consent of the subjects used in this research was obtained as required by Air Force Regulation (AFR) 169-9 and approval from the Institutional Review Boards of both WHMC and The University of Texas Health Science Center at San Antonio.

In a separate study, the following was observed. (a) In uninfected European Americans (EA), the median number of CCL3L1 gene copies was 2, and possession of <2 CCL3L1 gene copies was associated with an enhanced risk of acquiring HIV-1 infection. (b) In uninfected African Americans (AA), the median CCL3L1 gene copy numbers was 4, and possession of <4 CCL3L1 gene copies was associated with an enhanced risk of acquiring HIV-1 infection. (c) In uninfected Hispanic Americans (HA), the median CCL3L1 gene copy numbers was 3, and possession of <3 CCL3L1 gene copies was associated with an enhanced risk of acquiring HIV-1 infection. (d) The median number of CCL3L1 gene copies in the HIV+ EAs and AAs was 2 and 3, respectively, and possession of <2 and <3 CCL3L1 gene copies was associated with rapid disease progression to AIDS and death in EAs and AAs, respectively. (e) In infected children of Argentine descent (5), the median number of CCL3L1 gene copies is 2, and possession of <2 CCL3L1 gene copies was associated with an increased risk of acquiring HIV-1 infection.

These findings provided the rational basis to dichotomize the CCL3L1 genotypes into the groups designated as “CCL3L1^{low}” denoting possession of CCL3L1 gene copies lower than the population-specific median, and “CCL3L1^{high}” denoting CCL3L1 gene copies equal to or greater than the population-specific median into two categories (Table 7).

An evolutionary based strategy was used to organize a group of SNPs in the non-coding region of CCR5 along with the CCR5-Δ32 mutation and the CCR2-64I polymorphism in the coding regions of CCR5 and CCR2, respectively, into unique CCR5 haplotypes and to predict the relationships among these haplotypes (6). This strategy classifies CCR5 haplotypes that share a common evolutionary history into one of seven groups of human haplotypes [i.e., so-called human haplogroups (HH)], and is illustrated in Table 7. Defining these haplogroups (HHA-HHG) facilitated our genotyping of the WHMC cohort of adults, children exposed perinatally to HIV-1 infection, and world-wide populations (2, 3, 5). The phylogenetic network of CCR5 haplotypes helps to lessen haplogroup/haplotype misclassification (6-8) and facilitates genotype-phenotype analyses. This nomenclature has also been adopted by other investigators (9-11).

Several CCR5 haplotype pairs have been found to be associated with altered rates of disease progression in HIV-1-infected adults, and the haplotypes and haplotype pairs that influenced the rate of HIV-1 disease progression in EAs were distinct from those in AAs, suggesting a population-specific effect (2). Additionally, the transmission-influencing effects of CCR5 haplotypes/haplotype pairs in children exposed perinatally to HIV-1 (5) has been described. The transmission-influencing effects of CCR5 haplotypes in the WHMC cohort have not been described previously, and in adults the CCR5 genotype associated with altered risk of acquiring HIV-1 is mostly for those that contain the CCR5-Δ32 mutation (12).

Relative to CCR5, CCL3L1 is an easier gene system to categorize (e.g., < or ≧ than population median), with larger numbers of subjects in each category. In contrast, with 9 haplogroups of CCR5 (HHA through HHG*2), a total 45 theoretical haplogroup pair (genotype) combinations exist, and as indicated only a few exhibit disease retardation or acceleration, and the rest are not associated with any major disease-influencing phenotype (2, 6). Thus, the use of a single or pair of CCR5 genotypes that is associated with maximal disease-influencing effects may underpower the overall effects and contributions associated with variation in CCR5 (because of the small numbers of subjects that have these genotypes). This formed the rational basis to dichotomize these 45 CCR5 genotypes into two groups: those associated with a disease-accelerating effect, i.e., are detrimental (det) versus those that are not, i.e., are non-detrimental (non-det). These two groups are designated as CCR5^{det} and CCR5^{non-det}, respectively.

To accomplish this dichotomization of CCR5 genotypes, the overall 95% CI around the median time-to-event was determined, in this case AIDS and death for the HIV+ EA, AA, or Hispanic (HA)-American subjects regardless of their CCR5 genotype (i.e., overall median). We then determined the median time-to-event (AIDS and death) for each CCR5 haplotype pair in these populations. Those population-specific genotypes whose median time-to-event (AIDS and death) were below the lower limit of the 95% CI around the overall median time to event were combined. This group of CCR5 genotypes associated with a rapid rate of disease progression to both AIDS and death was designated as the “detrimental” CCR5 genotypes, i.e., CCR5^{det}, and the others were classified as CCR5^{non-det}. Note, the CCR5 genotype selected by this analysis had to be associated with a faster rate of progression to AIDS and death to be included in the CCR5^{det} category, i.e., the association had to be consistent for both events, thus increasing the likelihood that there is a consistent association between these genotypes and an accelerated disease course.

Illustrating this approach, the overall median time (95% CI) to AIDS in EAs and AAs regardless of the CCR5 genotype was 7.79 yr (6.95-8.45) and 8.37 yr (7.35-10.31), respectively, and the median time (95% CI) to death in EAs and AAs was 8.71 yr (8.16-9.26) and 9.58 yr (8.61-11.34). It was found that in EAs CCR5-HHE/HHE, -HHD/HHE, -HHC/HHG*1, -HHA/HHA, -HHE/HHF*1 and -HHF*2/HHG*1 (n=100, 16.3%) had median times to AIDS and death that were lower than this overall EA population-specific median, and they were therefore classified as CCR5^{det} (Table 2). Previously (2), we found that the repertoire of genotypes that influenced disease progression in EAs and AAs was not identical, and in AAs, the detrimental CCR5 genotypes were CCR5-HHC/HHE, -HHC/HHC, -HHC/HHD, -HHB/HHC, -HHA/HHF*1, -HHD/HHG*1 and -HHE/HHG*2 (n=84, 20.7%; Table 2).

Possession of the CCR5-HHE/HHE has been shown to be associated with the maximal rates of disease acceleration in EAs (2). In contrast, in AAs, CCR5-HHC/HHC and CCR5-HHC/HHD haplotype pairs were associated with the maximal rate of disease progression in this population (2). To ensure the appropriate grouping of detrimental CCR5 genotypes, a comparison was done of the disease course associated the haplotype pairs associated with maximal rates of disease progression that we had reported on before with the other genotypes that also had an accelerated disease course in either EAs or AAs, and had been included in the CCR5^{det} category. In EAs, the accelerated disease course associated with possession of CCR5-HHE/HHE was similar to the other genotypes whose median time to events (AIDS and death) was lower than the overall EA population-specific median. Similarly, in AAs, the disease course associated with possession of CCR5-HHC/HHC or -HHC/HHD was similar to the other genotypes whose median time to event (AIDS and death) was lower than the overall AA population-specific median. The findings observed with CCR5^{det} and CCR5^{non-det} were consistent with those obtained when only the population-specific CCR5 genotypes associated with maximal rates of disease progression were included in the analyses. In children exposed perinatally to HIV-1, possession of the CCR5-HHE haplotype is associated with an increased risk of acquiring HIV-1 (5), and is thus designated as CCR5^{det}.

Based on the possession of population-specific detrimental CCR5 genotypes and/or CCL3L1 gene copy numbers lower than population-specific median, four mutually exclusive genotypic groups exist: (a) possession of neither CCL3L1 gene copies lower than the population-specific median or detrimental CCR5 genotypes (CCL3L1^{high}CCR5^{non-det}). This is the reference group; (b) possession of detrimental CCR5 genotypes, but not CCL3L1 gene copies lower than the population-specific median (CCL3L1^{high}CCR^{det}); (c) possession of CCL3L1 gene copies lower than the population-specific median, but not detrimental CCR5 genotypes (CCL3L1^{low}CCR5^{non-det}); and (d) possession of both CCL3L1 gene copies lower than the population median and detrimental CCR5 genotypes (CCL3L1^{low}CCR5^{det}).

In FIGS. 1 and 2, after stratifying the cohort into (a) CCL3L1^{low} and CCL3L1^{high}; (b) CCR5^{det} and CCR5^{non-det}; and (c) the four CCL3L1/CCR5 genotypic groups, survival analyses (time to AIDS (1987 criteria)) and death for these different genotypic categories were conducted. KM survival curves and the log-rank test were used for between-group analysis. A Cox proportional hazards model was used to estimate the relative hazards (with 95% CI). The validity of Cox proportional hazards modeling assumptions using Schoenfeld residuals was tested. The data obtained using time to AIDS or death were very similar and hence only the findings pertaining to time to AIDS as an endpoint are shown.

The survival analyses were also conducted after stratifying the cohort based on rate of CD+ T cell decline or baseline viral loads (viral setpoint). In this study the terms baseline VL and setpoint are used interchangeably. (HIV-1 RNA levels were determined in the plasma samples of the acutely seroconverting component of the WHMC cohort. These plasma samples corresponded to the first sample available at time of diagnosis of seroconversion.) The change in the distribution frequency of CCL3L1/CCR5 genotypic groups in individuals with varying AIDS-free survival times was assessed using a chi-square test for linear trend.

For the analyses shown in FIG. 3, the optimal genetic risk scoring/stratification system was used. Using the optimized risk scoring system, survival analyses were conducted for progression to AIDS in the entire cohort before and after stratifying for baseline CD4+ T cell counts. Using the optimized risk scoring system, survival analyses were also conducted for progression to AIDS in the seroconverting portion of the cohort before and after stratifying for baseline viral loads (viral setpoint). Finally, the rates of decline of CD4+ T cells and CD8+ T cells were estimated, before and after stratifying for baseline CD4+ T cells and the viral loads. Cox proportional hazards regression analyses were conducted after adjusting for the baseline CD4+ T cell counts as a time-independent, and viral setpoint as a time-dependent covariate in seroconverters as well as the EA and AA portions of the entire cohort (FIG. 31). To assess the risk of acquiring HIV associated with the combination of CCR5 and CCL3L1 genotypes, unconditional logistic regression in the setting of both vertical and horizontal transmission was used.

The predictive value of the CCL3L1/CCR5 genotypic group, baseline CD4+ T cell count and viral set point in the prognosis of HIV-infected EA and AA adult subjects from the WHMC cohort was directly compared by estimating the likelihood ratios, as well as three additional complementary statistical approaches which were as follows. (a) To estimate the amount of explained variation in survival analysis, Cox proportional hazard models were used after assessing the validity of the assumption of proportional hazards; (b) To estimate the accuracy/precision of prognostic prediction by the covariates, parametric failure-time regression for time to AIDS (1987 criteria) was used; and (c) To estimate the information content in predicting the risk of AIDS, unconditional logistic regression for the risk of AIDS was used. For each of these three approaches, seven models using different combinations of the three covariates were used:

(i) viral load only (V only)
(ii) baseline CD4+ T cell count only (C only)
(iii) genetic risk groups only (G only)
(iv) viral load and baseline CD4+ T cell count (V+C)
(v) viral load and genetic risk groups (V+G)
(vi) baseline CD4+ T cell count and genetic risk groups (C+G) and,
(vii) all the covariates (V+C+G).

The measurements of HIV-1 RNA setpoints were available in the seroconverting individuals (n=515, and of these there are 301 EAs (58.45%), 172 AAs (33.40%), and 28 HAs (5.44%), and 14 other racial categories). Of these 515 subjects, complete genotypic data were available on 495 subjects (EA=296; AA=171; and HA=28). Therefore, for a direct comparison of the predictive value of viral set point, baseline CD4+ T cell count and genetic risk groups data from the seroconverters were used.

In the WHMC cohort, the majority of the individuals are either EAs or AAs. Thus, for simplicity, the analyses are for the combined analyses for the EA and AA portions of the infected WHMC cohort. There are two exceptions. First, is for FIG. 2, panels E to H. In the latter instance, the distribution of CCL3L1/CCR5 genotypic groups in HIV+ and HIV-negative individuals that are ethnically matched was compared and, as indicated both cohorts are derived from members of the U.S. Air Force. The proportion of individuals who are EAs, HAs and AAs in the HIV-negative cohort is similar to that of the HIV+ cohort. However, in the HIV-negative WHMC cohort, the EAs and HAs are included in a single category, i.e., non-AAs. Hence, in FIG. 2, panels E-H the analyses are for the EAs, HAs and AAs using appropriate population-specific CCR5 and CCL3L1 detrimental genotypes (lower than the population-median; Tables 1 and 2). The second exception is where the entire epidemiological data of the WHMC cohort from all ethnic groups was captured. Stata 7.0 (Stata Corp., College Station, Tex.) software was used for all statistical analyses.

To optimize the number of genetic risk groups that have prognostic value based on CCR5 genotypes and CCL3L1 gene dose the critical χ² statistic defined as the model χ² divided by its degrees of freedom was used. This statistic indicates the average predictive performance of the number of risk groups included in a multivariate regression model.

As indicated, based on the possession of population-specific CCR5 genotypes and CCL3L1 gene copy numbers, four mutually exclusive combinations exist. These four genetic risk groups were used in following two ways.

(a) Four genetic risk group system is the same as described in FIG. 2A.
(b) Three genetic risk group system classified subjects as high risk if they possessed CCL3L1^{low}CCR5^{det}; moderate risk if a subject possessed either CCL3L1^{high}CCR5^{det} or CCL3L1^{low}CCR5^{non-det}; and low risk if the subject possessed CCL3L1^{high}CCR5^{non-det}.

The predictive value of the risk groups was determined using multivariate Cox proportional hazards regression by comparing each risk group with subjects possessing CCL3L1^{high}CCR5^{non-det}. The risk group system that gave consistently the highest critical χ² was chosen as the most informative with respect to prognostic value. In the context of time to AIDS and time to death for the entire HIV+ WHMC cohort as well as the seroconverting component of the cohort it was observed that the three genetic risk group system was the optimal choice.

The method of Generalized Estimating Equations (GEE) (13-16) was used to estimate the rate of change in the counts for the following leukocyte subsets that include surrogate markers of disease progression in HIV-1 infected adults: total lymphocyte count, CD3+ T cell count, CD4+ T cell count, CD8+ T cell count, % CD3+ T cells and % CD4+ T cells. The CD3− T cell count was used as a negative control to assess the association of the CCR5/CCL3L1 genetic groups with rates of change for these markers. The GEE method is used to estimate population averaged panel-data models (17-24). This approach is an extension of the generalized linear model (GLM) and utilizes the within-subject correlation structure. Using this method, the average monthly rates of decline for the disease markers were estimated. In FIG. 3, positive numbers indicate an overall decline whereas negative numbers indicate an overall increase.

The Likelihood Ratio (LR) is the likelihood that a given test result would be expected in a patient with the target disorder, in this case AIDS compared to the likelihood that that same result would be expected in a patient without the target disorder. LR are frequently employed in clinical settings to assess the utility of the result of a diagnostic test (25-28). Especially in the setting of tests reporting the results in multiple categories (e.g., CD4+ counts <200, 200-349, 350-699 and ≧700), LRs have the advantage of quantifying the diagnostic utility of each test result (26).

If p₁ is the proportion of the n₁ diseased subjects who show a particular test result and p₀ is the proportion of the n₀ non-diseased subjects who show the same test result, then the likelihood ratio is defined as LR=p1/p0. A 95% confidence interval around the likelihood ratio can be estimated

$${e}^{ln ( LR ) \pm 1.96 \sqrt{( \frac{1 - {p}_{1}}{{p}_{1} {n}_{1}} ) + ( \frac{1 - {p}_{0}}{{p}_{0} {n}_{0}} )}}$$

LR confidence intervals straddling unity are indicative of a clinically meaningless test result. Significant departures from unity show an increased (LRs >1) or decreased (LRs <1) likelihood of the disease for a given test result.

The likelihood ratios for different strata of baseline CD4+ T cell count, viral setpoint and genetic risk groups were estimated. To assess the prognostic independence of genetic risk groups, the likelihood ratios for the genetic risk groups were estimated in the context of differing baseline CD4+ T cell counts and viral set-points. Finally, to assess the time-insensitiveness of the LRs, they were estimated at the end of each year of follow-up and spline-smoothed curves were plotted to depict the relationship of LRs with time.

Likelihood ratios are usually interpreted in a diagnostic test evaluation scenario where the target disease status is evaluated at the time of testing (25). In this context, LR is used to predict the existing disease state from the test result. This use of the LR is different from the way the LRs are used above, which was to quantify the predictive ability of baseline CD4+ T cell count, viral setpoint and genetic risk groups for the future development of AIDS. Thus, to complement the aforementioned studies, additional analyses were conducted using a nested case-control design. All ‘cases’ were selected that developed AIDS during follow-up in the WHMC cohort and chose controls from those who did not develop AIDS. Nested case-control studies are, by definition (29), matched studies where the controls are selected so as to match the time of occurrence of AIDS. In addition, ethnic background was used as another matching variable. Given that ˜40% of the cohort developed AIDS during follow-up, one control was selected for each case. Stata 7.0 command (sttocc) was used to create a nested case-control data set. Using the selection criteria stated above, a case-control sample of 434 AIDS cases and 433 non-AIDS HIV-infected control subjects was generated. Using this retrospective data the LRs were estimated and the results compared with those obtained from the prospective cohort. The same analyses were also conducted after stratifying for different baseline CD4+ count and viral load strata.

The significance of association of a covariate with time to event in survival analyses is commonly based on the results of Cox proportional hazards models. These results, however, may not capture the extent to which each of the covariates is contributing prognostically. In these situations, the amount of explained variation is a better measure of the predictive value of a covariate. In generalized linear modeling, the following definition of R² (here referred to as R_{M}²) is defined as:

R_{M}²=1−(L_{R}/L_{U})^{2/n},

where L_{R} denotes the model likelihood without (restricted) covariates and L_{U} represents the model likelihood with (unrestricted) covariates. This definition is equivalent to 1-exp(likelihood ratio χ²/n). If the assumptions of Cox proportional hazards modeling are met and the n represents total observations (rather than number of censored observations) then R_{M}² can be used as a reliable measure of explained variation in survival analyses (30). This measure is also comparable with other measures of explained variation like Schemper's V (30-33), and Kent and O'Quigley's ρ²_{w}. (34). As a rule, the explained variation in survival analyses is low based on this definition (35). This definition of R_{M}² was used to estimate the variation in time to AIDS explained by CD4+ T cell count, viral set point and genetic risk groups based on the CCR5 and CCL3L1 genotypes after testing the validity of Cox proportional hazards modeling assumptions using the Schoenfeld residuals.

Parametric accelerated failure-time regression was used to directly compare the prognostic information content of three covariates: initial viral load (viral set point), baseline CD4+ T cell count and genetic risk groups based on CCR5 and CCL3L1 genotypes. A linear combination of covariates was regressed onto the cube root of the time to event assuming lognormal distribution by using a model of the following form:

Ln(tⱼ^{1/3})=xⱼβ*+zⱼ

where zⱼ follows an extreme-value distribution scaled by an ancillary parameter σ. The ancillary parameter is assumed to be distributed as N(0, 1). An important property of the ancillary parameter is that it takes a value of 0 when there is no error in prediction. Thus, the smaller the value of σ implies a more precise prognostic prediction. The lognormal regression is implemented by setting μⱼ=xⱼβ and treating the standard deviation σ as the ancillary parameter to be estimated from the data. The lognormal hazard, survival and density functions are (36-39):

$$\begin{matrix}h ( i ) = \frac{\frac{1}{i σ \sqrt{2 π}} exp [ \frac{- 1}{2 {σ}^{2}} {\{ ln ( i ) - μ \}}^{2} ]}{1 - Φ \{ \frac{ln ( i ) - μ}{σ} \}} , \\ s ( i ) = 1 - Φ \{ \frac{ln ( i ) - μ}{σ} \} , and \\ f ( i ) = \frac{1}{i σ \sqrt{2 π}} exp [ \frac{- 1}{2 {σ}^{2}} {\{ ln ( i ) - μ \}}^{2} ] .\end{matrix}$$

Nested regression models (Tables 4, 5 and 6) were used to estimate the changes in σ and identify the model that has minimum value of σ associated with it. For the purpose of this analysis the HIV RNA load and baseline CD4+ T cell count were not categorized and these variables were used on an “as is” basis so as to maximize the clinical information contained in these tests.

Treating occurrence of AIDS during the follow-up time as a binary response variable, the association of viral setpoint, baseline CD4+ T cell count and genetic risk groups with the risk of AIDS were logistically modeled. The model-fit of these regression models was tested using receiver-operating characteristic curves which plot sensitivity of prediction against 1-specificity for various cut-off values of the predictor variable. The area under the ROC curve (AUC) represents the overall predictive accuracy and has been shown to be equivalent to the Hosmer-Lemeshow c statistic (40, 41). The c statistic has been previously used by many authors to quantify the predictive value. This statistic was further transformed into a number to ease interpretation based on Hartz's overlap index (42). Given that AUC of 0.5 indicates maximum diagnostic uncertainty and a 1 indicates maximum diagnostic certainty, Hartz's overlap index is equivalent to 1-2(AUC-0.5).

The complement of this index (that is 2(AUC-0.5)), which is equivalent to the Youden's index (43), was used to capture the prognostic information content of the covariates and it was expressed as a percentage. To include the role of time to event in the study cohort, the logistic regression analysis was conducted at three different time points: 3 years of follow-up, 7 years of follow-up and end of study. For each of these analyses only the censored and uncensored observations till the respective time point were included.

### REFERENCES FOR EXAMPLE II

1. S. Mummidi et al., Nat Med 4, 786 (1998).
2. E. Gonzalez et al., Proc Natl Acad Sci USA 96, 12004 (1999).
3. E. Gonzalez et al., Proc Natl Acad Sci USA 98, 5199 (2001).
4. E. Gonzalez et al, Proc Natl Acad Sci USA 99, 13795 (2002).
5. A. Mangano et al., J Infect Dis 183, 1574 (2001).
6. S. Mummidi et al., J Biol Chem 275, 18946 (2000).
7. D. H. McDermott et al., Lancet 352, 866 (1998).
8. M. P. Martin et al, Science 282, 1907 (1998).
9. J. Tang et al., J Virol 76, 662 (2002).
10. J. Tang et al., AIDS Res Hum Retroviruses 18, 403 (2002).
11. P. A. Ramaley et al., Nature 417, 140 (2002).
12. S. J. O'Brien, J. P. Moore, Immunol Rev 177, 99 (2000).
13. A. C. Ghani et al., J Acquir Immune Defic Syndr 28, 226 (2001).
14. K. L. Fielding et al., Stat Med 14, 1365 (1995).
15. S. R. Lipsitz, G. Molenberghs, G. M. Fitzmaurice, J. Ibrahim, Biometrics 56, 528 (2000).
16. J. Roy, X. Lin, L. M. Ryan, Biostatistics 4, 371 (2003).
17. S. L. Zeger, K. Y. Liang, Biometrics 42, 121 (1986).
18. S. L. Zeger, K. Y. Liang, Stat Med 11, 1825 (1992).
19. J. Rochon, Stat Med 17, 1643 (1998).
20. B. C. Supradhar, K. Das, Biometrics 56, 622 (2000).
21. S. R. Lipsitz, G. M. Fitzmaurice, E. J. Orav, N. M. Laird, Biometrics 50, 270 (1994).
22. T. Park, Stat Med 12, 1723 (1993).
23. J. M. Williamson, S. R. Lipsitz, K. M. Kim, Comput Methods Programs Biomed 58, 25 (1999).
24. A. Hadgu, G. Koch, J Biopharm Stat 9, 161 (1999).
25. D. L. Simel, G. P. Samsa, D. B. Matchar, J Clin Epidemiol 46, 85 (1993).
26. N. J. Birkett, J Clin Epidemiol 41, 491 (1988).
27. E. J. Gallagher, Ann Emerg Med 31, 391 (1998).
28. K. L. Radack, G. Rouan, J. Hedges, Arch Pathol Lab Med 110, 689 (1986).
29. B. Langholz, D. C. Thomas, Am J Epidemiol 131, 169 (1990).
30. M. Schemper, J. Stare, Stat Med 15, 1999 (1996).
31. M. Schemper, R. Henderson, Biometrics 56, 249 (2000).
32. M. Schemper, Stat Med 22, 2299 (2003).
33. M. Schemper, Stat Med 12, 2377 (1993).
34. J. T. Kent, J. O'Quigley, Biometrika 75, 525 (1988).
35. E. L. Kom, R. Simon, Stat Med 9, 487 (1990).
36. J. L. Fahey et al., Aids 12, 1581 (1998).
37. M. Shi, J. M. Taylor, A. Munoz, Lifetime Data Anal 2, 31 (1996).
38. M. Shi et al., J Acquir Immune Defic Syndr Hum Retrovirol 12, 309 (1996).
39. Stata Reference Manual Release 7 (Stata Press, College Station, 2001), pp. 1-10.
40. P. M. Ridker, N. Rifai, L. Rose, J. E. Buring, N. R. Cook, N Engl J Med 347, 1557 (2002).
41. D. W. Hosmer, S. Taber, S. Lemeshow, Am J Public Health 81, 1630 (1991).
42. A. J. Hartz, Arch Pathol Lab Med 108, 65 (1984).
43. M. Greiner, J Immunol Methods 185, 145 (1995).

### Example III

### CCL3L1 Gene-Containing Segmental Duplications in HIV-1/AIDS Susceptibility

Segmental duplications in the human genome are selectively enriched for genes involved in immunity, although the phenotypic consequences for host defense are unknown. This study shows that there are significant interindividual and interpopulation differences in the copy number of a segmental duplication encompassing the gene encoding CCL3L1 (MIP-1 αP), a potent HIV-1-suppressive chemokine and ligand for the HIV coreceptor CCR5. Possession of a CCL3L1 copy number lower than the population average is associated with markedly enhanced HIV/AIDS susceptibility. This susceptibility is even greater in individuals who also possess disease-accelerating CCR5 genotypes. This relationship between a variable CCL3L1 dose and altered HIV/AIDS susceptibility points to a central role for CCL3L1 in HIV/AIDS pathogenesis, and indicates that differences in the dose of immune response genes may constitute a genetic basis for variable responses to infectious diseases.

Duplicated host defense genes that are known to have dosage effects are thought to contribute to the genetic basis of some complex diseases, although direct evidence for this is lacking. Human chromosome 17q encompasses two CC chemokine genes, CC chemokine ligand 3-like 1 (CCL3L1; other names, MIP-1 αP and LD78β) and CCL4L1 (MIP-1β-like), which represent the duplicated isoforms of CCL3 and CCL4, respectively (1-3). As a consequence, the copy number of CCL3L1 and CCL4L1 varies among individuals ((2, 3). This is relevant because CCL3L1 is the most potent of the known ligands for CC chemokine receptor 5 (CCR5), the major coreceptor for HIV, and a dominant HIV-suppressive chemokine (3).

An initial determination was made of the distribution of chemokine gene-containing segmental duplications in 1,064 humans from 57 populations, and 83 chimpanzees (4). An analysis of 4,308 HIV-1-positive (HIV⁺) and -negative (HIV⁻) individuals from different ethnic groups was done to determine if the risk of first, acquiring HIV, and second, the rate at which HIV disease progressed were sensitive to differences in the dose of CCL3L1 gene-containing segmental duplications ((4).

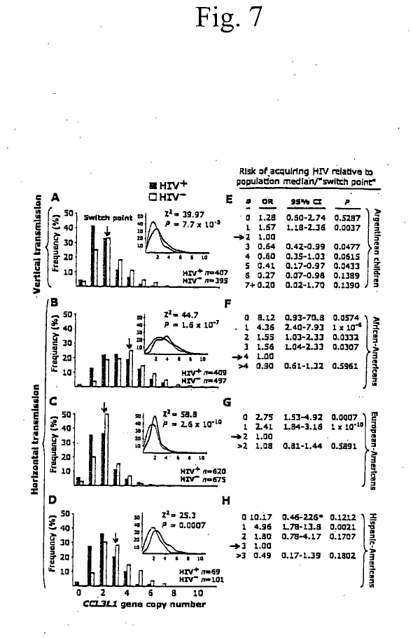

African populations possessed a significantly greater number of CCL3L1 gene copies than non-Africans (FIG. 1). The geographic region-of-origin explained nearly 35% of the total variation in the distribution of CCL3L1 gene copies (analysis of variance: F=94.41, df=6, 1037; P=1.23×10⁻⁹⁴). Corroborating this, in separate cohorts of HIV⁻ subjects, there were significant inter-individual and inter-population differences in CCL3L1 copy numbers. The median copy number in HIV⁻ Argentinean children was two, and in HIV⁻ African (AA)-, European (EA)-, and Hispanic (HA)-American adults, it was four, two, and three, respectively (FIG. 7, A to D, open bars). This demonstrated that variations in CCL3L1 gene dose exist among populations.

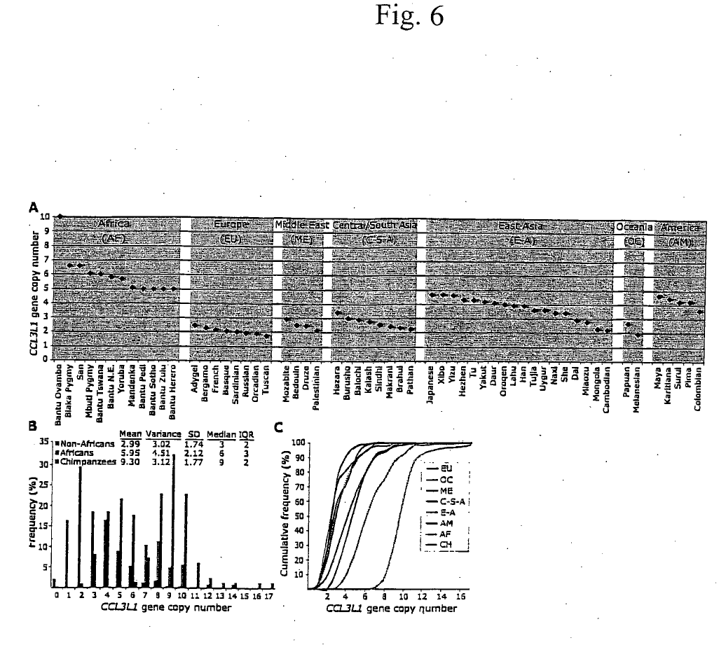

The duplicated region encoding human CCL3L1 has an ancestral correlate in chimpanzee. There were significant differences between species and among human populations in the frequency of chemokine gene-containing segmental duplications. Nevertheless, the dispersion around the average copy number was similar in both human populations and chimpanzees (FIG. 6B). Based on this observation, the plausibility that it is not the absolute copy number per se, but rather the gene dose relative to the average copy number in each population that confers HIV/AIDS susceptibility was considered.

Several lines of evidence indicated that possession of a low CCL3L1 copy number was a major determinant of enhanced HIV susceptibility among individuals from four different human populations and in the setting of two different modes of acquiring HIV: mother-to-child and adult-to-adult. Individuals with a low CCL3L1 gene copy number were overrepresented among the HIV⁺ compared with HIV⁻ subjects shift to the left in FIG. 7, A to D). Based on the consistency, strength and significance of differences in the distribution of CCL3L1 copy numbers in the HIV⁺ and HIV⁻ individuals in the cohorts studied, the null hypothesis of no association between risk of acquiring HIV and CCL3L1 gene copy number was rejected (FIG. 7, A to D).

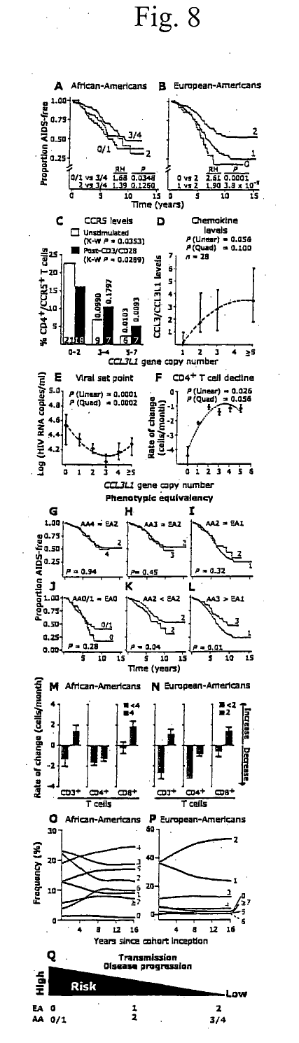

The strength of the association between CCL3L1 gene copy number and risk of acquiring HIV (FIG. 7, E to H) was next determined. In the initial analyses, the population-specific median copy number in the uninfected group was chosen as a reference point to compute the risk of acquiring HIV. Compared with possession of two CCL3L1 gene copies, children possessing <2 or >2 copies had significantly higher or lower risks, respectively, of acquiring HIV (FIG. 7E). This association was evident in the analysis for the entire cohort of children with (Table 8) or without (FIG. 7E) adjustments for receipt of zidovudine prophylaxis given to reduce the risk of transmission, as well as for those who received no prophylaxis (Table 8). Notably, with each increase in CCL3L1 copy number above the median, there was a dose-dependent, step-wise decrease in the risk for acquiring HIV (FIG. 8). The findings depicted in FIG. 7 (F to H), as well as those derived from a separate analysis in another ethnically matched cohort of 1,133 HIV⁻ individuals, indicated that adults who possessed CCL3L1 gene copy numbers lower than the population-specific median were at a higher risk of acquiring HIV. Thus, in each population, the median number of CCL3L1 gene copies served as the transition point at which the balance tilted in favor of protection against acquiring HIV.

The risk of acquiring HIV across the continuum of population-specific high to low CCL3L1 copy numbers was also estimated. Depending on the study population, each CCL3L1 copy lowered the risk of acquiring HIV by 4.5-10.5%, indicating that the population-specific high and low CCL3L1 gene copies are at the polar ends of HIV susceptibility. Substantiating this, relative to possession of the population-specific high CCL3L1 copy numbers, those who had low copy numbers had between 69 and 97% higher risk of acquiring HIV.

In addition to influencing HIV acquisition, CCL3L1 gene copies were associated with variable rates of disease progression. In the adult HIV⁺ cohort, a gene dose lower than the overall cohort median or population-specific median was associated with a dose-dependent increased risk of progressing rapidly to AIDS or death (FIGS. 8, A and B). A disease-influencing effect of CCL3L1 dose was not detected in the HIV⁺ children, suggesting that either the roles of CCL3L1 in HIV⁺ adults and children differ, or that the short follow-up time in the pediatric cohort was insufficient to detect an effect.

Increasing CCL3L1 doses were positively associated with CCL3/CCL3L1 secretion and negatively associated with the proportion of CD4⁺ T cells that express CCR5 (FIG. 8, C and D) (2). Additionally, there was a dose-dependent association between CCL3L1 copy number and the viral set point and rate of change in CD4⁺ T cell counts, two well-established predictors of clinical outcome (5); low CCL3L1 doses were associated with a higher viral set point and greater subsequent T cell loss (FIG. 8, E and F).

Human populations differ in their CCL3L1 gene content (FIG. 6). Accordingly, it was important to determine whether an absolute gene copy number has similar transmission- and/or disease-influencing phenotypic effects in different populations. To do so, the associated phenotypic effects and the changes in the frequency distribution of copy numbers in HIV⁺ EAs and AAs were compared over time. The findings indicated that in HIV⁺ EAs and AAs, the CCL3L1 gene copy numbers that correspond to the population-specific median, half-median and low doses (i) conferred comparable rates of disease progression or changes in CD3⁺, CD4⁺ or CD8⁺ T cell counts (FIG. 8, G to N, and Table 9), and (ii) had similar trajectories with respect to the changes in their distribution profiles over time (FIG. 8, O and P). These findings support the concept that different CCL3L1 gene doses among populations are associated with phenotypically equivalent effects (FIG. 8Q). They also imply that the phenotypic effects associated with CCL3L1 gene dosage cannot be estimated by knowing only the absolute CCL3L1 copy number. This value in a given individual is meaningful only if compared to the distribution of CCL3L1 copy numbers in the population in which the individual was sampled.

In the context of a prospective longitudinal study in which subjects are recruited at an early stage of infection, the association between CCL3L1 gene dose and HIV/AIDS susceptibility in adults indicates that the following pattern will be discernable. Initially, the HIV⁺ cohort will be enriched for individuals with CCL3L1 gene copy numbers lower than the population-specific median. Over time, the prevalence of these individuals will decrease because of their rapid progression to AIDS/death. As a result, the prevalence of HIV⁺ subjects with CCL3L1 copy numbers equal to or greater than the population-specific median will increase. Thus, with increasing follow-up times, the distribution of CCL3L1 gene copy numbers will become more similar to that found in HIV⁻ subjects. The value of testing this prediction is that it combines, into a single analytical model, analyses of (i) the susceptibility of individuals with different CCL3L1 gene copies, and (ii) the time-to-equilibrium between the virus and CCL3L1 genotype-dependent events in the infected host. Consistent with this prediction, our results highlight a dynamic evolution in the frequency distribution of CCL3L1 gene copy numbers in the setting of HIV infection (FIGS. 8, O and P). These observations underscore that this infection may exert a selective pressure affecting the population-specific distribution of CCL3L1 copy numbers over time.

CCR5 haplotypes, including CCR5 promoter polymorphisms and coding polymorphisms in CCR2 (CCR2-V64I) and CCR5 (Δ32) have been shown to influence the risk of transmission and disease progression (12-15). However, CCR5 is part of a complex virus—CCR5-CCR5 ligand system, complicating the analysis of in vivo contributions of CCR5 genotypes if gene-gene interactions are not considered. This is made all the more apparent by the observation that CCR5 protein expression levels are influenced not only by variations in the CCR5 gene (16, 17), but also by CCL3L1. Thus, virus—CCR5—CCL3L1 interactions in vivo and the phenotypic effects associated with CCR5 genotypes could be dependent, in part, on the genetic background conferred by CCL3L1 dose. To test this, the independent phenotypic effects attributable to CCL3L1 gene dose or CCR5 haplotype pairs (genotypes) and their combined effects were determined.

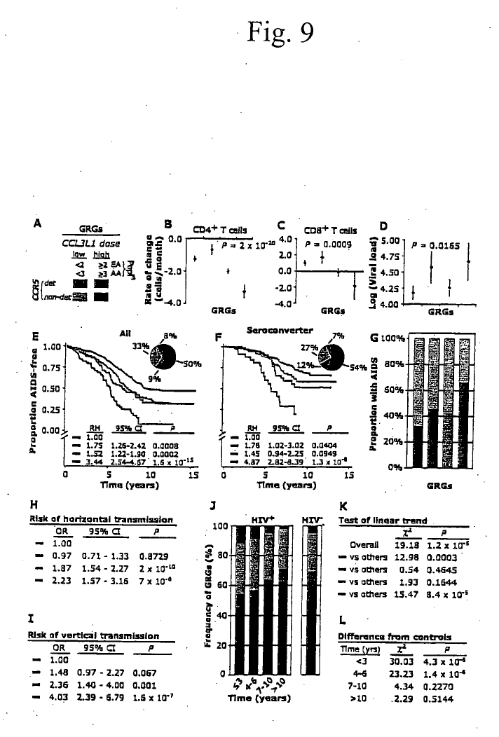

The HIV⁺ adult cohort was stratified into four mutually exclusive genetic risk groups (GRGs; FIG. 9A). Of the four GRGs, CCL3L1^{high}CCR5^{non-det} and CCL3L1^{low}CCR5^{det} were at the two extremes of HIV/AIDS susceptibility (FIG. 4, B to I). Relative to possession of CCL3L1^{high}CCR5^{non-det}, CCL3L1^{low}CCR5^{det} was associated with an ≧3-fold greater risk of progressing rapidly to 8 of 12 AIDS-defining illnesses (Table 10). By contrast, the CCL3L1^{high}CCR5^{det} and CCL3L1^{low}CCR5^{non-det} genotypes were associated with a ≦3-fold higher risk of progressing to three or four of these 12 illnesses, respectively (Table 10).

The trajectory of the frequency distribution profiles of the four CCL3L1/CCR5 GRGs in individuals with varying follow-up times were also revealing in that they closely tracked those recorded previously for CCL3L1 gene copies (compare FIG. 9, J to L vs FIG. 8, O and P). Thus, significant changes occurred only in the frequencies of the two GRGs that contained CCL3L1^{low} or CCL3L1^{high}CCR5^{non-det}, such that over time the distribution of the GRGs in surviving HIV⁺ subjects approached ever closer to the values observed in the HIV population (FIG. 9, J to L).

Thus, in the context of a well-characterized prospective cohort comprising HIV⁺ EAs and AAs, the CCL3L1/CCR5-based genomic signature for HIV/AIDS susceptibility was CCL3L1^{low}CCR5^{det}>CCL3L1^{low}CCR5^{non-det}≧CCR3L1^{high}CCR5^{det}>CCL3L1^{high}CCR5^{non-det}. This implied that CCL3L1^{low} may play a more dominant role than disease-accelerating, detrimental CCR5 genotypes in influencing HIV/AIDS pathogenesis in these two ethnic populations. Additionally, these findings suggest that a population-specific low CCL3L1 dose provides a permissive genetic background for the full expression of the phenotypic effects associated with detrimental CCR5 genotypes. This was apparent because (i) relative to genotypes that contained only CCR5^{det}, those that contained CCL3L1^{low} with or without CCR5^{det} were associated with a higher risk of acquiring HIV; and (ii) the maximal disease-accelerating effects associated with detrimental CCR5 genotypes occurred mainly in individuals who also possessed low CCL3L1 gene copies relative to the population-specific average (compare Kaplan-Meier plots for CCL3L1^{high}CCR5^{det}, and CCL3L1^{low}CCR5^{det} in FIG. 9, E and F).

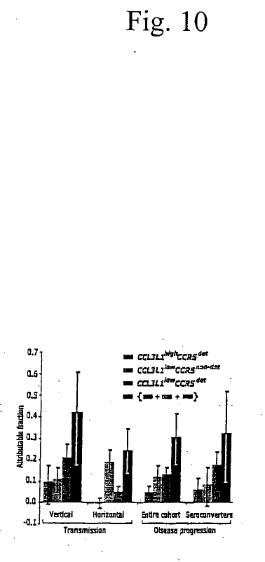

In the populations examined, up to 42% of the burden of infection and ˜30% of the accelerated rate of progression to AIDS were attributable to variations in CCL3L1/CCR5 (black bars in FIG. 10). The largest contributor to the burden of HIV/AIDS was possession of population-specific low CCL3L1 gene copy numbers (FIG. 10). These findings established the hierarchy of CCL3L1≧CCR5 in influencing the epidemiology of HIV in the populations examined. These results also substantiate the observation that the disease-accelerating effects associated with variation in CCR5 depend, in part, on the genetic background of CCL3L1 copy number.

### REFERENCES FOR EXAMPLE III

1. J. A. Bailey et al., Science 297, 1003 (2002).
2. J. R. Townson, L. F. Barcellos, R. J. Nibbs, Eur J Immunol 32, 3016 (2002).
3. P. Menten, A. Wuyts, J. Van Damme, Cytokine Growth Factor Rev 13, 455 (2002).
5. J. W. Mellors et al., Ann Intern Med 126, 946 (1997).
6. L. Wu et al., J Exp Med 185, 1681 (1997).
7. D. Zagury et al., Proc Natl Acad Sci USA 95, 3857 (1998).
8. A. Garzino-Demo et al., Proc Natl Acad Sci USA 96, 11986 (1999).
9. J. Reynes et al. J Acquir Immune Defic Syndr 34, 114 (2003).
10. H. Ullum et al., J Infect Dis 177, 331 (1998).
11. W. A. Paxton et al., J Infect Dis 183, 1678 (2001).
12. J. Tang, R. A. Kaslow, Aids 17 Suppl 4, S51 (2003).
13. M. P. Martin et al., Science 282, 1907 (1998).
14. E. Gonzalez et al., Proc Natl Acad Sci USA 96, 12004 (1999).
15. A. Mangano et al., J Infect Dis 183, 1574 (2001).
16. S. Mummidi et al., J Biol Chem 275, 18946 (2000).
17. J. R. Salkowitz et al., Clin Immunol 108, 234 (2003).
18. R. V. Samonte, E. E. Eichler, Nat Rev Genet. 3, 65 (2002).
19. D. L. Weed, Hematol Oncol Clin North Am 14, 797 (2000).
20. A. L. DeVico, R. C. Gallo, Nat Rev Microbiol 2, 401 (2004).
21. J. L. Heeney et al., Proc Natl Acad Sci USA 95, 10803 (1998).
22. R. K. Ahmed et al., Clin Exp Immunol 129, 11 (2002).

### Example IV

### CCL3L1 Gene-Containing Segmental Duplications in HIV-1/AIDS Susceptibility

The CCL3L1 gene copy number was determined in individuals who comprise the cohorts shown in the flow chart in Table 11, which are described in greater detail below.

The human CCL3L1 gene copy distribution was determined in the following study populations. First, the CCL3L1 gene copy number was determined in individuals who comprise the HGDP-CEPH Human Genome Diversity Cell Line panel. This panel comprised 1,065 individuals from 57 human populations with minimal admixture. The characteristics of this panel are as described previously (1, 2). The CCL3L1 gene copy numbers in 1,044 of 1,065 individuals from this panel were derived (Table 12).

The second human study population came from three major sources:

(a) HIV-1-positive (HIV⁺) or HIV-1-negative (HIV⁻) European (EA)-, African (AA)-, and Hispanic (HA)-American subjects from WHMC, San Antonio,
(b) HIV⁻ adult subjects from sources other than WHMC, and
(c) a cohort of Argentinean HIV⁺ and HIV⁻ children born to HIV-infected mothers.

CCL3L1 gene copy numbers were available from 4,308 of the 4,493 individuals that comprise these cohorts and the characteristics of each of these cohorts are as follows.

Adult patients with HIV-1 participating in the U.S. Air Force (USAF) portion of the Military HIV Program Natural History Project contributed samples for this study. WHMC is the referral hospital for all USAF personnel who develop infection with HIV-1. The voluntary, fully informed consent of the subjects used in this research was obtained as required by Air Force Regulation 169-9 and with approval from the Institutional Review Board (IRB) of University of Texas Health Science Center, San Antonio, Tex. A total of 1,132 HIV⁺ adult patients were evaluated, including 515 seroconverting individuals. The demographic background of this cohort was 55% EA, 36% AA, 6% HA, and 3% “other.” The median age at the time of diagnosis was 28 years (range, 18-70 years), and 94% of the subjects were male. The median follow-up time was 6.2 years for the entire cohort and 6.6 years for the seroconvertors, using as the initial time point the estimated seroconversion date (the midpoint between the last negative and first positive HIV test). The median time from the last negative HIV-1 test to estimated seroconversion was 10.8 months. Forty percent of this cohort progressed to AIDS (1987 criteria), and 39% died during the study period that ended December 1999.

Additional epidemiological features of the HIV-1-infected cohort are as described previously (3-6). Of special note is that this cohort has a racially balanced composition. It represents one of the largest cohorts of HIV seropositive patients followed prospectively at a single medical center. Also, because of the unique nature of the cohort, additional factors that confound genotype-phenotype studies (e.g., unequal access to medical care and anti-retroviral therapy, length of follow-up and loss to follow-up) are minimized.

A cohort of children from Buenos Aires, Argentina exposed perinatally to HIV-1 was also studied. DNA was available from 802 children perinatally exposed to HIV-1 between 1986 and 2003, of whom 395 were born HIV⁻ and 407, HIV⁺. The major epidemiological features of this cohort are as described previously (7). Briefly, Argentina is widely regarded to have one of the most European-like populations of all Latin American countries, with the vast majority of Argentineans being descendants of individuals from southern Europe, primarily from Spain and Italy. There is little admixture with Amerindians and no substantial population of individuals of African origin (7). In this light; and in conjunction with the fact that the vast majority of the children were from hospital sources in Buenos Aires, the HIV⁺ and HIV⁻ children studied were demographically and ethnically very similar.

The HIV-1-infected children are followed at a tertiary care, academic, pediatric hospital (Hospital de Pediatria “J. P. Garrahan”) in Buenos Aires. Physicians from different medical centers, primarily in Buenos Aires, refer to this hospital children under the age of 18 months for early diagnosis or over 18 months when the child has an illness compatible with a diagnosis of HIV infection and/or needs specialized medical care. Thus, in this cohort, we recruited the following subjects: (a) all children (either HIV⁺ or HIV⁻) born to HIV⁺ mothers in two maternity hospitals that are closely affiliated with this tertiary care center, (b) additional HIV⁺ children (born to HIV⁺ mothers) who were referred to this tertiary care center. The nearly equal proportion of infected:uninfected children is not indicative of transmission rate since ascertainment was skewed toward infected children. The makeup of this cohort is similar to cohorts of highly HIV-exposed adults, some of whom remain uninfected, whereas others become infected (8).

HIV-1 infection status, AIDS definition, and stage of immune suppression were established according to the 1994 criteria of the Centers for Disease Control and Prevention (CDC) classification for children. The zidovudine (ZDV) prophylaxis provided (or available) to mother-infant pairs was according to the ACTG 076 protocol (9) and was considered complete in 181 (161 uninfected and 20 infected children), partial (mother or child) in 26 (6 uninfected and 20 infected), and absent in 475 children (156 uninfected and 319 infected). For statistical analysis, mother-infant pairs that received complete or partial ZDV prophylaxis were pooled. Information regarding ZDV prophylaxis was unavailable in 120 mother-child pairs (72 uninfected and 48 infected), and was not included in the statistical analyses that adjusted for the effects of ZDV. After 1992, all infected children received antiretroviral therapy according to the recommended guidelines. The longitudinal follow-up data used in this paper corresponds to that previously published (7) and is comprised of 347 HIV⁺ children. The median follow-up of these infected children was 4.08 years; 55.6% of this cohort progressed to AIDS, and 7.2% died during the study period, which ended Jan. 1, 1999. Informed written consent was obtained from parents or legal guardians, and the study was approved by the local Institutional Review Board. The clinical care of the patients was under the supervision of a single medical-care provider (R.B).

The first HIV⁻ cohort from which CCL3L1 gene copy numbers was obtained was comprised of 1,274 control unlinked EA, AA, and HA normal blood donors. The AA and EA components of this cohort were derived from normal blood donors from San Antonio, Tex.; Winston-Salem, N.C. and Columbus, Ohio. The 102 HAs were normal blood donors from San Antonio (3, 5). This cohort is designated the non-WHMC HIV-uninfected comparison (control) group.

A second cohort of 1,133 seronegative samples was obtained from HIV-1-negative Air Force personnel to serve as an additional reference population for comparison of CCL3L1 gene copy distribution with the HIV-infected WHMC cohort. Specifically, excess/discarded blood samples from 4,000 sequential Air Force military trainees at Lackland Air Force Base were obtained, and 1,300 randomly selected with an ethnic distribution very similar to the HIV-infected WHMC cohort. Individual samples were associated with race, gender and age of donor, but were not linked to an identifiable donor. For entry into the recruit training program from which the samples were obtained, each donor had tested HIV negative recently. The protocol for this study was approved by the Institutional Review Board at WHMC. In this cohort of HIV-1 uninfected individuals, HAs were categorized with EAs. Thus, in the statistical analyses using this cohort of uninfected individuals to determine the association between variable CCL3L1 gene copy numbers and risk of acquiring HIV-1 infection, the EAs and HAs were placed within a single group and compared to ethnically matched HIV⁺ WHMC cohort subjects. The analyses from the HIV⁻ WHMC cohort subjects are shown in FIGS. 9H, and J to L.

The gene copy numbers of the chimpanzee CCL3-like (CCL3L) orthologs from 83 animals were determined. Forty-eight chimpanzees out of the total genotyped were unrelated. There were no differences in the mean (SD)/median number of CCL3L1 gene copies in the chimpanzees that were related [mean=9.5 (SD=2.03)/median=9] and those that were unrelated [mean=9.1 (SD=1.56)/median=9].

Genotyping was performed according to the method of Townson et al. (10), with few modifications. Briefly, real-time PCR was performed by using an ABI/PRISM7700 or 7900 Sequence Detector System (PE—Applied Biosystems) detecting emitted fluorescence as FAM (6-carboxyfluorescein, 6-FAM) from the probe detecting CCL3L1 (or CCL3L in chimpanzee) and VIC from the probe detecting the β-globin gene during amplification. CCL3L1 primer sequences are as follow: sense primer 5′-tctccacagcttcctaaccaaga (SEQ ID NO: ______; antisense primer 5′-ctggacccactcctcactgg (SEQ ID NO: ______; probe 5′-FAM-aggccggcaggtctgtgctga-TAMRA (6-carboxy-tetramethyl-rhodamine, TAMRA) (SEQ ID NO: ______. For β-globin primer sequences are as follows: sense primer 5′-ggcaaccctaaggtgaaggc (SEQ ID NO: ______; antisense primer 5′-ggtgagccaggccatcacta (SEQ ID NO: ______; and the probe 5′-VIC-catggcaagaaagtgctcggtgcct-TAMRA (SEQ ID NO: ______) (synthesized by Applied Biosystems). This assay used to determine human CCL3L1 gene dose discriminates between human CCL3L1 and CCL3 (10), but not between CCL3L1 and CCL3LΨ. The same assay was used to probe chimpanzee genome, and does not differentiate between the two orthologs.

The cycle number at which the fluorescence reached a fixed threshold, termed the threshold cycle (C_{T}), was determined (C_{T} is proportional to the amount of initial target sequence). Five serial 1:2 dilutions (25-1.56 ng) of genomic DNA from A431 cells [known to have two copies of CCL3L1 per diploid genome (pdg) by Southern blot densitometry (10)] were used to generate standard curves of C_{T} value against the log [DNA] on each PCR tray/plate (96 or 384 wells) for β-globin (present at two copies pdg) and for the CCL3L1 gene. For each test sample, duplicate wells were set up for CCL3L1 and β-globin, C_{T} determined, and converted into template quantity using the standard curves. Copy number is the ratio of the template quantity for CCL3L1 to the template quantity for β-globin, multiplied by two.

The method by which the raw data for CCL3L1 gene copy numbers were handled and how they were converted to the nearest integer is described below. Each standard curve dilution was run in triplicate per PCR for both CCL3L1 and β-globin. A correlation coefficient (R²) for a standard curve <96% was considered inadequate, and the corresponding PCR tray of DNA samples was repeated. When a result of zero copies/genome was obtained, the sample was checked by conventional PCR using a pair of primers specific for CCL3L1: sense primer 5′-gatgctattcttggatatcctgag (SEQ ID NO: ______, and antisense primer 5′-gtgcagagaggacctggttg (SEQ ID NO: ______. As a control, the following primers were used to detect CCL3L1 and CCL3: sense primer 5′-cctagattctcatacctggagac (SEQ ID NO: ______, and antisense primer 5′-aatcatgcaggtctccactg (SEQ ID NO: ______).

The values of the slopes obtained for both the target gene CCL3L1 in humans (and CCL3L in chimpanzee) and the normalizer gene namely β-globin were very similar. This makes the β-globin gene a good normalizer to estimate the copy numbers of CCL3L1 and CCL3L genes in humans and chimpanzee, respectively.

Fresh chimpanzee (Pan troglodytes troglodytes) peripheral blood mononuclear cells (PBMCs) were from the Southwest Foundation for Biomedical Research at San Antonio using approved IACUC protocols. Cells were stimulated for 72 hours with anti-CD3/CD28, mRNA isolated, and reverse transcribed. Degenerate PCR primers were designed based on available human and non-human primate CCL3 and CCL3L1 mRNA sequences. These primers were designed to amplify sequences with homology to both human CCL3 and CCL3L1. The first PCR was with: forward: 5′-ATG CAG GTC TCC ACT GCT GC-3′ (SEQ ID NO: ______) and reverse 5′-TCA GGC ACT CYG CTC YAG GTC-3′ (SEQ ID NO: ______). To obtain additional specificity for amplification of CCL3-like sequences, the PCR product was subjected to nested PCR with the following primer set: forward: 5′-CTG CCC TTG CYG TCC TCC TCT G-3′ (SEQ ID NO: ______) and reverse 5′-AGG TCR CTG ACR TAT TTC TG-3′ (SEQ ID NO: ______). PCR conditions were 92° C. for 2 minutes, 35 cycles of 92° C. for 30 seconds, 55° C. for 30 seconds and 72° C. for 30 seconds, and a final extension of 72° C. for 5 minutes. PCR amplicons were cloned in pcrTOPO2.1 vector (Invitrogen) and sequences obtained aligned by using CLUSTALW. 5′- and 3′-end sequences that were encompassed within the PCR primers were confirmed by comparison to the sequences available in the chimpanzee genome. PBMCs derived from normal human donors were stimulated with anti-CD3/CD28 for 72 hours, cDNA was prepared, and PCR using the aforementioned primers was used to amplify and then clone CCL3 and CCL3L1 cDNAs. Sequences from 81 and 101 CCL3/CCL3L1 transcripts from 8 humans and 13 chimpanzees, respectively, were analyzed.

PBMCs were isolated from normal seronegative donors from the local blood bank. The hypothesis was that a higher CCL3L1 gene copy number was positively correlated with enhanced production of CCL3L1 protein, and inversely related to the percentage of CD4+ cells expressing CCR5, due in part to ligand-induced receptor down-regulation (11-15). In brief, immobilized antibodies to CD3 (anti-CD3) and CD28 (anti-CD28) were used to promote long-term polyclonal proliferation of CD4⁺ T cells and enhanced production of CC chemokines. Briefly, anti-CD3 and -CD28 antibodies (Pharmingen) were resuspended in PBS and used to coat 12-well flat-bottom polystyrene plates for 2 h at 37° C. Some wells were incubated with PBS only, and the supernatants of PBMCs cultured in these wells constituted our unstimulated PBMC culture samples (designated as “unstimulated” in FIG. 8C). PBMCs were added at a concentration of one million cells/ml and cultured for 48 h, and then their culture supernatants were used to measure levels of CCL3 or the cells were used to perform antibody labeling and FACS analysis.

A commercially available pair of antibodies was used to measure the total levels of CCL3 (CCL3L1 and CCL3) (R&D Systems, Minneapolis) as previously described by Townson et al (10). For FACS analysis, after addition of the relevant antibodies, cells were incubated for 15- to 30-min at room temperature. For CCR5 labeling, the antibody clone 2D7 from Pharmingen (BD Biosciences, San Diego, Calif.) and an appropriate isotype control was used. After washing the unbound antibodies, cells were analyzed using FACSCalibur™. Finally, the results of FACS analysis are reported as a percentage of CD4+ cells expressing CCR5 in the cell cultures. The percentage of CCR5 expressing cells were derived from the comparison of samples stained with the isotype control or anti-human CCR5 antibody. The total CCL3L1 and CCL3 measured by ELISA in each sample was corrected for the number of cells determined by using a colorimetric method, and the optical density for each sample was used as the normalizing factor. Thus, the units reported in FIG. 3D are arbitrary units reflecting total CCL3/CCL3L1 chemokine production.

HIV-1 RNA levels were determined in the plasma samples of the acutely seroconverting component of the HIV⁺WHMC cohort. These plasma samples corresponded to the first sample available at time of diagnosis of seroconversion. Plasma samples stored as 1 mL aliquots at −70° C. were thawed, and 1 ml was aliquoted to a 9 mL NucliSens® lysis buffer tube (Organon Teknika, Boxtel, Netherlands) for RNA extraction. HIV-1 RNA was amplified and quantified by NucliSens® protocol. With the 1 mL input, the lower limit of detection of the NucliSens® assay is at least 80 copies/mm³ of blood plasma. Plasma samples below detection levels were assigned a value of 50 copies/mm³ for statistical analyses.

In this study, based on the findings in FIGS. 7 and 8, the following was observed. (a) In uninfected European Americans (EA), the median number of CCL3L1 gene copies was 2, and possession of <2 CCL3L1 gene copies was associated with an enhanced risk of acquiring HIV-1 infection. (b) In uninfected AA, the median CCL3L1 gene copy numbers was 4, and possession of <4 CCL3L1 gene copies was associated with an enhanced risk of acquiring HIV-1 infection. (c) In uninfected Hispanic Americans (HA), the median CCL3L1 gene copy numbers was 3, and possession of <3 CCL3L1 gene copies was associated with an enhanced risk of acquiring HIV-1 infection. (d) The median number of CCL3L1 gene copies in the HIV⁺ EAs and AAs was 2 and 3, respectively, and possession of <2 and <3 CCL3L1 copies was associated with a rapid rate of disease progression to AIDS and death in EAs and AAs, respectively. (e) In infected children of Argentinean descent (7), the median number of CCL3L1 gene copies is 2, and possession of <2 CCL3L1 gene copies was associated with an increased risk of acquiring HIV infection.

These findings provided the rational basis to dichotomize the CCL3L1 genotypes into the groups designated as “CCL3L1^{low}” denoting possession of CCL3L1 gene copies lower than the population-specific median, and “CCL3L1^{high}” denoting CCL3L1 gene copies equal to or greater than the population-specific median. Thus, for example, in FIG. 91, in children exposed to perinatally to HIV, possession of <2 CCL3L1 gene copies corresponds to the CCL3L1^{low} that is included in the GRGs (Table 13).

To assess whether the strategy of dichotomizing the CCR5 genotypes and CCL3L1 gene copies was robust to sampling variations, bootstrap samples from the entire WHMC cohort were used, and a determination was made regarding whether the disease-influencing effects observed with the CCR5 and CCL3L1 risk groups in the entire cohort versus 1,000 bootstrap samples derived from 70% of the entire cohort (n=792) were similar. The 95% confidence intervals for the relative hazards (RHs) for the risk of progressing rapidly to AIDS for the entire cohort and those for the bias-corrected estimates from the bootstrap samples were similar, suggesting that this approach of dichotomizing CCR5 genotypes and CCL3L1 gene copy numbers was both valid and robust (Table 14).

A genetic risk stratification system was developed to determine the combined effects of CCL3L1 gene copy numbers and CCR5 genotypes, i.e., CCL3L1/CCR5 GRGs (FIG. 9A). Based on the possession of population-specific detrimental CCR5 genotypes and/or CCL3L1 gene copy numbers lower than population-specific median, four mutually exclusive GRGs exist:

(a) Possession of neither CCL3L1 gene copies lower than the population-specific median or detrimental CCR5 genotypes (CCL3L1^{high}CCR5^{non-det}). This is the reference group.
(b) Possession of detrimental CCR5 genotypes, but not CCL3L1 gene copies lower than the population-specific median (CCL3L1^{high} CCR5^{det}).
(c) Possession of CCL3L1 gene copies lower than the population-specific median, but not detrimental CCR5 genotypes (CCL3L1^{low}CCR5^{non-det}), and
(d) Possession of both CCL3L1 gene copies lower than the population-specific median and detrimental CCR5 genotypes (CCL3L1^{low}CCR5^{det}).

Thus, for example, in the cohort of children from Argentina who are exposed perinatally to HIV-1, the CCL3L1/CCR5 GRGs are as follows:

CCL3L^{low}CCR5^{det} corresponds to possession of <2 CCL3L1 gene copies and all CCR5 genotypes that contain the CCR5-HHE haplotype.
CCL3L1^{low}CCR5^{non-det} corresponds to possession of <2 CCL3L1 gene copies, and CCR5 genotypes that lack the CCR5-HHE haplotype.
CCL3L1^{high}CCR5^{det} corresponds to possession of ≧2 CCL3L1 gene copies and CCR5 genotypes that contain the CCR5-HHE haplotype.
CCL3L1^{high}CCR5^{non-det} corresponds to possession of ≧2 CCL3L1 gene copies and CCR5 genotypes that lack the CCR5-HHE haplotype.

Unless noted otherwise, all statistical analyses were conducted using STATA 7.0 (Stata Press, College Station, Tex.).

First, methods were developed to assure quality control for the PCR-based determination of CCL3L1 gene copy numbers (Table 15).

The distribution of CCL3L1 gene copy numbers in human populations was then determined (FIG. 6). This allowed a determination of whether the CCL3L1 gene copies among different human populations were randomly or non-randomly distributed. To determine the association between copy number and continent-of-origin in the CEPH-cohort analysis of variance was used as an overall analytical strategy. Pair-wise post-hoc comparisons were not conducted. A determination was also made regarding whether an ancestral correlate of CCL3L1 gene dosage existed in chimpanzee (FIG. 6).

The cohorts of HIV-infected adults and children each represent one of the largest cohorts followed at a single medical center. As noted, this reduces significantly several confounding factors in genetic epidemiological studies, and because of the unique nature of the cohorts there is equal access to health care and medications. Also, because of the standard nature in the health care delivered and diagnostic criteria used under the direction of a stable and limited number of supervising physicians, the phenotypic end-points (e.g., AIDS-defining illnesses) have been well defined and characterized.

The association between CCL3L1 gene copies and two distinct endpoints was determined: risk of acquiring HIV-1 and rates of disease progression (AIDS and death) (FIG. 7 and FIG. 8, A and B; Table 16).

The risk of acquiring HIV-1 was examined in two settings, namely risk of vertical or horizontal transmission. To validate these results with respect to the association between CCL3L1 gene copies and risk of acquiring HIV in adults, two large and different HIV-negative adult cohorts were analyzed (WHMC and non-WHMC sources controls) (FIG. 7). The reason for using two different cohorts was to demonstrate if the pattern of association for the comparison of HIV-positive and HIV-negative subjects was similar across two different HIV-negative cohorts. For these studies, analyses were conducted within (not across) each ethnic group. The statistical methods used to determine the association between CCL3L1 gene copy number and risk of acquiring HIV-1 were with multivariate logistic regression models which inherently adjust for the multiple comparisons of different copy numbers with the population-specific reference CCL3L1 gene copy number.

To determine the association between CCL3L1 gene copy number and rate of progression to AIDS multivariate Cox proportional hazards models were used which minimize the problem of multiple comparisons (FIGS. 8, A and B).

To address the issue of biological plausibility, the relationship between CCL3L1 dose and chemokine and CCR5 protein expression was determined (FIG. 8. C and D).

In addition to the clinical endpoints, an association was determined between CCL3L1 gene copies and baseline HIV-1 RNA levels (viral set point) and CD4+ T cell decline, two other well-established surrogate markers of HIV-1 disease progression (29, 30) (FIG. 3, E and F). In this analysis, pair-wise comparisons were not conducted, but second order polynomial curves were fitted to assess the overall association of the CCL3L1 gene dose with rates of changes in CD4+ T cell counts or the viral set point.

In each of the different settings, i.e., risk of acquiring HIV-1 (vertical or horizontal), disease progression in adults, rate of CD4⁺ T cell decline, or influence on the viral set point, the directions of the effects observed were similar. It was consistently observed that possession of CCL3L1 gene copies lower than the population-specific median was associated with enhanced HIV/AIDS susceptibility. Furthermore, as indicated above, the robustness of the data is increased as the statistical analyses were conducted using a single model, minimizing the concerns related to multiple comparisons, and limiting the possibility that the findings were due to chance in the different clinical settings examined.

It was found that human populations differed in their CCL3L1 gene content (FIG. 6). Thus, it was important to determine whether despite these differences in CCL3L1 gene dose at the population level, distinct population-specific gene copy numbers had similar phenotypic effects. This concept of phenotypic equivalency associated with varying CCL3L1 gene copy numbers was assessed in different populations using different endpoints, i.e., phenotypic equivalency for clinical endpoints such as rate of progression to AIDS and also biological endpoints such as rate of change (increase/decrease) in leukocyte subsets such as CD4+ or CD8+ T cells (FIG. 8, G to N, and Table 9).

In this analysis, multiple comparisons within and across the populations were conducted. However, no correction was made for the P values for the multiple comparisons for the following two reasons: a) In these analyses, the goal was to determine if there was equivalence rather than difference across ethnic groups. In this context, the Bonferroni corrected P values (which inflate after correction) are likely to favor equivalence. Thus, not correcting for multiple comparisons provided a more stringent test of demonstrating the equivalence. b) Where differences were detected (for example, AA3 vs EA1 in FIG. 8L), they are recorded for illustrative purposes rather than for formal test of hypothesis.

To further validate the concept of phenotypic equivalency of different population-specific CCL3L1 gene copies, Markov-modeling was used to simulate the changes in the frequency distribution of CCL3L1 gene copies over time in EAs and AAs in infinite sized cohorts (FIGS. 8, O and P, Table 16). If phenotypic equivalency between different population-specific CCL3L1 gene copies existed, then the evolution or trajectory of the changes in the distribution of individuals who possess CCL3L1 gene copies that are lower than, equal to or greater than the population-specific median should be similar.

These Markov modeling analyses were complemented by studies in which a determination was made of the actual trajectory of the changes in the distribution of HIV-positive individuals with different CCL3L1 gene copies. To do this, the HIV⁺ WHMC cohort was stratified based on varying follow-up times, and the trajectory of the changes in the frequency distribution of CCL3L1 gene copies was determined.

Markov modeling was also used to determine when the distribution of HIV-infected EA and AA cohorts would approximate that of the uninfected populations. It was observed that the HIV-infected cohort approximated the HIV-uninfected cohort in ˜6 years. Considering the annual comparison with the HIV-uninfected subjects, corrections were made for these multiple comparisons.

Taken together, these analyses shown in FIG. 8, O and P also allowed for a test of the hypothesis that within the context of a prospective longitudinal study conducted in a well-characterized HIV⁺ cohort the overall CCL3L1 gene copy number distribution of the cohort will evolve over time. The advantages of testing this hypothesis is that it telescopes into a single analytical model the analyses of (i) susceptibility of individuals with variable CCL3L1 gene copies, and (ii) time-to-equilibrium between host CCL3L1 genotype-dependent events and HIV-1 pathogenesis.

To study gene-gene interactions within the context of virus—CCR5—CCL3L1 interactions in vivo, the study subjects were stratified into four mutually exclusive CCL3L1/CCR5 GRGs (FIG. 9A; and Tables 13 and 17). To determine the validity of using this stratification system, bootstrap analyses were conducted (Table 14).

The association between possession of these CCL3L1/CCR5 GRGs and risk of acquiring HIV, rate of disease progression, CD4+ and CD8+ T cell loss, the viral set point and risk of developing AIDS was determined in the HIV⁺ WHMC cohort (FIG. 9 B-H). The association between possession of these CCL3L1/CCR5 GRGs and the risk of transmission in children exposed perinatally to HIV-1 was also determined (FIG. 91).

In constructing the GRGs using population-specific CCR5 genotypes associated with an accelerated disease progression and population-specific cut-offs for CCL3L1 gene copies, the underlying ethnic background was accounted for. This provided statistical power for further analysis without ignoring the ethnic-specific phenotypic effects associated with the CCR5 and CCL3L1 genotypes.

Multivariate regression models were used (logistic regression for the risk of acquiring HIV-infection and Cox proportional hazards for rate of progression to AIDS), thus minimizing concerns related to multiple comparisons. Also, when the association of the GRGs and the rates of change of CD4+ and CD8+ T cell counts was determined, only one test of hypothesis was conducted: the rates of CD4+ and CD8+ T decline in the CCL3L1^{high}CCR5^{nondet} genotype would be minimal compared to those associated with the CCL3L1^{low}CCR5^{det} genotype (FIG. 9, B and C). For testing this association, the Student's t-test was used on the GEE regression coefficients, and a single comparison was made. However, the P values obtained for the aforementioned comparison were so small that even after correcting for the six comparisons (theoretically possible comparisons when considering four GRGs) the inferences would not change. The same strategy was used to test the association of the GRGs with the viral load (FIG. 9D).

The trajectory of the changes in the frequency distribution of individuals with different GRGs over time was also determined. For this, the HIV⁺ WHMC cohort was stratified based on varying follow-up times, and the trajectory of the changes in the distribution of CCL3L1/CCR5 GRGs over time in the HIV⁺ WHMC cohort was determined (FIG. 9, J to L). Second, each of these time groups was compared with the HIV-uninfected subjects. Thus, four comparisons were made (shown in FIG. 4L) and correcting for these four comparisons (at an α-error rate of 0.0125) did not change the interpretation. For simplicity, in this instance, P values are presented without correction for multiple comparisons.

AIDS is a conglomerate complex of various defining illnesses and the association of the CCL3L1/CCR5 GRGs and rate of progression to distinct AIDS-defining illnesses in the HIV-infected WHMC cohort was determined (Table 10). A determination was made regarding whether the CCL3L1^{low}CCR5^{det} genotype is consistently associated with an accelerated disease course for each of the different AIDS defining illnesses. Multivariate Cox proportional hazards models were run for each AIDS defining illness as an outcome and the P values for each model were reported (that is for each AIDS defining illness). Nocomparisons across the AIDS defining illnesses were made and thus, the issue of multiple comparisons is not relevant in this context.

We estimated the public health impact of these CCL3L1/CCR5 GRGs by calculating their AF for the risk of acquiring HIV-1 (in the context of vertical and horizontal transmission) and rate of disease progression (FIG. 10).

We found that the robustness of the findings reported is increased as (a) the analyses were conducted using different cohorts that reflect different modes of acquiring HIV; (b) several different endpoints were examined, that included the risk of acquiring HIV/AIDS, rate of disease progression, viral set points, and rate of change in T cell counts; (c) all associations were in the same direction and all of the tests attained significance (or very near-significance); (d) we accounted for genetic stratification by examining different HIV⁺ and HIV-negative cohorts from different populations; (e) we used statistical models that minimize concerns for multiple comparisons, and the issues related to multiple comparisons are discussed; (f) this number of significant tests exceeds the proportion that could be explained by chance; (g) there is a well-established in vitro experimental biological plausibility for the hypothesis tested; and finally, (h) the findings related to the association between CCL3L1 dose and risk of acquiring HIV-1 were analyzed in accordance with the Bradford Hill criteria (31, 32) for causality.

The fact that the 1:2 serial dilution amplification curves overlap each other at each dilution step, and that these curves are clearly demarcated from each other, makes this assay robust to distinguishing genomes that differ in CCL3L1 gene copy numbers within the same order of magnitude.

We analyzed the results from the Real-Time PCR (TaqMan) assay for reproducibility and consistency. First, we ran all samples in duplicate. The within-sample variation was measured as: Vᵢ(%)=100(Rᵢ₁−Rᵢ₂/Rᵢ)=200(Rᵢ₁−Rᵢ₂)/(Rᵢ₁+Rᵢ₂), where Rᵢ₁ and Rᵢ₂ represent the duplicate readings for the ith sample and Rᵢ represents the mean of the duplicate readings. We then plotted a control chart to identify the samples with wide intra-sample variation. The average intra-sample variation (Vᵢ) was estimated to be 27.5% and the upper 3-standard deviation limit of the variation was observed to be 43.23%. Thus, we repeated all samples with Vᵢ values outside the range of 43.23% variation in duplicate.

For each sample included in the final analysis (i.e., if the value of Vᵢ was within the acceptable limit of less than 43.23%) then the CCL3L1 gene copy number was estimated by rounding the mean value (Rᵢ) to the closest integer. Arguably, the process of rounding can lead to a loss of information. We undertook rounding for two reasons. First, logically as well as biologically an integer gene copy number is intuitively interpretable. Second, if the loss of information because of rounding is not substantial, then rounding can be retained for the sake of simplicity and interpretability. Given that a simplification of the data by rounding can lead to categorization with a large number of ties across categories, we foresaw that further statistical analysis may be heavily influenced by the process of rounding.

To consider the effects of rounding of the Taqman assay estimates, we conducted two different analyses. First, we plotted the frequency of the raw estimates from Taqman assay for 4,308 subjects, and found that the frequency distribution of the raw estimates, and the peaks were invariably close to integer scores indicating that, in general, the assay detected close-to-integer copy numbers. Second, we summarized the rounded and raw Taqman estimates for the various populations studied (Table 15) and observed that rounding did not lead to any substantial systematic error. The estimate of the actual number of CCL3L1 gene copies in an individual was, thus, taken to be the rounded average of the duplicate estimates.

We used several complementary approaches to determine the precision and reproducibility of our estimates of the CCL3L1 gene copy numbers. We determined the intra- and inter-assay variability and also took into account different methods of aliquoting the PCR reagents and genomic DNA (manual vs. robot) as well as the use of different thermocyclers, variables that could potentially influence gene copy number estimation.

Since we determined the CCL3L1 gene copy estimate in duplicates, we first assessed the intra-assay agreement of these estimates. A very high degree of intra-assay consistency in copy number estimates was observed. The slope of the regression line was very close to 1 indicating that the replicate estimates for the same DNA from a subject run in duplicate are nearly identical.

We formally tested for the intra-assay agreement using two statistical methods. We first estimated weighted multi-category Cohen's kappa. Each category for this analysis was defined by a unique copy number obtained from a single replicate for each sample. We observed a kappa value of 0.9367 with a negligibly small P value (z=77.95). Second, we generated bootstrap confidence intervals around the intraclass correlation coefficient (ICC) between the two estimates of the copy numbers derived from the two replicates. The estimate of the ICC thus obtained was 0.937 (95% confidence interval: 0.932-0.941). Together, these analyses revealed that our assay had significantly low intra-assay variability.

Next, we assessed the inter-assay agreement of the CCL3L1 gene copy estimates on a randomly chosen sub-sample of 68 subjects, using different thermocyclers and methods for aliquoting DNA and PCR master mixes. For this set of analyses, we ran the assays on three PCR machines: a 96-well plate real-time PCR thermocycler (Applied Biosystems, 7700; designated as Machine #1) and two 384-well plate thermocyclers (Applied Biosystems, Prism 7900HT Sequence Detection Systems; designated as Machine #2 and Machine #3). We aliquoted the samples manually on the 96 well plate and with a robot (Tecan Evo™) onto the 384 well plates. In each case, we ran the assay in duplicates. Thus, we had six readings (gene copy estimates) on each of the 68 subjects. Again, to start with we assessed whether all the three experimental conditions gave similar results over the range of copy numbers. To address this, we used modified Bland-Altman (B-A) plots, which graphically depict the difference between the estimates obtained by the duplicates plotted against the average of these estimates.

The mean differences in the average CCL3L1 gene copy estimates were −0.047 (95% CI: −0.149 to 0.054), 0.095 (95% CI: −0.013 to 0.203) and −0.040 (−0.132 to 0.051) for machines #1, #2 and #3, respectively. This indicated that there was a close agreement between replicates within each experimental condition. The intra-assay variability as assessed by Pitman's test of equal variances was very low (P=0.354, 0.238 and 0.099 for machines #1, #2 and #3, respectively).

We then compared the average estimates of the CCL3L1 gene copy number obtained by the three PCR machines. For these analyses, we used two approaches. First, we estimated the multi-category weighted Cohen's kappa.

These results indicated that there was an excellent concordance between each pair of estimates. We further tested this by generating bootstrap confidence intervals around the correlation coefficients by repeatedly (1,000 repetitions) drawing sub-samples so as to overcome the potential pitfall of small sample size. Given the categorical nature of the CCL3L1 gene copy number we used intraclass correlation coefficient (ICC) as the measure of correlation.

These results indicate that the method used for estimating CCL3L1 gene copy numbers was (i) sensitive, such that it can discriminate accurately over a wide range of copy numbers; (ii) robust to different experimental conditions; and (iii) has a low intra-assay and inter-assay variability.

The association between CCL3L1 gene copy numbers or CCL3L1/CCR5 GRGs and the risk of acquiring HIV-1 infection in children and adults was also examined (FIG. 7, and FIG. 9, Table 16). We estimated the risk of acquiring HIV-1 by comparing the distribution of CCL3L1 gene copy numbers in individuals with and without HIV-1 infection. We used multivariate logistic regression to estimate the risk of acquiring HIV-1 associated with each category of CCL3L1 gene copy number by comparing it with a reference category which was the population-specific median gene copy number (FIG. 7) or population-specific high CCL3L1 gene copy numbers using a single regression model. The copy number was coded as a categorical variable (0-≧7 copies representing each category) and the reference category was fixed by including the remaining categories into the regression model. Odds ratios greater than unity indicated an increased risk of acquiring HIV-1 compared to the reference category, whereas odds ratios lower than one were interpreted as a reduced risk of acquiring HIV-1 compared to the reference category. Population-specific transition/switch points (which corresponded with the population-specific median copy numbers in HIV-uninfected subjects was used as the reference categories in FIG. 7 (4 copies for AAs, 2 for EAs, 3 for HAs).

To estimate the cumulative effect of decreasing CCL3L1 gene copy numbers on the risk of acquiring HIV-1 infection, we used the copy numbers where the cumulative curves for the distribution of CCL3L1 gene copies in HIV⁺ and HIV-uninfected individuals approximated as the reference category. When either of the off-diagonal cell counts in a two-by-two contingency table is zero it is not possible to directly derive an estimate of the relative risk. In such situations, a correction (Jewell's correction) (33) of 0.5 is added to all the cells and the table, and the OR and its 95% confidence interval (by Cornfield's methods) are estimated. In the HIV-uninfected HAs there were no subjects who were null for CCL3L1. Thus, we used Jewell's correction and estimated the odds ratio by comparing with possession of three copies as the reference category in FIG. 7H. For all the other copy numbers we used multivariate logistic regression.

To assess the risk of acquiring HIV associated with the combination of CCR5 and CCL3L1 genotypes (GRGs), we also used multivariate logistic regression in the setting of both vertical and horizontal transmission (FIG. 9, H and I).

The association between CCL3L1 gene copy numbers or CCL3L1/CCR5 GRGs and the rate of progression to AIDS, death and AIDS-defining illnesses was also examined (FIGS. 8, A and B; FIG. 8, G to L; FIG. 9, E to F; Table 10).

We conducted survival analysis for three outcomes: time to AIDS (1987 criteria), time to AIDS-related death, and AIDS-defining illnesses in the HIV⁺ individuals from the WHMC cohort. For the Argentinean children cohort we used only one end-point (time to AIDS, 1994 criteria) because of the small number of AIDS-related deaths in the cohort over the follow-up period. Kaplan-Meier (KM) survival curves and the log-rank test were used for between-group analysis. We used a Cox proportional hazards model to estimate the RHs (with 95% CI) associated with the specific genotypes. We tested for the assumption of proportional hazards by plotting the Schoenfeld residuals and used the program stphtest (Stata 7.0) to formally test the assumption (34). Schoenfeld residuals were calculated for each Cox proportional hazards model studied by using the Breslow-Peto approach.

In FIG. 8C, the overall association between CCL3L1 gene copies and CCR5 expression levels was determined by an overall Kruskal-Wallis test, and as this test was significant, the Mann-Whitney test was used post-hoc to determine the relationship between different CCL3L1 gene doses and CCR5 expression levels.

In FIG. 8D, the total CCL3L1 and CCL3 measured by ELISA in each sample was corrected for the number of cells using optical density as the normalizing factor. The association between CCL3L1 copy numbers and CCL3/CCL3L1 levels was conducted using the method of quantile regression, a non-parametric method. This method is preferred over multiple linear regression because of its robustness by circumventing the distributional assumptions (35-37). We estimated the median, lower quartile range and upper quartile range of chemokine production for each copy of the CCL3L1 gene. We then estimated the standard deviation of the median by using the following formula (38, 39):

$$SD = \frac{0.926 R}{\sqrt{N}} ,$$

where, R is the quartile range and N is the sample size. Note, in FIG. 8D, we plotted the confidence intervals around the median by using 1.7 standard deviations as described previously (38, 39). To determine whether there was a relationship between possession of CCL3L1 gene dose and chemokine production we fitted a second order polynomial curve. The model being fit to the data was:

Chemokine production=β₀+β₁*CCL3L1 copy number+β₂*CCL3L1 copy number²

The significance of β₁ and β₂ (the regression parameters) was tested and reported as the linear and quadratic terms, respectively. Non-linear association was interpreted if the quadratic term approached statistical significance. A non-linear association is inferred when the quadratic term in the regression equation approaches statistical significance.

We used a log transform on the HIV RNA copy number and then used non-parametric methods to ensure robustness. For the analysis presented in FIG. 8E, we estimated the median, lower quartile range and upper quartile range of the continuous variable (initial viral load) for each CCL3L1 gene copy and estimated the standard deviation of the median.

To assess a potential non-linear association between the median values of the initial viral load and possession of different copy numbers, we fitted a second order polynomial curve.

The following model was fitted: Log (Initial viral load)=β₀+β₁*CCL3L1 copy number+β₂*CCL3L1 copy number². The significance of β₁ and β₂ (the regression parameters) was tested and reported as the linear and quadratic terms, respectively. Again, non-linear association was interpreted if the quadratic term approached statistical significance.

In FIG. 9D, to determine the associations between GRGs and initial viral load, we estimated the median and 1.7SD of the median. We then compared the median log viral RNA copy number for the CCL3L1^{high}CCR5^{nondet} group with that for the CCL3L1CCR5^{det} group using the Mann—Whitney test.

The association between CCL3L1 gene copy numbers or CCL3L1/CCR5 GRGs and rate of change in leukocyte subsets, including CD4+ T cells in the HIV-positive WHMC cohort was also examined (FIGS. 8 F, M, and N, 9 B and C, and Table 9). We used the method of Generalized Estimating Equations (GEE) (40-43) to estimate the rate of change in leukocyte subsets. The GEE method is used to estimate population-averaged panel-data models (44-51). Using this method, we estimated the average monthly rates of change for the leukocyte subsets; positive numbers indicate an overall increase, whereas negative numbers indicate an overall decline in the rate of change in cell counts. Thus, for example, relative to a low negative number (e.g., −4.0), a higher negative number (e.g., −1.0) reflects a slower rate of decline in CD4⁺ or CD8⁺ T cells in FIGS. 9 B and C.

For the analysis presented in FIG. 8F, we estimated the rate of CD4+ T cell counts [±1.96× standard error (SE)] for each CCL3L1 copy number. We then used a second-order polynomial regression, as described in section 4.5, to determine the association between CCL3L1 gene dose and the rate of change of CD4+ T cell counts. For FIGS. 8 M and N and Table 9, we determined the association between CCL3L1 gene copies with rates of change in the following: total lymphocyte count, CD3+ T cell count, CD4+ T cell count, CD8+ T cell count, % CD3+ T cells and % CD4+ T cells; for these analyses, we used the CD3− T cell count as a negative control.

Separate models were used for subjects who possessed CCL3L1 gene copy numbers that were lower than or equal to the population median, and the comparisons were made on the basis of 95% CIs around the estimated rates of change. In a similar manner, we determined the association between CCL3L1/CCR5 GRGs and rate of change in CD4+ and CD8+ T cells (FIG. 9, B and C).

Analyses of modeling the changes in the frequency distribution of CCL3L1 gene copy numbers (FIG. 8, O and P) or CCL3L1/CCR5 GRGs (FIG. 9, J to L) over time in HIV-selecting settings were also carried out. These analyses were conducted to substantiate the concept of phenotypic equivalency. Additionally, these analyses permitted us to determine the possibility that HIV-1 might exert a selection force on the frequency distribution of CCL3L1 gene copy numbers in a HIV-positive cohort. That is, if CCL3L1 gene dose is a major determinant of HIV/AIDS susceptibility, then at the level of a well-characterized cohort, we would anticipate that the distribution of CCL3L1 gene copies in HIV⁺ and HIV⁻ individuals should vary significantly initially and then become increasingly similar over time. To study this possibility, we used two strategies.

First, we determined the frequency distribution of CCL3L1 gene copy numbers in the HIV-positive WHMC cohort at different lengths of follow-up period. We determined the changes in the distribution of CCL3L1 gene copies at the level of the entire cohort, i.e., combined analyses of EAs and AAs, and then separately in the EA and AA components of the cohort. The median number of CCL3L1 gene copies in the entire HIV⁺ WHMC cohort was two, regardless of ethnicity. We therefore trichotomized our dataset into classes of subjects with less than, equal to, and more than two CCL3L1 gene copies. We then categorized the follow-up time as <3, 3-4, 5-6, 7-8, and ≧9 years. We conducted a χ² test for linear trend on each of the three classes of CCL3L1 gene copy numbers across increasing lengths of follow-up. We also conducted comparisons of each follow-up time category with the HIV-uninfected controls to assess the time point at which the χ² becomes non-significant; this would reflect the time point at which the CCL3L1 gene copy frequency distributions in the HIV⁺ and HIV⁻ subjects are similar.

We found that the pattern in the changes in frequency distribution of CCL3L1 gene copies in the HIV⁺ individuals over time in the entire cohort or after stratification based on ethnicity was similar. The frequency distributions of the CCL3L1 gene copy numbers between the HIV⁺ and HIV⁻ individuals were most dissimilar in HIV⁺ individuals with a short follow-up time, with perceptible changes occurring with increasing lengths of follow-up, and eventually the CCL3L1 gene distribution in HIV⁺ individuals followed for ≧9 years was very similar to the uninfected individuals at the level of the entire cohort or after stratification based on ethnicity. A similar analysis was conducted using CCL3L1/CCR5 GRGs in FIG. 4 J-L.

Second, we conducted Markov model simulations of the health-state transitions within the infinite-sized cohort (FIG. 8, O and P). We used eight categories of the CCL3L1 gene copy numbers (0-≧7) and one health state, i.e., AIDS. The transitions are depicted in the state-transition diagram shown in Fig. S21. We used the DATA 3.5® software package for Markov model analysis. The details of the technique of Markov modeling have been reviewed previously (52-54).

To first assess whether the model correctly predicted our findings, we imposed the sample-size constraints on the observations and assessed the χ² test for differences between the model predicted frequency distribution of the CCL3L1 gene copy numbers and the observed frequencies in the HIV-uninfected controls. We then predicted the frequency distribution in infinite-sized EA and AA cohorts by plotting the expected frequency of each copy number as the cohort was followed over time (FIG. 8, O and P). The results of Markov modeling corroborated with the intuitive expectations that (a) the frequencies of different CCL3L1 gene copy numbers in a cohort of HIV⁺ individuals evolve such that eventually they mimic the HIV-uninfected subjects, and (b) the changes in frequency for any given CCL3L1 gene copy number over time are maximum for the population-specific median copy number (FIGS. 8, O and P).

In epidemiological studies assessing associations, the formula of AF is commonly employed for risk factors that have a dichotomized, i.e., all-or-none representation of exposure. The extension of this formula to the more common situation of multiple category risk factors is less well practiced. This statistical issue has been described in detail by Hanley et al., Miettinen et al., Kleinbaum et al., and Schlesselman (55-58). Here, we describe how we adapted these methods of estimating the AFs for the CCL3L1/CCR5 GRGs for the risk of acquiring HIV-1 and rapid rate of disease progression.

If rᵢ represents the disease risk associated with the ith genotype, and fᵢ is its frequency in the population, then the overall attributable fraction of the GRGs (all groups considered together) is given by

$${AF}_{overall} = \frac{\sum ( {r}_{i} - 1 ) {f}_{i}}{1 + ( {r}_{i} - 1 ) {f}_{i}} .$$

One can then estimate the genotype-specific attributable fraction by using the formula

$${AF}_{i} = \frac{( {r}_{i} - 1 ) {f}_{i}}{1 + \sum ( {r}_{i} - 1 ) {f}_{i}} ,$$

so that AFₒᵥₑᵣₐₗₗ=ΣAFᵢ. For estimating the risk of acquiring HIV-1, we used the odds ratios associated with each GRG, whereas for estimating the risk of accelerated progression to AIDS after HIV transmission, we used the hazard ratios from Cox proportional hazards models. We used the uninfected cohort frequencies of the genotypes for the calculation of attributable fractions. Using the confidence intervals around the odds ratios (or hazard ratios), we then estimated 95% confidence intervals around the point estimate of the attributable fractions.

Because in all populations examined, possession of <2 gene copies was associated with an enhanced risk of acquiring HIV-1, and since possession of <2 gene copies was also associated with an accelerated disease course in infected EA and AA adults, a “low” CCL3L1 gene copy number could be theoretically defined as <2 copies regardless of ethnic background.

In essence, this definition would imply that the absolute number of CCL3L1 gene copies (in this case 2 copies) rather than the gene copy number relative to the distribution of CCL3L1 gene copies in a population could be a criterion for defining the low copy number. While such a definition would somewhat reduce/minimize the complexity of the analyses, this definition would be valid only if the absolute copy number across ethnic groups can be considered to be associated with similar risk profiles. To address this issue, we conducted the following analyses.

First, we examined the distribution of the CCL3L1 gene copies across the HIV-negative populations that we used in comparative analyses to the HIV-positive subjects (FIG. 7). Analyses of the HIV-negative subjects indicated that there was significant inter-individual variation in CCL3L1 gene copy numbers, with a range of 0-11 copies (FIG. 7, A to D; open bars). For example, of the 1,668 HIV-negative individuals whose findings are shown in FIG. 7, -2% were null for CCL3L1. The frequency of individuals who were null for CCL3L1 was highest in EAs (2.81%) and least in AAs (0.20%). Conversely, a greater proportion of AAs had higher CCL3L1 gene copy numbers than any other population studied herein (FIG. 7B). Indeed, analysis of variance indicated that ethnicity accounted for ˜20% of the variation in CCL3L1 copy numbers in HIV-negative adult subjects (F=53.87, P=3.6×10⁻⁵⁰). In HIV-negative EAs and HAs as well as HIV-1-negative Argentinean children there was a sharp peak in the number of individuals who had two CCL3L1 copies (FIG. 7, A, C and D). In striking contrast, in HIV-negative AAs the distribution profile of CCL3L1 was more flat and skewed to the right as a greater proportion of HIV-negative AAs had between 2 to 5 copies with ˜19%, ˜19%, ˜25% and ˜15% possessing 2, 3, 4 and 5 CCL3L1 copies, respectively (FIG. 7B).

Thus, there were distinct inter-population differences in the distribution of CCL3L1 gene copy numbers that might preclude using a uniform cut-off point, since such a cut-off might not capture the range of individuals who, for a given CCL3L1 gene copy number might have quite different transmission- and disease-influencing phenotypic effects. That is, the phenotypic effects associated with 2 copies in EAs might not be the same as possession of 2 copies in AAs. Thus, slicing different populations with the same yardstick (absolute copy number) might capture individuals who although possessing the same copy numbers, may have different HIV-1-transmission/disease-influencing phenotypic effects.

Second, we examined the distribution of CCL3L1 gene copies in HIV-negative and HIV⁺ individuals in FIG. 7. We found that the distribution of CCL3L1 gene copy numbers in HIV-positive individuals was shifted to the left relative to those for the HIV-negative individuals. This shift to the left indicated that individuals with low CCL3L1 gene copy numbers were overrepresented among HIV-positive individuals relative to those who were HIV-negative. For example, in HIV-negative EAs, the median CCL3L1 gene copy number was 2, with ˜24%, 51% and ˜25% possessing <2, 2 and >2 CCL3L1 gene copies. In contrast, in HIV-positive EAs, 42%, ˜38% and ˜20% possessed <2, 2 and >2 CCL3L1 gene copies, respectively. Thus, in HIV-positive EA subjects there was a nearly 2-fold enrichment in individuals who possessed <2 copies as compared to the HIV-negative EA subjects. As another example, the median CCL3L1 copy number in this cohort of HIV AAs was four, whereas it was three in the HIV⁺ AAs (FIG. 7B).

Additionally, inspection of the histograms revealed a striking pattern in the distribution in the ratio of HIV⁺/HIV⁻ individuals across the spectrum of CCL3L1 copy numbers. There was a distinct CCL3L1 gene copy number at or above which the ratio of HIV⁺/HIV⁻ individuals was ≦1 (i.e., reduced or no difference in risk of infection; right of vertical arrow in FIG. 7) and below which the ratio switched to being consistently >1 (i.e., enhanced risk of infection; left of vertical arrow). Notably, the CCL3L1 gene copy number at which this switch or transition from enhanced to reduced or no change in susceptibility (i.e., HIV⁺/HIV⁻ ratio=1) corresponded very closely to the median gene copy numbers in the different groups of HIV-negative individuals (at the vertical arrow in FIG. 7).

Thus, the copy number at which the HIV⁺/HIV⁻ ratio switched from >1 to ≦1 served as a relevant reference point to compute the relative risk (odds ratio) of acquiring HIV-1. Relative to the CCL3L1 gene copy numbers that served as the population “switch or transition point,” CCL3L1 copy numbers below this switch point were associated with a higher risk of acquiring HIV-1 infection. In contrast, relative to this switch point, possession of CCL3L1 copy numbers greater than the population median were associated with either a reduced, or no statistical difference in the risk of acquiring HIV-1 infection (FIG. 7, E to H). For example, in AAs relative to possession of four CCL3L1 gene copies—the AA population average—possession of <4 CCL3L1 copies was associated with a significantly higher risk of acquiring infection [OR=1.84; CI=1.31-2.58; P=0.0004]. Specifically, relative to possession of four CCL3L1 gene copies, AAs who possessed 0, 1, 2, and 3 copies had an 8.12-, 4.36-, 1.55-, and 1.56-fold higher risk, respectively, of acquiring HIV-1 infection (FIG. 7F). In EA adults, relative to the population average of two CCL3L1 gene copies, possession of <2 CCL3L1 copies was associated with a 2.45-fold higher (CI, 1.90-3.17) risk of acquiring HIV-1 (P=9.8×10⁻¹²; FIG. 7G). A similar association between possession of CCL3L1 gene copies and risk of acquiring HIV-1 was also observed in HAs (FIG. 7H).

Third, we considered whether the likelihood of acquiring HIV-1 infection is identical for the corresponding copy numbers across populations. For this analysis, we used the lintrend software program and estimated the log(odds) of acquiring HIV for each copy number in different ethnic groups. We compared the HIV-infected subjects from the WHMC cohort with two different cohorts of HIV-uninfected subjects (non-WHMC and WHMC HIV-uninfected controls/reference groups).

The indicated uninfected population refers to the reference group. We found that possession of <2 copies of the CCL3L1 gene is associated with an increased risk of acquiring HIV irrespective of ethnic background, as indicated by the almost parallel lines between 0 and 2 gene copies. However, a careful comparison of the lines (which indicate trends) and the points (which indicate the copy-specific log [odds]) illustrates an important point. In individuals null for CCL3L1, the risk of acquiring HIV-1 is nearly 2 to 3 times higher in AAs as compared with EAs. Thus, while being null for CCL3L1 (zero copies) is associated with an increased risk in all populations, the strength of the increased risk is not equal across all ethnic groups. A similar pattern is observed for possession of one CCL3L1 gene copy among EAs and AAs.

Fourth, we estimated the increased risk of acquiring HIV associated with possession of <2 copies across ethnic groups. These findings suggest that while possession of less than 2 copies of CCL3L1 increases the risk of acquiring HIV, the strength of this association is not the same across ethnic backgrounds as in AAs this risk is nearly twice that observed in non-AAs. To assess whether these differences in the odds ratios are statistically significant across ethnic groups we used the Breslow-Day (B-D) test of homogeneity. We observed that the P values for homogeneity were consistently less than 0.2, demonstrating that the risk of HIV-acquisition in subjects possessing less than 2 copies of CCL3L1 could be different across ethnic groups.

One explanation for this significant difference is the switch point. While it is true that the switch points may simply be a statistical average, it might also represent a biologically and evolutionarily relevant genetic state. This is suggested by the following findings. If we dichotomize the AAs and EAs based on possession of < or ≧ the population-specific median, i.e., AAs possessing <4 and ≧4 copies and the EAs possessing <2 and ≧2 CCL3L1 gene copies, then the heterogeneity of odds ratios is no longer evident, substantiating our thesis of phenotypic equivalency (i.e., 2 copies in EAs is equivalent to 4 copies in AAs). By accounting for the ethnic background in this manner (i.e., by accounting for the population-specific switch point) the results of the Breslow-Day (B-D) test showed that the odds ratio estimates are now comparable across ethnic groups.

Taken together, these analyses suggest that the heterogeneity of association for the risk of acquiring HIV-1 across populations is mainly because of the different “CCL3L1 genetic fulcrum” points or differing medians in different populations and hence indicate that using a uniform cut-off of <2 CCL3L1 copies across all ethnic groups may not be an accurate representation of the phenotypic effects associated with variable CCL3L1 gene copies in the different populations.

In addition to these aforementioned analyses, there were two additional reasons why an arbitrary cut-off of two copies for statistical analyses was not used.

(i) To subdivide a population into two groups (e.g., high and low) based on some measured variable, there is no a priori reason for selecting any dividing point other than the median or other average value. Furthermore, based on the aforementioned findings, we surmised that it might be inappropriate to ignore the fact that the medians are very different in the clearly identifiable subsets of EAs and AAs.
(ii) There are clear precedents for population (race)-specific differences in the effect of particular genotypes on HIV transmission/disease-influencing phenotypes. In addition to our own work (3, 7), this inference is supported by other large studies from MACS investigators (21, 59, 60). From this perspective also, it might not be appropriate to make the tacit assumption that the phenotypic effect of CCL3L1 gene copy number would be independent of ethnicity/continent of origin. Thus, to remain consistent with our previous work on the identification of population-specific CCR5 genotypes that influence HIV/AIDS susceptibility (3, 7) and our above analyses, we have used population-specific median number of CCL3L1 gene copy numbers in our studies, and not an arbitrary cut-off.

Several sets of criteria for inference of a causal relation between a causative factor and a disease using epidemiological studies have been suggested. One such set is that of A. B. Hill, which includes criteria such as strength of association, dose-response relation, consistency of association, temporally correct association, specificity of association, and biological plausibility (31, 32). Among these criteria, the criterion of dose response reinforces the evidence in favor of causality. The analyses conducted to determine the dose-response relationship between possession of CCL3L1 gene copy number and risk of HIV acquisition are outlined below.

Using unconditional logistic regression (model #1), we regressed the CCL3L1 gene copy number as an ordinal variable onto the risk of acquiring HIV infection in the settings of vertical or horizontal transmission. The results of these analyses can be interpreted as the overall risk/protection associated with possession of each additional CCL3L1 gene copy. A statistically significant value of the odds ratio indicates a monotonically decreasing/increasing risk of acquiring HIV-1 infection associated with increasing/decreasing CCL3L1 gene copies. The results indicate that there is a consistent and statistically significant protective effect associated with each additional CCL3L1 gene copy in the different clinical settings.

Witte and Greenland (61) showed that a potential spuriousness/confounding in such models can be assessed by using a nested model approach. They suggested that a quadratic dose-response can be assessed by including a square-term in the regression models. We therefore used a nested model (model #2) with the copy number and squared copy number as the predictors in the logistic models.

We observed that in the context of vertical transmission and in HAs, there remained a strong linear relationship even after including the square term. In contrast, in EAs and AAs, we observed that there was a significant or nearly significant quadratic relationship in addition to a significant linear association. These findings corroborate the inferences derived by examining the distribution of cumulative frequencies of the CCL3L1 gene copy numbers, suggesting that there is a dose-response relationship between CCL3L1 gene dose and risk of acquiring HIV-1, and that the nature of this relationship is either linear or hemiparabolic, i.e., with a threshold effect.

We used alternative approaches to substantiate this dose-response relationship. A common method used to assess the dose-response association in comparative studies is probit analysis (62-65). A probit model uses a binary dependent variable (e.g., acquisition of HIV infection, development of AIDS) and is described as: Pr(yⱼ≠|xⱼ)=Φ(xⱼb), where xⱼb represents a composite probit score or index. This index is assumed to follow a normal distribution, and therefore the interpretation of probit results needs to be in light of the relative deviate (z value) domain. A major use of probit models is to estimate dose thresholds, e.g., ED50 and LD50 (64)

To assess the dose-response relationship between possession of varying CCL3L1 gene copy numbers and risk of acquiring HIV infection, we used logistic regression as well as probit repression. While comparing these results, it is important to recognize that the dependent variable in the logistic regression is log odds, whereas in probit regression, it is the probability of the binary outcome. Moreover, the independent variables in logistic regression are untransformed, whereas in probit analysis, they are transformed to a composite index. Thus, the direction of the effect, rather than the numerical quantity, needs to be compared. As the results of the analyses were essentially very similar, we have reported the results of logistic regression analysis.

A comparison of the model fits (based on likelihood ratio χ²) for these models revealed again that both the logistic and probit models performed similarly.

Therefore, we used results from the logistic regression analyses (which are simpler to interpret than those of probit regression). Logistic regression equation for the results can be denoted by the following formula:

Probability of binary outcome=e^{(β0+β1*copies)}/[1+e^{(β0+β1*copies)}].

Using this formula, we estimated the probability of acquiring HIV infection conditional upon the estimated values of regression coefficients (β0 and β1) for possession of 0-≧7 copies of the CCL3L1 gene. We then fitted a least-squares line through these estimates. The slope of the fitted line gives the average change in the probability of HIV acquisition for each incremental copy of the CCL3L1 gene. These analyses indicated that each copy of CCL3L1 was associated, in general, with a 7.54% reduced risk of acquiring HIV-1 in the setting of mother-to-child transmission (95% CI 7.01%-8.07%, P=4.3×10⁻⁹). In AAs, EAs, and HAs each CCL3L1 gene copy number was associated with a 6% (95% CI 5.8%-6.1%, P=1×10⁻¹⁰), 4.5% (95% CI 4.3%-4.7%, P=5.7×10⁻¹⁰), and 10.5% (95% CI 8.6%-12.4%, P=1.1×10⁻⁵) lower risk of acquiring HIV-1, respectively. Using the same results from logistic regression analyses, we estimated the differences in the risk of infection between those who possessed population-specific highest and lowest CCL3L1 gene copy numbers, and they were 54% in EAs, 63% in AAs, ˜100% in HAs, and 79% in children exposed perinatally to HIV.

Causation is an essential concept in epidemiology, although clearly a difficult one to substantiate. For several decades, the Bradford-Hill criteria for causality have been used widely to elucidate a causal relationship with an observed “association” (31, 32). Notably, within the populations we examined, several of the essential criteria (italics) for causality between CCL3L1 dose and risk of acquiring HIV-1 were met. These include temporality (preexisting genetic state prior to infection), strength of association (FIG. 7, Table 16), dose-response, experimental evidence and consistency across two different modes of acquiring infection as well as populations of African and European descent. The highly potent anti-HIV-1 effects of CCL3L1 (66, 67) in conjunction with the association between increased chemokine/reduced CCR5 expression (FIG. 8, C and D), a previously established correlate of protection (68-74), and high CCL3L1 dose provide biological plausibility.

### REFERENCES FOR EXAMPLE IV

1. N. A. Rosenberg et al., Science 298, 2381 (2002).
2. H. M. Cann et al., Science 296, 261 (2002).
3. E. Gonzalez e tal., Proc Natl Acad Sci USA 96, 12004 (1999).
4. E. Gonzalez et al., Proc Natl Acad Sci USA 98, 5199 (2001).
5. E. Gonzalez et al., Proc Natl Acad Sci USA 99, 13795 (2002).
6. S. Mummidi et al., Nat Med 4, 786 (1998).
7. A. Mangano et al., J Infect Dis 183, 1574 (2001).
8. P. A. Zimmerman et al., Mol Med 3, 23 (1997).
9. R. S. Sperling et al, N Engl J Med 335, 1621 (1996).
10. J. R. Townson, L. F. Barcellos, R. J. Nibbs, Eur J Immunol 32, 3016 (2002).
11. P. Menten, A. Wuyts, J. Van Damme, Cytokine Growth Factor Rev 13, 455 (2002).
12. A. Amara et al., J Exp Med 186, 139 (1997).
13.1. Aramori et al., Embo J 16, 4606 (1997).
14. M. Mack et al., J Exp Med 187, 1215 (1998).
15. R. Sabbe et al., J Virol 75, 661 (2001).
16. R. G. Carroll et al., Science 276, 273 (1997).
17. J. L. Riley et al., J Immunol 158, 5545 (1997).
18. P. Secchiero et al., J Immunol 164, 4018 (2000).
19. S. Mummidi et al., J Biol Chem 275, 18946 (2000).
20. D. H. McDermott et al., Lancet 352, 866 (1998).
21. M. P. Martin et al., Science 282, 1907 (1998).
22. C. G. Anastassopoulou, L. G. Kostrikis, Curr HIV Res 1, 185 (2003).
23. J. Tang, R. A. Kaslow, Aids 17 Suppl 4, S51 (2003).
24. J. Tang et al., J Virol 76, 662 (2002).
25. J. Tang et al., AIDS Res Hum Retroviruses 18, 403 (2002).
26. P. A. Ramaley et al., Nature 417, 140 (2002).
27. S. J. O'Brien, J. P. Moore, Immunol Rev 177, 99 (2000).
28. B. M. Neale, P. C. Sham, Am J Hum Genet 75, 353 (2004).
29. J. W. Mellors et al., Ann Intern Med 126, 946 (1997).
30. J. W. Mellors et al., Science 272, 1167 (1996).
31. A. B. Hill, Proc R Soc Med 58, 295 (1965).
32. D. L. Weed, Hematol Oncol Clin North Am 14, 797 (2000).
33. S. D. Walter, R. J. Cook, Biometrics 47, 795 (1991).
34. J. M. Garrett, Stata Technical Bulletin 35, 9 (1997).
35. R. Koenker, J. G. Bassett, Econometrica 50, 43 (1982).
36. S. C. Narula, J. F. Wellington, International Statistical Review 50, 317 (1982).
37. W. H. Rogers, Stata Technical Bulletin 13, 18 (1993).
38. R. McGill, J. Tukey, W. Larsen, Am Stat 32, 12 (1978).
39. Minor and trace elements in breast milk. Report of a Joint WHO/ILEA Collaborative Study, WHO, Geneva (1989), pg. 9-10.
40. K. L. Fielding et al., Stat Med 14, 1365 (1995).
41. A. C. Ghani et al., J Acquir Immune Defic Syndr 28, 226 (2001).
42. S. R. Lipsitz, G. Molenberghs, G. M. Fitzmaurice, J. Ibrahim, Biometrics 56, 528 (2000).
43. J. Roy, X. Lin, L. M. Ryan, Biostatistics 4, 371 (2003).
44. S. L. Zeger, K. Y. Liang, Biometrics 42, 121 (1986).
45. S. L. Zeger, K. Y. Liang, Stat Med 11, 1825 (1992).
46. J. Rochon, Stat Med 17, 1643 (1998).
47. B. C. Supradhar, K. Das, Biometrics 56, 622 (2000).
48. S. R. Lipsitz, G. M. Fitzmaurice, E. J. Orav, N. M. Laird, Biometrics 50, 270 (1994).
49. T. Park, Stat Med 12, 1723 (1993).
50. J. M. Williamson, S. R. Lipsitz, K. M. Kim, Comput Methods Programs Biomed 58, 25 (1999).
51. A. Hadgu, G. Koch, J Biopharm Stat 9, 161 (1999).
52. P. Hougaard, Lifetime Data Anal 5, 239 (1999).
53. D. S. Kucey, World J Surg 23, 1227 (1999).
54. S. D. Ramsey et al., Hematol Oncol Clin North Am 14, 925 (2000).
55. J. A. Hanley, J Epidemiol Community Health 55, 508 (2001).
56. O, S. Miettinen, Theoretical epidemiology: principles of occurrence research in medicine (Wiley, New York, 1985), pp. 254-6.
57. D. G. Kleinbaum, L. L. Kupper, H. Morgenstern, Epidemiologic research: principles and quantitative methods (Lifetime Learning Publications, Belmont (CA), 1982), pp. 160-4.
58. J. J. Schlesselman, Case control studies: design, conduct, analysis (Oxford University Press, New York, 1982), pp. 220-6.
59. P. An et al., Proc Natl Acad Sci USA 99, 10002 (2002).
60. H. D. Shin et al., Proc Natl Acad Sci USA 97, 14467 (2000).
61. J. S. Witte, S. Greenland, Ann Epidemiol 7, 188 (1997).
62. P. J. Catalano, Stat Med 16, 883 (1997).
63. M. Coleman, H. Marks, J Food Prot 61, 1550 (1998).
64. R. L. Prentice, Biometrics 32, 761 (1976).
65. M. M. Regan, P. J. Catalano, Biometrics 55, 760 (1999).
66. R. J. Nibbs, J. Yang, N. R. Landau, J. H. Mao, G. J. Graham, J Biol Chem 274, 17478 (1999).
67. S. Aquaro et al., J Virol 75, 4402 (2001).
68. W. A. Paxton et al., Virology 244, 66 (1998).
69. D. Zagury et al., Proc Natl Acad Sci USA 95, 3857 (1998).
70. W. A. Paxton et al., Nat Med 2, 412 (1996).
71. A. Garzino-Demo et al., Proc Natl Acad Sci USA 96, 11986 (1999).
72. J. Reynes et al., J Acquir Immune Defic Syndr 34, 114 (2003).
73. H. Ullum et al., J Infect Dis 177, 331 (1998).
74. W. A. Paxton et al., J Infect Dis 183, 1678 (2001).
75. P. Proost et al., Blood 96, 1674 (2000).
76. P. Menten et al., J Clin Invest 104, R1 (1999).
77. F. Cocchi et al., Science 270, 1811 (1995).
78. R. J. Nibbs et al., J Biol Chem 272, 32078 (1997).
79. S. Struyf et al, Eur J Immunol 31, 2170 (2001).
80. S. D. Blacksell, L. J. Gleeson, R. A. Lunt, C. Chamnanpood, Rev Sci Tech 13, 687 (1994).
81. A. P. Morton et al, J Qual Clin Pract 21, 112 (2001).
82. F. E. Nelson, M. K. Hart, R. F. Hart, J Am Acad Nurse Pract 6, 17 (1994).
83. D. Stamm, J Clin Chem Clin Biochem 20, 817 (1982).
84. E. Swenson-Britt et al., Jt Comm J Qual Improv 27, 540 (2001).
85. J. O. Westgard, P. L. Barry, M. R. Hunt, T. Groth, Clin Chem 27, 493 (1981).
86. W. A. Shewhart, Economic control of quality of manufactured product. (D. Van Nostrand Company, New York, 1931).
87. E. W. Steyerberg et al., J Clin Epidemiol 56, 441 (2003).
88. M. Schumacher, N. Hollander, W. Sauerbrei, Stat Med 16, 2813 (1997).
89. B. Liquet, C. Sakarovitch, D. Commenges, Biometrics 59, 172 (2003).
90. A. C. Davison, D. V. Hinkley, Bootstrap methods and their applications. (Cambridge University Press, Cambridge, 1997).

### Example V

### Genetic Variations in the Receptor-Ligand Pair, CCR5 and CCL3L1, are Important Determinants of Susceptibility to Kawasaki Disease

Kawasaki disease (KD) is an enigmatic, self-limited vasculitis of childhood complicated by development of coronary artery aneurysms (CAA). The high incidence of KD in Asian versus European populations prompted a search for genetic polymorphisms that are both differentially distributed among these populations and influence KD susceptibility. Here a striking, inverse relationship between the world-wide distribution of the CC chemokine receptor 5 (CCR5)-Δ32 allele and the incidence of KD is demonstrated. In 164 KD case-parent trios, four CCR5 haplotypes, including the CCR5-Δ32 allele were differentially transmitted from heterozygous parents to affected children. However, the magnitude of the reduced risk of KD associated with the CCR5-Δ32 allele and certain CCR5 haplotypes was significantly greater in individuals who also possessed a high copy number of the gene encoding CCL3L1, the most potent CCR5 ligand. These findings, derived from the largest genetic study of any systemic vasculitis, suggest a central role of CCR5-CCL3L1 gene-gene interactions in KD susceptibility and the importance of gene modifiers in infectious diseases.

All patients with KD or a history of KD who met 4 of the 5 standard clinical criteria [1,5] or 3 of the 5 criteria plus coronary artery abnormalities documented by echocardiography and for whom both biologic parents agreed to donate DNA samples were entered into the study after obtaining informed consent. This protocol was reviewed and approved by the Institutional Review Board at UCSD and Boston Children's Hospital.

Clinical data including gender, ethnicity, race, age of disease onset, response to intravenous gamma globulin therapy and coronary artery status were recorded for all subjects. All echocardiograms during the first two months following disease onset were recorded as either normal (all three vessels within 2 standard deviations of the mean internal diameter for body surface area of patient according to the Newburger criteria) or abnormal [16]. For abnormal echocardiograms (z score >2), the z score was recorded and the patient classified as “dilated” (z score >2.0 but <4.0 and returns to <2.0 within 2 mos. follow-up period) or “aneurysm/ectasia” (focal or persistent dilatation of coronary artery segment with z score >4.0).

For children <6 yrs., 3 cc of blood was collected into tubes containing EDTA and DNA was extracted using the using the Wizard Genomic DNA extraction kit (Promega) as previously published [17]. This procedure routinely yielded 25-75 μg of PCR-quality genomic DNA. For parents and KD children over the age of 6 yrs., 10 cc of Scope mouthwash was be used to collect shed buccal cells for DNA extraction [18]. The yield was between 10-200 μg of PCR-amplifiable genomic DNA.

The methods for genotyping CCR5 polymorphisms and generation of CCR5 haplotypes are as described previously [19, 20]. CCL3L1 gene copy number was estimated as described recently and very extensive methods are described in the Supplementary online material accompanying that paper [20].

Polymorphism data was subjected to Mendelian checks using the Pedcheck software. Where appropriate, haplotypes were inferred with the Genehunter software and double crossovers within genes or families of genes on the same chromosome were flagged for examination. When available, prior information regarding linkage disequilibrium among polymorphisms in the same gene or family of genes in the same chromosomal region was used to identify potential genotyping errors. If the error could not be resolved by repeated genotyping, then that triad was deleted from the study with the assumption that either an error occurred in collection or labeling one of the three samples in the triad or that one of the parents is not the biological parent. Following this protocol, only 4 families were deleted from the data set.

The correlation between CCR5Δ32 mutation frequency and KD incidence was compared using Spearman's correlation coefficient. We used the Transmission disequilibrium test (TDT) [21] to assess the transmission of each CCR5 haplotype in the trios. We used Stata 7.0 (Stata Corp, College Station, Tex.) software package (command: symmetry) for conducting the TDT analyses.

A limitation of the TDT method is that it is suited to only those marker loci that are biallelic. We have shown previously that the CCR5 focus is essentially multiallelic (with 9 haplotypes). Thus, we used the extended TDT (E-TDT) for multiallelic loci [22]. It has been demonstrated that the E-TDT has sufficient power when the linkage disequilibrium is strong. For conducting this analysis we used the program ETDT provided for public use by Dave Curtis. We used the case: pseudocontrol analysis described by Cordell and colleagues [23]. This approach, while retaining several of the advantages of family-based designs, concurrently accounts for the effects of maternal genotype and parent-of-origin (imprinting). In this approach, the pseudocontrols are generated from the three untransmitted parental genotypes and conditional logistic regression is used to test the association of the genotypes with disease. We conducted this analysis since it allows a multivariate estimation of the phenotypic effects of the genotypes and could therefore be used in the context of the multiallelic CCR5 locus. For the assessment of the phenotypic effects of CCR5 on the genetic background conferred by CCL3L1, we considered three genetic backgrounds conferred by the CCL3L1 gene copy number: <2 copies, 2 copies and >2 copies[1]. Within each of these categories we conducted the TDT, E-TDT and case:pseudo control analyses to assess if the genetic background conferred by CCL3L1 gene copy number influenced the phenotypic effects associated with the CCR5 haplotypes.

Results of these studies demonstrate a striking inverse correlation between the incidence of KD [8, 10, 24-30] and the frequency of the CCR5Δ32 allele [5, 31-36] in different geographic regions. Thus, countries with the lowest incidence of KD had the highest frequency of the CCR5-Δ32 allele. Conversely, in Japan, the country with the highest KD incidence in the world, the prevalence of the CCR5-Δ32 allele was virtually zero. While many polymorphisms differ in frequency between Asians and other populations, this inverse relationship between KD and CCR5-Δ32 prompted us to test whether the CCR5-Δ32 allele confers protection against developing KD in a family-based study. This cohort of 164 KD children and their biologic parents is, to the best of our knowledge, the largest number of affected cases for any reported genetic study of KD. We employed three complementary statistical approaches: the transmission disequilibrium test (TDT) [21], the multiallelic or extended version of the TDT (E-TDT) [37], and the case/pseudocontrol analysis [23].

For a marker locus with two alleles, the TDT compares the transmission of either allele from heterozygous parents to the affected offspring, and this test and the family-based design eliminates the risk of spurious associations due to population stratification, a common confounder in case/control association studies [38]. By TDT, we found asymmetric transmission of the CCR5-Δ32 allele from 46 heterozygous parents to their affected children (P=0.027; transmitted: not transmitted=15:31, Table 18, model 1), supporting the hypothesis that CCR5-Δ32 allele might protect against developing KD.

However, CCR5 is a multi-allelic gene, and in addition to the CCR5-Δ32 allele, we have described previously eight CCR5 haplotypes that take into consideration polymorphisms in the promoter of CCR5 and coding regions of CCR5 and CCR2[39]. These haplotypes are categorized into CCR5 human haplogroups A (HHA) to HHG*2, with the haplotype bearing CCR5-Δ32 designated as HHG*2.

Complete CCR5 haplotypes were obtained from 164 KD case-parent trios. The CCR5 locus in the study subjects was in Hardy-Weinberg equilibrium (multiallelic likelihood ratio test χ²=35.42, df=28, P=0.1579). The E-TDT analyses indicated that there was an overall significant association between asymmetric transmission of the CCR5 haplotypes from parents to their affected children (Likelihood ratio χ² 18.03, df=7, P=0.0119). Because of this overall association, we next determined which specific haplotype(s) accounted for this association. Three CCR5 haplotypes were associated with a significantly reduced risk of KD (Table 18, model 2). They were (i) HHA, (ii) HHC, and (iii) HHG*2, the CCR5-Δ32-containing haplotype, which was associated with a nearly 50% reduction in the risk of KD (95% CI 0.25-0.93). By contrast, the haplotype on which the CCR5-Δ32 mutation arose [39], namely CCR5—HHG*1, was associated with an increased risk of developing KD (Table 18, model 2).

We next determined if CCR5 haplotypes were associated with altered risk of coronary artery damage in children with KD. For this analysis we compared 83 children with normal echocardiograms with 88 children who had either coronary artery dilatation or frank aneurysms. There was a trend between possession of the CCR5-HHG*2 haplotype and a reduced risk of coronary artery dilatation or aneurysm (OR 0.36, 95% CI 0.12-1.07, P=0.067), which is notable considering the small sample size.

We also tested whether the KD-influencing effects associated with CCR5 haplotypes were modified by CCL3L1 gene dosage. In this analysis, we stratified KD cases into three groups: those who possessed CCL3L1 gene copy numbers that were less than, equal to, or greater than two, which was the median copy number for the entire cohort. TDT analyses for each CCL3L1 gene dose strata showed that the KD-influencing effects of the CCR5-Δ32 allele and CCR5 haplotypes were most evident in the context of certain CCL3L1 gene dose strata (Table 18, models 3 and 4). Specifically, the effects of CCR5-HHG*2, and —HHA were more significant in individuals who also possessed median or high CCL3L1 gene copy numbers, respectively. Thus, individuals who possessed both CCR5-HHG*2 and 2 copies of CCL3L1 had a nearly 80% lower risk of developing KD (Table 18, models 3 and 4). An association between KD susceptibility and possession of HHG*1 was not detected in this stratified analysis, suggesting that the effect of HHG*1 was distributed across the different CCL3L1 gene dose strata (Table 18, models 3 and 4).

Recently, Cordell and colleagues proposed a unified framework for genetic association testing, based on conditional logistic regression analysis of cases and matched pseudocontrols derived from the genotypes of the cases and parents [23]. This method allows nuclear family data to be analyzed in a very similar manner to case/control data, but by using conditional logistic regression models. This approach complements the TDT and E-TDT analyses, and the findings further underscore the influence of CCR5 haplotypes on KD susceptibility as well as the notion that these effects are most evident within the context of a specific genetic background that is dependent on CCL3L1 copy number (Table 19).

Taken together, these TDT analyses demonstrate that genetic variation in CCR5 plays an influential role in KD susceptibility and may influence coronary artery outcome in affected children. Striking parallels were noted between CCR5 genotype and susceptibility and outcome in both KD and HIV/AIDS: (i) CCR5-HH4 is the ancestral CCR5 haplotype [39], and is associated with resistance to KD as well as a reduced risk of progressing rapidly to AIDS in specific populations [19]; (ii) CCR5-HHG*2, the CCR5-Δ32-carrying allele, is associated with reduced KD susceptibility and protection from coronary artery aneurysms as well as a reduced risk of acquiring HIV and progressing rapidly to AIDS; (iii) the CCR5-HHG*1 haplotype is associated with increased KD susceptibility as well as an increased rate of disease progression to AIDS [42]; and (iv) the HIV/AIDS- and KD-influencing effects associated with CCR5 haplotypes are influenced by CCL3L1 gene dose [1].

### REFERENCES FOR EXAMPLE V

1. Gonzalez E, Kulkarni H, Bolivar H, et al. The Influence of CCL3L1 Gene-Containing Segmental Duplications on HIV-1/AIDS Susceptibility. Science 2005
2. Menten P, Wuyts A and Van Damme J. Macrophage inflammatory protein-1. Cytokine Growth Factor Rev 2002; 13:455-81
3. O'Brien S J, Moore J P. The effect of genetic variation in chemokines and their receptors on HIV transmission and progression to AIDS. Immunol Rev 2000; 177:99-111
4. Dean M, Carrington M, Winkler C, et al. Genetic restriction of HIV-1 infection and progression to AIDS by a deletion allele of the CKR5 structural gene. Hemophilia Growth and Development Study, Multicenter AIDS Cohort Study, Multicenter Hemophilia Cohort Study, San Francisco City Cohort, ALIVE Study. Science 1996; 273:1856-62
5. Lucotte G, Dieterlen F. More about the Viking hypothesis of origin of the delta32 mutation in the CCR5 gene conferring resistance to HIV-1 infection. Infect Genet Evol 2003; 3:293
6. Johnston J B, Barrett J W, Chang W, et al. Role of the serine-threonine kinase PAK-1 in myxoma virus replication. J Virol 2003; 77:5877-88
7. Galvani A P, Slatkin M. Evaluating plague and smallpox as historical selective pressures for the CCR5-Delta 32 HIV-resistance allele. Proc Natl Acad Sci USA 2003; 100:15276-9
8. Holman R C, Cums A T, Belay E D, Steiner C A and Schonberger L B. Kawasaki syndrome hospitalizations in the United States, 1997 and 2000. Pediatrics 2003; 112:495-501
9. Bronstein D E, Dille A N, Austin J P, Williams C M, Palinkas L A and Burns J C. Relationship of climate, ethnicity and socioeconomic status to Kawasaki disease in San Diego County, 1994 through 1998. Pediatr Infect Dis J 2000; 19:1087-91
10. Yanagawa H, Yashiro, M., Oki, I., Nakamura, Y., Zhang, T. Thirty-year observation of the incidence rate of Kawasaki disease in Japan. Pediatr Res 2002; 53:158
11. Burns J C, Glode M P. Kawasaki syndrome. Lancet 2004; 364:533-44
12. Uehara R, Yashiro M, Nakamura Y and Yanagawa H. Kawasaki disease in parents and children. Acta Paediatr 2003; 92:694-7
13. Hirata S, Nakamura Y and Yanagawa H. Incidence rate of recurrent Kawasaki disease and related risk factors: from the results of nationwide surveys of Kawasaki disease in Japan. Acta Paediatr 2001; 90:40-4
14. Mori M, Miyamae T, Kurosawa R, Yokota S and Onoki H. Two-generation Kawasaki disease: mother and daughter. J Pediatr 2001; 139:754-6
15. Newburger J W, Takahashi M, Gerber M A, et al. Diagnosis, treatment, and long-term management of Kawasaki disease: a statement for health professionals from the Committee on Rheumatic Fever, Endocarditis and Kawasaki Disease, Council on Cardiovascular Disease in the Young, American Heart Association. Circulation 2004; 110:2747-71
16. de Zorzi A, Colan S D, Gauvreau K, Baker A L, Sundel R P and Newburger J W. Coronary artery dimensions may be misclassified as normal in Kawasaki disease. J Pediatr 1998; 133:254-8
17. Quasney M W, Bronstein D E, Cantor R M, et al. Increased frequency of alleles associated with elevated tumor necrosis factor-alpha levels in children with Kawasaki disease. Pediatr Res 2001; 49:686-90
18. Heath E M, Morken N R, Campbell K A, Tkach D, Boyd E A and Strom D A. Use of buccal cells collected in mouthwash as a source of DNA for clinical testing. Arch Pathol Lab Med 2001; 125:127-33
19. Gonzalez E, Bamshad M, Sato N, et al. Race-specific HIV-1 disease-modifying effects associated with CCR5 haplotypes. Proc Natl Acad Sci USA 1999; 96:12004-9
20. Gonzalez E, H K, H B, et al. The influence of CCL3L1 gene-containing segmental duplications on HIV-1/AIDS susceptibility. Science 2005; In press
21. Spielman R S, McGinnis RE and Ewens W J. Transmission test for linkage disequilibrium: the insulin gene region and insulin-dependent diabetes mellitus (IDDM). Am J Hum Genet 1993; 52:506-16
22. Sham P C, Curtis D. An extended transmission/disequilibrium test (TDT) for multi-allele marker loci. Ann Hum Genet 1995; 59:323-336
23. Cordell H J, Barratt B J and Clayton D G. Case/pseudocontrol analysis in genetic association studies: A unified framework for detection of genotype and haplotype associations, gene-gene and gene-environment interactions, and parent-of-origin effects. Genet Epidemiol 2004; 26:167-85
24. Royle J A, Williams K, Elliott E, et al. Kawasaki disease in Australia, 1993-95. Arch Dis Child 1998; 78:33-9
25. Pelkonen P, Salo E. Epidemiology of Kawasaki disease. Clin Exp Rheumatol 1994; 12 Suppl 10:S83-5
26. Park Y W, Park I S, Kim C H, et al. Epidemiologic study of Kawasaki disease in Korea, 1997-1999: comparison with previous studies during 1991-1996. J Korean Med Sci 2002; 17:453-6
27. Du Z D, Zhang T, Liang L, et al. Epidemiologic picture of Kawasaki disease in Beijing from 1995 through 1999. Pediatr Infect Dis J 2002; 21:103-7
28. Harnden A, Alves B and Sheikh A. Rising incidence of Kawasaki disease in England: analysis of hospital admission data. Bmj 2002; 324:1424-5
29. Schiller B, Fasth A, Bjorkhem G and Elinder G. Kawasaki disease in Sweden: incidence and clinical features. Acta Paediatr 1995; 84:769-74
30. Lue H C, Philip S, Chen M R, Wang J K and Wu M H. Surveillance of Kawasaki disease in Taiwan and review of the literature. Acta Paediatr Taiwan 2004; 45:8-14
31. Feng T, Ni A, Yang G, Galvin S R, Hoffman I F and Cohen M S. Distribution of the CCR5 gene 32-base pair deletion and CCR5 expression in Chinese minorities. J Acquir Immune Defic Syndr 2003; 32:131-4
32. Gonzalez E, Dhanda R, Bamshad M, et al. Global survey of genetic variation in CCR5, RANTES, and MIP-1 alpha: impact on the epidemiology of the HIV-1 pandemic. Proc Natl Acad Sci USA 2001; 98:5199-204
33. Li C, Yan Y P, Shieh B, Lee C M, Lin R Y and Chen Y M. Frequency of the CCR5 delta 32 mutant allele in HIV-1-positive patients, female sex workers, and a normal population in Taiwan. J Formos Med Assoc 1997; 96:979-84
34. Oh M D, Kim S S, Kim E Y, et al. The frequency of mutation in CCR5 gene among Koreans. Int J STD AIDS 2000; 11:266-7
35. Martinson J J, Chapman N H, Rees D C, Liu Y T and Clegg J B. Global distribution of the CCR5 gene 32-basepair deletion. Nat Genet 1997; 16:100-3
36. Ansari-Lari M A, Liu X M, Metzker M L, Rut A R and Gibbs R A. The extent of genetic variation in the CCR5 gene. Nat Genet 1997; 16:221-2
37. Sham P C, Curtis D. An extended transmission/disequilibrium test (TDT) for multi-allele marker loci. Ann Hum Genet 1995; 59 (Pt 3):323-36
38. Ewens W J, Spielman R S. The transmission/disequilibrium test: history, subdivision, and admixture. Am J Hum Genet 1995; 57:455-64
39. Mummidi S, Bamshad M, Ahuja S S, et al. Evolution of human and non-human primate CC chemokine receptor 5 gene and mRNA. Potential roles for haplotype and mRNA diversity, differential haplotype-specific transcriptional activity, and altered transcription factor binding to polymorphic nucleotides in the pathogenesis of HIV-1 and simian immunodeficiency virus. J Biol Chem 2000; 275:18946-61
40. Mangano A, Gonzalez E, Dhanda R, et al. Concordance between the CC chemokine receptor 5 genetic determinants that alter risks of transmission and disease progression in children exposed perinatally to human immunodeficiency virus. J Infect Dis 2001; 183:1574-85
41. Anastassopoulou C G, Kostrikis L G. The impact of human allelic variation on HIV-1 disease. Curr HIV Res 2003; 1:185-203
42. Mummidi S, Ahuja S S, Gonzalez E, et al. Genealogy of the CCR5 locus and chemokine system gene variants associated with altered rates of HIV-1 disease progression. Nat Med 1998; 4:786-93

### Example VI

### Essential Role for CCL3L1/CCR5 Genotypes in Evaluation of AIDS Vaccine Endpoints and the Dynamics of HIV Epidemics

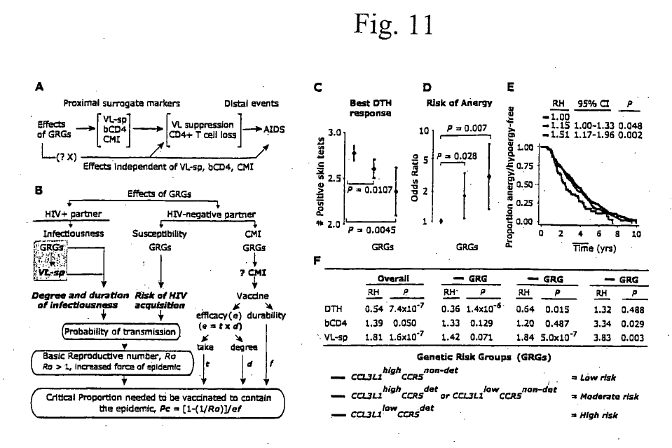

A large, well characterized cohort of HIV⁺ adults who have been followed prospectively at Wilford Hall Medical Center (WHMC), San Antonio, Tex. from the very early stages of their infection (9) were used to test four conceptual constructs by which the transmission and/or disease-influencing effects associated with the genetic risk groups (GRGs) derived from the gene dose of CCL3L1 and polymorphisms in CCR5 might confound the analyses of vaccine endpoints. First, by influencing cell-mediated immunity (CMI) or other immunological processes, CCL3L1/CCR5 variants affect critical proximal determinants of disease progression, such as the magnitude of initial loss in CD4⁺ cells and the VL setpoint (VL-sp) (FIG. 11A). Second, CCL3L1/CCR5 variants have disease-influencing effects that are independent of their influence on the VL-sp or baseline CD4⁺ cells (bCD4) (FIG. 11A). Third, if the first two conceptual constructs are credible, then CCL3L1/CCR5 GRGs will influence the assessment of vaccination programs as they will affect the computation of the (i) different components that comprise the Pc (e.g., Ro, FIG. 11B and Table 20), an important estimate of the critical proportion of the population-based vaccination coverage that will be required to limit the epidemic (12, 13), and (ii) dynamics of the epidemics in host population groups that differ based on their GRG. Finally, failure to account for the transmission-influencing effects of the GRGs will result in imprecise vaccine efficacy estimates and failed randomization.

Each of these conceptual constructs is shown to be valid, as the GRGs affected each of the four categories of vaccine endpoints and conveyed HIV disease prognostication that was over and beyond that afforded by parameters currently used to monitor HIV disease status. To confirm the verity of these constructs, we replicated, in two separate cohorts, the key genotype-phenotype relationships identified herein and previously by us (6-9).

The delayed type hypersensitivity (DTH) skin test provides an in vivo estimate of CMI and immunogenicity of vaccines (19, 20). In accord with the findings from earlier studies in the WHMC cohort (21-24), the current analysis, which used a substantially larger number of subjects, confirmed that DTH responses are highly predictive of time to AIDS and a sensitive predictor of CMI. However, the magnitude of the DTH responses (FIG. 11C), as well as the risk of developing anergy (FIG. 1D) and progressing rapidly to anergy or hypoergy (FIG. 11E) was least, intermediate, and greatest in HIV⁺ adults possessing the low, moderate and high risk GRGs, respectively. Additionally, as compared to those with the low risk GRG, the risk (odds ratio) of being anergic within the first six months of entry into the cohort was elevated by a factor of 2.14 (95% CI: 1.14-4.02, P=0.018) and 1.57 (CI: 1.05-2.34, P=0.026) in those possessing the high and moderate risk GRGs, respectively. In a multivariable-adjusted model, DTH responses, VL-sp and bCD4 were each independent predictors of the rate of progression to AIDS (“overall” in FIG. 11F and Table 22). However, the strengths of the associations for each of these prognostic markers were not the same across the GRGs (FIG. 11F and Table 22), indicating that the prognostic value of these markers is highly dependent on the underlying GRGs.

Together, the findings in FIG. 11 (C to F) have relevance with respect to the scientific strategic plan proposed by the Global HIV/AIDS Vaccine Enterprise which prioritizes research on vaccines eliciting CMI (2) as they suggest that (i) the GRGs are a predictor of CMI status in not only the distal but also proximal phases of HIV disease, and (ii) the efficacy and durability of vaccines that rely on CMI for their protective effects might vary in infected vaccinees with different GRGs.

The VL-sp can vary by more than 1.000-fold among individuals. The inverse relationship between the initial extent of viral replication, as reflected by the VL-sp, and CD4⁺ cell depletion is a critical determinant of the observed wide interindividual differences in disease outcome (17). This provides the basis for considering the VL-sp as an important surrogate endpoint in vaccine efficacy trials (15). In a previous study (9), we found that GRGs might affect this relationship between VL-sp and CD4⁺ lymphocyte depletion by influencing the magnitude of the VL-sp. Here, we examined whether, even when the extent of initial virus replication is similar, CD4⁺ cell loss might occur to varying degrees in subjects with different GRGs, i.e., over and above their influence on the initial magnitude of VL-sp, and the GRGs serve as an independent determinant of CD4⁺ cell depletion.

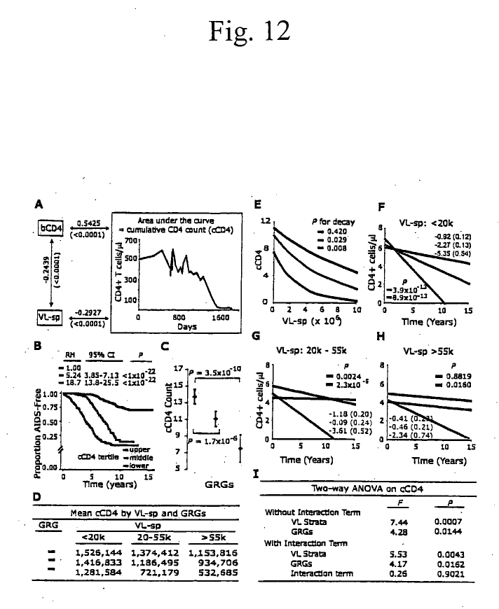

Intuitive clinical experience and findings from non-human primate studies indicate that the relationship between VL-sp and CD4⁺ cell loss is not linear (25). To account for this, we developed a novel epidemiological marker termed as the cumulative CD4⁺ cell count (cCD4) which concurrently factors in bCD4, CD4⁺ cell loss and the unit of time (FIG. 12A). The validity of cCD4 as a valuable quantitative, non-linear parameter to dissect the relationship between VL-sp and CD4⁺ cell depletion is described. This includes correlations between cCD4 and surrogate markers of disease progression such as the nadir CD4⁺ cell count (nCD4), bCD4 and VL-sp (FIG. 12A and Table 23) as well as an extremely robust association between cCD4 and rate of progression to AIDS (FIG. 12B). However, despite this strong association, GRGs stratified subjects into those with significantly different cCD4 counts (FIG. 12C), and the following findings affirmed this. First, at a given level of VL-sp, individuals possessing low, moderate or high risk GRGs differed with respect to their cCD4 counts (FIG. 12D), as well as rates of decline in cCD4 (FIG. 12E) and CD4⁺ cells (FIG. 12, F to H). Second, a two-way ANOVA showed that GRGs and VL-sp were both independent predictors of cCD4 (FIG. 12I).

Together, these findings indicate that the VL-sp fails to capture the independent relationship between the GRGs and CD4⁺ cell loss. This implies that (i) it is not the viral burden per se that drives progressive CD4⁺ cell loss, a possibility that has received increasing scrutiny recently (26, 27); and (ii) the GRGs independently influence CD4⁺ cell loss via mechanisms that are operative during early-stage disease, but are not yet defined and therefore currently not monitored.

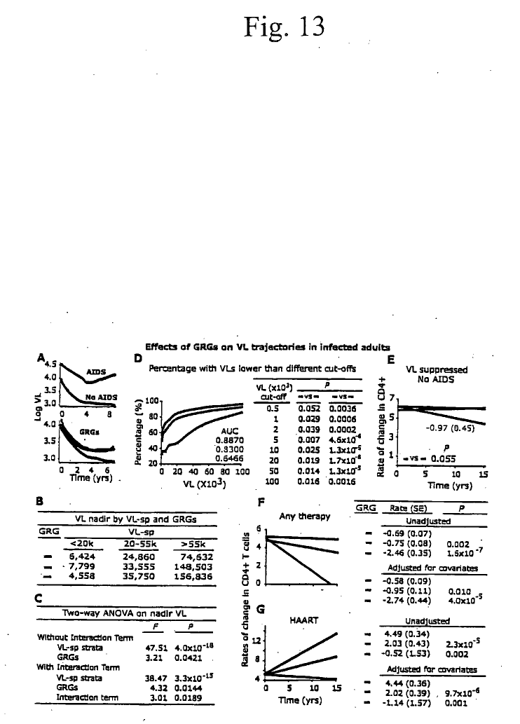

From a broad public health perspective, the primary goal of anti-retroviral therapy (ART) and a therapeutic vaccine are similar, i.e., to lower VLs, (e.g., FIG. 13A, upper). However, several lines of evidence indicated that the GRGs are an independent determinant of ART-induced changes in VL trajectories (FIG. 13A, lower) and the consequent recovery in CD4⁺ cells.

Although the nadir of the VL (nVL) was highly predictive of AIDS and had a very high positive correlation with the VL-sp (ρ=0.7640; P<0.001), in subjects with a given level of VL-sp, those with the low, moderate and high risk GRGs had different VL nadirs (FIG. 13B). Additionally, the VL-sp and GRGs were each independent predictors of the magnitude of the nVL (FIG. 13C). Thus, we inferred that the magnitude of the VL-sp—a proximal event—dictates, in part, the nVL achieved—an ART-induced distal event, and that the GRGs independently affect the magnitude of both the VL-sp (9) and the nVL.

Further supporting this inference were two findings. First, compared with those possessing the low risk GRG, HIV⁺ adults with the moderate and high risk GRGs were more likely to have VLs higher than a wide-array of viremia cut-off points (FIG. 13D), and these effects of the GRGs on the degree of VL reductions were, in general, independent of the VL-sp. Second, the GRGs influenced attainment of the highly protective state of VL suppression, defined here as a VL lower than the detectable limit on at least one occasion during the disease course. The proportion of subjects who failed to achieve VL suppression increased in a step-wise manner in subjects with the low, moderate, and high risk GRG (X² for linear trend=5.84, P=0.0157), and occurred on an average two years earlier in those possessing the low risk GRG. Additionally, even after adjustment for the VL-sp, subjects with the high risk GRG had a nearly 70% (RH=0.32; 95% CI=0.12-0.89; P=0.029) lower likelihood of achieving VL suppression than those possessing the low risk GRG. Thus, there was a GRG-dependent gradient of risk for not achieving VL suppression.

Consistent with the aforementioned findings, the GRGs also served as a genetic basis for the wide intersubject variation observed in CD4⁺ cell recovery that occurs during receipt of therapy (28-30) as: (i) despite attaining VL suppression and not progressing to AIDS, subjects with the high risk GRG experienced progressive CD4⁺ cell loss (FIG. 13E); and (ii) even after adjustment for the effects of several variables that are known to influence CD4⁺ cell recovery (e.g., nCD4), the rate of change in CD4⁺ cell counts during the therapy era was highly dependent on the GRGs (FIG. 13, F and G). Notably, during the HAART era, the maximal rebound in CD4⁺ cells occurred in subjects with the low risk GRG (FIG. 13G).

Thus, it is possible that in some instances of discordant clinical responses such as a declining CD4⁺ cell count in the face of stable/declining VL, or non-suppressed VL might be misattributed to viral resistance or other factors (e.g., non-compliance), when in fact the deterioration might be explained, in part, by CCL3L1/CCR5 GRGs. Also, failure to account for these effects of the GRGs on VL trajectories might influence the computation of the population-level effects of vaccines or ART on the reduction of communicability which is heavily dependent on the viral burden (31).

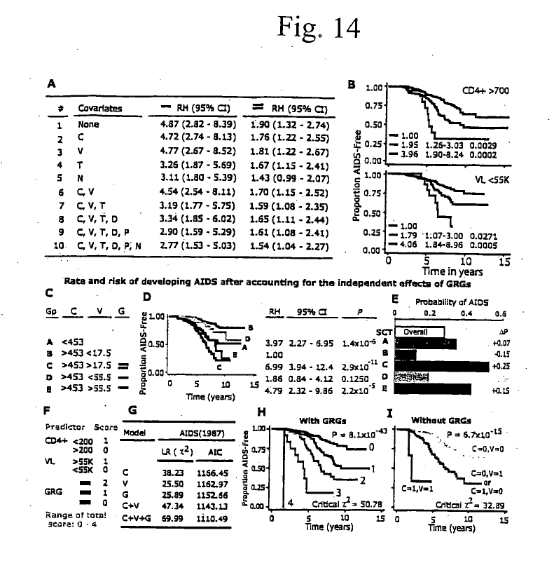

Based on whether a subject possesses a low/high CCL3L1 dose and/or detrimental/non-detrimental CCR5 genotype, the cohort can be divided into two groups of nearly equal proportions (9). After adjustment, individually or in unison, for the disease-influencing effects of several explanatory variables that are in themselves highly predictive of disease outcome (e.g., bCD4, baseline CD4%, nadir CD4, VL-sp, receipt of ART, DTH responses), we found that the combination of moderate and high risk GRGs independently predicted both risk and rate of progression to AIDS (FIG. 14A and Table 24). Thus, the simple stratification of the cohort into two broad CCL3L1/CCR5 genetic-based groups afforded independent predictive capacity that is not solely because of the strong and independent effects associated with the high risk GRGs (FIG. 14A and Table 24). Additionally, the GRGs had independent predictive capacity even during the early phases of the disease such as when the bCD4 is high and the VL-sp is low (FIG. 14B). These findings corroborated the factor analyses, which also indicated that CCL3L1/CCR5 genotypes are tracking a unique aspect of AIDS risk that cannot be fully captured by assessing VL- or CD4-dependent measures of risk (Table 25).

If, as indicated by our findings, HIV disease prognostication by CCL3L1/CCR5-based GRGs is (i) not fully accounted for by estimating an in vivo correlate of CMI or by the established biomarkers, and (ii) evident in instances when the laboratory markers predict a contrary likelihood of developing of AIDS, then the GRGs should provide additive and independent prognostic information. We used two approaches to test this.

In the first approach, classification and regression trees (CART) were used to model bCD4, VL-sp and the GRGs to predict both risk and rate of development of AIDS (FIG. 14, C to E). The advantage of the CART approach is that the algorithm generates a decision-tree that is free of any preconceived bias with respect to predefined cut-offs for VL and CD4⁺ cells. The five independent risk groups identified by CART (groups A to E in FIG. 14C) had more refined risk-profiles than the groups that did not include the GRGs (FIG. 14, D and E). These findings indicate that the GRGs can aid AIDS prognostication in early-stage stage disease and at CD4+ T cell cut-offs (greater than 450 cells/μl) that are well above the point at which ART is currently recommended (18).

The second risk-stratification schema used an empiric risk scoring system with cut-offs for CD4⁺ cells and VLs that are oft-times employed to make decisions regarding when ART should be initiated (FIG. 14F) (18). The prognostic value of the GRGs in the risk-scoring system was evaluated by two strategies: nested Cox proportional hazards models (FIG. 14G) and survival curves for time to progression to AIDS or death (FIG. 14, H and I). We found that the prognostic performance of these nested models and survival curves that contained the GRGs along with VL and CD4⁺ cells were superior to those that only accounted for these two biomarkers (indicated by higher likelihood ratio χ² and lower Akaike information criterion (AIC) values in FIG. 14G and compare survival curves and critical χ² values in FIG. 14, H and I). Thus, the findings of these two complementary approaches demonstrated that, along with VLs and CD4⁺ cells, the GRGs can provide a more accurate means of gauging and stratifying the continuum of HIV disease risk, and by extension, the disease-influencing effects of a therapeutic vaccine.

The aforementioned analyses defined the relationship between the GRGs and the immunological, viral, and clinical categories of vaccine endpoints (15). The fourth category—epidemiological endpoints—provides a bridge between these population-based genotype-phenotype relationships and public health practice and programs. We first examined whether the GRGs associated with enhanced HIV susceptibility and/or communicability might promote the epidemic. For this, we used highly conservative assumptions to model the effects of the GRGs on Ro, a component of Pc (FIG. 11B and Table 20). Ro here denotes the average number of new infected individuals generated annually by each currently infected individual (FIG. 11B). The “epidemic threshold” or “tipping point” at which the epidemic is sustained occurs when the Ro is >1.

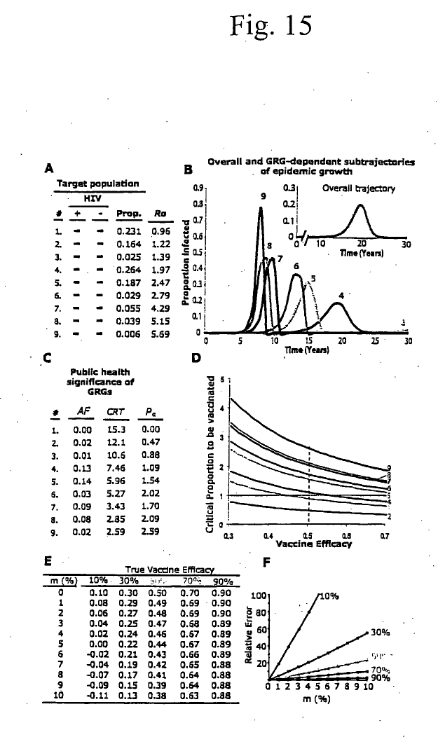

Based on the GRGs of the infected and uninfected partner pair, the population can be divided into nine core groups and we found that in all but one of these nine groups, the Ro was greater than unity (FIG. 15A and Table 26). Since Ro is a critical concept in the explanation of the emergence and persistence of epidemics, we next asked if these GRG-dependent differences in the Ro might translate into differences in the rate of epidemic growth (the number of new infected cases per unit of time) among the infected-uninfected partner pairs (FIG. 15B). In a model that assumes a closed population, the simulated overall trajectory of the epidemic growth took ˜10 years to emerge, and without an influx of a susceptible pool of individuals, predictably, the simulated epidemic eventually died out (FIG. 15B, inset). However, in each of the nine GRG-based core groups, the simulated trajectories were strikingly different (FIG. 15B). This difference was highly dependent on the infecting partner's GRG, and was evident at three levels: (i) the time at which an increase in the epidemic growth began; (ii) proportion of subjects infected; and (iii) duration for which the epidemic growth persisted (FIG. 15B). Remarkably, the epidemic growth was negligible when the infected partner possessed the low risk GRG (FIG. 15B). Thus, under the umbrella of an overall trajectory of the simulated HIV epidemic, the GRGs discriminate for several subtrajectories, and of these, only a few appear to be critical in sustaining the epidemic.

These nine groups together contributed approximately 52% to the overall epidemic growth (FIG. 15C, attributable fraction, AF), and the greatest contribution was from infected subjects possessing the moderate or high risk GRG, i.e., subjects who are likely to be more infectious. This implies that a reduction in the population-level VL-sp by a vaccine or ART will have the largest benefit in abating the spread of the epidemic when applied to these subjects, especially since they comprise nearly 50% of the population (FIG. 15A). Additionally, the critical response time (CRT), defined as the time interval within which the number of epidemic cases remains stationary (so that interventions implemented within CRT may be the most effective or least costly), varied across the GRGs from 2.59 to 15.3 years (FIG. 15C). This indicated that the time available to implement control measures against spread of the infection is also highly dependent on the GRGs. This genetic information might be especially useful in public health settings where the index case is highly infectious and has increased high-risk sexual contacts (32).

Finally, we considered the effect of the GRGs on the Pc. Sensitivity analyses indicated that the Pc was more sensitive to changes in the values of Ro than to those of vaccine take (t) or durability (d) (FIG. 11B). This suggested that the conservative estimates for t and f that we used, even if not completely representative of the effects of the GRGs on vaccine take and durability, would not impact significantly on the Pc estimate. Within the constraints of the assumptions made, the Pc estimates were lower than unity in the population groups in which the infected partner belonged to the low risk GRG (FIG. 15C). Pc was greater than unity in the other six groups, suggesting that to contain the epidemic, such population groups might require repeated immunizations, especially if the vaccine efficacies are low. Reinforcing this possibility, FIG. 15D shows that except for population groups 1 and 2, with decreasing vaccine efficacies the magnitude of Pc rises above unity for all the other groups.

The aforementioned analyses have all considered the effects of the GRGs on disease endpoints. However, variations in CCL3L1 and CCR5 also influence risk of acquiring HIV infection (8, 9). It is important to account for this since ongoing vaccine trials recruit HIV-negative subjects at increased risk for acquiring HIV, and thus may inadvertently select for some individuals who have been exposed to the virus, but have a genetic basis to resist acquiring infection. Consequently, in vaccine trials, especially those with smaller sample sizes (500-1000 subjects), failure to randomize for an individual's CCL3L1/CCR5 genotype might mask the true efficacy estimates of a vaccine that partially blocks transmission (FIG. 15E).

We recognized that an absolute prerequisite for accepting the implications of the findings of this population-based study is the replication of the central genetic associations detected for CCL3L1 copy number and CCR5 genotype (33). In addition to our prior replication studies (6-9), new replication studies were conducted in populations of European descent who were from the European American (EA) component of the San Diego, Calif. site for the Acute Infection and Early Disease Research Program (AIEDRP) (34) and Argentinean children exposed perinatally to HIV-1 (8, 9).

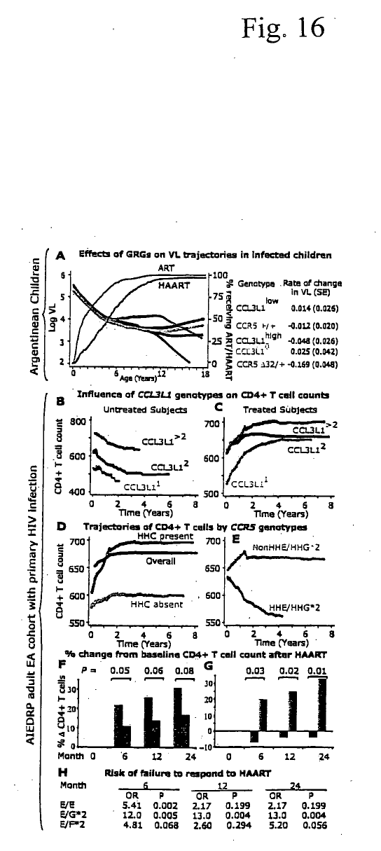

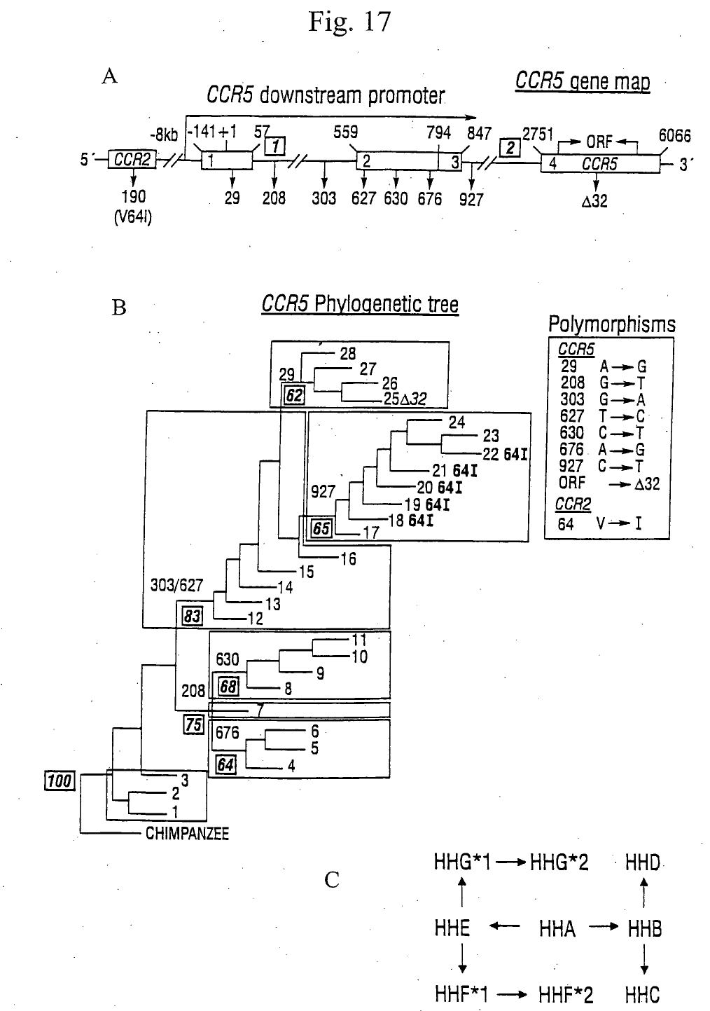

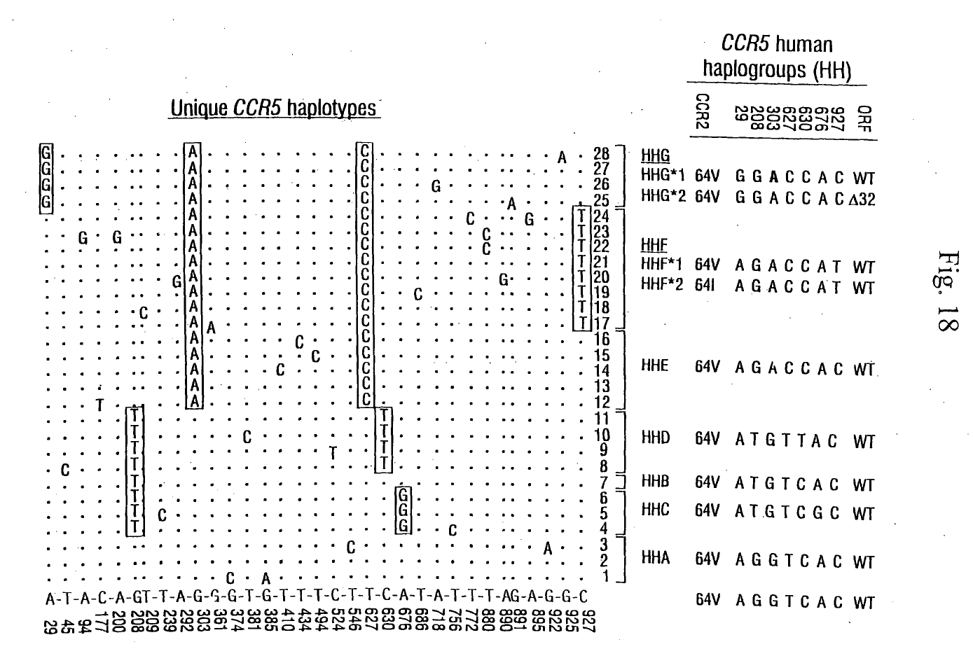

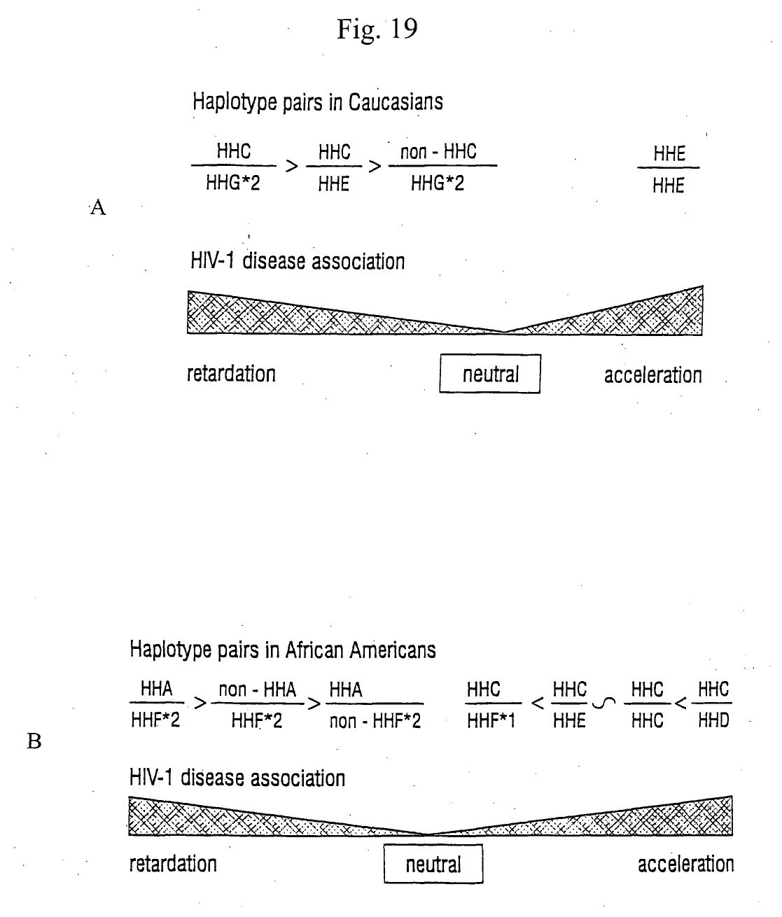

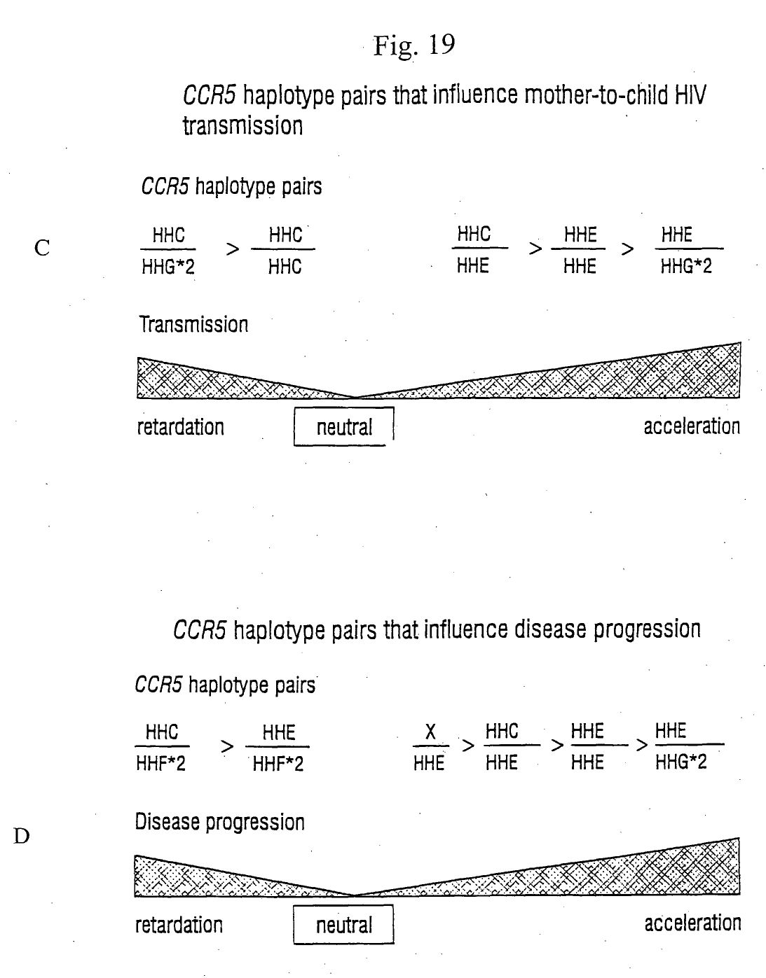

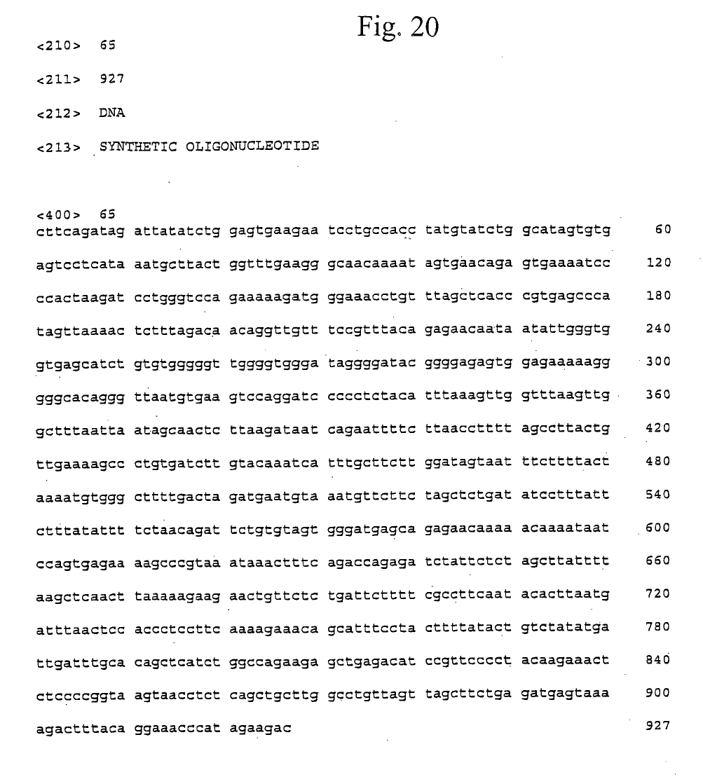

The direction (e.g., protective vs. detrimental) of the disease-influencing effects associated with the individual components of the GRGs that we had found here and previously (6-9) were relatively impervious to first, the considerable constraints imposed by the strikingly contrasting epidemiological features of the three cohorts and endpoints analyzed, and second, settings in which one would have expected obliteration of any possibility of detecting such associations, e.g., after administration of HAART in the early stages of the disease in the AIEDRP cohort subjects. The details of these replication studies include the affirmation that CCL3L1 copy number and CCR5 haplotype/haplotype pairs are determinants of (i) VL trajectories during receipt of ART/HAART (FIG. 16A); (ii) baseline CD4⁺ counts (FIG. 16, B and C); and (iii) CD4⁺ cell recovery following receipt of HAART (FIG. 16, D to H). Further highlighting the robustness of these associations, and their generalizability to other cohorts, we show that a complementary method of categorizing the detrimental versus non-detrimental CCR5 genotypes for inclusion into the GRGs is flexible and provides comparable results with respect to stratification of subjects with different CD4⁺ cell recoveries or rates of progression to AIDS.

We draw special attention to replication studies related to CCR5 genotypes that contain the CCR5-Δ32 mutation, a polymorphism that, because of is biological and evolutionary importance has received extensive scrutiny (5). This mutation is widely believed to be associated with protective effects. However, in all three cohorts studied, the “protective” effects associated with the CCR5-Δ32 containing haplotype (HHG*2) are highly dependent on its partner allele. Thus, when partnered with a disease-accelerating CCR5 haplotype (HHE), the outcome of these CCR5-Δ32-containing genotypes is not favorable (FIG. 16, E, G and H). Along with the CCR5 HHE/HHE genotype, possession of HHE/HHG*2 and HHE/HHF*2 had among the highest risks of failure to respond to HAART (FIG. 16H). This indicated that a similar effect of the partner allele is also observed for the CCR5 HHF*2 haplotype which contains another highly scrutinized polymorphism (CCR2-64I) that is also widely considered to be “protective” (5). These findings underscore our previous contention that CCR5-based risk stratification in both HIV and non-HIV diseases requires knowledge of both haplotypes (7, 8).

Assessing disease risk of infected individuals, outside and within the context of vaccine trials, especially during early-stage disease is vitally important, but diagnostically challenging. Our findings indicate that the CCL3L1/CCR5-based GRGs provide independent prognostication during early- and late-stage disease, and also when the laboratory markers predict a contrary risk. Therefore, the GRGs can add materially to clinical decision-making, e.g., whether ART should be initiated early, before irreversible immune dysfunction occurs, or alternatively, delayed to spare toxicity of the therapies. Furthermore, the GRGs provide a genetic basis for why some individuals have a poor immunological recovery despite receipt of HAART, even when administered during the early stages of disease. Thus, we suggest that in a manner analogous to the use of HIV genotype, when applied judiciously along with knowledge of the clinical and laboratory parameters, CCL3L1/CCR5 host genotype has practical utility in guiding the care of the infected individual, and clinical and vaccine research.

In vaccine efficacy trials, at the time of recruitment or statistical analysis, knowledge of the GRGs might aid discrimination between the effects that are attributable to the HIV vaccine versus host genotype, and consequently, a more accurate estimate of the true vaccine efficacy. This discrimination can also assist in the design of both smaller, affordable vaccine efficacy trials with a reduced likelihood of failed randomization, and more effective prevention programs.

Collectively, our findings do not contradict the established view that CD4⁺ cell counts and VLs are good indicators of immune and viral status, respectively. Rather, they indicate that CCL3L1 copy number and/or CCR5 genotypes are linked to essential, but differing elements of disease pathology that mediate variable rates of T cell loss and/or immune reconstitution. This possibility is supported by recent evidence linking CCR5 and its ligands to T-cell costimulation and differentiation (35, 36). Thus, CCL3L1/CCR5-based vaccine pharmacogenomics can provide important insights for the design of novel HIV vaccines. From a broader perspective, our findings show that the inherent variability among individuals and, by extension, among populations in host genes that influence HIV/AIDS susceptibility are an important, but hitherto underestimated and overlooked biological factor to include in the quest for an effective HIV/AIDS vaccine.

For these studies we used CCL3L1 gene copy number (2) and CCR5 genotype data (3-6) from a HIV⁺ and HIV-negative cohort derived from Wilford Hall Medical Center (WHMC), San Antonio, Tex. The key genotype-phenotype associations for CCL3L1 copy number and CCR5 genotype derived from the WHMC cohort were replicated in two cohorts: (i) a cohort of HIV-infected children exposed perinatally to HIV-1 (2, 7); and (ii) the European American (EA) component of the University of California, San Diego site for the Acute Infection and Early Disease Research Program (AIEDRP) (8).

Adult patients with HIV-1 participating in the U.S. Air Force (USAF) portion of the Military HIV Program Natural History Project contributed samples for this study. WHMC is the referral hospital for all USAF personnel who develop infection with HIV-1. The voluntary, fully informed consent of the subjects used in this research was obtained as required by Air Force Regulation 169-9 and with approval from the Institutional Review Board (IRB) of the University of Texas Health Science Center, San Antonio, Tex. A total of 1,132 HIV⁺ adult patients were evaluated, including 515 seroconverting individuals. The demographic background of this cohort was 55% EA, 36% AA, 6% HA, and 3% “other.” The median age at the time of diagnosis was 28 years (range, 18-70 years), and 94% of the subjects were male. The median follow-up time was 6.2 years for the entire cohort and 6.6 years for the seroconvertors, using as the initial time point the estimated seroconversion date (the midpoint between the last negative and first positive HIV test). The median time from the last negative HIV-1 test to estimated seroconversion was 10.8 months. Forty percent of this cohort progressed to AIDS (1987 criteria), and 39% died during the study period that ended December 1999.

Of note is that this cohort has a racially balanced composition. It represents one of the largest cohorts of HIV seropositive patients followed prospectively at a single medical center. Also, because of the unique nature of the cohort, additional factors that confound genotype-phenotype studies (e.g., unequal access to medical care and anti-retroviral therapy, length of follow-up and loss to follow-up) are minimized. Detailed characteristics of the cohort and the number of subjects available for each of the statistical analyses are shown in Table 21.

1,133 seronegative samples were obtained from HIV-1-negative Air Force personnel to serve as a reference population for comparison of CCL3L1/CCR5 genetic risk group (GRG) frequency distribution with the HIV-infected WHMC cohort and are as described previously (2).

The characteristics of this cohort have been described previously (2, 7). Serial (n=3,967 total measurements) plasma viral loads (for data shown in FIG. 16A) were available from HIV⁺ Argentinean children (n=321) born to HIV-1 positive mothers.

This cohort comprised of 178 EA adults recruited during early or primary infection at the University of California at San Diego, USA. The AIEDRP is sponsored by the National Institute of Allergy and Infectious Diseases, Division of AIDS.

Subjects with signs or symptoms of an acute retroviral syndrome or evidence of recent HIV infection presenting to the UCSD Antiviral Research Center in San Diego, Calif. were evaluated for study entry. Acute HIV-1 infection was defined by a detectable HIV RNA (>5,000 copies/mL) at baseline in the presence of a negative HIV enzyme immunoassay (EIA) and followed by subsequent HIV seroconversion or a positive HIV EIA but indeterminate Western Blot. Recent HIV infection was defined by a positive HIV EIA and an HIV-1 DT-EIA (9) of ≦1.0 (defined as sample OD-negative control OD/positive control OD), in the presence of a CD4 cell count >200/mm³ or CD4%>14 or a documented negative HIV EIA in the 30-365 days prior to the date of HIV EIA seroconversion. Subjects were excluded if they had received more than 7 days of antiretroviral therapy at any time prior to study entry.

The baseline characteristics of this cohort are detailed in Table 21 and some of the features of the subjects in enrolled in the UCSD site for the AIEDRP have been described previously (8, 10, 11).

Based on the possession of population-specific detrimental CCR5 genotypes and/or CCL3L1 gene copy numbers lower than population-specific median, we previously showed that four mutually exclusive GRGs exist (2):

(a) Possession of neither CCL3L1 gene copies lower than the population-specific median or detrimental CCR5 genotypes (CCL3L1^{high}CCR5^{non-det}).
(b) Possession of detrimental CCR5 genotypes, but not CCL3L1 gene copies lower than the population-specific median (CCL3L1^{high}CCR5^{det}).
(c) Possession of CCL3L1 gene copies lower than the population-specific median, but not detrimental CCR5 genotypes (CCL3L1^{low}CCR5^{non-det}), and
(d) Possession of both CCL3L1 gene copies lower than the population-specific median and detrimental CCR5 genotypes (CCL3L1^{low}CCR5^{det}). The superscripts low and high denote less than and equal to or more than the population-specific median copies of the CCL3L1 gene, respectively (2). Whereas the superscripts det and non-det denote detrimental and non-detrimental CCR5 genotypes (2). The methods used for the categorization of CCR5 genotypes into CCR5^{det} and CCR5^{non-det} are as described previously (2).

As indicated, based on the possession of population-specific CCR5 genotypes and CCL3L1 gene copy numbers four mutually exclusive combinations exist. We used these four genetic risk groups in following three ways.

(a) Four genetic risk group system is the use of the four GRGs as described above and previously (2).
(b) Three genetic risk group system classified subjects as:

high risk if they possessed CCL3L1CCR5^{non-det};

moderate risk if a subject possessed either CCL3L1^{high}CCR5^{det} or CCL3L1^{low}CCR5^{non-det};

low risk if the subject possessed CCL3L1^{high}CCR5^{non-det}.

(c) Two genetic risk group system classified subjects into CCL3L1^{high}CCR5^{non-det} versus the rest of the genotypes.

To optimize the number of genetic risk groups that have prognostic value based on CCR5 genotypes and CCL3L1 gene dose we made use of two statistical parameters: the critical χ² statistic defined as the model χ² divided by its degrees of freedom and the Akaike information criterion (AIC; Akaike, 1973) (12, 13). AIC is a popular method for comparing the adequacy of multiple, possibly nonnested models. The critical χ² statistic indicates the average predictive performance of the number of risk groups included in a multivariate regression model whereas the AIC summarizes the prognostic information content within a multivariate regression model as AIC=−2*log likelihood+2*number of covariates. A higher value of critical χ² indicates a better prognostic performance while a low value of the AIC indicates more prognostic information.

The predictive value of the risk stratification system was determined using multivariate Cox proportional hazards regression by comparing each risk group with subjects possessing CCL3L1^{high}CCR5^{non-det} (reference group). The stratification system that gave consistently high critical χ² and low AIC was chosen as the most informative with respect to prognostic value. We found that in the context of time to AIDS and time to death for the entire HIV⁺ WHMC cohort as well as the seroconverting component of the cohort, the three genetic risk group system was the most optimal choice (the only exception was the critical χ² test for seroconverters for time to AIDS). Hence, for all the further analyses we chose the three group system and designated the genetic risk groups (GRGs) as

low risk: CCL3L1^{high}CCR5^{non-det},

moderate risk: CCL3L1^{high}CCR5^{det} or CCL3L1^{low}CCR5^{non-det}, and

high risk: CCL3L1^{low}CCR5^{det}.

Given the implications of the findings derived from the WHMC cohort, one of the main purposes of the replication studies reported here was to demonstrate that the disease-influencing effects associated with CCR5 haplotypes/haplotype pairs have a high degree of consistency across different cohorts that have a similar ethnic composition. If this is true, then an alternative strategy of categorizing CCR5 genotypes into the CCR5^{det} and CCR5^{non-det} groups should be possible, and consequently, the GRGs that contain this alternative CCR5^{det} and CCR5^{non-det} groups will provide a similar level of risk stratification as that for the GRGs described.

We probed the EA component of the AIEDRP cohort to determine if similar CCR5 genotype-phenotype relationships were evident. Since most of these subjects received highly active antiretroviral therapy (HAART), and that too in the early stages of their disease, the endpoints for these analyses was not rate of progression to AIDS. Instead, the endpoint for analyses was changes in CD4⁺ counts following initiation of HAART. Highlighting the consistency in the effects of CCR5 haplotypes/haplotypes across cohorts, in the AIEDRP cohort we replicated the following findings: (a) possession of HHC was also associated with a rebound in CD4⁺ cells; (b) possession of HHE/HHE was associated with a muted CD4⁺ recovery; (c) consistent with the notion that the partner allele of HHG*2 or HHF*2 matters in the ultimate disease-influencing phenotype, we found that possession of HHE/HHG*2 and HHE/HHF*2 was associated with an increased risk of failing to manifest a CD4⁺ cell recovery following receipt of HAART; and (d) the disease-influencing effects associated with the HHE/HHG*2 vs. non-HHE/HHG*2 genotypes was discordant, and only the latter with protective effects.

We confirmed that the disease-influencing effects associated with the HHE/HHG*2 vs non-HHE/HHG*2 genotypes was also discordant in these two cohorts.

Even though CCL3L1 copy number does not influence rate of progression to AIDS in HIV⁺ children (2), we replicated that it is a determinant of VL trajectories (FIG. 16A).

In the AIED cohort, CCL3L1 copy number was associated with a gradient of baseline CD4⁺ counts (FIG. 16, B and C). Affirming that copy number and CCR5 genotype influences CD4⁺ cell recovery (FIG. 13, F and G), in the AIED cohort the changes in CD4⁺ counts in those who did or did not receive HAART also differed as a function of both the copy number (FIG. 16, A and B) and CCR5 genotype (FIG. 16, D to H).

The consistency in the direction of genotype-phenotype effects associated with CCL3L1 copy number or CCR5 genotype is relatively impervious to the considerable constrains imposed by three cohorts with strikingly contrasting epidemiological features (e.g., horizontal vs. vertical transmission), age (adults vs. children) and endpoints (CD4⁺ loss vs. rebound as in the AIEDRP cohort) as well as in settings where one would have expected that receipt of HAART would obliterated any possibility of detecting such associations. Highlighting the robustness of these associations, we show below that a complementary method of categorizing the detrimental versus non-detrimental CCR5 genotypes into CCR5^{det} or CCR5^{non-det} for inclusion into the CCL3L1/CCR5 GRGs provided comparable results with respect to stratification of subjects with different CD4⁺ cell recoveries or rates of progression to AIDS.

These replication findings indicated that the disease-influencing phenotypic effects associated with the CCR5 haplotypes/haplotype pairs are robust across cohorts. This robustness implied that there might be flexibility in how the genotypes that comprise CCR5^{det} and CCR5^{non-det} groups are selected. Based on the replication data found in this study, logically, one would expect the non-HHC/non-HHC and non-HHG*2/non-HHG*2 genotypes are associated with an accelerated rate of disease progression in EAs. Therefore, in this alternative classification system, the following definitions were used to combine the CCR5 genotypes: CCR5^{non-det} was defined in EA subjects as possession of HHC-containing haplotypes and/or HHG*2-containing genotypes that lack HHE. All the remaining CCR5 genotypes were combined into the group designated as CCR^{det}. Thus, the HHE/HHG*2 subjects were not included in the CCR5^{non-det} group. Then, based on the possession of the varying copies of the CCL3L1 gene an alternative risk scoring system was designed as follows:
alternative low risk: CCL3L1^{high}CCR5^{non-det} contains HHC-containing genotypes and HHG*2-containing genotypes that lack HHG*2/HHE AND 2 or more copies of CCL3L1)
alternative moderate risk: CCL3L1^{high}CCR5^{det} or CCL3L1^{low}CCR5^{non-det} groups are those that possess either less than 2 copies of CCL3L1 OR non-HHC/non-HHC and non-HHG*2/HHG*2 genotypes, and
alternative high risk: CCL3L1^{low}CCR5^{det} are those that possess less than 2 copies of CCL3L1 AND non-HHC/non-HHC and non-HHG*2/HHG*2 genotypes.

This alternative system of classification of the CCR5 genotypes in the EA populations has the advantage of simplicity. We first assessed how the predictive performance of this alternative system compares to the classification system described herein. The estimates of the Cohen's kappa indicate that there was a strong agreement (P<1×10⁻²²) between the two different systems of classification. The alternative risk scoring system performed well in terms of prognosticating for the rate of disease progression in the HIV-infected EA adults in the WHMC cohort. We also found that the direction of the gradient of the risk associated with these low, moderate and high risk GRGs determined by this alternative manner was similar in the EA component of the AIEDRP cohort with respect to CD4⁺ T cell recovery.

The outcomes analyzed were those that might influence AIDS vaccine endpoints, or previously validated measures of clinical status/outcome, and included rate of progression to AIDS or death, baseline CD4+ T cell counts (bCD4) and viral load set points (VL-sp), nadir viral loads (nVL), nadir CD4+ T cell counts (nCD4), % CD4+ T cell counts (% CD4), cumulative CD4 counts (cCD4), four antigen panel skin test to detect delayed type of hypersensitivity (DTH) responses, nadir viral loads (nVL) and anti-retroviral therapy (ART). The bCD4 and VL-sp strata used (e.g., CD4+ T cell counts of either <200; CD4% as a cut-off at 14%) were those that are currently used to assist in making decisions regarding when to initiate ART (16-18). The manner in which ART was used as an outcome is described. The nCD4 and nVL was defined as the lowest CD4+ T cell count or VL, respectively observed during the clinical course in an adult HIV-infected individual. The 1987 criteria for AIDS was used in the analyses shown. The 1993 criteria for AIDS were used as one outcome and time to death was used as another outcome.

The outcomes analyzed in this cohort were time trends in CD4+ T cell counts before and after initiation of HAART and the risk of failure to respond to HAART. Time trends of CD4+ T cells were modeled using Loess curves. Percentage change in CD4+ T cell count from the pretreatment baseline was also studied at six months, one year and two years after treatment. Failure to respond to HAART was defined as stable or declining CD4+ T cell counts post-treatment and was studied at three time points: six months, one year and two years from initiation of therapy.

CMI responses are required for a better clinical outcome, and possibly also for an effective AIDS vaccine (19, 20). Hence, we initiated our analyses by addressing a fundamental question: Do the GRGs influence the magnitude of CMI responses in vivo? The skin test for delayed-type hypersensitivity (DTH), which reflects CD4+ T helper cell-dependent, antigen (Ag)-specific events, is among the only in vivo assay available for assessing CMI responses in humans, and has importance as a vaccine endpoint. For example, in the cancer vaccine field, the generation of a DTH response is often used as the primary measure of the ability to immunize a patient to a tumor cell or specific tumor Ag (21). In the HIV vaccine field, DTH, responses might facilitate detecting immunogenicity and efficacy of HIV vaccines as infected subjects can mount HIV envelope-specific DTH responses despite the inability to detect lymphoproliferative responses to the same Ag in vitro (22). Furthermore, DTH responses have relevance for assessing disease status as (i) in contrast to an absolute CD4+ cell count, DTH assays are an in vivo surrogate for impaired T cell function; and (ii) we and others showed previously that DTH responses have significant predictive value for survival time, independent of CD4+ cell counts (23-26). Also, we found that the magnitude of DTH responses correlated positively with in vitro IL-2 production, a measure of T cell function (25).

We used additional extensive DTH readings performed prospectively in a highly standardized manner (23-25, 27) in the HIV⁺ WHMC cohort as an outcome. Each patient at enrollment and then prospectively received the standard Mantoux type of intradermal skin test. The protocols for conducting the DTH skin tests are highly standardized and were as described previously. The antigens and concentrations used were as follows: mumps (Connaught), 40 colony-forming units per milliliter full strength until unavailability as of July 2003; trichophyton (Holister-Stier), 1:500 dilution until removed from the market by the FDA in June 1996; candida (Walter Reed Army Institute of Research, 200 PNU/mL), 1:100 dilution; and tetanus toxoid (Lederle, 1.6 Lf/mL), 1:100 dilution. Test results were assessed at 48 hours. Skin test results were considered positive when 5×5 mm or greater induration was present.

The DTH responses were coded as categoric variables based on the number of positive skin tests (e.g., zero, one, two positive results). In some instances subjects were categorized into three groups based on their DTH response: (i) zero or one positive skin tests (out of four) pooled into one group and referred as anergy/hypoergy; (ii) two positive skin tests out of four; and (iii) three or four positive skin test pooled into a single group

Additional terminologies used to categorize the DTH responses are as follows. (a) “Initial” DTH response indicates the first DTH reactions detected at enrollment; (b) The “best” DTH responses refers to the maximum number of positive skin tests detected at any time during the disease course. In the analyses shown, we also sought to determine if the GRGs can stratify individuals into different risk groups even when they have the best DTH responses. We surmised that it would be more difficult for the GRGs to provide prognostic information in the face of robust CMI responses, and thus we sought to favor a clinical setting in which the possibility of obtaining a result in which the GRGs stratified DTH responses was low. Another advantage of using the best DTH responses was that within the constraints of HIV-infection they might provide as close an estimate as possible of the CMI that was present during the uninfected state.

We organized our analyses based on the central conceptual models shown in FIGS. 11A and B. The premise of this work is as follows. Variations in CCL3L1 and CCR5 affect HIV disease at two levels. First, these variants influence the level of the VL set point and baseline CD4+ T cell count established. That is they are linked to mechanistic processes operative during the early stage of disease that establish the threshold levels of these parameters. Second, these variations affect disease over and beyond their influence VL set point and baseline CD4+ T cell counts, i.e., they have effects that are independent of those used to currently monitor HIV disease status.

The data are derived from the main cohorts, and is the WHMC cohorts (FIGS. 11 to 15), and the replication cohorts, which are the pediatric cohort and infected adults from the AIEDRP cohort (FIG. 16). In the WHMC cohort the majority of the individuals are either EAs or AAs (Table 21). After accounting for the population-specific criteria to categorize individuals into different GRGs (2), the results shown are for the combined analyses for the EA and AA portions of the infected WHMC cohort. We used CCL3L1/CCR5-based genotypes described previously (2) to determine their (i) influence on four AIDS vaccine endpoints: CD4 counts, VL, clinical and epidemiological; and (ii) effects on AIDS vaccine endpoints that are independent of the VL set point (VL-sp), and CD4-measures of risk such as the baseline and cumulative CD4 counts. We also determined if the direction of the effects (protective or detrimental) associated with variations in CCR5 and CCL3L1 are also evident when subjects had received antiretroviral therapy (ART) or highly active antiretroviral therapy (HAART).

FIG. 11 shows the relationship between GRGs and DTH responses in infected patients. First, we used the extensive DTH data available for the WHMC cohort to determine their association with progression to AIDS. We then determined if subjects with the GRGs had different responses. These are shown in FIG. 11 and they included determination of the association between the GRGs and the initial and best DTH responses, anergy at presentation or rate of progression to anergy in those that did not present with anergy. For some of these analyses, Loess curves (28) were also used to determine the relationship between time of progression to AIDS and other DTH-related time-trends. The possibility that the GRGs might influence DTH responses in uninfected subjects was also considered and analyzed. In FIG. 11, we also determined if the association between different VL-sp, bCD4 and DTH strata was similar across subjects who possessed distinct GRGs.

In FIG. 12 we show the data regarding the relationship between CD4+ T cell loss in subjects with a given VL strata. For these analyses, two parameters for CD4+ T cell loss were considered, cumulative CD4 (cCD4) and rates of CD4+ T cell decline. To assess the importance of GRGs as an independent predictor of the CD4+ T cell loss, we used two-way analysis of variance with cCD4 as the outcome variable and the VL-sp strata and GRGs as the predictors. The same model was also run after inclusion of the interaction term. An exponential decay model was also derived to determine the relationship between cCD4 and VL set points in subjects with the different GRGs. Finally, rates of CD4+ T cell decline (shown in FIG. 12, G to I and expressed as cells/month) were determined using the generalized estimating equations (GEE) analyses.

In FIG. 13 we show the data regarding the relationship among GRGs, VL and CD4+ T cell trajectories. The trends over time in VL trajectories were depicted by a solid, smooth line generated by the loess procedure. We also determined the influence of GRGs on VL estimates during the therapy era. Rates of changes in CD4+ T cell counts as a function of the GRGs before and after accounting for the indicated covariates were estimated in different therapy eras.

In FIG. 14, we assessed the independence of the GRGs from other established surrogate markers of disease progression. This was conducted as follows. (i) Cox proportional hazards regression models were also used to determine whether GRGs are an independent predictor of disease progression when controlling for the different outcomes (FIG. 14A). (ii) To validate and extend these analyses, progression to AIDS (1987 criteria) was determined in subjects with specific levels of the surrogate markers prior to accounting for the GRGs and after accounting for the disease-influencing effects of the GRGs. In these analyses, the effects of anti-retroviral therapy (ART) before and after accounting for GRG were also determined. In these analyses, we accounted for a) bCD4, nCD4 and % CD4; b) VL-sp; c) DTH responses and d) receipt of ART (FIG. 14B). (iii) As a complimentary approach, we also used factor analysis to assess the independence and uniqueness of the GRGs from the currently used standard-of-care surrogate markers for disease progression.

Then we directly compared the predictive value of the CCL3L1/CCR5 genotypic groups, bCD4 count and VL-sp in the prognosis of HIV-infected EA and AA adult subjects from the WHMC cohort by several means (FIG. 14, C to I). First, we used a classification tree model. Second, we complemented the results of the classification tree analyses by developing an additive risk scoring system using traditionally used cut-off points for CD4 and VL. Third, we also estimated the prognostic likelihood ratios (LRs) to assess the prognostic importance of various combinations of the bCD4 and VL-sp strata before and after considering the GRGs. Fourth, to estimate the amount of explained-variation in survival analyses, we used Cox proportional hazard models after assessing the validity of the assumption of proportional hazards and computed the measure of R_{M}² as described.

In FIG. 15, we determined the influence of GRGs on epidemiologic endpoints such as Ro, attributable fraction (AF), Pc, critical response time (CRT) and predicted epidemic trajectories. Using simulated trials, we also determined if the failure to account for the effects of the GRGs on risk of acquiring HIV might result in failed randomization.

In FIG. 16, we show the influence of variations in CCL3L1 and CCR5 are replicated across independent cohorts. In the HIV⁺ children and subjects from the AIEDRP, instead of using compound CCL3L1/CCR5 GRGs, we used a reductionist approach towards the choice of genotypes analyzed. In this instance, we elucidated the effects of the copy number of CCL3L1 or different CCR5 haplotypes. The endpoint in the infected children was VL trajectories and in infected adults from the AIEDRP cohort, the endpoint was CD4+ T cell changes. In both of these replication cohorts, we accounted for the ART/HAART used, and in the AIEDRP cohort we determined if the CCL3L1 and CCR5 variants are associated with different risks of failing HAART based on CD4+ T cell recovery. Based on the replication data, for the reasons indicated above, we also tested the association between an alternative GRG classification and the endpoints in the AIEDRP cohort.

We used Stata 7.0 (Stata Corp., College Station, Tex.) software for all statistical analyses and the program DTREG (Brentwood, Tenn.) for generation of the classification trees.

Survival analyses were conducted for time to AIDS (1987 criteria), and where indicated for time to AIDS (1993 criteria) and AIDS-related death in the HIV⁺ individuals from the WHMC cohort. Kaplan-Meier (KM) survival curves were constructed to graphically illustrate progression to AIDS and the log-rank test was used for between-group analysis. We used a Cox proportional hazards model to estimate the RHs (with 95% CI) associated with the specific genotypes. We tested the assumption of proportional hazards by plotting the Schoenfeld residuals and used the program stphtest (Stata 7.0) to formally test the assumption (34). Schoenfeld residuals were calculated for each Cox proportional hazards model studied by using the Breslow-Peto approach.

Similar to previous studies (29-31), we used calendar time as a proxy for the introduction of antiretroviral therapy (ART) in the population. In concordance with these studies, calendar time was partitioned as follows: Jan. 1, 1990 to December 1992 represented “monotherapy use; January 1993 to December 1995 represented “combination therapy use”; and Jan. 1, 1996 to December 1999 represented “HAART use.” We used two dates (Jan. 1, 1990 and Jan. 1, 1996) to categorize the cohort members into six exclusive groups. We observed that these six groups were associated with significantly different rates of progression to AIDS in the entire cohort as well as in the sero-converting subset of the cohort (data not shown).

To simplify the analysis, we further reduced the number of therapy eras to two by combining subjects who received no or minimal therapy into one group designated as “no therapy era” and subjects who—to a large extent—received some form of therapy into another group designated as “therapy era.” As would be predicted, a very strong beneficial effect of the therapy could be discerned in the entire cohort as well as in seroconverters. We used these proxy variables “no therapy era” or “therapy era” in further analyses.

We used Loess curves that estimate the regression in local windows to graphically demonstrate the non-linear time trends of lymphocyte subset counts. This technique has the advantage of being relatively unaffected by the extreme outliers. The basic idea was to move a window along the x-axis of a scatterplot, calculate a fitted value at each window position and then join the fitted values to form the loess curve. The Stata 7.0 command ksm, which achieves kernel smoothing using loess procedure, was used. We used the default bandwidth of 0.8 for generation of all the loess curves. Finally, we used the method of Generalized Estimating Equations (GEE) as described previously (2) to estimate the rate of change in CD4+ counts. The difference between GEE estimates of slope for different GRGs was assessed by using Student's T test.

Classification trees are commonly used as a method of deductive reasoning for the purposes of data mining and extracting relationships among the predictor variables (32-36). When the outcome (or the target) variable is categorical in nature, classification trees are used. We thus used a classification tree to predict the AIDS status of a subject. The software program, after pruning through a set of potential candidate trees, chose the tree best fitting the data. The tree was based on a series of binary diagnostic decisions that best described the cohort data.

The full tree contained 83 nodes (74 terminal nodes or ‘leaves’) while the final pruned tree contained only 9 nodes (with 5 leaves). This tree was initially generated only from the EA plus HA component of the WHMC cohort (N=690) and was tested on the entire WHMC cohort which included subjects with EA, HA and AA ethnicity. As an additional indication of the robustness of the tree generated, we plotted the time to AIDS (1987) criteria at each nodal split. We found that each nodal split generated two groups that were statistically significantly different in terms of both the risk of developing AIDS and the rate at which the HIV-1 infection progressed to AIDS. For generation of the classification trees we used the DTREG (Brentwood, Tenn.) software.

Likelihood ratios (LR) are frequently employed in clinical settings to assess the utility of the result of a diagnostic test (37-39). Especially in the setting of tests where the results can be reported in multiple categories (e.g., CD4 <200, 200-349, 350-499, 500-699 and ≧700), LRs have the advantage of quantifying the diagnostic utility of each test result (37).

If p1 is the proportion of the n, diseased subjects who show a particular test result and p₀ is the proportion of the n₀ non-diseased subjects who show the same test result, then likelihood ratio is defined as LR=p1/p0. A 95% confidence interval around the likelihood ratio can be estimated as

$${e}^{ln ( Lr ) \pm 1.96 \sqrt{( \frac{1 - {p}_{1}}{{p}_{1} {n}_{1}} ) + ( \frac{1 - {p}_{0}}{{p}_{0} {n}_{0}} )}}$$

LR confidence intervals straddling unity are indicative of a test result that might not be clinically meaningful. Significant departures from unity show an increased (LRs >1) or decreased (LRs <1) likelihood of the disease for a given test result.

We estimated the LRs for different strata of baseline CD4+ T cell count and viral load set point and the three GRGs separately. To assess the prognostic independence of genetic risk groups we estimated the likelihood ratios for the GRGs in the context of differing baseline CD4+ T cell counts and VL set points or combinations thereof. Finally, to assess the time-sensitiveness of the LRs we estimated the LRs at the end of each year of follow-up and plotted spline-smoothed curves to depict the relationship of LRs with time. These were done separately for three CD4+ T cell and VL strata and the three GRGs.

The significance of association of a covariate with time to event in survival analyses is commonly based on the results of Cox proportional hazards models. These results, however, may not be able to capture the extent to which each of the covariates contribute prognostically. In such situations, the amount of explained variation is a better measure of the predictive value of a covariate. In generalized linear modeling, the following definition of R² (here referred to as R_{M}²) is defined as:

R_{M}²=1−(L_{R}/L_{U})^{2/n}

where L_{R} denotes the model likelihood without (restricted) covariates and L_{U} represents the model likelihood with (unrestricted) covariates. This definition is equivalent to 1−exp(likelihood ratio χ²/n). Schemper and Stare (40) argue that if the assumptions of Cox proportional hazards modeling are met and the n represents total observations (rather than number of censored observations) then

R_{M}² can be used as a reliable measure of explained variation in survival analyses.

This measure is also comparable with other measures of explained variation like Schemper's V (40-43), and Kent and O'Quigley's ρ²_{w}. (44). As a rule, the explained variation in survival analyses is low based on this definition. (45). We used this definition of R_{M}² in our study to estimate the variation in time to AIDS explained by CD4⁺ T cell count, VL set point and genetic risk groups based on the CCR5 and CCL3L1 genotypes after testing the validity of Cox proportional hazards modeling assumptions using the Schoenfeld residuals.

To assess the overlapping prognostic value of the surrogate markers of disease progression in HIV infection, we conducted principle component factor analysis. We used six explanatory variables: baseline CD4+ T cell count (bCD4), cumulative CD4+ T cell count (cCD4), nadir CD4+ T cell count (nCD4), viral load set point (VL-sp), nadir plasma viral load (nVL) and the GRGs. We extracted the factors using principal components. The scree plot (46) which plots the eigen values by the serially extracted factors, indicated that up to three factors could best explain the correlations among the explanatory variables. However, using a criterion of a minimum eigen value of 1, the analysis retained only the first two factors—referred here as factor 1 and 2. To optimize the factor loadings (the degree to which the explanatory variables predict the hypothetical factors) we rotated the results of the principal components analysis using the varimax rotation (Table 25). bCD4, cCD4 and nCD4 loaded predominantly on the first factor while VL-sp and nVL loaded strongly on the second factor. GRGs loaded minimally on either factors indicating the independence of this variable from the other variables used in factor analysis. Since factors are a linear combination of the explanatory variables, ‘uniqueness’ can be estimated and interpreted as the portion of the explanatory variable's variation that remains unexplained after considering the factor loadings. We found that the GRGs had maximum uniqueness.

For these analyses, we used the conceptual and mathematical frameworks provided by previously elaborated epidemiological models of HIV/AIDS vaccination (47, 48). These models rely on computing the Pc, which is extensively used as an estimate of the critical proportion of the population- or cohort-based vaccination coverage required to limit the epidemic. This estimate has three main components (Ro, e and f), which are shown in the equation below.

Pc=[1−(1/Ro)]/ef (1).

Thus, Pc is a function of i) Ro, the basic reproduction number which measures the average number of secondary infections generated by one primary case of infection in a susceptible population; ii) e, the vaccine efficacy; and iii) f, the fraction of vaccinated subjects in whom the vaccine effect does not wane over the period of infectiousness, i.e., the duration of protection afforded by the vaccine. These three parameters are shown in FIG. 11B, and we have termed f in this figure as vaccine durability.

We approached modeling of the influence of GRGs on Pc with the notion that (i) these are proof-of-principle studies with the modeling conducted based on data derived from the WHMC cohort; and (ii) GRGs will influence the Pc by influencing infectiousness, susceptibility, duration of the infectiousness (time from HIV acquisition to time to AIDS), and other components of Pc that rely on CMI responses, i.e., vaccine take and durability.

Hence, the modeling is focused towards those vaccines that might have some influence on transmission and whose effectiveness post-infection is based on slowing disease progression and relies on the generation of a robust CMI response. These CMI responses might also influence vaccine take and durability. (see below). The methods we used to determine to calculate Ro, e and f, and thus Pc after accounting for the effects of the GRGs are described herein, and the definitions of the various parameters studied herein are shown in Table 20.

It is assumed that the transmission probability (β), background death rate (μ) and proportion of the HIV-infected subjects progressing annually to AIDS (σ) together determine the parameter basic reproductive number (Ro) in the following manner:

Ro=β/(μ+σ) (2).

The parameter Ro is of great interest in predicting the epidemic behavior or trajectory since it captures the number of secondary cases per unit time. Thus, Ro is a measure of the product of infectiousness and susceptibility, which are important determinants of the threshold or tipping point of the epidemic. A Ro that exceeds unity favors an epidemic whereas a value lower than unity, favors conditions that will limit the epidemic. In the estimation of Ro, we assumed a background death rate of 0.025 (48)) and a σ of 0.043. We estimated σ using data from the seroconverting component of the WHMC cohort. Since the denominator in equation 2 is constant, in essence, the behavior or trajectory of the epidemic will be determined by the transmission probability (13).

Based on the findings presented in FIGS. 11 to 14, we surmised that the transmission probability (β) should vary by the GRGs, and to calculate this parameter we did the following. After considering the GRGs of the partners in a heterosexual setting of HIV transmission, the population was divided into nine groups (shown in FIG. 15A and Table 26). To calculate Ro in each of these nine groups, the following assumptions were made regarding the infected partner.
(i) GRGs can influence VL set points.
(ii) VL set points can determine the degree of infectiousness (49, 50). For example, Gray et al has demonstrated that the per sexual contact probability of HIV transmission can be estimated based on the viral load (50). They showed that a reduction in log viral RNA from 4.58 to 3.23 is associated with a 23-fold decrease in the transmission probability.
(iii) We assumed the duration of infectiousness from time of infection to time of development of AIDS. Thus, the rate of disease progression to AIDS will influence the duration of infectiousness.
(iv) Previously (2) we had shown that the GRGs influence the rate of disease progression by influencing VLs. Here we found that over and above their influence on the VL set points, GRGs can independently alter the duration of infectiousness. This is based, for example, on the findings shown in FIG. 14A, model 8, where after adjusting for VLs, CD4+ T cell counts and several other factors, GRGs independently influenced the rate of disease progression.

To calculate Ro in each of these nine groups, the following assumptions were made regarding the uninfected partner. Based on findings shown in FIG. 14, H and I in Gonzalez et al (2) which showed that that GRGs influence risk of HIV acquisition in adults and in the setting of vertical transmission, we assumed that GRGs will influence susceptibility in the uninfected susceptible partners within the nine population groups.

With these assumptions in mind, we calculated the Ro in each of these nine groups as follows.
(i) We first factored in the effect that the GRGs will have on the probability of transmission from the infected partner by virtue of their effects on the VL set point. This is supported by findings showing that higher VLs are the principal determinant of heterosexual transmission (49-54). We calculated the annual probability of transmission from the infected partner as a function of the influence of the GRGs on the VL set point, and this parameter is designated here as βᵤ. To calculate βᵤ, we first estimated the mean log HIV RNA load within the three GRGs (derived from data published in (2)). Then using the equation provided by Gray et al (50), we estimated the per sexual contact probability of HIV transmission for each of the GRGs. Then, assuming an average coital frequency every two days, we estimated the annual transmission probability within each GRG. The βᵤ for the nine population groups is shown in Table 26.
(ii) To factor in the effect that the GRGs will have on the duration and degree of infectiousness together, we next calculated a parameter that we designate as βᵢ. βᵢ takes into account both the disease-accelerating effects of GRGs independent of VLs (this is a measure of the duration of infectiousness) and the effects of GRGs on VL, i.e., βᵤ (degree of infectiousness). We used the adjusted RHs shown in FIG. 14A, model 8 for the three GRGs as a measure of the duration of infectiousness that is attributable directly to the GRGs as these RHs reflected the rate of disease progression to AIDS independent of VLs. The product of the adjusted RHs and the βᵤ is βᵢ. Note, the effects of VLs on duration of infectiousness are not considered here because their effects had been incorporated into Bᵤ.
(iii) We next factored in the effect that the GRGs will have on susceptibility of the uninfected partner, and for this we calculated a parameter that we designate as βₐ. To estimate the probability of transmission in a specific population group (βₐ) we factored in both the transmission probability βᵢ as obtained in (ii) and the odds ratio (OR) of HIV-acquisition based on the susceptible partner's GRG. Based on data from Gonzalez et al (2) we found that the ORs of HIV-acquisition were 1.00, 1.62 and 2.23 in the low, moderate and high risk GRGs in adults. Thus if β is the probability of transmission from an infected partner then the probability of transmission to the susceptible partner will be dictated by the OR of the GRG of the susceptible partner in the following way: βₐ=bi/[1−βᵢ(1+OR)]. The values for βₐ are shown in Table 26.
(iv) Finally, to obtain Ro, we factored in the background death rate and annual incidence rate of AIDS in the HIV-infected subjects into the βₐ for each of the nine population groups. For this, we divided the transmission probability βₐ by the sum of background death rate and annual incidence rate of AIDS in HIV-infected subjects (μ and σ) to obtain the population group-specific estimate of Ro (Table 26).
(v) These calculations further assume that even though the WHMC cohort is a predominantly male cohort (˜94%) the results are applicable to general population and that the transmission probabilities are only minimally affected by the gender.
(vi) Lastly, these calculations will be applicable to clade B HIV-1 infections that are prevalent in the U.S.

Vaccine efficacy is composed of two components: vaccine take and degree (47), designated as t and d, respectively, in FIG. 11B. As defined by Blower and colleagues (47), take specifies the fraction of vaccinated individuals that show a protective immunological response to the vaccine. Degree specifies the degree of vaccine-induced protection against HIV infection experienced by the individuals in which the vaccine takes. Based on their influence on CMI responses shown in FIG. 11, C to E, GRGs can be thought to influence both the take and degree, however, for simplicity here we assumed the degree to be constant across GRGs.

As a conservative measure of the influence of GRGs on the initial vaccine ‘take’, we estimated the relative vaccine take (parameter t in Tables 20 and 26 and FIG. 11B) by normalizing the best DTH responses for each GRG. Data in FIG. 11C show that the average number of the best positive skin tests was 2.77, 2.60 and 2.37 in the low, moderate and high risk GRGs, respectively. If we normalize the average number of positive skin tests in the low risk group to 100%, then the relative vaccine takes in the three GRGs can be estimated as 100%, 94% (95% CI 88%-100%) and 86% (95% CI 74%-98%).

These percentages appear to be relatively conservative based on the known variability of DTH responses in normal individuals. For example, a study of DTH responses in normal Australians showed that 3% and 5.6% of men and women, respectively were anergic (no positive responses), and 10.6% and 9.4% of men and women, respectively fell into the “hypoergic” category (55).

We also modeled the influence of the GRGs on the duration of the vaccine protection. This parameter has been referred to as f by Blower et al (47). In HIV infected subjects, DTH responses decline over time and the degree of this decline can be a function of the GRGs. In turn, this suggests that the duration of vaccine protection can also vary across GRGs. We sought to use this information as a means to model waning vaccine durability over time as a function of the GRGs. In accord with Anderson and Hanson (26), we assumed that the duration of vaccine protection will be 10 years, and as such assumed that this duration of vaccine durability for the low risk GRG. This translates to an annual probability of a waning in the vaccine effect of 0.1. In the next step, we estimated the risk of anergy (complete absence of DTH response) across GRGs.

We observed that compared to the low risk GRG, the moderate and high risk GRGs had an increased likelihood of anergy. The odds ratio for anergy was 1.91 (95% CI 1.07-3.39, P=0.028) in the moderate risk group and 3.10 (95% CI 1.36-7.05, P=0.007) in the high-risk GRGs. Using these estimates of odds ratios, we estimated that the annual probability of loss of vaccine effect will be 0.1, 0.18 and 0.26 in the low, moderate and high risk groups. In other words, 90%, 82% (95% CI 73%-88%) and 74% (95% CI 56%-87%) of the vaccinated subjects can be expected annually not to fail the vaccine.

Since our estimates of Ro, t and f were projections, we assessed the relative importance of these parameters on the estimate of Pc. To this end, we conducted one-way sensitivity analyses. We assumed the following baseline (range) values for these parameters: Ro, 2.0 (1.0-10.0); t, 0.8 (0.6-1.0) and f, 0.9 (0.6-1.0). Using these values we conducted sensitivity analyses.

The results demonstrate that over the selected range the Pc estimate is most sensitive to the Ro. In one way sensitivity analysis, when we fixed the baseline values of t and f, we observed that a Ro greater than 3.57 leads to a Pc value exceeding unity and thus implies the need for mass repeated vaccinations. By contrast, for the baseline value of Ro and f, variation of t over the indicated range did not entail a need of vaccination while for the baseline values of Ro and t, a value of ≦0.625 for f suggested a need for mass vaccination. Considering the values of t and f used in our analyses (Table 20) it is clear that Ro is the only critical determinant of Pc.

Another outcome of public health interest in epidemics is the critical response time, defined as the minimum available time for planning and implementing the preventive public health actions so as to prevent an impending epidemic (56). This response time is inversely proportional to the probability of transmission and is estimated as 1/β. We estimated the CRT (FIG. 15C) within each stratum of the target population based on the estimated probability of HIV-1 transmission described.

In the epidemiologic literature, AF is commonly used to estimate the burden of disease that can be attributed to the presence of a putative risk factor. In accord with this concept, we estimated the burden of the projected epidemic that is attributable to the GRGs of the infected and the susceptible partners in the target population. Since Ro is the critical parameter dictating the epidemic behavior for a fixed value of t and f we used the estimated value of Ro as a measure of the strength of association between the population stratum (determined on the basis of the GRGs of infected and susceptible partners) and the severity of the potential epidemic in each of these population groups. Then, using the method of estimating AFs for a multiple category risk factor as described previously (1) we estimated the AF for the entire target population and for each stratum within the target population (FIG. 15C). For computing the AF, we used the estimated frequency of the populations groups and the Ro shown in FIG. 15A.

In our mathematical modeling, we assumed that the risk behavior is not influenced by the GRGs.

Having estimated the Ro within each population stratum defined by the GRGs of the infected and susceptible partner, we conducted proof-of-principle studies to determine the effects of the GRGs on the epidemic trajectories. For this purpose, we predicted the epidemic trajectory within each population stratum. As indicated, based on the GRGs of the HIV-positive index partner and susceptible partner, the population can be subdivided into nine strata. A discrete-time, compartmental, susceptible-infected-removed (SIR) model of epidemics was used (57-59). As indicated above, the estimates of Ro were derived from annual probability of transmission. Therefore, we predicted the time course of the epidemic in years since the beginning of the epidemic. For this analysis we assumed a closed and non-growing population and thus allowing the epidemics to die out naturally. Also, we assumed a relatively homogeneous population within each stratum. An initial population size of 1 million was assumed and the epidemic was assumed to have been initiated by a single index case.

We assessed the association between cCD4 and VL-sp using an exponential regression. For this regression model, the cCD4 data were log-transformed and the VL-sp was regressed on the transformed cumulative CD4 count. Using these estimates we generated a projected curve between cCD4 and VL-sp that depicts the exponential nature of the relationship between these two variables in each GRG (FIG. 12E).

Even after varying the cut-off for the viral loads (FIG. 13D) from 500-100,000 copies/ml we observed that the proportion of subjects below the value of the chosen cut-off was consistently least in the high risk GRG, intermediate in the moderate risk GRG and greatest in the low risk GRG, suggesting a relationship between the depth of VL reduction and GRGs. To summarize this relationship we made use of the area under the curve notion. We normalized the cut-off points on a scale of 0-1 (0 representing no viral load while 1 representing a viral load of 100,000 copies/ml. Thus, the plot shown in FIG. 13D was translated into a unit square with an area of 1. We then estimated the area under each of the curves (specific to each GRG) using the trapezoidal rule. This area represents the overall probability of viral suppression within each GRG irrespective of the definition of viral suppression. This estimate is shown as AUC in FIG. 13D.

In the AIEDRP cohort, 135 EA subjects received HAART and of these genotyping information was available on 123 subjects. We studied the efficacy of HAART in these individuals as function of CCL3L1 and CCR5 genotype. The endpoint we used in this analyses was the fold-change from the baseline (pretreatment) CD4+ T cell count as a measure of the HAART efficacy. For estimating this fold-change we used the following strategy. First, we estimated the baseline CD4+ T cell count as a geometric mean of all the CD4+ T cell count estimates available on an individual prior to the initiation of the treatment. Second, after the initiation of the therapy we estimated the geometric mean of the CD4+ T cell counts measured up to three time points: six months, one year and two years. Thus, our strategy made use of all the available measurements on CD4+ T cell counts. Third, the ratio of post-HAART average CD4+ T cell counts (up to six months, 1 year and 2 years) and the baseline average CD4+ T cell count provided an estimate of the fold-change from the baseline CD4+ T cell count. The fold-change was then converted to % change in CD4+ T cell counts as (fold change-1)×100. This outcome was compared in subjects with different CCR5 genotypes in FIG. 16, F and G.

We then defined failure to respond to HAART as a fold-change in CD4+ T cell counts of ≦1. Since this was a dichotomous variable, we used unconditional logistic regression to predict the risk of failure to HAART response based on genotypes (results shown in FIG. 16H). This analysis was also conducted at three time points after initiation of the therapy: six months, one year and two years.

We also exclude any possibility of bias of with respect to receipt of HAART and host CCR5 genotype. In the AIEDRP cohort, the decision to treat was not based on the CCR5 genotypes since the genotyping was conducted after the cohort was assembled and followed-up. We examined the association between the likelihood being treated (by ART or HAART) and the CCR5 genotypes using a multivariate logistic regression model. We observed that the choice of therapy—whether ART or HAART—was oblivious to the CCR5 genotypes of the study subjects.

The CD4+ T cell-related parameters that are traditionally evaluated in the context of understanding the relationship between virus and CD4 changes in epidemiological or clinical studies are baseline CD4+ (bCD4), nadir CD4+ (nCD4) or rate of change in CD4+ T cell counts (rCD4). Additionally, computation of the latter parameter assumes that the rate of change in CD4+ T cell counts over time is linear. However, an analysis of actual patient trajectories in the context of both cohort studies or clinical practice suggests that this is not the case, and rather the trajectories over time are non-linear.

With the aforementioned considerations in mind, we developed a new parameter that we designate as cumulative CD4+ T cell count (cCD4). The intention was to develop a more informative epidemiological marker for assessing changes in the CD4+ T cell pool in HIV-infected subjects over their disease course. This measure was estimated by calculating the area under the CD4+ T cell count trajectory of an individual over the disease course. The area was estimated using the trapezoidal rule. We also posited that this parameter would permit us to further probe the relationship between early time-point events such as bCD4 and VL-sp and more distal events such as CD4+ T cell loss.

The cCD4 estimate assumes a continuous, dynamic association of CD4+ T cell counts over time in HIV-infected subjects. We considered six types of construct validity to demonstrate that the cCD4 is a valid parameter that reflects the dynamic disease process in HIV-infected individuals.

1. This measure has the face validity in that it can be conceived to be tracking the entire CD4+ T cell pool over time, which in the untreated HIV-infected host is known to get depleted over time. Smaller values of cCD4 indicate a smaller total CD4+ T cell pool over the disease course and we anticipated that this would be associated with a severe or faster disease progression. Thus, the measure can be thought of as a parameter of the disease progression.

A possible bias in this analysis could be related to the length of the follow-up of an individual since a longer follow-up is likely to over-estimate the cCD4. For this reason, we also estimated cCD4 corrected for the length of the follow-up (c-cCD4). We observed that there was a strong correlation between cCD4 and c-cCD4 (Spearman's ρ=0.7581, P<0.0001). Additionally, even after correcting for the length of follow-up the strengths of association of cCD4 and c-cCD4 with various other established surrogate markers were very similar. We therefore chose to use the uncorrected measure (i.e., cCD4) for all the analyses.

2. In terms of the content validity, arguably, this measure differs from the other CD4+ T cell count-based surrogate markers with regard to the pathogenesis-related information content. cCD4 concurrently takes into account three contents: baseline CD4 T cell counts, change in CD4+ T cell counts and the unit of time. The figure shown below depicts some of these conceptual issues regarding content provided by cCD4. For instance, panel A shows that for the same value of the baseline CD4+ T cell count three different trajectories designated as T1, T2, and T3 of the CD4+ T cell count over time will lead to different estimates of cCD4. Similarly, panel B shows that even though the nadir CD4 counts were same for the three theoretical trajectories, the estimates of cCD4 can be different based on the trajectory of the CD4+ T cell time trend. Moreover, if one were to fit least-squares regression lines to these individual trajectories, then the slopes of the regression lines also do not completely correlate with cCD4.

This demonstrates the conceptual difference of cCD4 from other markers based on CD4+ T cell count. Thus, cCD4 permits the combination of the information contained in bCD4, nCD4 and rates of changes in CD4+ T cell counts (rCD4) without making the assumption of a linear loss of CD4+ T cell counts over time in HIV-infected subjects.

These conceptual differences among the CD4+ T cell count-related parameters of bCD4, nCD4, rCD4 was also supported by actual data from the WHMC cohort. Analysis of covariance identified that bCD4, nCD4 and rCD4 together accounted for only ˜30% of the variation in cCD4 (F=156.68, df=3,1091; P=1.9×10⁻⁸⁴). Thus, a major proportion of the cCD4 variance remained unexplained by the potential correlations with other CD4+ T cell count-based measures. Moreover, the rate of progression to AIDS was independently predicted by all these four measures when they were included in a single multivariate model (the P values were: 0.0173 for bCD4, 5.2×10⁻⁹ for rCD4, and <1×10⁻²² for nCD4 as well as cCD4) again emphasizing the conceptual difference between cCD4 and the other traditionally used CD4+ T cell count parameters.

3. The predictive validity of cCD4 is documented by the fact that the mean cCD4 is subjects who developed AIDS in the WHMC cohort was 756,523 cell-days/ml whereas in subjects who did not develop AIDS the mean cCD4 was 1,496,342 cell-days/ml. This ˜2 fold difference in cCD4 was statistically highly significant. (Mann-Whitney P value <1×10⁻²²).

4. The concurrent validity of the proposed cCD4 measure is evident in FIG. 12B which shows that the tertiles of cCD4 were associated with strikingly different rates of progression to AIDS. The annual incidence rate of AIDS was 0.02, 0.08 and 0.19 in the subjects belonging to higher, middle and lower tertile groups, respectively. The cCD4 tertile categories also clearly stratified progression to death (logrank test χ²=676.56, df=2, p=1.2×10⁻¹⁴⁷). Moreover, there was a statistically significant correlation between the rate of CD4+ T cell loss (estimated using best-fitting least squares regression) and cCD4 (Table S4). Thus, the cCD4 concurred with the established measures of disease progression in HIV-infected subjects.

5. The convergent validity of cCD4 is exemplified by the correlation matrix shown in Table

23. cCD4 correlated strongly with all the markers that use the CD4+ T cell counts—bCD4, nCD4 and rCD4. The results of the factor analysis also point to the convergent validity of cCD4. Thus, each of these determinants of CD4+ T cell loss though correlated provide an independent measure of CD4+ T cell-based AIDS prognostication.

6. Discriminant validity relates to a measure's ability to not correlate with other measures that are known to be unrelated with the construct. In the WHMC cohort we observed that the cCD4 was not associated with gender (Mann-Whitney P value=0.5381), ethnic background (Kruskal-Wallis P value=0.3558) or a random variable like personal identification number (Spearman's rho=−0.008, P=0.7898).

Taken together, these arguments and observations strongly indicate that cCD4 is valid both conceptually and operationally as a marker for HIV disease. However, given the nature of this estimate its greatest value is for a retrospective analysis of prospectively obtained CD4+ T cell counts.

Both the viral load set point and rate of CD4+ T cell decline display variations of over several orders of magnitude among patients (1, 4). Despite intensive research, the host and virus factors that are responsible for the observed variation remain poorly understood. Additionally, although they are important clinical tools, these laboratory markers have four significant limitations with respect to risk-assessment of infected patients.

First, not all persons at high risk of an accelerated disease course are identified by these laboratory markers. For example, in the analyses of 1,132 HIV-infected subjects followed prospectively at the WHMC, although baseline CD4+ T cell counts or viral loads (viral set point) had prognostic value in predicting risk of rapid disease progression, infected individuals having similar levels of these two laboratory markers displayed highly variable rates of disease progression. Exemplifying this variability, ˜30% of subjects with baseline CD4+ T above 700 cells/μl developed AIDS at the same rate as did individuals with baseline counts lower than 350 cells/μl. Similarly, 40% of individuals with low viral set points (<20,000 copies/ml) progressed to AIDS in ˜5 years. These findings indicate that a low baseline CD4+ T cell count or high viral set point favors heavily the possibility of an increased risk of progressing rapidly to AIDS, but the converse is not true, i.e., a high baseline CD4+ T cell count or low viral set points does not exclude the possibility of an accelerated disease course.

Second, baseline CD4+ T cell counts and viral loads (Spearman rho=−0.2439, P<0.0001) or rate of CD4+ T cell decline (rho=−0.1763, P<0.0001), and viral load and rate of CD4+ T cell decline (rho=−0.1904, P=0.0006) are correlated in this cohort of infected adults. The latter findings indicate that these laboratory markers capture overlapping components of AIDS risk.

Third, by computing the log likelihood from the Cox proportional hazards models to estimate the amount of variation (R_{M}²) in the rate of progression to AIDS that is explained by baseline CD4+ T cell counts and viral loads, we found that the R_{M}² values were comparably low for these two markers. These findings indicate that despite statistically significant, and sometimes impressive relative hazards for the association between different baseline CD4+ T cell counts and viral load strata, these markers of disease progression explain only a small fraction of the overall variation in clinical course of an HIV+ individual (18), emphasizing the need to identify additional independent markers of disease progression.

Finally, clinical decision-making in HIV-1 medicine oftentimes hinges more on the serial assessments of CD4+ T-cell counts or viral loads over time. Thus, single time-point estimates of these two laboratory markers although providing a snapshot in the disease process, may not correlate fully with the future trajectory of the clinical course of patients.

Collectively, these findings and conceptual underpinnings support the urgent need for population-based data to identify host-centric risk factors that can (i) predict the future risk of AIDS independent of CD4+ T cell counts and viral loads; and/or (ii) provide clues into the immune correlates of the observed variation in T cell loss and the viral set point. We surmised that knowledge of these host-centric vulnerability factors will not only aid in the global risk assessment and clinical management of infected patients. The host centric factors that we focused on here were CCL3L1 gene dose and CCR5 genotypes.

The findings in FIG. 14 demonstrated that the extent of the disease-influencing effects associated with the GRGs was not fully captured by simply measuring the laboratory markers. If this was true, then GRGs should discriminate for a lower or higher likelihood of developing AIDS when the laboratory markers do not. To test this, we used likelihood ratios (LRs), which are used to assess how informative a diagnostic test result is (39).

The three GRGs and different strata of CD4+ T cell counts or VL set points are associated with predictable LRs such that a LR>1 or <1 indicates a higher or lower likelihood of developing AIDS, respectively. However, across a range of CD4+ and VL strata, including those that would have predicted a reduced risk of developing AIDS, the low and high risk GRGs consistently tracked individuals with a lower and higher likelihoods of developing AIDS, respectively. For example, the LR for a CD4+ T cell count of <350 was 2.44, but in this setting, the low and high risk GRGs had LRs of 0.69 and 13.28, respectively. Similarly, the LR for the strata characterized by CD4+ T cells ≧350 cells/μl and VLs <55,000 copies/ml was 0.64. However, the LR for the high risk GRG in this CD4/VL stratum was 3.28, which is a swing of susceptibility of nearly 8-fold. A similar, but slightly weaker discriminatory effect was evident for the moderate risk GRG. Additionally, whether ART should be initiated can challenging in certain CD4/VL strata (e.g., CD4+>350 or VL between 20-55,000 copies/ml), and in these strata, the LRs for the laboratory markers were non-informative, whereas those for the GRGs were.

The LRs for the biomarkers were higher early after infection and then decreased. By contrast, the LRs for the three GRGs were stable over several years, demonstrating the time-insensitivity of their prognostic value that contrasts with the need to make serial measurements of the laboratory markers. Notably, in this longitudinal analysis, the LRs for the three GRGs were quantitatively comparable to those for the three different CD4+ and VL strata that are typically used as cut-offs when making considerations for initiation of ART (16, 18), highlighting the possible utility of GRGs in this oftentimes, difficult clinical decision making.

We generated KM plots for the time to AIDS in the subjects belonging to each of the terminal nodes in the final tree (FIG. 14D). The logrank test (χ²=60.35, p=2.4×10⁻¹²) indicated that the tree generated groups that were significantly different statistically in terms of the rate of progression to AIDS. We then determined the probability of developing AIDS in subjects belonging to each of the terminal node. For this we made use of the concept of prognostic likelihood ratios. The pre-test and post-test odds of an outcome are related by the following well-known equation: post-test odds=pre-test odds×likelihood ratio. Using the overall probability of developing AIDS in the seroconverting cohort (that is without considering any of the three prognostic predictors: VL-sp, bCD4 and GRGs) as a measure of the pre-test probability of AIDS, we estimated the post-test probability of AIDS for subjects belonging to the terminal nodes of the final classification tree (FIG. 14D). The reason for using likelihood ratios in this setting is to provide a clinical measure in settings where the pre-test probabilities can differ.

To assess the importance of the GRGs in the classification tree, we generated a subtree by artificially removing the GRGs from the tree shown in FIG. 14B (the reduced tree is shown in FIG. 16D). This subtree had four terminal nodes—i) bCD4≦453 cells/μl, ii) bCD4>453 cells/μl and HIV RNA≦17500 copies/ml iii) bCD4>453 cells/μl and HIV RNA 17,500-55,500 copies/ml and iv) bCD4>453 cells/μl and HIV RNA >55,500 copies/ml. We analyzed this subtree in the same manner as described above.

We also used an additive prognostic risk scoring system to determine the practical utility of the GRGs in AIDS prognostication. In contrast to the decision tree analyses shown in FIG. 14 (C to E), the prognostic risk scoring system used cut-offs that are currently clinically used for decision making. We used the three laboratory markers: baseline CD4+ T cell count, viral load set point and the GRGs based on CCR5 and CCL3L1 genotypes—to construct a simple additive risk scoring system. We dichotomized CD4+ T cell counts based on cut-off value of 200 cells/μl and viral load set point based on a cut-off value of 55,000 copies/ml. We coded the GRGs as 0, 1, and 2 for low-, moderate- and high-risk groups.

Thus, the theoretical range of score is 0-4 with higher scores indicating higher risk. We then assessed and validated the prognostic use of this risk scoring system in three ways. Similar to the approach taken in analyzing the decision tree (FIG. 14, B to D), we assessed the importance of the GRGs by excluding and including them in the risk scoring system.

First, we compared the likelihood ratio χ² statistic from the nested Cox regression models. We used five Cox regression models using following covariates: i) dichotomized baseline CD4+ T cell count only (C), ii) dichotomized viral set point only (V), iii) GRGs only (G), iv) dichotomized baseline CD4+ T cell count combined with dichotomized viral set point (C+V), and v) all the three components of the risk scoring system together (C+V+G). With these analyses we addressed two questions: i) Used individually, how do the GRGs perform vis-á-vis baseline CD4+ T cell count and VL set point in prognosticating the HIV-1 infected subjects; and ii) If the GRGs are combined with the baseline CD4+ T cell count and viral set point, can such a system provide additional prognostic information that is not sufficiently captured without the inclusion of GRGs into the prognostic system for HIV-1 infected subjects?

Second, using Kaplan-Meier plots and log-rank test we determined the association of the risk score with time to AIDS (1987 diagnostic criteria), time to AIDS (1993 criteria) and time to death in the seroconverting component of the WHMC cohort. We also conducted these analyses in the subgroup of seroconverters who were recruited into the cohort after January 1990, thus allowing us to assess the importance of the risk scores in those who had some opportunity to receive therapy.

Third, we estimated the proportion of subjects developing AIDS within each risk score category in all the seroconverters as well as in the subgroup of serocoverters who were recruited after January 1990. One of the clinical criteria sometimes used for initiating the HAART is the estimated probability of AIDS within three years exceeding 30% (60, 61). Therefore, we estimated the probability of AIDS within three years of seroconversion using parametric survival regression analyses. We used Gompertz distribution and predicted the probability of AIDS at three years as:

$$P ( AIDS \vert t = 3 ) = 1 - {e}^{- \frac{{e}^{λ}}{γ} ( {e}^{3 γ} - 1 )}$$

where, the parameter λ was estimated from the Gompertz regression coefficients and the parameter γ was reported by Stata 7.0 as an ancillary parameter estimated from the data.

In nested Cox proportional hazards models, the prognostic performance of the model that contained the GRGs along with VL and CD4+ T cells was superior (indicated by higher likelihood ratio χ² and lower Akaike information criterion [AIC] values). Further illustrating the independent effects of GRGs, subjects could be partitioned into an increased number of risk groups for rate of progression to AIDS that more accurately classified the rate of disease progression and death compared to a model that only considered VL and CD4+ cell counts. Finally, the GRGs provided an independent measure of the risk of developing AIDS.

Randomization is resorted to in clinical/preventive trials in an attempt to achieve a balanced distribution of the known and unknown confounding variables. However, randomization is likely to fail or be inadequate especially if the sample size of a trial is small. We simulated a typical two-arm trial design to examine the influence of the genotypic imbalance across trail arms on the estimates of HIV vaccine efficacy.

To start with, let us assume that the potential role of genotypes on the risk of acquisition of HIV as well as the distribution of the genotypes in the trial sample is not known. Thus, the relative risk (r) of infection is defined as a/v÷c/u and the vaccine efficacy (ê) is estimated as 1-r. Now, let us assume that we know the possession of a particular genotype increases the risk of acquiring HIV infection. In our case, for example, the possession of either the CCR5 detrimental genotype or of less than population-specific median copies of the CCL3L1 gene (or both) can increase the risk of HIV-acquisition 1.72-folds (95% CI 1.44-2.04, p=8.8×10⁻¹⁰). If randomization is proper and adequate, then we expect that the proportion of vaccinees with the low risk GRG will be same as the proportion of unvaccinated subjects possessing the low risk GRG and the general population prevalence of the genotypes (pₒ, in the case of WHMC cohort pₒ=0.5). When, however, partial misallocation occurs then the subjects the estimate of vaccine efficacy can be expected to be biased because of the unequal risk of acquiring HIV across GRGs and because of the unequal distribution of the genotypes across trial arms.

Let Iᵤ be the incidence of the HIV-infection in unvaccinated subjects and let Iᵥ be incidence of HIV-infection in the vaccinees. If e is the true vaccine efficacy, then e=1−(Iᵥ/Iᵤ), and alternatively

Iᵥ=(1−e)Iᵤ. (1)

The incidence of infection occurring in the each trail arm can be considered as a weighted (based on the prevalence of respective GRGs) average of the risk of acquiring HIV-acquisition across the GRGs. Thus, in the unvaccinated subjects, if pₒᵤ is the prevalence of the low risk GRG, then the expected number of infected subjects at the end of the trial will be

nIᵤ[pₒᵤ+(1−pₒᵤ)r], (2)

where, n is the number of subjects recruited in the trial. Similarly, if pₒᵥ is the prevalence of the low risk GRG in the vaccinated subjects, then the expected number of cases in the vaccinated group will be

nIᵥ[pₒᵥ+(1−pₒᵥ)r], (3)

Substituting the value of Iv from (1), we get

n(1−e)Iᵤ[pₒᵥ+(1−pₒᵥ)r], (4)

The estimated vaccine efficacy (ê) can then be calculated as

$$\hat{e} = 1 - \{ \frac{1 - {ep}_{ov} + ( 1 - {p}_{ov} ) r}{{p}_{ou} + ( 1 - {p}_{ou} ) r} \} ,$$

since n and Iᵤ cancel out.
If there is no misallocation, then pₒᵥ=pₒᵤ and ê=e.

If m represents the fraction of the trial subjects misallocated so that there is an enrichment of subjects with high/moderate risk GRG in the vaccinated subjects at the cost of the low risk GRGs in the unvaccinated subjects and ρ represents the ratio of vaccinated to unvaccinated subjects enrolled in (and who completed) the study, then it can be shown that both pₒᵥ and pₒᵤ are functions of po, m and ρ and can be estimated as

$$\begin{matrix}{P}_{ov} = \frac{ρ ( {p}_{o} - m ) - m}{ρ} and & ( 6 ) \\ {P}_{ou} = {P}_{o} + m ( 1 + ρ ) & ( 7 )\end{matrix}$$

In the cases of equal size of the trial arms, ρ=1 and pₒᵥ=pₒ−2m while pₒᵤ=pₒ+2m. In words this means that the prevalence of the low risk GRG is reduced in the vaccinated group and increased in the unvaccinated group by a factor proportional to the fraction of subjects misallocated. Consequently, one can expect an excess (than expected) of HIV-infections in the vaccinated group and a reduction (than expected) of HIV-infections in the unvaccinated subjects. This, in turn, can be expected to lead to a decreased estimate of the vaccine efficacy. If, we substitute equations (6) and (7) into equation (5), we get

$$\hat{e} = 1 - \{ \frac{( 1 - e ) [ {p}_{o} ( 1 - r ) - m ( 1 + \frac{1}{ρ} ) ( 1 - r ) + r ]}{{p}_{o} ( 1 - r ) + m ( 1 + ρ ) ( 1 + r ) + r} \} \qquad ( 8 )$$

This equation capture the direct relationship between the degree of misallocation (m) and the estimated vaccine efficacy (ê).

Using the estimates of pₒ and r from the WHMC data, assuming a trial of equal sized arms (that is ρ=1) and varying the true vaccine efficacy we assessed the influence of the degree of misallocation on the estimates of the vaccine efficacy that would have resulted from a trial with inadequate randomization. The results are shown in FIG. 15E.

The left panel in FIG. 15E shows the estimates of vaccine efficacy (ê) for varying values of m (shown as percentage) and true vaccine efficacy. The right panel shows the difference between the true vaccine efficacy and the estimated vaccine efficacy as a percentage of the true vaccine efficacy for varying values of m.

We observed that the vaccine efficacy-reducing influence of misallocation was magnified if the true vaccine efficacy was low. Based-on the results shown in panel B, the relative error in estimates of vaccine efficacy can vary between 0.2%-19% based on vaccine efficacy for a very small misallocation rate of 1%. In the scenario of HIV-vaccines it is expected that most of the candidate vaccines will have partial protective effect. As an example, in a trial of a 50% efficacious vaccine on 500 subjects, misallocation of only 5% (25) subjects will lead to an estimate of 44% for vaccine efficacy (95% confidence interval 41%-45%)—a relative error of ˜12% in the estimate. Therefore, randomization based on genotypic information can be expected to ameliorate the confounding in the estimates of vaccine efficacy. As an alternative, stratified statistical analysis based on the genotypic information (e.g., Mantel-Haenszel test) can overcome the confounding in the estimates of vaccine efficacy. Therefore, whether at the time of recruitment or statistical analysis, knowledge of the GRGs of the study subjects will refine the estimates of vaccine efficacy.

### REFERENCES FOR EXAMPLE VI

1. J. S. Altshuler, D. Altshuler, Nature 429, 478 (2004).
2. E. Gonzalez et al., Science 307, 1434 (2005).
3. S. Mummidi et al., Nat Med 4, 786 (1998).
4. E. Gonzalez et al., Proc Natl Acad Sci USA 96, 12004 (1999).
5. E. Gonzalez et al., Proc Natl Acad Sci USA 98, 5199 (2001).
6. E. Gonzalez et al., Proc Natl Acad Sci USA 99, 13795 (2002).
7. A. Mangano et al., J Infect Dis 183, 1574 (2001).
8. S. J. Little et al., N Engl J Med 347, 385 (2002).
9. R. S. Janssen et al., Jama 280, 42 (1998)
10. M. C. Strain et al., J Infect Dis 191, 1410 (2005).
11. D. D. Richman et al., Proc Natl Acad Sci USA 100, 4144 (2003).
12. W. Zucchini, J Math Psychol 44, 41 (2000).
13. J. K. Lindsey, B. Jones, Stat Med 17, 59 (1998).
14. M. P. Martin et al., Science 282, 1907 (1998).
15. R. A. Kaslow, T. Dorak, J. J. Tang, J Infect Dis 191 Suppl 1, S68 (2005).
16. M. Dybul et al., MMWR Recomm Rep 51, 1 (2002).
17. P. G. Yeni et al, Jama 292, 251 (2004).
18. P. G. Yeni et al., Jama 288, 222 (2002).
19. G. J. Nabel, Nature 410, 1002 (2001).
20. D. A. Garber, G. Silvestri, M. B. Feinberg, Lancet Infect Dis 4, 397 (2004).
21. M. L. Disis et al., Clin Cancer Res 6, 1347 (2000).
22. F. Hladik et al., J Immunol 166, 3580 (2001).
23. S. P. Blatt et al., Ann Intern Med 119, 177 (1993).
24. D. L. Birx et al., J Acquir Immune Defic Syndr 6, 1248 (1993).
25. M. J. Dolan et al., J Infect Dis 172, 79 (1995).
26. F. M. Gordin et al., J Infect Dis 169, 893 (1994).
28. G. E. Cleveland W S, Shyu W M., Local regression models. H. T. In:Chambers J M, eds., Ed., Statistical models (Chapman & Hall, S. London, 1993), pp. 309-76.
29. R. Detels et al., Jama 280, 1497 (1998).
30. P. M. Tarwater et al, Am J Epidemiol 154, 675 (2001).
31. S. Perez-Hoyos et al., Aids 17, 353 (2003).
32. M. I. Langdorf et al., Eur J Emerg Med 9, 115 (2002).
33. S. C. Lemon et al., Ann Behav Med 26, 172 (2003).
34. L. Li et al., Stat Med 23, 271 (2004).
35. M. A. Province, W. D. Shannon, D. C. Rao, Adv Genet 42, 273 (2001).
36. A. Vlahou et al., Clin Breast Cancer 4, 203 (2003).
37. N. J. Birkett, J Clin Epidemiol 41, 491 (1988).
38. D. L. Simel, G. P. Samsa, D. B. Matchar, J Clin Epidemiol 46, 85 (1993).
39. E. J. Gallagher, Ann Emerg Med 31, 391 (1998).
40. M. Schemper, J. Stare, Stat Med 15, 1999 (1996).
41. M. Schemper, R. Henderson, Biometrics 56, 249 (2000).
42. M. Schemper, Stat Med 22, 2299 (2003).
43. M. Schemper, Stat Med 12, 2377 (1993).
44. J. T. Kent, J. O'Quigley, Biometrika 75, 525 (1988).
45. E. L. Kom, R. Simon, Stat Med 9, 487 (1990).
46. P. M. Bentler, K. H. Yuan, Br J Math Stat Psychol 49 (Pt 2), 299 (1996).
47. S. Blower, E. J. Schwartz, J. Mills, AIDS Rev 5, 113 (2003).
48. R. Anderson, M. Hanson, J Infect Dis 191 Suppl 1, S85 (2005).
49. T. C. Quinn et al., N Engl J Med 342, 921 (2000).
50. R. H. Gray et al, Lancet 357, 1149 (2001).
51. E. A. Operskalski et al., Am J Epidemiol 146, 655 (1997).
52. M. A. Pedraza et al., J Acquir Immune Defic Syndr 21, 120 (1999).
53. U. S. Fideli et al., AIDS Res Hum Retroviruses 17, 901 (2001).
54. M. J. Wawer et al., J Infect Dis 191, 1403 (2005).
55. C. Hickie et al., Int J Immunopharmacol 17, 629 (1995).
56. A. L. Rivas et al, Can J Vet Res 67, 307 (2003).
57. F. Ball, P. Neal, Math Biosci 180, 73 (2002).
58. A. van Nes, Vet Q 23, 21 (2001).
59. D. Wang, X. Zhao, Beijing Da Xue Xue Bao 35 Suppl, 72 (2003).
60. C. C. Carpenter et al, Jama 283, 381 (2000).
61. R. B. Geskus et al., J Acquir Immune Defic Syndr 32, 514 (2003).

The foregoing is considered as illustrative only of the principles of the invention. Further, since numerous modifications and changes will readily occur to those skilled in the art, it is not desired to limit the invention to the exact construction and operation shown and described herein. Therefore, accordingly, all suitable modifications and equivalents fall within the scope of the invention.

All publications, patent applications, patents, patent publications and other references cited herein are incorporated by reference in their entireties for the teachings relevant to the sentence and/or paragraph in which the reference is presented.

### Tables for Examples I & II

TABLE 1
Selection of CCL3L1 gene copies for purposes of determining their
associated disease-and/or transmission-modifying effects.

| | | Protective |
|---|---|---|
| | Detrimental | CCL3L1 |
| | CCL3L1 gene | gene copies |
| Clinical setting | copies (CCL3L1^{low}) | (CCL3L1^{high}) |
| Risk of vertical transmission | <2 copies | 2 copies |
| Risk of horizontal transmission | <2 copies | 2 copies |
| in non-AAs (combined analysis | | |
| for EAs plus HAs) | | |
| Risk of horizontal transmission | <4 copies | 4 copies |
| in AAs (note, median in HIV− | | |
| is 4) | | |
| Rae of disease progression in | <2 copies | 2 copies |
| EAs | | |
| Rate of disease progression in | <3 copies | 3 copies |
| AAs (note, median in HIV+ is | | |
| 3) | | |

TABLE 2
Selection of CCR5 genotypes for purposes of determining their associated disease-
and/or transmission-modifying effects

| | | | Log-rank P for |
|---|---|---|---|
| | Detrimental CCR5 genotypes | Non-detrimental CCR5 | CCR5^{det} vs. |
| Setting | (CCR5^{det}) | genotypes (CCR5^{non-det}) | CCR5^{non-det} |
| Rate of disease | HHE/HHE HHD/HHE, | Implies all CCR5 | |
| progression in EAs | HHC/HHG*1, HHA/HHA, | genotypes that lack | |
| | HHE/HHF*1, HHF*2/HHG*1 | CCR5^{det} | |
| Median time to AIDS | 5.71 years | 7.92 years | 0.0128 |
| Median time to death | 6.85 years | 8.94 years | 0.0039 |
| Rate of disease | HHC/HHE, HHC/HHC, | Implies all CCR5 | |
| progression in AAs | HHE/HHG*2, HHC/HHD, | genotypes that lack | |
| | HHD/HHG*1, HHB/HHC, | CCR5^{det} | |
| | HHA/HHF*1 | | |
| Median time to AIDS | 5.35 years | 10.31 years | 2.610⁻⁷ |
| Median time to death | 6.70 years | 11.22 years | 9.910⁻⁶ |
| Rate of disease | HHE/HHE, HHD/HHE, | Implies all CCR5 | |
| progression in HAs | HHE/HHG*2 | genotypes that lack | |
| | | CCR5^{det} | |
| Median time to AIDS | 4.15 years | 8.42 years | 0.0113 |
| Median time to death | 5.69 years | 9.20 years | 0.0174 |
| Rate of disease | Combination of population- | Implies all population- | |
| progression in EAs + AAs | specific CCR5^{det} genotypes | specific CCR5 genotypes | |
| | | that lack CCR5^{det} | |
| Median time to AIDS | 5.63 years | 8.78 years | 2.710⁻⁷ |
| Median time to death | 6.70 years | 9.57 years | 9.410⁻⁷ |
| Rate of disease | Combination of population- | Implies all population- | |
| progression in EAs + HAs + | specific CCR5^{det} genotypes | specific CCR5 genotypes | |
| AAs | | that lack CCR5^{det} | |
| Median time to AIDS | 5.59 years | 8.47 years | 3.210⁻⁸ |
| Median time to death | 6.59 years | 9.57 years | 1.510⁻⁷ |

TABLE 3
Boot-strapping analyses of risk of developing AIDS (relative
hazards and 95% CI).

| | | Bias-corrected bootstrap estimates |
|---|---|---|
| Genetic variable | Entire Cohort | (n = 792, Repetitions = 1000) |
| CCR5^{det} | 1.82 (1.46-2.28) | 1.82 (1.37-2.37) |
| CCL3L1^{low} | 1.61 (1.33-1.96) | 1.62 (1.28-1.99) |

TABLE 4
Predictive value of laboratory and genetic markers of HIV-1
infection prior to stratifying for different
baseline CD4+ T cell and viral load strata.

| | Cox PH | | 1-Hartz's index (%) | | |
|---|---|---|---|---|---|
| Model | R_{M}² | Sigma | Overall | 3 years | 7 years |
| VL | 0.0458 | 0.2590 | 35.96 | −34.26 | 33.24 |
| CD4⁺ | 0.0366 | 0.2749 | 12.64 | 46.50 | 13.22 |
| Gene | 0.0485 | 0.2922 | 23.00 | 13.36 | 25.26 |
| VL + CD4⁺ | 0.0748 | 0.2471 | 29.78 | 57.10 | 29.64 |
| VL + Gene | 0.0901 | 0.2528 | 38.92 | −1.54 | 37.26 |
| CD4⁺ + Gene | 0.0756 | 0.2707 | 27.86 | 50.24 | 31.48 |
| VL + CD4⁺ + Gene | 0.1079 | 0.2423 | 37.90 | 48.72 | 37.34 |

VL, viral load setpoint;
CD4⁺, baseline CD4⁺ T cells;
Gene, CCL3L1/CCR5 genotype;
PH, proportional hazard

TABLE 5
Predictive value of laboratory and genetic
markers of HIV-1 infection after stratifying for
different CD4⁺ T cell strata

| | | | 1-Hartz | | | 1-Hartz |
|---|---|---|---|---|---|---|
| Model | Cox PH R_{M}² | Sigma | index (%) | Cox PH R_{M}³ | Sigma | index (%) |
| | CD4⁺ T cells < 200 | | | CD4⁺ T cells < 350 | | |
| VL | 0.1745 | 0.1781 | 42.86 | 0.0218 | 0.2526 | 15.84 |
| CD4⁺ | 0.0004 | 0.2617 | 32.64 | 0.2482 | 0.2457 | 27.46 |
| Gene | 0.1544 | 0.2533 | 28.58 | 0.0687 | 0.3503 | 15.12 |
| VL + CD4⁺ | 0.1836 | 0.1624 | 42.86 | 0.2452 | 0.2056 | 39.00 |
| VL + Gene | 0.2834 | 0.1376 | 83.34 | 0.1204 | 0.2434 | 10.82 |
| CD4⁺ + Gene | 0.1646 | 0.2514 | 42.00 | 0.4063 | 0.2181 | 56.24 |
| VL + CD4⁺ + Gene | 0.2871 | 0.1347 | 100.00 | 0.3598 | 0.1579 | 43.02 |
| | CD4⁺ T cells 350-700 | | | CD4⁺ T cells > 700 | | |
| VL | 0.0606 | 0.2238 | 34.94 | 0.0288 | 0.2920 | 42.66 |
| CD4⁺ | 0.0010 | 0.2554 | 9.48 | 0.0157 | 0.2731 | 7.62 |
| Gene | 0.0468 | 0.2479 | 19.68 | 0.0474 | 0.2788 | 30.22 |
| VL + CD4⁺ | 0.0706 | 0.2226 | 29.82 | 0.0374 | 0.2816 | 41.16 |
| VL + Gene | 0.0939 | 0.2213 | 32.04 | 0.0938 | 0.2860 | 53.44 |
| CD4⁺ + Gene | 0.0476 | 0.2481 | 22.98 | 0.0549 | 0.2688 | 35.02 |
| VL + CD4⁺ + Gene | 0.1032 | 0.2207 | 30.90 | 0.0975 | 0.2770 | 53.50 |

VL, viral load setpoint;
CD4+, baseline CD4+ T cells;
Gene, CCL3L1/CCR5 genotype

TABLE 6
Predictive value of laboratory and genetic markers of HIV-1 infection after
stratifying for different viral load strata.

| | VL > 55k | | | VL 20k-55k | | | VL < 20k | | |
|---|---|---|---|---|---|---|---|---|---|
| | | | 1-Hartz | | | 1-Hartz | | | 1-Hartz |
| Model | Cox PH R_{M}² | Sigma | index (%) | Cox PH R_{M}³ | Sigma | index (%) | Cox PH R_{M}³ | Sigma | index (%) |
| VL | 0.0112 | 0.2324 | 3.26 | 0.0400 | 0.1719 | 22.94 | 0.0156 | 0.3137 | 13.96 |
| CD4⁺ | 0.0000 | 0.2798 | 9.44 | 0.0750 | 0.1509 | 10.70 | 0.0279 | 0.3033 | 17.52 |
| Gene | 0.0639 | 0.2852 | 14.88 | 0.1305 | 0.1732 | 39.46 | 0.0150 | 0.3186 | 10.58 |
| VL + CD4⁺ | 0.0113 | 0.2317 | 16.24 | 0.1322 | 0.1481 | 24.12 | 0.0389 | 0.2990 | 20.22 |
| VL + Gene | 0.0670 | 0.2187 | 17.28 | 0.1496 | 0.1695 | 46.30 | 0.0312 | 0.3106 | 21.60 |
| CD4⁺ + Gene | 0.0641 | 0.2672 | 21.88 | 0.1579 | 0.1500 | 40.50 | 0.0362 | 0.3072 | 20.26 |
| VL + CD4⁺ + | 0.0675 | 0.2184 | 22.44 | 0.1811 | 0.1461 | 45.64 | 0.0472 | 0.3007 | 26.02 |
| Gene | | | | | | | | | |

VL, viral load setpoint;
CD4⁺, baseline CD4⁺ T cells;
Gene, CCL3L1/CCR5 genotype

TABLE 7

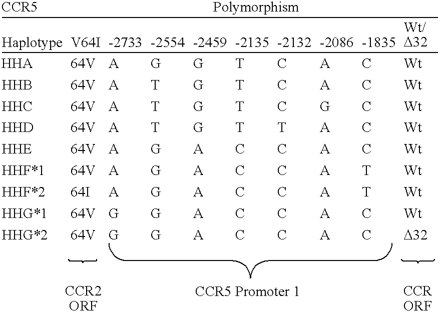

### Tables for Examples III and IV

TABLE 8
Association of CCL3L1 gene copy number with the risk
of vertical transmission in the absence of, or after
adjustment for the effects of, zidovudine (ZDV).

| | Adjusted for ZDV | | | In non-recipients of ZDV | | |
|---|---|---|---|---|---|---|
| CCL3L1 | (n = 682) | | | (n = 475) | | |
| gene copies | OR | 95% CI | P | OR | 95% CI | P |
| 0 | 0.76 | 0.33-1.75 | 0.520 | 0.71 | 0.30-1.69 | 0.434 |
| 1 | 1.58 | 1.06-2.37 | 0.025 | 2.08 | 1.24-3.49 | 0.005 |
| 2 | 1.00 | — | — | 1.00 | — | — |
| 3 | 0.59 | 0.35-0.99 | 0.045 | 0.61 | 0.33-1.14 | 0.122 |
| 4 | 0.50 | 0.26-0.96 | 0.038 | 0.55 | 0.27-1.15 | 0.113 |
| 5 | 0.60 | 0.23-1.60 | 0.310 | 0.47 | 0.14-1.54 | 0.212 |
| 6 | 0.34 | 0.07-1.46 | 0.147 | 0.47 | 0.09-2.42 | 0.367 |

TABLE 9
Changes in leukocyte subsets in HIV-positive EAs and AAs with CCL3L1 gene
copies that are lower than, or equal to the population-specific median

| | | |
|---|---|---|
| | Lower than population- | Population-specific |
| | specific median number | median number of |
| Rate of decline of | of CCL3L1 gene copies | CCL3L1 gene copies |

HIV⁺ AA (median = 4)

| | | | | |
|---|---|---|---|---|
| Lymphocyte count (cells/mth) | −3.52 | (−4.26, −2.78)* | −0.17 | (−1.04, 0.71) |
| CD3+ T cell count (cells/mth) | −1.29 | (−2.03, −0.54) | 1.26 | (0.64, 1.88) |
| CD4+ T cell count (cells/mth) | −1.66 | (−1.90, −1.41) | −1.24 | (−1.53, −0.95) |
| CD8+ T cell count (cells/mth) | −0.21 | (−0.80, 0.34) | 1.71 | (1.14, 2.28) |
| CD4/CD8 ratio (×10⁻⁴/mth) | −16.60 | (−19.79, −13.42) | −8.72 | (−13.49, −3.94) |
| CD3− T cell count | 0.04 | (−0.20, 0.28) | 0.48 | (0.19, 0.77) |
| % of CD3+ T cells (×10⁻²) | −3.96 | (−4.93, −2.98) | −0.56 | (−1.25, −0.12) |
| % of CD4+ T cells (×10⁻²) | −7.25 | (−7.94, −6.55) | −6.44 | (−7.55, −5.33) |

HIV⁺ EA (median = 2)

| | | | | |
|---|---|---|---|---|
| Lymphocyte count (cells/mth) | −3.77 | (−4.25, −3.11) | 0.01 | (−0.54, 0.54) |
| CD3+ T cell count (cells/mth) | −2.67 | (−3.27, −2.08) | 1.03 | (0.52, 1.53) |
| CD4+ T cell count (cells/mth) | −3.08 | (−3.21, −2.94) | −0.93 | (−1.11, −0.74) |
| CD8+ T cell count (cells/mth) | −0.62 | (−1.14, −0.11) | 1.41 | (1.01, 1.81) |
| CD4/CD8 ratio (×10⁻⁴/mth) | −26.17 | (−27.17, −25.17) | −10.25 | (−12.62, −7.88) |
| CD3− T cell count | 0.44 | (0.28, 0.60) | 0.39 | (0.22, 0.57) |
| % of CD3+ T cells (×10⁻²) | −5.20 | (−6.11, −4.29) | −0.79 | (−1.41, −0.18) |
| % of CD4+ T cells (×10⁻²) | −12.47 | (−12.94, −11.99) | −5.75 | (−6.35, −5.14) |

*numbers in parenthesis indicate 95% CI;
−, negative value denotes decline in parameter;
mth, indicates month.

TABLE 10
Risk of AIDS-defining illness with CCL3L1/CCR5 GRGs.

| AIDS-defining | | CCL3L1^{high}CCR5^{det} | | | CCL3L1^{low}CCR5^{non-det} | | | CCL3L1^{low}CCR5^{det} | | |
|---|---|---|---|---|---|---|---|---|---|---|
| Illness* | N | RH | 95% CI | P | RH | 95% CI | P | RH | 95% CI | P |
| CMV infection | 100 | 1.53 | 0.71-3.30 | 0.278 | 1.60 | 1.00-2.58 | 0.051 | 6.21 | 3.63-10.63 | 2.7 × 10⁻¹¹ |
| Cryptococcosis | 33 | 3.27 | 0.98-10.87 | 0.053 | 2.46 | 1.00-6.02 | 0.048 | 8.11 | 2.93-22.46 | 5.6 × 10⁻⁵ |
| Cryptosporidiosis | 24 | 1.21 | 0.27-5.47 | 0.802 | 1.21 | 0.49-3.00 | 0.686 | 1.63 | 0.36-7.37 | 0.526 |
| HAD | 54 | 2.05 | 0.82-5.13 | 0.126 | 1.65 | 0.87-3.11 | 0.124 | 3.18 | 1.33-7.60 | 0.009 |
| Herpes simplex | 26 | 1.78 | 0.50-6.41 | 0.375 | 1.22 | 0.49-3.04 | 0.668 | 1.66 | 0.36-7.53 | 0.513 |
| Histoplasmosis | 20. | 3.32 | 0.83-13.30 | 0.090 | 2.81 | 1.02-7.74 | 0.045 | 1.56 | 0.19-13.01 | 0.682 |
| Kaposi sarcoma | 74 | 1.76 | 0.76-4.05 | 0.186 | 1.66 | 0.96-2.86 | 0.069 | 3.86 | 1.90-7.83 | 2.0 × 10⁻⁴ |
| Lymphoma | 37 | 2.87 | 1.10-7.48 | 0.031 | 1.42 | 0.66-3.08 | 0.369 | 3.38 | 1.21-9.43 | 0.020 |
| MAC | 92 | 2.22 | 1.09-4.55 | 0.029 | 1.73 | 1.05-2.87 | 0.032 | 5.13 | 2.79-9.45 | 1.5 × 10⁻⁷ |
| PCP | 196 | 2.13 | 1.33-3.42 | 0.002 | 1.71 | 1.22-2.39 | 0.002 | 2.95 | 1.84-4.75 | 7.8 × 10⁻⁶ |
| PML | 18 | 1.72 | 0.36-8.10 | 0.494 | 1.27 | 0.44-3.67 | 0.657 | 2.41 | 0.51-11.43 | 0.268 |
| Toxoplasmosis | 27 | 1.49 | 0.32-6.91 | 0.610 | 1.69 | 0.67-4.25 | 0.268 | 5.34 | 1.77-16.07 | 0.003 |

The reference GRG for statistical analysis is CCL3L1^{high}CCR5^{non-det} (RH = 1).
The AIDS defining illnesses with sufficient events for statistical analyses recorded in the adult HIV+ cohort are shown.
*CMV, Cytomegalovirus; HAD, HIV-associated dementia; MAC, Mycobacterium avium complex; PCP, Pneumocystis carinii pneumonia; PML, Progressive multifocal leukoencephalopathy;
N, number of individuals with the indicated AIDS-defining illness.

TABLE 11
Human Study Populations
(HDGP, Human diversity genome panel; WHMC, Wilford Hall Medical Center; EA, AA, and HA
indicates European-, African- and Hispanic American)

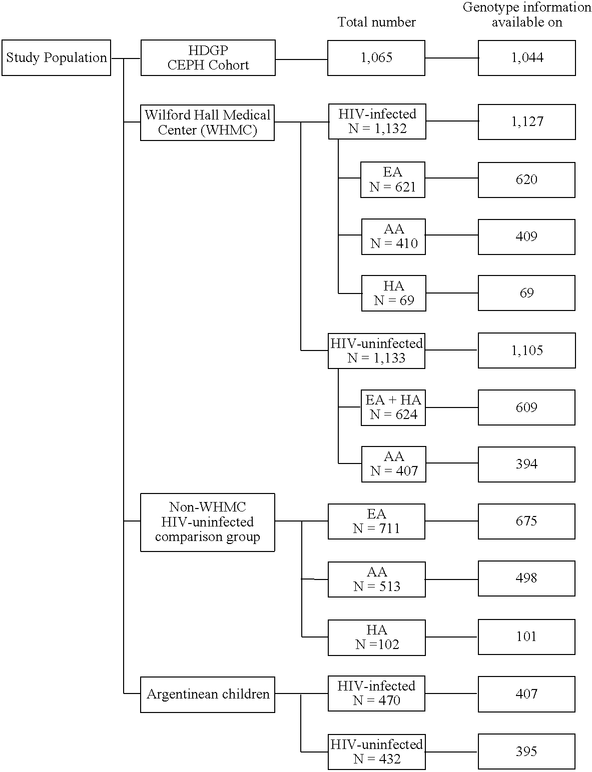

TABLE 12

| | Participants of HGDP- | | |
|---|---|---|---|
| | CEPH genotyped for | | |
| | CCL3L1 gene copies | | Genotype |
| Geographic origin | Population | n* | available |
| Total | | 1,064 | 1,046 |
| SUBSAHARAN AFRICA | | 127 | 127 |
| Central African Republic | Biaka Pygmy | 36 | 36 |
| Democratic Republic of | Mbuti Pygmy | 15 | 15 |
| Congo | | | |
| Senegal | Mandenka | 24 | 24 |
| Nigeria | Yoruba | 25 | 25 |
| Namibia | San | 7 | 7 |
| Kenya | Bantu N.E. | 12 | 12 |
| S. Africa Bantu S.E. | Bantu S.E. Pedi | 1 | 1 |
| S. Africa Bantu S.E. | Bantu S.E. Sotho | 1 | 1 |
| S. Africa Bantu S.E. | Bantu S.E. Tswana | 2 | 2 |
| S. Africa Bantu S.E. | Bantu S.E. Zulu | 1 | 1 |
| S. Africa Bantu S.W. | Bantu S.W. Herero | 2 | 2 |
| S. Africa Bantu S.W. | Bantu S.W. Ovambo | 1 | 1 |
| | All Bantu Speakers | 20 | 20 |
| NORTH AFRICA | | 30 | 30 |
| Algeria (Mzab) | Mozabite | 30 | 30 |
| MIDDLE EAST | | 148 | 148 |
| Israel (Negev) | Bedouin | 49 | 49 |
| Israel (Carmel) | Druze | 48 | 48 |
| Israel (Central) | Palestinian | 51 | 51 |
| ASIA | | 451 | 450 |
| Pakistan | Brahui | 25 | 24 |
| Pakistan | Balochi | 25 | 25 |
| Pakistan | Hazara | 25 | 25 |
| Pakistan | Makrani | 25 | 25 |
| Pakistan | Sindhi | 25 | 25 |
| Pakistan | Pathan | 25 | 25 |
| Pakistan | Kalash | 25 | 25 |
| Pakistan | Burusho | 25 | 25 |
| China | Han | 45 | 45 |
| China | Tujia (minority) | 10 | 10 |
| China | Yizu (Yi) (minority) | 10 | 10 |
| China | Miaozu (Miao) | 10 | 10 |
| | (minority) | | |
| China | Oroqen (minority) | 10 | 10 |
| China | Daur (minority) | 10 | 10 |
| China | Mongola (minority) | 10 | 10 |
| China | Hezhen (minority) | 10 | 10 |
| China | Xibo (minority) | 9 | 9 |
| China | Uygur (minority) | 10 | 10 |
| China | Dai (minority) | 10 | 10 |
| China | Lahu (minority) | 10 | 10 |
| China | She (minority) | 10 | 10 |
| China | Naxi (minority) | 10 | 10 |
| China | Tu (minority) | 10 | 10 |
| Siberia | Yakut | 25 | 25 |
| Japan | Japanese | 31 | 31 |
| Cambodia | Cambodian | 11 | 11 |
| OCEANIA | | 39 | 39 |
| New Guinea | Papuan | 17 | 17 |
| Bougainville | NAN Melanesian | 22 | 22 |
| EUROPE | | 161 | 161 |
| France | French (various | 29 | 29 |
| | regions) | | |
| France | Basque | 24 | 24 |
| Italy | Sardinian | 28 | 28 |
| Italy | from Bergamo | 14 | 14 |
| Italy | Tuscan | 8 | 8 |
| Orkney Islands | Orcadian | 16 | 16 |
| Russia Caucasus | Adygei | 17 | 17 |
| Russia | Russian | 25 | 25 |
| AMERICA | | 108 | 91 |
| Mexico | Pima (relative pairs) | 25 | 25 |
| Mexico | Maya (relative pairs) | 25 | 25 |
| Colombia | Piapoco and Curripaco | 13 | 12 |
| Brazil | Karitiana (relative | 24 | 16 |
| | pairs) | | |
| Brazil | Surui (relative pairs) | 21 | 13 |

*n, number of individuals

TABLE 13
CCL3L1 gene copies included in the CCL3L1/CCR5 GRGs
for purposes of determining their associated disease- and/or
transmission-influencing effects.

| | Detrimental | Protective |
|---|---|---|
| | CCL3L1 gene | CCL3L1 gene |
| Clinical setting | copies (CCL3L1^{low}) | copies (CCL3L1^{high}) |
| Risk of vertical | <2 copies | ≧2 copies |
| transmission | | |
| Risk of horizontal | <2 copies | ≧2 copies |
| transmission in non-AAs | | |
| (combined analysis for EAs | | |
| plus HAs) | | |
| Risk of horizontal | <4 copies | ≧4 copies |
| transmission in AAs | | |
| (note, median in HIV⁻ is 4) | | |
| Rate of disease progression | <2 copies | ≧2 copies |
| in EAs | | |
| Rate of disease progression | <3 copies | ≧3 copies |
| in AAs | | |
| (note, median in HIV⁺ is 3) | | |

TABLE 14
Bootstrapping analysis of the risk of progressing rapidly to AIDS.

| | | Bias-corrected bootstrap |
|---|---|---|
| | | estimates (n = 792; |
| | Entire cohort | repetitions = 1,000) |
| Genetic variable | RH (95% CI) | RH (95% CI) |
| CCR5^{det} | 2.04 (1.64-2.55) | 2.04 (1.56-2.63) |
| CCL3L1^{low} | 1.48 (1.22-1.80) | 1.48 (1.20-1.86) |

TABLE 15
Effect of rounding the estimates derived from Real-time PCR
(Taqman) assays used to determine CCL3L1 gene copy numbers
to the closest integers in the various populations studied.

| | | | Rounded | |
|---|---|---|---|---|
| | Raw estimate | | estimate | |
| Study Group (N) | Mean | SD | Mean | SD |
| HIV⁺ AAs (409) | 3.25 | 1.64 | 3.25 | 1.69 |
| HIV⁻ AAs (498) | 3.97 | 1.74 | 3.96 | 1.77 |
| HIV⁺ EAs (620) | 1.87 | 1.16 | 1.89 | 1.21 |
| HIV⁻ EAs (675) | 2.10 | 1.09 | 2.13 | 1.08 |
| HIV⁺ HAs (69) | 2.15 | 1.15 | 2.17 | 1.16 |
| HIV⁻ HAs (101) | 3.14 | 1.57 | 3.08 | 1.39 |
| HIV⁺ Argentinean children (407) | 1.84 | 1.13 | 1.85 | 1.14 |
| HIV⁻ Argentinean children (395) | 2.40 | 1.44 | 2.42 | 1.46 |
| HIV⁻ controls from WHMC (1,105) | 2.76 | 1.61 | 2.82 | 1.59 |
| Total (4,308) | 2.83 | 1.89 | 2.85 | 1.89 |

TABLE 16
Estimates of the initial frequency distribution and the
transition-rates used for Markov modeling of the CCL3L1 gene copy
copy numbers in the HIV-positive WHMC cohort.
Transition

| | AAs | | | | EAs | | | |
|---|---|---|---|---|---|---|---|---|
| | Initial | Transition rates, yr | | | Initial | Transition rates, yr | | |
| Copy # | frequency | 5 | 10 | 15 | frequency | 5 | 10 | 15 |
| 0 | 0.0123 | 0.1602 | 0.1346 | 0.0000 | 0.0568 | 0.2392 | 0.0900 | 0.0000 |
| 1 | 0.1259 | 0.1989 | 0.0547 | 0.0000 | 0.3653 | 0.1898 | 0.0720 | 0.0108 |
| 2 | 0.2198 | 0.2172 | 0.0469 | 0.0181 | 0.3766 | 0.1244 | 0.0470 | 0.0000 |
| 3 | 0.2296 | 0.1848 | 0.0565 | 0.0000 | 0.1153 | 0.1419 | 0.0685 | 0.0000 |
| 4 | 0.1901 | 0.1435 | 0.0433 | 0.0000 | 0.0487 | 0.1810 | 0.0439 | 0.0000 |
| 5 | 0.1284 | 0.1339 | 0.0543 | 0.0000 | 0.0244 | 0.2711 | 0.0490 | 0.0000 |
| 6 | 0.0568 | 0.1245 | 0.0670 | 0.0288 | 0.0081 | 0.2358 | 0.0817 | 0.0000 |
| ≧7 | 0.0370 | 0.0415 | 0.0758 | 0.0000 | 0.0049 | 0.0000 | 0.0392 | 0.0000 |

TABLE 17
CCR5 genotypes included in the CCL3L1/CCR5 GRGs for purposes of
determining their associated disease- and/or transmission-influencing effects (FIG. 4).*

| | | | Log-rank P |
|---|---|---|---|
| | Detrimental CCR5 | Nondetrimental CCR5 | for |
| | genotypes | genotypes | CCR5^{det} vs |
| Setting | (CCR5^{det}) | (CCR5^{non-det}) | CCR5^{non-det} |
| Rate of disease | HHE/HHE, HHC/HHG*1, | Implies all CCR5 genotypes | |
| progression in EAs | HHA/HHA, HHE/HHF*1, | that lack CCR5^{det} | |
| | HHF*2/HHG*1, | | |
| | HHG*1/HHG*2 | | |
| Median time to AIDS | 5.53 years | 7.94 years | 0.0024 |
| Median time to death | 6.59 years | 9.01 years | 0.0018 |
| Rate of disease | HHC/HHE, HHC/HHC, | Implies all CCR5 genotypes | |
| progression in AAs | HHE/HHG*2, HHC/HHD, | that lack CCR5^{det} | |
| | HHD/HHG*1, | | |
| | HHB/HHC, HHA/HHF*1, | | |
| | HHE/HHF*1 | | |
| Median time to AIDS | 5.21 years | 10.31 years | 1.5 × 10⁻⁸ |
| Median time to death | 6.34 years | 11.34 years | 2.6 × 10⁻⁷ |
| Rate of disease | HHE/HHE, HHD/HHE, | Implies all CCR5 genotypes | |
| progression in HAs | HHA/HHA, | that lack CCR5^{det} | |
| | HHF*2/HHF*2 | | |
| Median time to AIDS | 4.78 years | 8.42 years | 0.0107 |
| Median time to death | 6.53 years | 9.66 years | 0.0166 |
| Rate of disease | Combination of | Implies all population- | |
| progression in EAs + | population-specific | specific CCR5 genotypes | |
| AAs | CCR5^{det} genotypes | that lack CCR5^{det} | |
| Median time to AIDS | 5.35 years | 8.83 years | 3.8 × 10⁻⁹ |
| Median time to death | 6.40 years | 9.62 years | 3.2 × 10⁻⁸ |
| Rate of disease | Combination of | Implies all population- | |
| progression in EAs + | population-specific | specific CCR5 genotypes | |
| HAs + AAs | CCR5^{det} genotypes | that lack CCR5^{det} | |
| Median time to AIDS | 5.35 years | 8.78 years | 2.4 × 10⁻¹⁰ |
| Median time to death | 6.53 years | 9.62 years | 2.9 × 10⁻⁹ |

*In the pediatric cohort, possession of the CCR5 HHE haplotype is designated as CCR5^{det}. The median time to AIDS in HIV⁺ children possessing CCR5 HHE was 26 months (2.2 years) compared to 63 months (5.3 years) in children who lack this haplotype (P = 0.0002).

TABLE 18
Results of the TDT analysis in 198 trios*

| | | | |
|---|---|---|---|
| CCR5 | | | |
| genotype/haplotype | | | |
| and CCL3L1 gene | | | |
| dose | OR | 95% CI | P |

Model 1

| | | | |
|---|---|---|---|
| CCR5-Δ32 | 0.49 | 0.25-0.93 | 0.027 |

Model 2

| | | | |
|---|---|---|---|
| HHA | 0.57 | 0.33-0.97 | 0.036 |
| HHC | 0.67 | 0.47-0.97 | 0.031 |
| HHG*1 | 2.25 | 0.98-5.17 | 0.050 |
| HHG*2 | 0.49 | 0.25-0.93 | 0.027 |

Model 3

| | | | |
|---|---|---|---|
| CCR5-Δ32, <2 | 0.50 | 0.11-1.87 | 0.243 |
| CCL3L1 copies | | | |
| CCR5-Δ32, 2 | 0.21 | 0.04-0.77 | 0.008 |
| CCL3L1 copies | | | |
| CCR5-Δ32, >2 | 1.20 | 0.31-4.97 | 0.763 |
| CCL3L1 copies | | | |

Model 4

| | | | |
|---|---|---|---|
| <2 CCL3L1 copies | 0.44 | 0.17-1.07 | 0.050 |
| HHC | | | |
| 2 CCL3L1 copies | 0.21 | 0.04-0.77 | 0.008 |
| HHG*2 | | | |
| >2 CCL3L1 copies | 0.29 | 0.09-0.73 | 0.004 |
| HHA | | | |

*Models 1 and 3, TDT analyses for association between possession of the CCR5-Δ32 allele or haplotypes and KD susceptibility; Models 2 and 4, E-TDT analyses for association of CCR5-Δ32 allele or haplotypes in the context of different CCL3L1 gene dose strata. For models 3 and 4 only significant associations are shown.

TABLE 19
Results of the case/pseudocontrol analysis
(n = 162 matched sets, total n = 648)*

| | | | |
|---|---|---|---|
| CCR5 | | | |
| genotype/haplotype | | | |
| and CCL3L1 gene | | | |
| dose | OR | 95% CI | P |

Model 1

| | | | |
|---|---|---|---|
| CCR5-Δ32 | 0.69 | 0.34-1.38 | 0.295 |

Model 2

| | | | |
|---|---|---|---|
| CCR5-Δ32, <2 | 0.67 | 0.19-2.36 | 0.530 |
| CCL3L1 copies | | | |
| CCR5-Δ32, 2 | 0.27 | 0.07-0.99 | 0.049 |
| CCL3L1 copies | | | |
| CCR5-Δ32, >2 | 2.14 | 0.55-8.36 | 0.273 |
| CCL3L1 copies | | | |

Model 3

| | | | |
|---|---|---|---|
| HHA | 0.46 | 0.24-0.89 | 0.022 |
| HHG*1 | 3.31 | 1.22-8.97 | 0.019 |

Model 4

| <2 CCL3L1 copies | No significant | | |
|---|---|---|---|
| | CCR5 haplotype | | |
| 2 CCL3L1 copies | 0.27 | 0.07-0.99 | 0.049 |
| HHG*2 | | | |
| >2 CCL3L1 copies | 0.24 | 0.08-0.70 | 0.009 |
| HHA | | | |

*Models 1 and 2, conditional logistic regression analysis association between possession of the CCR5-Δ32 allele or haplotypes and KD susceptibility; Models 2 and 4, stepwise conditional logistic regression with an entry criterion of P = 0.1 for association of CCR5-Δ32 allele or haplotypes in the context of different CCL3L1 gene dose strata. For models 3 and 4 only significant associations are shown. All analyses were repeated after resolving the parent-of-origin of the alleles and identical results were observed.

### Tables for Example VI

TABLE 20
Summary of parameters used to model the influence of GRGs on vaccine-related
epidemiological endpoints.

| | | Range of Values |
|---|---|---|
| | | assumed (*) or |
| | | obtained from |
| Parameter | Description | data |
| βᵤ | Annual transmission probability based on the influence of | 0.0655-0.1620 |
| | the GRGs on VL set points, and is a measure of the degree | |
| | of infectiousness in each GRG. | |
| βₐ | Annual transmission probability that factors in the | 0.0655-0.3866 |
| | following: (i) βᵤ; (ii) the duration of infectiousness and for | |
| | this we used the adjusted RHs for progression to AIDS for | |
| | GRGs of the infected partner; and (iii) the risk of HIV | |
| | acquisition based on the GRGs of the susceptible partner. | |
| R₀ | Basic reproductive number estimated from βa and | 0.96-5.69 |
| | accounting for an assumed background death rate of 0.025 | |
| | and an estimated incidence 0.043 for AIDS. | |
| e | Vaccine efficacy is the product of the take and degree. We | 0.3-0.7* |
| | only accounted for the influence of the GRGs on the take | |
| | (t). | |
| t | Relative vaccine take across the GRGs. Estimated from the | 1, 0.94 and 0.86 |
| | mean best DTH responses across GRGs (data shown in FIG. | for low, moderate |
| | 1G) and normalizing them to the mean DTH response of | and high risk |
| | subjects belonging to the low risk group. | GRGs |
| f | Fraction of subjects in whom the vaccine efficacy does not | 0.9, 0.82 and 0.74 |
| | wane i.e. vaccine durability. Estimated by assuming that | for low, moderate |
| | the duration of vaccine protection in the low risk GRG is | and high risk |
| | 10 years and by estimating the odds of anergy (failure to | GRGs. |
| | respond to vaccine) in the other GRGs. The values of f in | |
| | the adjacent column to the right indicates that the duration | |
| | of vaccine protection is 10, 5.6 and 3.9 years in the low, | |
| | moderate and high risk GRGs, respectively. | |
| Pc | Critical proportion of the population- or cohort-based | 0-2.59 |
| | vaccination coverage required to limit the epidemic. | |

TABLE 21
Characteristics of the study subjects in the WHMC cohort

| | |
|---|---|
| Number of subjects recruited | 1,132 |
| Number of seroconverters recruited (n, %) | 515 (45.5%) |
| Ethnicity (n, %) | |
| European Americans (EA) | 621 (54.9%) |
| African Americans (AA) | 410 (36.3%) |
| Hispanic Americans (HA) | 69 (6.1%) |
| Others (OT) | 32 (2.7%) |
| Maximum length of follow-up (yrs) | 15.47 |
| Total follow-up (person years) | 7123.5 |
| Age at cohort inception (mean ± SD yrs) | 29.9 ± 7.2 |
| Proportion of males (n, %) | 1060 (93.8%) |
| Baseline CD4+ T cell count (mean ± SE cells/μl) | 605.20 ± 8.96 |
| Viral set point (median copies/ml, IQR) | 18000, 44600 |
| Number of AIDS events over follow-up (%) | 450 (39.8%) |
| Number of AIDS-related deaths over follow-up (%) | 440 (38.9%) |
| Distribution of GRGs (n, %) | |
| Low risk GRG | 555 (50.3%) |
| Moderate risk GRG | 462 (41.9%) |
| High risk GRG | 86 (7.87%) |

TABLE 21
Number (percentage) of subjects used in the analyses. Unless indicated otherwise
these numbers are for the HIV+ adult subjects from the WHMC cohort.

| Figure # | N (%) |
|---|---|
| 1C, 1D | 550 (50.1), 461 (42.0), 86 (7.9) in low, moderate and high risk GRG, |
| | respectively. |
| 1E | 523 (50.1), 440 (42.2), 80 (7.7) in low, moderate and high risk GRG, |
| | respectively, and excluding 54 subjects who were initially anergic. |
| 1F | 401 (seroconverters overall); 211 (53.8), 152 (38.8), 29 (7.4) in low, moderate |
| | and high risk GRG, respectively. |
| 2A | Correlation between bCD4 and VL-sp: 403 (35.6) |
| | Correlation between bCD4 and cCD4: 1110 (98.1) |
| | Correlation between cCD4 and VL-sp: 400 (35.3) |
| 2B | 1094 (96.6) |
| 2C | 549 (50.7), 449 (41.5), 84 (7.8) in low, moderate and high risk GRG, |
| | respectively. |
| 2D, 2E, 2F | 391 (34.5) overall; 212 (54.2), 150 (38.4), 29 (7.4) in low, moderate and high |
| | risk GRG, respectively. |
| 2G | Low risk group 124 (2,570 measurements); Moderate risk group 80 (1,456 |
| | measurements); High risk group 8 (150 measurements). |
| 2H | Low risk group 50 (935 measurements); Moderate risk group 34 (665 |
| | measurements); High risk group 9 (84 measurements). |
| 21 | Low risk group 39 (691 measurements); Moderate risk group 41 (709 |
| | measurements); High risk group 12 (101 measurements). |
| 3A | 150 (803 measurements) who developed AIDS; 486 (4,560 measurements) |
| | who did not develop AIDS; 357 (3,131 measurements) in low risk GRG; 235 |
| | (1,993 measurements) in moderate risk GRG and 36 (159 measurements) in |
| | high risk GRG. |
| 3B | 629 (55.6), 311 (49.4%) achieved viral suppression overall. The proportion of |
| | subjects attaining viral suppression within the GRGs was: 51.7% low risk |
| | GRG, 47.4% in moderate risk GRG and 30.6% in high risk GRG. |
| 3C | 170(61.6), 98(35.5), 8(2.9) in low, moderate and high risk GRG. |
| 3D, 3E | 126(51.0), 97(39.3), 24(9.7) in low, moderate and high risk GRG. |
| 3F | Unadjusted analysis: |
| | 323 (6,444 measurements), 205 (3,992 measurements), 24 (322 measurements) |
| | in low, moderate and high risk GRG, respectively. |
| | Adjusted analysis: |
| | 171 (3,649 measurements), 113 (2,437 measurements), 17 (245 measurements) |
| | in low, moderate and high risk GRG, respectively. |
| 3G | Unadjusted analysis: |
| | 55 (654 measurements), 29 (325 measurements), 2 (30 measurements) in low, |
| | moderate and high risk GRG, respectively. |
| | Adjusted analysis: |
| | 10 (153 measurements), 5 (82 measurements), 1 (17 measurements) in low, |
| | moderate and high risk GRG, respectively. |
| 4A | Model 1: 502 (44.3); Model 2: 501 (44.2); Model 3: 394 (34.8); Model 4: 502 |
| | (44.3); Model 5: 501 (44.2); Model 6: 394 (34.8); Model 7: 394 (34.8); Model |
| | 8: 392 (34.6); Model 9: 385 (34.0); Model 10: 385 (34.0). |
| 4B | Upper panel, 141 (39.5), 167 (46.8), 49 (13.7) in low, moderate and high risk |
| | GRG, respectively; Lower panel, 39 (42.8), 40(44.0), 12 (13.2) in low, |
| | moderate and high risk GRG, respectively. |
| 4C to 4I | 394 (34.8) |
| 6A | 321 Argentinean children, 3,967 VL measurements |
| 6B | 8 (89 measurements), 19 (241 measurements) and 15 (218 measurements) in |
| | subjects with <2, 2 or >2 copies of the CCL3L1 gene, respectively (AIEDRP |
| | cohort) |
| 6C | 26 (591 measurements), 74 (1698 measurements) and 33 (747 measurements) |
| | in subjects with <2, 2 or >2 copies of the CCL3L1 gene, respectively. |
| | (AIEDRP cohort) |
| 6D | 85 (2016 measurements) and 47 (980 measurements) in subjects with and |
| | without HHC allele, respectively. (AIEDRP cohort) |
| 6E | 8 (195 measurements) and 16 (359 measurements) in subjects with |
| | HHE/HHG*2 and Non-HHE/HHG*2 genotypes, respectively. (AIEDRP |
| | cohort) |
| 6F and 6G | 123 (69.1), (AIEDRP cohort) |
| 6H | 33 (26.8) and 90 (73.2) who failed and did not fail to respond to HAART, |
| | respectively. (AIEDRP cohort) |

TABLE 21
Characteristics of the adult EA subjects from the
AIEDRP cohort (N = 178).

| Characteristic | N | % |
|---|---|---|
| Age | | |
| <30 years | 26 | 14.6 |
| 30-39 years | 73 | 41.0 |
| 40-49 years | 58 | 32.6 |
| 50-59 years | 16 | 9.0 |
| ≧60 years | 5 | 2.8 |
| Gender | | |
| Males | 170 | 95.5 |
| Females | 8 | 4.5 |
| AIED class | | |
| A1 | 22 | 12.4 |
| A2 | 8 | 4.5 |
| E1 | 41 | 23.0 |
| E2 | 97 | 54.5 |
| E3 | 10 | 5.6 |
| Treatment received | | |
| Yes | 135 | 75.8 |
| No | 43 | 24.2 |
| Time to initiation of treatment since | | |
| estimated date of infection | | |
| Median (days) | 106.5 | |
| IQR (days) | 144 | |
| Time to initiation of treatment since | | |
| recruitment | | |
| Median (days) | 30 | |
| IQR (days) | 134 | |
| Subjects in whom therapy was initiated | 64 | 52.0 |
| within one month of recruitment | | |
| Subjects who received 1/2 NRTIs | 13 | 10.57 |
| Subjects who received HAART | 110 | 89.43 |
| First CD4+ T cell count | | |
| Median (cells/μl) | 516.5 | |
| IQR (cells/μl) | 246 | |
| Time to first CD4+ T cell measurement | | |
| since estimated date of infection | | |
| Median (days) | 85 | |
| IQR (days) | 40 | |
| First Log₁₀ HIV_RNA | | |
| Mean (copies/ml) | 5.00 | |
| SD (copies/ml) | 1.12 | |
| Time to first HIV RNA measurement | | |
| since estimated date of infection | | |
| Median (days) | 83 | |
| IQR (days) | 40 | |
| CCL3L1 Gene copy numbers | | |
| 1 | 35 | 19.7 |
| 2 | 94 | 52.8 |
| 3 | 34 | 19.1 |
| 4 | 8 | 4.5 |
| 5 | 7 | 3.9 |
| CCR5 allele frequencies | | |
| HHA | 0.0565 | |
| HHB | 0.0000 | |
| HHC | 0.3644 | |
| HHD | 0.0000 | |
| HHE | 0.3419 | |
| HHF*1 | 0.0169 | |
| HHF*2 | 0.0763 | |
| HHG*1 | 0.0367 | |
| HHG*2 | 0.1073 | |

TABLE 22
Multivariate Cox proportional hazards models using DTH, bCD4 and VL-sp as
predictors. (A) Coding schema used for variables included in multivariate Cox proportional
hazards models shown in FIG. 1F. As a result of this coding schema the RH values shown in FIG. 1F
indicate the increased or decreased relative hazard for a unit change in the code. For example, the
RH of 0.54 for DTH indicates that if the DTH response of 2 positive tests had a 46% reduced rate
of progression as compared to the subjects with 0/1 positive tests. Also, subjects with 3/4 positive
skin tests have a further reduced rate of disease progression by a factor 46% as compared to
subjects with 2 positive skin tests. Similar interpretations apply to bCD4 and VL-sp categories. (B)
Results of multivariate Cox proportional hazards models with DTH, bCD4 and VL-sp coded as
shown in A. This Table shows the results shown in FIG. 1F along with the 95% confidence intervals.
A

| Variable | Code |
|---|---|
| DTH | |
| 0/1 positive skin tests | 0 |
| 2 positive skin tests | 1 |
| 3/4 positive skin tests | 2 |
| bCD4 | |
| 700 cells/μl | 0 |
| 350-699 cells/μl | 1 |
| <350 cells/μl | 2 |
| VL - 5p | |
| <20,000 copies/ml | 0 |
| 20,000-55,000 copies/ml | 1 |
| >55,000 copies/ml | 2 |

B

| | Unaccounted | | | Low risk | | | Moderate | | | High risk | | |
|---|---|---|---|---|---|---|---|---|---|---|---|---|
| | for GRGs | | | GRG - | | | risk GRG - | | | GRG - | | |
| | RH | 95% CI | P | RH | 95% CI | P | RH | 95% CI | P | RH | 95% CI | P |
| DTH | 0.54 | 0.43-0.69 | 7.4 × 10⁻⁷ | 0.36 | 0.24-0.54 | 1.4 × 10⁻⁶ | 0.64 | 0.45-0.92 | 0.015 | 1.32 | 0.60-2.91 | 0.488 |
| CD4 | 1.39 | 1.00-1.92 | 0.050 | 1.33 | 0.77-2.28 | 0.129 | 1.20 | 0.72-1.98 | 0.487 | 3.34 | 1.13-9.81 | 0.029 |
| VL | 1.81 | 1.45-2.26 | 1.6 × 10⁻⁷ | 1.42 | 0.98-2.07 | 0.071 | 1.84 | 1.31-2.60 | 5.0 × 10⁻⁷ | 3.83 | 1.58-9.33 | 0.003 |

TABLE 23
Correlation matrix showing Spearman correlation coefficients
and their statistical significance between pairs of markers
of disease progression in HIV infection.

| | bCD4 | nCD4 | rCD4 | cCD4 | VL-sp |
|---|---|---|---|---|---|
| nCD4 | 0.4607 | | | | |
| | (<0.0001) | | | | |
| rCD4 | −0.1095 | 0.4361 | | | |
| | (<0.0001) | (<0.0001) | | | |
| cCD4 | 0.5425 | 0.3433 | 0.2242 | | |
| | (<0.0001) | (<0.0001) | (<0.0001) | | |
| VL-sp | −0.2439 | −0.2910 | −0.1139 | −0.2927 | |
| | (<0.0001) | (<0.0001) | (0.0234) | (<0.0001) | |
| nVL | −0.1502 | −0.2594 | −0.3750 | −0.3622 | 0.7640 |
| | (0.0067) | (<0.0001) | (<0.0001) | (<0.0001) | (<0.0001) |

TABLE 24
GRGs predict the development of AIDS independent of
several explanatory covariates in seroconverters. (A) Results
of multivariate Cox proportional hazards models for time to
development of AIDS. RH, relative hazard. (B)The outcome
variable for the analysis here was whether a person developed
or did not develop AIDS over follow-up. OR, odds ratio.
A Rate of developing AIDS

| # | Covariates | —RH (95% CI) | —RH (95% CI) | ═RH (95% CI) |
|---|---|---|---|---|
| 1 | None | 1.57 (1.06-2.32) | 4.87 (2.82-8.39) | 1.90 (1.32-2.74) |
| 2 | C | 1.44 (0.97-2.13) | 4.72 (2.74-8.13) | 1.76 (1.22-2.55) |
| 3 | V | 1.54 (1.02-2.33) | 4.77 (2.67-8.52) | 1.81 (1.22-2.67) |
| 4 | T | 1.44 (0.97-2.13) | 3.26 (1.87-5.69) | 1.67 (1.15-2.41) |
| 5 | N | 1.21 (0.82-1.80) | 3.11 (1.80-5.39) | 1.43 (0.99-2.07) |
| 6 | C, V | 1.43 (0.94-2.17) | 4.54 (2.54-8.11) | 1.70 (1.15-2.52) |
| 7 | C, V, T | 1.39 (0.92-2.11) | 3.19 (1.77-5.75) | 1.59 (1.08-2.35) |
| 8 | C, V, T, D | 1.42 (0.94-2.15) | 3.34 (1.85-6.02) | 1.65 (1.11-2.44) |
| 9 | C, V, T, | 1.40 (0.91-2.15) | 2.90 (1.59-5.29) | 1.61 (1.08-2.41) |
| | D, P | | | |
| 10 | C, V, T, | 1.34 (0.89-2.04) | 2.77 (1.53-5.03) | 1.54 (1.04-2.27) |
| | D, P, N | | | |

B Risk of developing AIDS

| # | Covariates | —OR (95% CI) | —OR (95% CI) | ═OR (95% CI) |
|---|---|---|---|---|
| 1 | None | 1.59 (1.23-2.05) | 4.13 (2.55-6.68) | 1.84 (1.44-2.35) |
| 2 | C | 1.54 (1.18-2.00) | 3.65 (2.23-5.98) | 1.76 (1.37-2.27) |
| 3 | V | 1.69 (1.03-2.76) | 4.21 (1.79-9.87) | 1.96 (1.23-3.13) |
| 4 | T | 1.28 (0.97-1.70) | 2.91 (1.72-4.94) | 1.45 (1.11-1.91) |
| 5 | N | 1.53 (1.13-2.07) | 4.88 (2.64-9.02) | 1.80 (1.35-2.41) |
| 6 | C, V | 1.64 (1.00-2.70) | 4.13 (1.76-9.69) | 1.92 (1.20-3.07) |
| 7 | C, V, T | 1.55 (0.92-2.60) | 3.35 (1.34-8.38) | 1.75 (1.07-2.87) |
| 8 | C, V, T, D | 1.56 (0.91-2.66) | 3.19 (1.25-8.13) | 1.75 (1.06-2.91) |
| 9 | C, V, T, | 1.66 (0.97-2.86) | 3.26 (1.27-8.36) | 1.85 (1.10-3.11) |
| | D, P | | | |
| 10 | C, V, T, | 1.33 (0.72-2.46) | 2.57 (0.89-7.43) | 1.48 (0.82-2.68) |
| | D, P, N | | | |

TABLE 25
Results of principal components factor analysis.

Factor

| | | | |
|---|---|---|---|
| Variable | 1 | 2 | Uniqueness |

Before Rotation

| | | | |
|---|---|---|---|
| bCD4 | −0.4800 | 0.5913 | 0.4199 |
| nCD4 | −0.3967 | 0.3558 | 0.7160 |
| cCD4 | −0.4906 | 0.4562 | 0.5511 |
| VL-sp | 0.8525 | 0.3868 | 0.1237 |
| nVL | 0.8434 | 0.4103 | 0.1205 |
| GRG | 0.3032 | −0.0888 | 0.9002 |

After Rotation

| | | | |
|---|---|---|---|
| bCD4 | −0.7542 | −0.1058 | 0.4199 |
| nCD4 | −0.5095 | −0.1561 | 0.7160 |
| cCD4 | −0.6440 | −0.1846 | 0.5511 |
| VL-sp | 0.1088 | 0.9298 | 0.1237 |
| nVL | 0.0841 | 0.9341 | 0.1205 |
| GRG | 0.2325 | 0.2138 | 0.9002 |

The results show the factor loadings before and after varimax rotation. Two factors were retained based on the criterion of minimum eigen value of 1.
bCD4, baseline CD4+ T cell count;
nCD4, nadir CD4+ T cell count;
cCD4, cumulative CD4+ T cell count;
VL-sp, viral load set point;
nVL, nadir viral load;
GRG, genetic risk groups.

TABLE 26
Results of mathematical modeling of the
influence of GRGs on vaccine related endpoints.

| | GRG in partners | | Estimated | | | | | | |
|---|---|---|---|---|---|---|---|---|---|
| Group | HIV+ | HIV− | Frequency | βᵤ | βₐ | Rₒ | t | f | P_{c} |
| 1 | Low | Low | 0.2310 | 0.0655 | 0.0655 | 0.96 | 1.00 | 0.90 | 0.00 |
| 2 | Low | Moderate | 0.1638 | 0.0829 | 0.0829 | 1.22 | 0.94 | 0.82 | 0.47 |
| 3 | Low | High | 0.0252 | 0.0946 | 0.0946 | 1.39 | 0.86 | 0.74 | 0.88 |
| 4 | Moderate | Low | 0.2640 | 0.0941 | 0.1340 | 1.97 | 1.00 | 0.90 | 1.09 |
| 5 | Moderate | Moderate | 0.1872 | 0.1191 | 0.1677 | 2.47 | 0.94 | 0.82 | 1.54 |
| 6 | Moderate | High | 0.0288 | 0.1359 | 0.1898 | 2.79 | 0.86 | 0.74 | 2.02 |
| 7 | High | Low | 0.0550 | 0.1121 | 0.2916 | 4.29 | 1.00 | 0.90 | 1.70 |
| 8 | High | Moderate | 0.0390 | 0.1420 | 0.3505 | 5.15 | 0.94 | 0.82 | 2.09 |
| 9 | High | High | 0.0060 | 0.1620 | 0.3866 | 5.69 | 0.86 | 0.74 | 2.59 |

Group, indicates subdivision of the population into 9 groups based on their GRGs. Estimated frequency indicates the proportion of the population groups from data derived from the WHMC cohort. The parameters are described in Table 20.

1. A method of identifying a subject at increased risk of developing a disorder associated with a detrimental CCL3L1/CCR5 genotype, comprising detecting in a subject the presence of a CCL3L1/CCR5 genotype associated with increased risk of developing a disorder associated with a detrimental CCL3L1/CCR5 genotype.

2. The method of claim 1, wherein the disorder is selected from the group consisting of human immunodeficiency virus (HIV) infection, acquired immune deficiency syndrome (AIDS), autoimmune diseases including but not limited to systemic lupus erythematosis (SLE), rheumatoid arthritis, Kawasaki disease (KD), infectious disorders such as tuberculosis, cardiovascular disorders such as atherosclerosis and coronary artery disease.

3. A method of identifying a subject at increased risk of infection with HIV, comprising: detecting in the subject the presence of a CCL3L1/CCR5 genotype correlated with increased risk of infection with HIV.

4. A method of identifying an HIV-infected subject at increased risk of developing acquired immune deficiency syndrome (AIDS), comprising detecting in the subject the presence of a CCL3L1/CCR5 genotype correlated with increased risk of developing AIDS.

5. A method of identifying an HIV-infected subject at increased risk of developing a disorder associated with AIDS, comprising detecting in the subject the presence of a CCL3L1/CCR5 genotype correlated with increased risk of developing a disorder associated with AIDS, such as Pneumocystis carinii pneumonia, Mycobacterium infection, cytomegalovirus infection.

6. A method of identifying an HIV-infected subject having an increased likelihood of a poor prognosis and/or reduced life expectancy, comprising detecting in the subject the presence of a CCL3L1/CCR5 genotype correlated with increased likelihood of a poor prognosis and/or reduced life expectancy.

7-52. (canceled)
